SEQUENCE LISTING (370 sequences)

SEQ ID NO: 1 - protein, 8 aa, Artificial Sequence
  CDR1
RSSFSNNA

SEQ ID NO: 2 - protein, 7 aa, Artificial Sequence
  CDR2
ITKGGVT

SEQ ID NO: 3 - protein, 8 aa, Artificial Sequence
  CDR3
NTIPFRSA

SEQ ID NO: 4 - protein, 8 aa, Artificial Sequence
  CDR1
GFTFSNYA

SEQ ID NO: 5 - protein, 8 aa, Artificial Sequence
  CDR2
INSDGGTA

SEQ ID NO: 6 - protein, 17 aa, Artificial Sequence
  CDR3
AANRGFCAGVRCLEYQY

SEQ ID NO: 7 - protein, 8 aa, Artificial Sequence
  CDR1
GRTFSINA

SEQ ID NO: 8 - protein, 10 aa, Artificial Sequence
  CDR2
IGGSGRVSST

SEQ ID NO: 9 - protein, 16 aa, Artificial Sequence
  CDR3
AARRDYLPFPPESYDY

SEQ ID NO: 10 - protein, 15 aa, Artificial Sequence
  CDR3
AAGRRTSTNGGDYDY

SEQ ID NO: 11 - protein, 8 aa, Artificial Sequence
  CDR2
ISRSGGAT

SEQ ID NO: 12 - protein, 16 aa, Artificial Sequence
  CDR3
AGTRRAFSTGLRDYDY

SEQ ID NO: 13 - protein, 8 aa, Artificial Sequence
  CDR1
GRTFSNST

SEQ ID NO: 14 - protein, 8 aa, Artificial Sequence
  CDR2
ISWSGGTY

SEQ ID NO: 15 - protein, 16 aa, Artificial Sequence
  CDR3
AAARKGWSTRGDDYDY

SEQ ID NO: 16 - protein, 8 aa, Artificial Sequence
  CDR1
GRTYNNYA

SEQ ID NO: 17 - protein, 8 aa, Artificial Sequence
  CDR2
ISRSGGMT

SEQ ID NO: 18 - protein, 16 aa, Artificial Sequence
  CDR3
AAYVGGFSTARRDYSY

SEQ ID NO: 19 - protein, 8 aa, Artificial Sequence
  CDR1
GRTFSSYA

SEQ ID NO: 20 - protein, 8 aa, Artificial Sequence
  CDR2
ISRIGGVT

SEQ ID NO: 21 - protein, 17 aa, Artificial Sequence
  CDR3
AAAGLVSISTTPNDYDY

SEQ ID NO: 22 - protein, 8 aa, Artificial Sequence
  CDR1
GNIFRLNG

SEQ ID NO: 23 - protein, 7 aa, Artificial Sequence
  CDR2
ITSGGNT

SEQ ID NO: 24 - protein, 7 aa, Artificial Sequence
  CDR3
NAIPFRL

SEQ ID NO: 25 - protein, 8 aa, Artificial Sequence
  CDR1
RNIFSLNP

SEQ ID NO: 26 - protein, 7 aa, Artificial Sequence
  CDR2
ITDGGST

SEQ ID NO: 27 - protein, 10 aa, Artificial Sequence
  CDR3
NRVGGLQTWA

SEQ ID NO: 28 - protein, 8 aa, Artificial Sequence
  CDR1
GRSFSNYG

SEQ ID NO: 29 - protein, 8 aa, Artificial Sequence
  CDR2
IGMVGGLT

SEQ ID NO: 30 - protein, 15 aa, Artificial Sequence
  CDR3
AAGRRFSTSSRDYDI

SEQ ID NO: 31 - protein, 8 aa, Artificial Sequence
  CDR1
GRTFDSRP

SEQ ID NO: 32 - protein, 8 aa, Artificial Sequence
  CDR2
VSWRGEST

SEQ ID NO: 33 - protein, 19 aa, Artificial Sequence
  CDR3
AAGEPYSGTYYYRGRDYDY

SEQ ID NO: 34 - protein, 8 aa, Artificial Sequence
  CDR1
GRTFSMYA

SEQ ID NO: 35 - protein, 8 aa, Artificial Sequence
  CDR2
ISGSARIT

SEQ ID NO: 36 - protein, 16 aa, Artificial Sequence
  CDR3
AASSTYTSTSGSSYNY

SEQ ID NO: 37 - protein, 8 aa, Artificial Sequence
  CDR1
GSIFSINA

SEQ ID NO: 38 - protein, 7 aa, Artificial Sequence
  CDR2
ITSGGST

SEQ ID NO: 39 - protein, 11 aa, Artificial Sequence
  CDR3
NALGGFVPSYG

SEQ ID NO: 40 - protein, 113 aa, Artificial Sequence
  FcRH5 VHH domain
QVQLQESGGGLVQAGGSLRLSCAASRSSFSNNAMGWYRQVPGKQRELVAFITKGGVTDYSDSVKGRFTISKDNAKNTVYLQMNSLKPEDTAVYYCNTIPFRSAWGQGTQVTVS

SEQ ID NO: 41 - protein, 123 aa, Artificial Sequence
  FcRH5-specific sdAb (VHH)
QVQLQESGGGSVQPGGSLSLSCAASGFTFSNYAMSWVRQAPGKGPEWVAVINSDGGTASSAGSVRGRFTISRDNAKNTLYLQMNRLKPEDTAVYYCAANRGFCAGVRCLEYQYWGQGTQVTVS

SEQ ID NO: 42 - protein, 124 aa, Artificial Sequence
  FcRH5-specific sdAb (VHH)
QVQLQESGGGLVQAGGSLIVSCAASGRTFSINAMAWFRQAPGKEREFVAAIGGSGRVSSTSYADFVKGRFTISRDNAKNTVYLRMNNLEPEDTAVYYCAARRDYLPFPPESYDYWGQGTQVTVA

SEQ ID NO: 43 - protein, 121 aa, Artificial Sequence
  FcRH5-specific sdAb (VHH)
QVQLQESGGGLVQAGGSLRLSCAASGRTFSTYAMAWFRQAPGKEREFVAAISGFGVVTYYADSVKGRFTISRDNAKNTLYLQMNGLKPEDTAVYYCAAGRRTSTNGGDYDYWGQGTQVTVS

SEQ ID NO: 44 - protein, 122 aa, Artificial Sequence
  FcRH5-specific sdAb (VHH)
QVQLQESGGGLVQAGGSLRLSCAASGRTFSTYGMGWFRQAPGKEREFVAAISRSGGATAYAASVKGRFTISRDDVKNTLYLQMNSLKPEDTAVYHCAGTRRAFSTGLRDYDYWGQGIQVTVS

SEQ ID NO: 45 - protein, 122 aa, Artificial Sequence
  FcRH5-specific sdAb (VHH)
QVQLQQSGGGLVQAGDSLRLSCAASGRTFSNSTMGWFRQAPGKERKFVAVISWSGGTYAYAESVKGRFTISRDNAKNTVYLQMNSLKPEDTAVYYCAAARKGWSTRGDDYDYWGQGTQVTVS

SEQ ID NO: 46 - protein, 122 aa, Artificial Sequence
  FcRH5-specific sdAb (VHH)
QVQLQESGGGLVQAGGSLRLSCAASGRTYNNYAMGWFRQAPGKEREFVAGISRSGGMTGYAESVKGRFTISRDNAKNMVFLQMNSLKPEDTAVYYCAAYVGGFSTARRDYSYWGQGTQVTVS

SEQ ID NO: 47 - protein, 123 aa, Artificial Sequence
  FcRH5-specific sdAb (VHH)
QVQLQESGGGLVQAGDSLRLSCAYSGRTFSSYAMGWFRQAPGKERVFVAAISRIGGVTTYAESVQGRFTISRDNAKNTLYLQMNALKPEDTAVYYCAAAGLVSISTTPNDYDYWGQGTQVTVS

SEQ ID NO: 48 - protein, 113 aa, Artificial Sequence
  FcRH5-specific sdAb (VHH)
QVQLQQSGGGLVQPGGSLRLSCAAPGNIFRLNGTGWYRQAPGKQRELVAHITSGGNTDYADSVKGRFTISRDNAKNTVYLQMNSLKPEDTAVYYCNAIPFRLSWGQGTQVTVS

SEQ ID NO: 49 - protein, 116 aa, Artificial Sequence
  FcRH5-specific sdAb (VHH)
QVQLQESGGGLVQAGGSLRLSCVVSRNIFSLNPMGWYRQAPGKQREMVAIITDGGSTNYADSVKGRFTISRDNVKNTVYLQMNALEPEDTAVYYCNRVGGLQTWAWGQGTQVTVSS

SEQ ID NO: 50 - protein, 121 aa, Artificial Sequence
  FcRH5-specific sdAb (VHH)
QVQLQQSGGGLVQAGGSLSLSCTASGRSFSNYGMGWFRQAPGKEREFVAAIGMVGGLTAYSNSAKGRFTISRDNAKNTLYLQMNSLKPEDTAVYLCAAGRRFSTSSRDYDIWGQGTQVTVS

SEQ ID NO: 51 - protein, 125 aa, Artificial Sequence
  FcRH5-specific sdAb (VHH)
QVQLQESGGGLVQAGDSLRLSCAASGRTFDSRPMGWFRQAPGKEREFVGAVSWRGESTYYPDSVKGRFTISRDNAKRTVYLQMNSLKPEDTAVYYCAAGEPYSGTYYYRGRDYDYWGQGTQVTVS

SEQ ID NO: 52 - protein, 122 aa, Artificial Sequence
  FcRH5-specific sdAb (VHH)
QVQLQESGGGLVQAEGSLRLSCAASGRTFSMYAMGWFRQAPGREREFVAAISGSARITYYGQSVKGRFTISRDNAKNTVYLQMNSLKPEDTAVYYCAASSTYTSTSGSSYNYWGQGTQVTVS

SEQ ID NO: 53 - protein, 116 aa, Artificial Sequence
  FcRH5-specific sdAb (VHH)
QVQLQESGGGLVQAGGSLRLSCAASGSIFSINAMGWYRQAPGKQRELVAFITSGGSTNYADSVKGRFTISRDNAKNTVYLQMNSLKPEDTAVYYCNALGGFVPSYGWGQGTQVTVS

SEQ ID NO: 54 - protein, 21 aa, Artificial Sequence
  CD8a transmembrane domain
IYIWAPLAGTCGVLLLSLVIT

SEQ ID NO: 55 - protein, 27 aa, Artificial Sequence
  CD28 transmembrane domain
FWVLVVVGGVLACYSLLVTVAFIIFWV

SEQ ID NO: 56 - protein, 24 aa, Artificial Sequence
  Tyrp-1 transmembrane domain
IIAIAVVGALLLVALIFGTASYLI

SEQ ID NO: 57 - protein, 234 aa, Artificial Sequence
  hinge-CH2CH3 of human IgG1
AEPKSPDKTHTCPPCPAPPVAGPSVFLFPPKPKDTLMIARTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKKD

SEQ ID NO: 58 - protein, 45 aa, Artificial Sequence
  human CD8 stalk
TTTPAPRPPTPAPTIASQPLSLRPEACRPAAGGAVHTRGLDFACD

SEQ ID NO: 59 - protein, 20 aa, Artificial Sequence
  human IgG1 hinge
AEPKSPDKTHTCPPCPKDPK

SEQ ID NO: 60 - protein, 237 aa, Artificial Sequence
  IgG1 Hinge-Fc
AEPKSPDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKKDPK

SEQ ID NO: 61 - protein, 236 aa, Artificial Sequence
  IgG1 Hinge - Fc modified to remove Fc receptor recognition motifs
AEPKSPDKTHTCPPCPAPPVAGPSVFLFPPKPKDTLMIARTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKKDPK

SEQ ID NO: 62 - protein, 185 aa, Artificial Sequence
  CD2 ectodomain
KEITNALETWGALGQDINLDIPSFQMSDDIDDIKWEKTSDKKKIAQFRKEKETFKEKDTYKLFKNGTLKIKHLKTDDQDIYKVSIYDTKGKNVLEKIFDLKIQERVSKPKISWTCINTTLTCEVMNGTDPELNLYQDGKHLKLSQRVITHKWTTSLSAKFKCTAGNKVSKESSVEPVSCPEKGLD

SEQ ID NO: 63 - protein, 259 aa, Artificial Sequence
  CD34 ectodomain
SLDNNGTATPELPTQGTFSNVSTNVSYQETTTPSTLGSTSLHPVSQHGNEATTNITETTVKFTSTSVITSVYGNTNSSVQSQTSVISTVFTTPANVSTPETTLKPSLSPGNVSDLSTTSTSLATSPTKPYTSSSPILSDIKAEIKCSGIREVKLTQGICLEQNKTSSCAEFKKDRGEGLARVLCGEEQADADAGAQVCSLLLAQSEVRPQCLLLVLANRTEISSKLQLMKKHQSDLKKLGILDFTEQDVASHQSYSQKT

SEQ ID NO: 64 - protein, 45 aa, Artificial Sequence
  COMP
DLGPQMLRELQETNAALQDVRELLRQQVREITFLKNTVMECDACG

SEQ ID NO: 65 - protein, 5 aa, Artificial Sequence
  truncated COMP
CDACG

SEQ ID NO: 66 - protein, 20 aa, Artificial Sequence
  truncated COMP
QQVREITFLKNTVMECDACG

SEQ ID NO: 67 - protein, 39 aa, Artificial Sequence
  CD28 endodomain
RSKRSRLLHSDYMNMTPRRPGPTRKHYQPYAPPRDFAAY

SEQ ID NO: 68 - protein, 37 aa, Artificial Sequence
  OX40 endodomain
RRDQRLPPDAHKPPGGGSFRTPIQEEQADAHSTLAKI

SEQ ID NO: 69 - protein, 42 aa, Artificial Sequence
  41-BB endodomain
KRGRKKLLYIFKQPFMRPVQTTQEEDGCSCRFPEEEEGGCEL

SEQ ID NO: 70 - protein, 114 aa, Artificial Sequence
  CD3 zeta endodomain
RSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 71 - protein, 153 aa, Artificial Sequence
  CD28Z
RSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 72 - protein, 189 aa, Artificial Sequence
  CD28OXZ
RSKRSRLLHSDYMNMTPRRPGPTRKHYQPYAPPRDFAAYRSRDQRLPPDAHKPPGGGSFRTPIQEEQADAHSTLAKIRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 73 - protein, 20 aa, Artificial Sequence
  signal peptide
METDTLLLWVLLLWVPGSTG

SEQ ID NO: 74 - protein, 402 aa, Artificial Sequence
  iCasp9
MLEGVQVETISPGDGRTFPKRGQTCVVHYTGMLEDGKKVDSSRDRNKPFKFMLGKQEVIRGWEEGVAQMSVGQRAKLTISPDYAYGATGHPGIIPPHATLVFDVELLKLESGGGSGVDGFGDVGALESLRGNADLAYILSMEPCGHCLIINNVNFCRESGLRTRTGSNIDCEKLRRRFSSLHFMVEVKGDLTAKKMVLALLELAQQDHGALDCCVVVILSHGCQASHLQFPGAVYGTDGCPVSVEKIVNIFNGTSCPSLGGKPKLFFIQACGGEQKDHGFEVASTSPEDESPGSNPEPDATPFQEGLRTFDQLDAISSLPTPSDIFVSYSTFPGFVSWRDPKSGSWYVETLDDIFEQWAHSEDLQSLLLRVANAVSVKGIYKQMPGCFNFLRKKLFFKTSAS

SEQ ID NO: 75 - protein, 157 aa, Artificial Sequence
  RQR8
MGTSLLCWMALCLLGADHADACPYSNPSLCSGGGGSELPTQGTFSNVSTNVSPAKPTTTACPYSNPSLCSGGGGSPAPRPPTPAPTIASQPLSLRPEACRPAAGGAVHTRGLDFACDIYIWAPLAGTCGVLLLSLVITLYCNHRNRRRVCKCPRPVV

SEQ ID NO: 76 - protein, 528 aa, Artificial Sequence
  CAR
MGTSLLCWMALCLLGADHADACPYSNPSLCSGGGGSELPTQGTFSNVSTNVSPAKPTTTACPYSNPSLCSGGGGSPAPRPPTPAPTIASQPLSLRPEACRPAAGGAVHTRGLDFACDIYIWAPLAGTCGVLLLSLVITLYCNHRNRRRVCKCPRPVVRAEGRGSLLTCGDVEENPGPMGWSCIILFLVATATGVHSQVQLQESGGGLVQAGGSLRLSCAASRSSFSNNAMGWYRQVPGKQRELVAFITKGGVTDYSDSVKGRFTISKDNAKNTVYLQMNSLKPEDTAVYYCNTIPFRSAWGQGTQVTVSSDPTPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 77 - protein, 538 aa, Artificial Sequence
  CAR
MGTSLLCWMALCLLGADHADACPYSNPSLCSGGGGSELPTQGTFSNVSTNVSPAKPTTTACPYSNPSLCSGGGGSPAPRPPTPAPTIASQPLSLRPEACRPAAGGAVHTRGLDFACDIYIWAPLAGTCGVLLLSLVITLYCNHRNRRRVCKCPRPVVRAEGRGSLLTCGDVEENPGPMGWSCIILFLVATATGVHSQVQLQESGGGSVQPGGSLSLSCAASGFTFSNYAMSWVRQAPGKGPEWVAVINSDGGTASSAGSVRGRFTISRDNAKNTLYLQMNRLKPEDTAVYYCAANRGFCAGVRCLEYQYWGQGTQVTVSSDPTPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 78 - protein, 539 aa, Artificial Sequence
  CAR
MGTSLLCWMALCLLGADHADACPYSNPSLCSGGGGSELPTQGTFSNVSTNVSPAKPTTTACPYSNPSLCSGGGGSPAPRPPTPAPTIASQPLSLRPEACRPAAGGAVHTRGLDFACDIYIWAPLAGTCGVLLLSLVITLYCNHRNRRRVCKCPRPVVRAEGRGSLLTCGDVEENPGPMGWSCIILFLVATATGVHSQVQLQESGGGLVQAGGSLIVSCAASGRTFSINAMAWFRQAPGKEREFVAAIGGSGRVSSTSYADFVKGRFTISRDNAKNTVYLRMNNLEPEDTAVYYCAARRDYLPFPPESYDYWGQGTQVTVSSDPTPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 79 - protein, 544 aa, Artificial Sequence
  CAR
MGTSLLCWMALCLLGADHADACPYSNPSLCSGGGGSELPTQGTFSNVSTNVSPAKPTTTACPYSNPSLCSGGGGSPAPRPPTPAPTIASQPLSLRPEACRPAAGGAVHTRGLDFACDIYIWAPLAGTCGVLLLSLVITLYCNHRNRRRVCKCPRPVVRAEGRGSLLTCGDVEENPGPMGWSCIILFLVATATGVHSQVQLQESGGGLVQAGGSLRLSCAASGRTFSTYAMAWFRQAPGKEREFVAAISGFGVVTYYADSVKGRFTISRDNAKNTLYLQMNGLKPEDTAVYYCAAGRRTSTNGGDYDYWGQGTQVTVSSDPTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRA

SEQ ID NO: 80 - protein, 539 aa, Artificial Sequence
  CAR
MGTSLLCWMALCLLGADHADACPYSNPSLCSGGGGSELPTQGTFSNVSTNVSPAKPTTTACPYSNPSLCSGGGGSPAPRPPTPAPTIASQPLSLRPEACRPAAGGAVHTRGLDFACDIYIWAPLAGTCGVLLLSLVITLYCNHRNRRRVCKCPRPVVRAEGRGSLLTCGDVEENPGPMGWSCIILFLVATATGVHSQVQLQESGGGLVQAGGSLIVSCATSGRTFSINAMAWFRQAPGKEREFVAAIGGSGRVSSTSYADFVKGRFTISRDNAKNTVYLRMNNLEPEDTAVYYCAARRNYLPFPPESYDYWGQGIQVTVSSDPTPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 81 - protein, 537 aa, Artificial Sequence
  CAR
MGTSLLCWMALCLLGADHADACPYSNPSLCSGGGGSELPTQGTFSNVSTNVSPAKPTTTACPYSNPSLCSGGGGSPAPRPPTPAPTIASQPLSLRPEACRPAAGGAVHTRGLDFACDIYIWAPLAGTCGVLLLSLVITLYCNHRNRRRVCKCPRPVVRAEGRGSLLTCGDVEENPGPMGWSCIILFLVATATGVHSQVQLQQSGGGLVQAGDSLRLSCAASGRTFSNSTMGWFRQAPGKERKFVAVISWSGGTYAYAESVKGRFTISRDNAKNTVYLQMNSLKPEDTAVYYCAAARKGWSTRGDDYDYWGQGTQVTVSSDPTPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 82 - protein, 537 aa, Artificial Sequence
  CAR
MGTSLLCWMALCLLGADHADACPYSNPSLCSGGGGSELPTQGTFSNVSTNVSPAKPTTTACPYSNPSLCSGGGGSPAPRPPTPAPTIASQPLSLRPEACRPAAGGAVHTRGLDFACDIYIWAPLAGTCGVLLLSLVITLYCNHRNRRRVCKCPRPVVRAEGRGSLLTCGDVEENPGPMGWSCIILFLVATATGVHSQVQLQESGGGLVQAGGSLRLSCAASGRTYNNYAMGWFRQAPGKEREFVAGISRSGGMTGYAESVKGRFTISRDNAKNMVFLQMNSLKPEDTAVYYCAAYVGGFSTARRDYSYWGQGTQVTVSSDPTPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 83 - protein, 538 aa, Artificial Sequence
  CAR
MGTSLLCWMALCLLGADHADACPYSNPSLCSGGGGSELPTQGTFSNVSTNVSPAKPTTTACPYSNPSLCSGGGGSPAPRPPTPAPTIASQPLSLRPEACRPAAGGAVHTRGLDFACDIYIWAPLAGTCGVLLLSLVITLYCNHRNRRRVCKCPRPVVRAEGRGSLLTCGDVEENPGPMGWSCIILFLVATATGVHSQVQLQESGGGLVQAGDSLRLSCAYSGRTFSSYAMGWFRQAPGKERVFVAAISRIGGVTTYAESVQGRFTISRDNAKNTLYLQMNALKPEDTAVYYCAAAGLVSISTTPNDYDYWGQGTQVTVSSDPTPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 84 - protein, 528 aa, Artificial Sequence
  CAR
MGTSLLCWMALCLLGADHADACPYSNPSLCSGGGGSELPTQGTFSNVSTNVSPAKPTTTACPYSNPSLCSGGGGSPAPRPPTPAPTIASQPLSLRPEACRPAAGGAVHTRGLDFACDIYIWAPLAGTCGVLLLSLVITLYCNHRNRRRVCKCPRPVVRAEGRGSLLTCGDVEENPGPMGWSCIILFLVATATGVHSQVQLQQSGGGLVQPGGSLRLSCAAPGNIFRLNGTGWYRQAPGKQRELVAHITSGGNTDYADSVKGRFTISRDNAKNTVYLQMNSLKPEDTAVYYCNAIPFRLSWGQGTQVTVSSDPTPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 85 - protein, 530 aa, Artificial Sequence
  CAR
MGTSLLCWMALCLLGADHADACPYSNPSLCSGGGGSELPTQGTFSNVSTNVSPAKPTTTACPYSNPSLCSGGGGSPAPRPPTPAPTIASQPLSLRPEACRPAAGGAVHTRGLDFACDIYIWAPLAGTCGVLLLSLVITLYCNHRNRRRVCKCPRPVVRAEGRGSLLTCGDVEENPGPMGWSCIILFLVATATGVHSQVQLQESGGGLVQAGGSLRLSCVVSRNIFSLNPMGWYRQAPGKQREMVAIITNGGSTNYADSVKGRFTISRDNVKNTVYLQMNALKPEDTAVYYCNRVGGLQTWAWGQGTQVTVSSDPTPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 86 - protein, 536 aa, Artificial Sequence
  CAR
MGTSLLCWMALCLLGADHADACPYSNPSLCSGGGGSELPTQGTFSNVSTNVSPAKPTTTACPYSNPSLCSGGGGSPAPRPPTPAPTIASQPLSLRPEACRPAAGGAVHTRGLDFACDIYIWAPLAGTCGVLLLSLVITLYCNHRNRRRVCKCPRPVVRAEGRGSLLTCGDVEENPGPMGWSCIILFLVATATGVHSQVQLQQSGGGLVQAGGSLSLSCTASGRSFSNYGMGWFRQAPGKEREFVAAIGMVGGLTAYSNSAKGRFTISRDNAKNTLYLQMNSLKPEDTAVYLCAAGRRFSTSSRDYDIWGQGTQVTVSSDPTPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 87 - protein, 540 aa, Artificial Sequence
  CAR
MGTSLLCWMALCLLGADHADACPYSNPSLCSGGGGSELPTQGTFSNVSTNVSPAKPTTTACPYSNPSLCSGGGGSPAPRPPTPAPTIASQPLSLRPEACRPAAGGAVHTRGLDFACDIYIWAPLAGTCGVLLLSLVITLYCNHRNRRRVCKCPRPVVRAEGRGSLLTCGDVEENPGPMGWSCIILFLVATATGVHSQVQLQESGGGLVQAGDSLRLSCAASGRTFDSRPMGWFRQAPGKEREFVGAVSWRGESTYYPDSVKGRFTISRDNAKRTVYLQMNSLKPEDTAVYYCAAGEPYSGTYYYRGRDYDYWGQGTQVTVSSDPTPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 88 - protein, 537 aa, Artificial Sequence
  CAR
MGTSLLCWMALCLLGADHADACPYSNPSLCSGGGGSELPTQGTFSNVSTNVSPAKPTTTACPYSNPSLCSGGGGSPAPRPPTPAPTIASQPLSLRPEACRPAAGGAVHTRGLDFACDIYIWAPLAGTCGVLLLSLVITLYCNHRNRRRVCKCPRPVVRAEGRGSLLTCGDVEENPGPMGWSCIILFLVATATGVHSQVQLQESGGGLVQAEGSLRLSCAASGRTFSMYAMGWFRQAPGREREFVAAISGSARITYYGQSVKGRFTISRDNAKNTVYLQMNSLKPEDTAVYYCAASSTYTSTSGSSYNYWGQGTQVTVSSDPTPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 89 - protein, 531 aa, Artificial Sequence
  CAR
MGTSLLCWMALCLLGADHADACPYSNPSLCSGGGGSELPTQGTFSNVSTNVSPAKPTTTACPYSNPSLCSGGGGSPAPRPPTPAPTIASQPLSLRPEACRPAAGGAVHTRGLDFACDIYIWAPLAGTCGVLLLSLVITLYCNHRNRRRVCKCPRPVVRAEGRGSLLTCGDVEENPGPMGWSCIILFLVATATGVHSQVQLQESGGGLVQAGGSLRLSCAASGSIFSINAMGWYRQAPGKQRELVAFITSGGSTNYADSVKGRFTISRDNAKNTVYLQMNSLKPEDTAVYYCNALGGFVPSYGWGQGTQVTVSSDPTPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 90 - protein, 13 aa, Artificial Sequence
  CDR1
KASGYTFTRYTMH

SEQ ID NO: 91 - protein, 16 aa, Artificial Sequence
  CDR2
INPSRGYTNYNQKFKD

SEQ ID NO: 92 - protein, 10 aa, Artificial Sequence
  CDR3
YYDDHYCLDY

SEQ ID NO: 93 - protein, 10 aa, Artificial Sequence
  CDR1
SASSSVSYMN

SEQ ID NO: 94 - protein, 11 aa, Artificial Sequence
  CDR2
RWIYDTSKLAS

SEQ ID NO: 95 - protein, 9 aa, Artificial Sequence
  CDR3
QQWSSNPFT

SEQ ID NO: 96 - protein, 242 aa, Artificial Sequence
  scFv
QVQLQQSGAELARPGASVKMSCKASGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSSGGGGSGGGGSGGGGSQIVLTQSPAIMSASPGEKVTMTCSASSSVSYMNWYQQKSGTSPKRWIYDTSKLASGVPAHFRGSGSGTSYSLTISGMEAEDAATYYCQQWSSNPFTFGSGTKLEINR

SEQ ID NO: 97 - protein, 8 aa, Artificial Sequence
  CDR1
GYSFTGYT

SEQ ID NO: 98 - protein, 8 aa, Artificial Sequence
  CDR2
INPYKGVS

SEQ ID NO: 99 - protein, 15 aa, Artificial Sequence
  CDR3
ARSGYYGDSDWYFDV

SEQ ID NO: 100 - protein, 6 aa, Artificial Sequence
  CDR1
QDIRNY

SEQ ID NO: 101 - protein, 3 aa, Artificial Sequence
  CDR2
YTS

SEQ ID NO: 102 - protein, 9 aa, Artificial Sequence
  CDR3
QQGNTLPWT

SEQ ID NO: 103 - protein, 245 aa, Artificial Sequence
  scFv
DIQMTQSPSSLSASVGNRVTITCRASQDIRNYLNWYQQKPGKAPKLLIYYTSRLESGVPSRFSGSGSGTDYTLTISSLQPEDFATYYCQQGNTLPWTFGQGTKVEIKSGGGGSGGGGSGGGGSEVQLVESGGGLVQPGGSLRLSCAASGYSFTGYTMNWVRQAPGKGLEWVALINPYKGVSTYNQKFKDRFTISVDKSKNTAYLQMNSLRAEDTAVYYCARSGYYGDSDWYFDVWGQGTLVTVSS

SEQ ID NO: 104 - protein, 8 aa, Artificial Sequence
  CDR1
GFTFSSFP

SEQ ID NO: 105 - protein, 8 aa, Artificial Sequence
  CDR2
ISTSGGRT

SEQ ID NO: 106 - protein, 12 aa, Artificial Sequence
  CDR3
AKFRQYSGGFDY

SEQ ID NO: 107 - protein, 8 aa, Artificial Sequence
  CDR1
SGNIENNY

SEQ ID NO: 108 - protein, 3 aa, Artificial Sequence
  CDR2
DDD

SEQ ID NO: 109 - protein, 9 aa, Artificial Sequence
  CDR3
HSYVSSFNV

SEQ ID NO: 110 - protein, 245 aa, Artificial Sequence
  scFv
DFMLTQPHSVSESPGKTVIISCTLSSGNIENNYVHWYQQRPGRAPTTVIFDDDKRPDGVPDRFSGSIDRSSNSASLTISGLQTEDEADYYCHSYVSSFNVFGGGTKLTVLSGGGGSGGGGSGGGGSEVQLLESGGGLVQPGGSLRLSCAASGFTFSSFPMAWVRQAPGKGLEWVSTISTSGGRTYYRDSVKGRFTISRDNSKNTLYLQMNSLRAEDTAVYYCAKFRQYSGGFDYWGQGTLVTVSS

SEQ ID NO: 111 - protein, 20 aa, Artificial Sequence
  signal peptide
METDTLLLWVLLLWVPGSTG

SEQ ID NO: 112 - protein, 21 aa, Artificial Sequence
  signal peptide
MGTSLLCWMALCLLGADHADG

SEQ ID NO: 113 - protein, 26 aa, Artificial Sequence
  IgG1 hinge
AEPKSPDKTHTCPPCPKDPKSGGGGS

SEQ ID NO: 114 - protein, 45 aa, Artificial Sequence; identical to SEQ ID NO: 58
  CD8 stalk

SEQ ID NO: 115 - protein, 7 aa, Artificial Sequence
  linker motif
SGGGSDP

SEQ ID NO: 116 - protein, 6 aa, Artificial Sequence
  linker
SGGGGS

SEQ ID NO: 117 - protein, 5 aa, Artificial Sequence
  linker
GGGGS

SEQ ID NO: 118 - skipped

SEQ ID NO: 119 - protein, 155 aa, Artificial Sequence
  4-1BB-CD3Z
KRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRA

SEQ ID NO: 120 - protein, 121 aa, Artificial Sequence
  VH domain
QVQLVESGGGVVQPGRSLRLSCAASGFTFTKAWMHWVRQAPGKQLEWVAQIKDKSNSYATYYADSVKGRFTISRDDSKNTLYLQMNSLRAEDTAVYYCRGVYYALSPFDYWGQGTLVTVSS

SEQ ID NO: 121 - protein, 112 aa, Artificial Sequence
  VL domain
DIVMTQTPLSLSVTPGQPASISCKSSQSLVHNNANTYLSWYLQKPGQSPQSLIYKVSNRFSGVPDRFSGSGSGTDFTLKISRVEAEDVGVYYCGQGTQYPFTFGSGTKVEIK

SEQ ID NO: 122 - protein, 121 aa, Artificial Sequence
  VH domain
QVQLVESGGGVVQPGRSLRLSCAASGFTFTKAWMHWVRQAPGKGLEWVAQIKDKSNSYATYYADSVKGRFTISRDNSKNTLYLQMNSLRAEDTAVYYCRGVYYALSPFDYWGQGTLVTVSS

SEQ ID NO: 123 - protein, 112 aa, Artificial Sequence
  VL domain
DIVMTQTPLSLSVTPGQPASISCKSSQSLVHNNGNTYLSWYLQKPGQSPQLLIYKVSNRFSGVPDRFSGSGSGTDFTLKISRVEAEDVGVYYCGQGTQYPFTFGGGTKVEIK

SEQ ID NO: 124 - protein, 8 aa, Artificial Sequence
  CDR1
GYTFTSYY

SEQ ID NO: 125 - protein, 8 aa, Artificial Sequence
  CDR2
IYPGNVNT

SEQ ID NO: 126 - protein, 13 aa, Artificial Sequence
  CDR3
TRSHYGLDWNFDV

SEQ ID NO: 127 - protein, 6 aa, Artificial Sequence
  CDR1
QNIYVW

SEQ ID NO: 128 - protein, 2 aa, Artificial Sequence
  CDR2
KA

SEQ ID NO: 129 - protein, 9 aa, Artificial Sequence
  CDR3
QQGQTYPYT

SEQ ID NO: 130 - protein, 120 aa, Artificial Sequence
  VH domain
QVQLVQSGAEVKKPGASVKVSCKASGYTFTSYYIHWVRQAPGQGLEWIGCIYPGNVNTNYNEKFKDRATLTVDTSISTAYMELSRLRSDDTAVYFCTRSHYGLDWNFDVWGQGTTVTVSS

SEQ ID NO: 131 - protein, 110 aa, Artificial Sequence
  VL domain
DIQMTQSPSSLSASVGDRVTITCHASQNIYVWLNWYQQKPGKAPKLLIYKASNLHTGVPSRFSGSGSGTDFTLTISSLQPEDFATYYCQQGQTYPYTFGGGTKVEIKRTV

SEQ ID NO: 132 - protein, 120 aa, Artificial Sequence
  VH domain
QVQLVQSGAEVVKPGASVKVSCKASGYTFTSYYIHWVRQAPGQGLEWIGSIYPGNVNTNYAQKFQGRATLTVDTSISTAYMELSRLRSDDTAVYYCTRSHYGLDWNFDVWGKGTTVTVSS

SEQ ID NO: 133 - protein, 107 aa, Artificial Sequence
  VL domain
DIQMTQSPSSLSASVGDRVTITCQASQNIYVWLNWYQQKPGKAPKLLIYKASNLHTGVPSRFSGSGSGTDFTLTISSLQPEDIATYYCQQGQTYPYTFGQGTKLEIK

SEQ ID NO: 134 - protein, 120 aa, Artificial Sequence
  VH domain
QVQLQESGPGLVKPSQTLSLTCTVSGFSLSDYGVHWVRQPPGKGLEWLGVIWAGGGTNYNPSLKSRKTISKDTSKNQVSLKLSSVTAADTAVYYCARDKGYSYYYSMDYWGQGTTVTVSS

SEQ ID NO: 135 - protein, 111 aa, Artificial Sequence
  VL domain
DIVLTQSPASLAVSPGQRATITCRASESVEYYVTSLMQWYQQKPGQPPKLLIFAASNVESGVPARFSGSGSGTDFTLTINPVEANDVANYYCQQSRKVPYTFGQGTKLEIK

SEQ ID NO: 136 - protein, 10 aa, Artificial Sequence
  CDR1
GFSLRTSGMG

SEQ ID NO: 137 - protein, 7 aa, Artificial Sequence
  CDR2
IWWDDDK

SEQ ID NO: 138 - protein, 10 aa, Artificial Sequence
  CDR3
AQINPAWFAY

SEQ ID NO: 139 - protein, 10 aa, Artificial Sequence
  CDR1
QSVDFDGDSF

SEQ ID NO: 140 - protein, 2 aa, Artificial Sequence
  CDR2
TT

SEQ ID NO: 141 - protein, 9 aa, Artificial Sequence
  CDR3
QQSNEDPYT

SEQ ID NO: 142 - protein, 118 aa, Artificial Sequence
  VH domain
QVTLKESGPGILQPSQTLSLTCSFSGFSLRTSGMGVGWIRQPSGKGLEWLAHIWWDDDKRYNPALKSRLTISKDTSSNQVFLKIASVDTADTATYYCAQINPAWFAYWGQGTLVTVSA

SEQ ID NO: 143 - protein, 111 aa, Artificial Sequence
  VL domain
DIVLTQSPASLAVSLGQRATISCKASQSVDFDGDSFMNWYQQKPGQPPKLLIYTTSNLESGIPARFSASGSGTDFTLNIHPVEEEDTATYYCQQSNEDPYTFGGGTKLELK

SEQ ID NO: 144 - protein, 10 aa, Artificial Sequence
  CDR1
GFSLSTSGMG

SEQ ID NO: 145 - protein, 10 aa, Artificial Sequence
  CDR3
ARINPAWFAY

SEQ ID NO: 146 - protein, 118 aa, Artificial Sequence
  VH domain
QVTLKESGPALVKPTQTLTLTCTFSGFSLSTSGMGVGWIRQPPGKALEWLAHIWWDDDKRYNPALKSRLTISKDTSKNQVVLTMTNMDPVDTATYYCARINPAWFAYWGQGTLVTVSS

SEQ ID NO: 147 - protein, 111 aa, Artificial Sequence
  VL domain
DIVMTQSPDSLAVSLGERATINCKASQSVDFDGDSFMNWYQQKPGQPPKLLIYTTSNLESGVPDRFSGSGSGTDFTLTIRPLQAEDVAVYYCQQSNEDPYTFGQGTKLEIK

SEQ ID NO: 148 - protein, 8 aa, Artificial Sequence
  CDR2
INPSGGST

SEQ ID NO: 149 - protein, 13 aa, Artificial Sequence
  CDR3
ARGSAYYYDFADY

SEQ ID NO: 150 - protein, 6 aa, Artificial Sequence
  CDR1
NIGSKN

SEQ ID NO: 151 - protein, 2 aa, Artificial Sequence
  CDR2
QD

SEQ ID NO: 152 - protein, 9 aa, Artificial Sequence
  CDR3
QVWDNYSVL

SEQ ID NO: 153 - protein, 120 aa, Artificial Sequence
  VH domain
QVQLVQSGAEVKKPGESLKVSCKASGYTFTSYYMHWVRQAPGQGLEWMGIINPSGGSTSYAQKFQGRVTMTRDTSTSTVYMELSSLRSEDTAVYYCARGSAYYYDFADYWGQGTLVTVSS

SEQ ID NO: 154 - protein, 106 aa, Artificial Sequence
  VL domain
SYVLTQPSSVSVAPGQTATISCGGHNIGSKNVHWYQQRPGQSPVLVIYQDNKRPSGIPERFSGSNSGNTATLTISGTQAMDEADYYCQVWDNYSVLFGGGTKLTVL

SEQ ID NO: 155 - protein, 8 aa, Artificial Sequence
  CDR1
GRTSSRAA

SEQ ID NO: 156 - protein, 8 aa, Artificial Sequence
  CDR2
ISWSGGTT

SEQ ID NO: 157 - protein, 15 aa, Artificial Sequence
  CDR3
AAARIFTTARNDYDH

SEQ ID NO: 158 - protein, 121 aa, Artificial Sequence
  FcRH5-specific sdAb (VHH)
QVQLQESGGGLVQAGGSLRLSCTASGRTSSRAAMGWFRQAPGKEREFVAVISWSGGTTAYANSVKGRFTISRDNAKNTLYLQMNSLKPEDTAVYYCAAARIFTTARNDYDHWGQGTQVTVS

SEQ ID NO: 159 - protein, 536 aa, Artificial Sequence
  CAR
MGTSLLCWMALCLLGADHADACPYSNPSLCSGGGGSELPTQGTFSNVSTNVSPAKPTTTACPYSNPSLCSGGGGSPAPRPPTPAPTIASQPLSLRPEACRPAAGGAVHTRGLDFACDIYIWAPLAGTCGVLLLSLVITLYCNHRNRRRVCKCPRPVVRAEGRGSLLTCGDVEENPGPMGWSCIILFLVATATGVHSQVQLQESGGGLVQAGGSLRLSCTASGRTSSRAAMGWFRQAPGKEREFVAVISWSGGTTAYANSVKGRFTISRDNAKNTLYLQMNSLKPEDTAVYYCAAARIFTTARNDYDHWGQGTQVTVSSDPTPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 160 - protein, 8 aa, Artificial Sequence
  CDR1
GNIFRLNA

SEQ ID NO: 161 - protein, 8 aa, Artificial Sequence
  CDR1
GSSFRLNG

SEQ ID NO: 162 - protein, 8 aa, Artificial Sequence
  CDR1
GRSVSINA

SEQ ID NO: 163 - protein, 8 aa, Artificial Sequence
  CDR1
GNNFRLNA

SEQ ID NO: 164 - protein, 8 aa, Artificial Sequence
  CDR1
ERIFRINA

SEQ ID NO: 165 - protein, 8 aa, Artificial Sequence
  CDR1
GFTFSTYW

SEQ ID NO: 166 - protein, 8 aa, Artificial Sequence
  CDR1
GNIFRING

SEQ ID NO: 167 - protein, 8 aa, Artificial Sequence
  CDR1
GSSFSNNA

SEQ ID NO: 168 - protein, 8 aa, Artificial Sequence
  CDR1
RSSFGNNA

SEQ ID NO: 169 - protein, 8 aa, Artificial Sequence
  CDR1
GRTFSTYG

SEQ ID NO: 170 - protein, 8 aa, Artificial Sequence
  CDR1
GTIERNNA

SEQ ID NO: 171 - protein, 8 aa, Artificial Sequence
  CDR1
GRTFRRYA

SEQ ID NO: 172 - protein, 5 aa, Artificial Sequence
  CDR1
GRTVI

SEQ ID NO: 173 - protein, 8 aa, Artificial Sequence
  CDR1
GRTFSRYA

SEQ ID NO: 174 - protein, 8 aa, Artificial Sequence
  CDR1
GRSFSINA

SEQ ID NO: 175 - protein, 8 aa, Artificial Sequence
  CDR1
GNIFSINA

SEQ ID NO: 176 - protein, 7 aa, Artificial Sequence
  CDR2
IDRSGNT

SEQ ID NO: 177 - protein, 8 aa, Artificial Sequence
  CDR2
IDGIGGIT

SEQ ID NO: 178 - protein, 8 aa, Artificial Sequence
  CDR2
IDNGGGTT

SEQ ID NO: 179 - protein, 7 aa, Artificial Sequence
  CDR2
ITRGGNT

SEQ ID NO: 180 - protein, 8 aa, Artificial Sequence
  CDR2
IGMVGGLP

SEQ ID NO: 181 - protein, 8 aa, Artificial Sequence
  CDR2
ISRGGGVS

SEQ ID NO: 182 - protein, 8 aa, Artificial Sequence
  CDR2
SSGSGGVT

SEQ ID NO: 183 - protein, 8 aa, Artificial Sequence
  CDR2
SNWSGGVT

SEQ ID NO: 184 - protein, 7 aa, Artificial Sequence
  CDR2
INGSGGT

SEQ ID NO: 185 - protein, 7 aa, Artificial Sequence
  CDR2
ITRGGST

SEQ ID NO: 186 - protein, 8 aa, Artificial Sequence
  CDR3
NTIPFRLS

SEQ ID NO: 187 - protein, 8 aa, Artificial Sequence
  CDR3
NTIPVRSA

SEQ ID NO: 188 - protein, 8 aa, Artificial Sequence
  CDR3
NTIPCRSA

SEQ ID NO: 189 - protein, 8 aa, Artificial Sequence
  CDR3
NTIPFSRA

SEQ ID NO: 190 - protein, 8 aa, Artificial Sequence
  CDR3
NTIPYSDS

SEQ ID NO: 191 - protein, 8 aa, Artificial Sequence
  CDR3
NAIPFRSA

SEQ ID NO: 192 - protein, 8 aa, Artificial Sequence
  CDR3
NAIPFRPS

SEQ ID NO: 193 - protein, 8 aa, Artificial Sequence
  CDR3
NAIPFRRS

SEQ ID NO: 194 - protein, 8 aa, Artificial Sequence
  CDR3
NALPFRLS

SEQ ID NO: 195 - protein, 8 aa, Artificial Sequence
  CDR3
NATPFRLS

SEQ ID NO: 196 - protein, 8 aa, Artificial Sequence
  CDR3
NSIPFRLS

SEQ ID NO: 197 - protein, 8 aa, Artificial Sequence
  CDR3
NAIPFRIS

SEQ ID NO: 198 - protein, 8 aa, Artificial Sequence
  CDR3
NAIPFRLY

SEQ ID NO: 199 - protein, 8 aa, Artificial Sequence
  CDR3
KAIPFRLS

SEQ ID NO: 200 - protein, 15 aa, Artificial Sequence
  CDR3
AAGRRFSTRSRDYDY

SEQ ID NO: 201 - protein, 15 aa, Artificial Sequence
  CDR3
AAGRRFSTSSRDYDY

SEQ ID NO: 202 - protein, 15 aa, Artificial Sequence
  CDR3
AAGRRFSTGSRDYDI

SEQ ID NO: 203 - protein, 15 aa, Artificial Sequence
  CDR3
AGGRRFSTSSRDYDI

SEQ ID NO: 204 - protein, 15 aa, Artificial Sequence
  CDR3
AAGRRFSTSSREYDI

SEQ ID NO: 205 - protein, 15 aa, Artificial Sequence
  CDR3
AAGRRLSTSSRDYDI

SEQ ID NO: 206 - protein, 15 aa, Artificial Sequence
  CDR3
AAGLRFSTGSRDYDI

SEQ ID NO: 207 - protein, 16 aa, Artificial Sequence
  CDR3
AAYVGGFSTTRRDYAY

SEQ ID NO: 208 - protein, 15 aa, Artificial Sequence
  CDR3
AAALTWSTRPSDFTS

SEQ ID NO: 209 - protein, 15 aa, Artificial Sequence
  CDR3
AAARIFTTTRNEYDH

SEQ ID NO: 210 - protein, 15 aa, Artificial Sequence
  CDR3
AAARIFSTARNDYDH

SEQ ID NO: 211 - protein, 15 aa, Artificial Sequence
  CDR3
AGGRIFRTSSRDYDI

SEQ ID NO: 212 - protein, 15 aa, Artificial Sequence
  CDR3
AAARFFTTARNDYDH

SEQ ID NO: 213 - protein, 11 aa, Artificial Sequence
  CDR3
NALGGFVPNYG

SEQ ID NO: 214 - protein, 11 aa, Artificial Sequence
  CDR3
NALGGFVPNDG

SEQ ID NO: 215 - protein, 11 aa, Artificial Sequence
  CDR3
NALGGFAPNYG

SEQ ID NO: 216 - protein, 11 aa, Artificial Sequence
  CDR3
NALGGFVPNYV

SEQ ID NO: 217 - protein, 11 aa, Artificial Sequence
  CDR3
NALGGFVLNYG

SEQ ID NO: 218 - protein, 11 aa, Artificial Sequence
  CDR3
NALGGLVPNYG

SEQ ID NO: 219 - protein, 11 aa, Artificial Sequence
  CDR3
NALGGFLPNYG

SEQ ID NO: 220 - protein, 11 aa, Artificial Sequence
  CDR3
NALGGFVTNYG

SEQ ID NO: 221 - protein, 13 aa, Artificial Sequence
  CDR3
ARNPTRGWYSTDY

SEQ ID NO: 222 - protein, 4 aa, Artificial Sequence
  CDR1
GRTV

SEQ ID NO: 223 - protein, 114 aa, Artificial Sequence
  VHH
QVQLQESGGGLVQPGGSLRLSCAAPGNIFRLNGTGWYRQAPGKQRELVAHITSGGNTDYADSVKGRFTISRDNAKNTVYLQMNSLKPEDTAVYYCNTIPFRLSWGQGTQVTVSS

SEQ ID NO: 224 - protein, 114 aa, Artificial Sequence
  VHH
QVQLQESGGGLVQPGGSLRLSCAAPGNIFRLNAMGWYRQAPGKQRELVAHITSGGNTDYADSVKGRFTISRDNAKNTVYLQMNSLKLEDTAVYYCNTIPFRLSWGQGTQVTVSS

SEQ ID NO: 225 - protein, 114 aa, Artificial Sequence
  VHH
QVQLQESGGGLAQAGGSLRLSCAASRSSFSNNAMGWYRQVQGKQRELVAFITKGGVTDYSVSGKGRFTISKDHAKNTVYLQMNSLKPRDTAVYYCNTIPVRSAWGQGTQVTVSA

SEQ ID NO: 226 - protein, 114 aa, Artificial Sequence
  VHH
QVQLQESGGGLVQAGGSLRLSCAASGSIFSINAMGWYRQAPGKQRELVAFITKGGVTDYSDSVKGRFTISKDNAKNTVYLQMNSLKPEDTAVYYCNTIPCRSAWGQGTQVTVSS

SEQ ID NO: 227 - protein, 114 aa, Artificial Sequence
  VHH
QVQLQESGGGLVQPGGSLRLSCAAPGSSFRLNGTGWYRQAPGKQRELVAHITSGGSTNYSDSVKGRLTISKDNAKNTVYLQMNSLKPEDTAVYYCNTIPFSRAWGQGTLVTVSS

SEQ ID NO: 228 - protein, 114 aa, Artificial Sequence
  VHH
QVQLQQSGGGLVQAGGSLRLSCAASGRSVSINAMGWYRQAPGKQRELVAIIDRSGNTDYADSVKGRFTISRDNAKKAVYLQMNSLKPEDTAVYYCNTIPYSDSWGQGTQVTISS

SEQ ID NO: 229 - protein, 115 aa, Artificial Sequence
  VHH
QVQLQESGGGLVQPGGSLRLSCAASGRTFSSYAMGWFRRAPGKGREFVATIDGIGGITSYAGSVKGRFTVSKDNAKNTVYLQMNSLKPEDTAVYYCNTIPFRSAWGQGTLVTVSS

SEQ ID NO: 230 - protein, 114 aa, Artificial Sequence
  VHH
QVQLQESGGGLVQAGGSLRLSCAASRSSFSNNAMGWYRQGPGKQRELVAFITKGGVTDYSDSVKGRFTISKDNAKNTVYLQMNSLKPEDTAVYYCNTIPFRSAWGQGTQVTVSS

SEQ ID NO: 231 - protein, 114 aa, Artificial Sequence
  VHH
QVQLQESGGGLVQAGGSLRLSCAASRSSFSNNAMGWYRQVPGKQRELVAFITKGGVTDYSDSVKGRFTISKDNAKNTVYLQMNSLKPEDTAVYYCNAIPFRSAWGQGTQVTVSS

SEQ ID NO: 232 - protein, 114 aa, Artificial Sequence
  VHH
QVQLQESGGGLVQPGGSLRLSCAAPGNNFRLNAMGWYRQAPGKQRELVAHITSGGNTDYADSVKGRFTISRDNAKNTVYLQMNSLKPEDTAVYYCNAIPFRPSWGQGTQVTVSS

SEQ ID NO: 233 - protein, 114 aa, Artificial Sequence
  VHH
QVQLQESGGGLVQPGGSLRLSCAAPGNIFRLNGTGWYRQAPGKQRELVAHITSGGNTDYADSGKGRFTISRDNAKNTVYLQMNSLKPEDTAVYYCNAIPFRRSWGQGTQVTVSS

SEQ ID NO: 234 - protein, 114 aa, Artificial Sequence
  VHH
QVQLQESGGGLVQAGGSLKLSCAASERIFRINAMGWYRQAPGKQRELVAHITSGGNTDYADSVKGRFTISRDNAKNTMYLQMNSLKPEDTAVYYCNAIPFRRSWGQGTQVTVSS

SEQ ID NO: 235 - protein, 114 aa, Artificial Sequence
  VHH
QVQLQQSGGGLVQPGGSLRLSCAAPGNIFRLNGTGWYRQAPGKQRELVAHITSGGNTDYADSVKGRFTISRDNAENTVYLQMNSLKPEDTAVYYCNAVPFRLSWGQGTQVTVSS

SEQ ID NO: 236 - protein, 115 aa, Artificial Sequence
  VHH
QVQLQESGGGLVQPGESLRLSCAASGFTFSTYWMSWVRQAPGKGPEGVSGIDNGGGTTTYADSVKGRFTISRDNAGNTVYLQMNSLKPEDTAVYYCNALPFRLSWGQGTQVTVSS

SEQ ID NO: 237 - protein, 114 aa, Artificial Sequence
  VHH
QVQLQESGGGLVQPGGSLRLSCAAPGNIFRLNGTGWYRQAPGKQGELVAIITSGGNTDYADSVKGRFTISRDNAKNTTYLQMNSLKPEDTAVYYCNATPFRLSWGQGTQVTVSS

SEQ ID NO: 238 - protein, 114 aa, Artificial Sequence
  VHH
QVQLQESGGGLVQPGGSLRLSCAAPGNIFRLNGTGWYRQAPGKQRELVAHITRGGNTDYADAVKGRFTISRDNAKNTVYLQMNSLKPEDTAVYYCNSIPFRLSWGQGTQVTVSS

SEQ ID NO: 239 - protein, 114 aa, Artificial Sequence
  VHH
QVQLQESGGGLVQPGGSLRLSCAAPGNIFRINGTGWYGQAPGKQRELVAHITSGGNTDYEDSVKGRFTISRDNAKNTTYLQMNSLKPEDTAVYYCNAIPFRISWGQGTQVTVSS

SEQ ID NO: 240 - protein, 114 aa, Artificial Sequence
  VHH
QVQLQQSGGGLVQPGGSLRLSCAAPGNNFRLNAMGWYRQAPGKQRELVAHITSGGNTDYADSVKGRFTISRDNAKNTTYLQMNSLKPEDTAVYYCNAIPFRLYWGQGTQVTVSS

SEQ ID NO: 241 - protein, 115 aa, Artificial Sequence
  VHH
QVQLQESGGGLVQPGGSLRLSCLASGFTFSTYWMSWVRQAPGKGPEWVSGIDNGGGTTTYADSVKGRFTISRDNAKNTLNLEMNNLKPEDTAGYYSKAIPFRLSWGQGTQVTVSS

SEQ ID NO: 242 - protein, 114 aa, Artificial Sequence
  VHH
QVQLQESGGGLVQPGGSLRLSCAASGSSFSNNAMGWYRQAPGKQRELVAFITKGGVTDYSDSVRGRFTISRDNAKNTVYLQMNSLKPEDTAVYYCNTIPFRSAWGQETQVTVSS

SEQ ID NO: 243 - protein, 115 aa, Artificial Sequence
  VHH
QVQLQQSGGGLVQPGGSLRLSCLASGFTFSTYWMSWVRQAPGKGPEWVSGIDNGGGTTTYADSVKGRFTISRDNAKNTVYLQMNSLKPEDTAVYYCNTIPFRSAWGQGTQVTVAS

SEQ ID NO: 244 - protein, 114 aa, Artificial Sequence
  VHH
QVQLQESGGGLVQPGGSLRLSCAAPGNIFRLNATGWYRQVPGKQRELVAFITKGGVTEHSDSVEGRFTISKDNAKNTVYLQMNSLKPEDTAVYYCNTIPFRSAWGQGTQVTVSS

SEQ ID NO: 245 - protein, 114 aa, Artificial Sequence
  VHH
QVQLQESGGGLVQTGGSLRLSCAASRSSFSNNAMGWYRQVPGKQRELVAFITKGGVTDYSDSVKGRFTISKDNAKNTVYLQMNSLKPEDTAVYYCNTIPFRSAWGQGTQVTVSS

SEQ ID NO: 246 - protein, 114 aa, Artificial Sequence
  VHH
QVQLQESGGGLVQAGGSLRLSCAASGSSFSNNAMGWYRQVPGKQRELVAFITKGGVTDYSDSVKGRFTISKDNAKNTVYLQMNSLKPEDTAVYYCNTIPFRSAWGQGTQVTVSS

SEQ ID NO: 247 - protein, 114 aa, Artificial Sequence
  VHH
QVQLQESGGGLVQAGGSLRLSCAASRSSFGNNAMGWYRQVPGKQRELVAFITKGGVTDYSDSVKGRFTISKDNAKNTVYLQMNSLKPEDTAVYYCNTIPFRSAWGQGTQVTVSS

SEQ ID NO: 248 - protein, 121 aa, Artificial Sequence
  VHH
QVQLQESRGGLVQAGGSLRLSCAASGTIERNNAMAWYRQAPGKQRELVAIITSGGSTNYSDSVKGRFTISRDNAKNTLYLQMNSLKPEDTAVYLWAAGRRFSTRSRDYDYWGQGTQVTVSS

SEQ ID NO: 249 - protein, 122 aa, Artificial Sequence
  VHH
QVQLQESGGGLVQAGGSLSLSCTASGRSFSNYGMGWFRQAPGKEREFVAAIGMVGGLTAYSNSAKGRFTISRDNAKNTVYLQMNSLKPEDTAVYLCAAGRRFSTSSRDYDYWGQGTQVTVSS

SEQ ID NO: 250 - protein, 124 aa, Artificial Sequence
  VHH
QVQLQESGGGLVQAGDSLRLSCAASGRTFSSYAMGWFRQAPGKEREFVAAISRIGGVTTYAGSVQGRFTISRDNAKNTLYLRMNALKPEDTAVYYCAAAGLVSISTTPNDYDYWGQGTQVTVSS

SEQ ID NO: 251 - protein, 122 aa, Artificial Sequence
  VHH
QVQLQESGGGLVQAGGSLSLSCTASGRSFSNYGMGWFRQAPGKEREFVDAIGMVGGLTAYSNSAKGRFTISRDNAKNTLYLQMNSLKPEDTAVYLCAAGRRFSTGSRDYDIWGQGTQVTVSS

SEQ ID NO: 252 - protein, 122 aa, Artificial Sequence
  VHH
QVQLQQSGGGLVQTGGSLRLSCAASGRTFSSYAMGWFRQAPGKEREFVAAISQFGGVTTYADSVQGRFTISRDNAKNTLYLRMNSLKPEDTAVYLCAAGRRFSTGSRDYDIWGQGTQVTVSS

SEQ ID NO: 253 - protein, 122 aa, Artificial Sequence
  VHH
QVQLQESGGGLVQAGGSLSLSCTASGRSFSNYGMGWFRQAPGKEREFVAAIGMVGGLTAYSNSAKGRFTISRDKAKNTLYLQMNSLKPEDTAGYCGAGGRRFSTSSRDYDIWGQGTQVTVSS

SEQ ID NO: 254 - protein, 122 aa, Artificial Sequence
  VHH
QVQLQESGGGLVQAGGSLSLSCTASGRSFSNYGMGWFRQAPGKEREFVAAIGMVGGLTAYSNSAKGRFTISRDNAKNTLYLQMNSLKPEDTAVYLCAAGRRFSTSSREYDIWGQGTQVTVSS

SEQ ID NO: 255 - protein, 122 aa, Artificial Sequence
  VHH
QVQLQESGGGLVQAGGSLSLSCTASGRSFSNYGMGWFRQAPGKEREFVAAIGMVGGLPAYSNSAKGRFTISQDNAKNPLYLQINSLKPEETDVYLCAAGRRLSTSSRDYDIWGQGTQVTVSS

SEQ ID NO: 256 - protein, 122 aa, Artificial Sequence
  VHH
QVQLQESGGGLVQAGGSLSLSCTASGRSFSNYGMGWFRQAPGKEREFVAAISRGGGVSAYSNSAKGRFTISRDNAKNTVYLQMNSLKPEDAAVYFCAAGLRFSTGSRDYDIWGQGTQVTVSS

SEQ ID NO: 257 - protein, 123 aa, Artificial Sequence
  VHH
QVQLQQSGGGLVQAGDSLRLSCAASGRTFRRYAMGWFHQAPGKDREFVAGISRSGGMTGYADSVKGRFTISRDNAKNMVFLQMNSLKPEDTAVYYCAAYVGGFSTTRRDYAYWGQGTQVTVSS

SEQ ID NO: 258 - protein, 123 aa, Artificial Sequence
  VHH
QVQLQESGGGLVQAGDSLRLSCAASGRTFSNSTMGWFHQAPGKERKFVAVISWSGGTTAYAESVKGRFTISRDNAKNTVYLQMNSLKPEDTAVYYCAAARKGWSTRGDDYDYWGQGTQVTVSS

SEQ ID NO: 259 - protein, 118 aa, Artificial Sequence
  VHH
QVQLQESGGGLVQTGGSLRLSCAASGRTVIGWFRQAPGKEREFVAVSSGSGGVTAYASSVEGRFTISRDNVKNIMYLQMNSLKPEDTAIYYCAAALTWSTRPSDFTSWGQGTQVTVSS

SEQ ID NO: 260 - protein, 123 aa, Artificial Sequence
  VHH
QVQLQQSGGGLVQAGGSLRLSCAASGRTYNNYAMGWFRQAPGKEREFVAGISRSGGMTGYAESVKGRFTISRDNAKNMVCLQMNSLKPEDKAVYYCAAYVGGFSTARRDYSYWGQGTQVTVSS

SEQ ID NO: 261 - protein, 119 aa, Artificial Sequence
  VHH
QVQLQQSGGGLVQTGGSLRLSCAASGRTVIGWFRQAPGKEREFVAVSNWSGGVTAYASSVEGRFTISRDNVKNIMYFQMNSLKPEDTAVYYCAAYVGGFSTARRDYSYWGQGTQVTVSS

SEQ ID NO: 262 - protein, 123 aa, Artificial Sequence
  VHH
QVQLQQSGGGLVQPGGSLRLSCAASGRTYNNYAMGWFRQAPGKEREFVAGISRSGGMTGYAESVKGRFTISRDNAKNMVFLQMNSLKPEDTAVYYCAAYVGGFSTARRDYSYWGQGTQVTVSS

SEQ ID NO: 263 - protein, 123 aa, Artificial Sequence
  VHH
QVQLQESGGGLVQAGGSLIVSCATSGRTFSINAMGWFRQAPGKEREFVAGISRSGGMTGYAESVKGRFTISRDNAKNMVFLQMNSLKPEDTAVYYCAAYVGGFSTARRDYSYWGQGTQVSVSS

SEQ ID NO: 264 - protein, 122 aa, Artificial Sequence
  VHH
QVQLQQSGGGLVQAGGSLRLSCAASGRTFSRYAMGWFRQAPGKEREFVAVINGSGGTTAYANSVKGRFTITRDNAKNTLYLQMNSLKPEDTAVYYCAAARIFTTTRNEYDHWGQGTQVTVSS

SEQ ID NO: 265 - protein, 122 aa, Artificial Sequence
  VHH
QVQLQEPGGGLVQPGGSLRLSCAASGFTFSTYWMSWVHQAPGKGPEWVSGIDNGGGTTTYADSVKGRFTISRDNAKNTLYLQMNSLKPEETAIYYWAAARIFSTARNDYDHWGQGTQVTVSS

SEQ ID NO: 266 - protein, 122 aa, Artificial Sequence
  VHH
QVQLQESGGGLVQAGGSLSLSCTASGRSFSNYGMGWFRQAPGKEREFVAAIGMVGGLTAYSNSAKGRFTISRDNAKNTLYLQMNSLKPEDTAVYLVAGGRIFRTSSRDYDIWGQGTQVTVSS

SEQ ID NO: 267 - protein, 121 aa, Artificial Sequence
  VHH
QVQLQESGGGLVQPGGSLRLYCAAPGNIFRLNGTGWYRQAPGKQRELVTHITSGGNTDYADSVKGRFTISRDNAKNTVYLQMNSLKPEDTAIYYCAAARFFTTARNDYDHWGQGTQVTVSS

SEQ ID NO: 268 - protein, 117 aa, Artificial Sequence
  VHH
QVQLQESGGGLVQAGGSLRLSCAASGSIFSINAMGWYRQAPGKQRELVAAITSGGSTNYADSVKGRFTISRDNAKNTVYLQMNSLKPEDTAVYYCNALGGFVPNYGWGQGTQVTVSS

SEQ ID NO: 269 - protein, 117 aa, Artificial Sequence
  VHH
QVQLQESGGGLVQPGGSLRLSCAAPGNIFRLNGTGWYRQAPGKQRELVAHITSGGNTDYADSVKGRFTISRDNAKNTVYLQMNSLKPEDTAVYYCNALGGFVPNYGWGQGTQVTVSS

SEQ ID NO: 270 - protein, 117 aa, Artificial Sequence
  VHH
QVQLQESGGGLVQAGGSLRLSCAASGSIFSINAMGWYRQAPGKQRELVAAITSGGSTNYADSVKGRFTTSRDNAKNTVYLQMNSLKPEDPAVYYCNALGGFVPNDGWGQGTQVTVSS

SEQ ID NO: 271 - protein, 117 aa, Artificial Sequence
  VHH
QVQLQESGGGLVQAGGSLRLSCAASGNNFRLNAMGWYRQAPGKQRELVAHITSGGNTDYADSVKGRFTISRDNAKNTTYLQMNSLKPEDTAVYYCNALGGFAPNYGWGQGTQVTVSS

SEQ ID NO: 272 - protein, 118 aa, Artificial Sequence
  VHH
QVQLQQSGGGLVQAGGSLSLSCTASGRSFSNYGMGWFRQAPGKEREFVAAIGMVGGLTAYSNSAKGRFTISRDNAKNTVYLQMNSLKPEDTAVYYCNALGGFVPNYVWGQGTQVTVSS

SEQ ID NO: 273 - protein, 117 aa, Artificial Sequence
  VHH
QVQLQESGGGLVQAGGSLSLSCTASGRSFSINAMGWYRQAPGKQRELVAAITSGGSTNYADSVKGRFTISRDNAKNTVYLQMNSLKPEDTAVYYCNALGGFVLNYGWGQGTQVTVSS

SEQ ID NO: 274 - protein, 117 aa, Artificial Sequence
  VHH
QVQLQESGGGLVQAGGSLRLSCAASGSIFSINAMGWYRQAPGKQRELVAAITSGGSTNYADPVKGRFTISRDNAKNTVYLQMNSLKPEDTAVYYCNALGGLVPNYGWGQGTQVTVSS

SEQ ID NO: 275 - protein, 117 aa, Artificial Sequence
  VHH
QVQLQESGGGLVQAGGSLRLSCAASGSIFSINAMGWYRQAPGKQRELVAAITSGGSTNYADSVKGRFTISRDNAKNTVYLQMNSLKPEDTAVYYCNALGGFLPNYGWGQGTQVTVSS

SEQ ID NO: 276 - protein, 117 aa, Artificial Sequence
  VHH
QVQLQESGGGLVQAGGSLRLSCAASGSIFSINAMGWYRQAPGKQRELVAAITSGGSTNYADSVKGRFTISRDNAKNTVYLQMNSLKPEDTAVYYCNALGGFVTNYGWGQGTQVAVSS

SEQ ID NO: 277 - protein, 117 aa, Artificial Sequence
  VHH
QVQLQESGGGLVQPGGSLRLSCAASGSIFSINAMGWYRQAPGKQRELVAFITSGGSTNYADSVKGRFTISRDNAKNTVYLQMNSLKPEDTAVYYCNALGGFVPSYGWGQGTQVTVSS

SEQ ID NO: 278 - protein, 113 aa, Artificial Sequence
  VHH
QVQLQQSGGGLVQTGGSLRLSCAASGRTVMGWYRQAPGKQRELVAVITRGGSTNYADSVKGRFTISRDNAKNTVYLQMNSLKPEDTAFYYCNALGGFVPNYGWGQGTQVTVSS

SEQ ID NO: 279 - protein, 117 aa, Artificial Sequence
  VHH
QVQLQESGGGLVQPGGSLRLSCAAPGNIFRLNGTGWYRQAPGKQRELVAHITSGGNTDYADSVKGRFTISRDNAKNTVYLQMNSLKPEDTAVYYCNALGGFVPSYGWGQGTQVTVSS

SEQ ID NO: 280 - protein, 117 aa, Artificial Sequence
  VHH
QVQLQESGGGLVQPGGSLRLSCAAPGNNFRLNAMGWYRQAPGKQRELVAHITSGGNTDYADSVKGRFTISRDNAKNTVYLQMNSLKPEDTAVYYCNALGGFVPSYGWGQGTQVTVSS

SEQ ID NO: 281 - protein, 117 aa, Artificial Sequence
  VHH
QVQLQQSGGGLVQAGGSLRLSCAASGSIFSINAMGWYRQAPGKQRELVAFITSGGSTNYADSVKGRFTISRDNAKNTVYLQMNSLKPEDTAVYYCNALGGFVPSYGWGQGTQVTVSS

SEQ ID NO: 282 - protein, 117 aa, Artificial Sequence
  VHH
QVQLQQSGGGLVQPGGSLRLSCAAPGNIFRLNGTGWYRQAPGKQRELVAHITSGGSTNYADSVKGRFTISRDNAKNTVYLQMNSLKPEDTAVYYCNALGGFVPSYGWGQGTQVTVSS

SEQ ID NO: 283 - protein, 117 aa, Artificial Sequence
  VHH
QVQLQESGGGLVQAGGSLRLSCAASGSIFSINAMGWYRQAPGKQRELVAAITSGGSTNYADSVKGRFTISRDNAKNTVYLQMNSLKPEDTAVYYCNALGGFVPSYGWGQGTQVTVSS

SEQ ID NO: 284 - protein, 118 aa, Artificial Sequence
  VHH
QVQLQESGGGLVQAGGSLSLSCTASGRSFSNYGMGWFRQAPGKEREFVAAIGMVGGLTAYSNSAKGRFTISRDNAKNTLYLQMNSLKPEDTAVYYCNALGGFVPSYGWGQGTQVTVSS

SEQ ID NO: 285 - protein, 117 aa, Artificial Sequence
  VHH
QVQLQESGGGLVQAGGSLRLSCAASGSIFSINAMGWYRQAPGKQRELVAFITSGGSTNYADSVKGRFTISRDNAKNTVYLQMNSLKPEDTAVYYCNALGGFVPSYGWGQGTLVTVSS

SEQ ID NO: 286 - protein, 117 aa, Artificial Sequence
  VHH
QVQLQESGGGLVQPGGSLRLSCAAPGNIFSINAMGWYRQAPGKQRELVAFITSGGSTNYADSVKGRFTISRDNAKNTVYLQMNSLKPEDTAVYYCNALGGFVPSYGWGQGTQVTVSS

SEQ ID NO: 287 - protein, 120 aa, Artificial Sequence
  VHH
QVQLQESGGGLVQPGGSLRLSCLASGFTFSTYWMSWVRQAPGKGPEWVSGIDNGGGTTTYADSVKGRFTISRDNAKNTLNLEMNNLKPEDTALYYCARNPTRGWYSTDYRGQGTQVTVSS

SEQ ID NO: 288 - protein, 120 aa, Artificial Sequence
  VHH
QVQLQESGGGLVQPGGSLRLSCAASGFTFSTYWMSWVRQAPGKGPEWVSGIDNGGGTTTYADSVKGRFTISRDNAKHTLYLQMNTLKPEDTALYYCARNPTRGWYSTDYRGQGTQVTVSS

SEQ ID NO: 289 - protein, 528 aa, Artificial Sequence
  CAR
MGTSLLCWMALCLLGADHADACPYSNPSLCSGGGGSELPTQGTFSNVSTNVSPAKPTTTACPYSNPSLCSGGGGSPAPRPPTPAPTIASQPLSLRPEACRPAAGGAVHTRGLDFACDIYIWAPLAGTCGVLLLSLVITLYCNHRNRRRVCKCPRPVVRAEGRGSLLTCGDVEENPGPMGWSCIILFLVATATGVHSQVQLQESGGGLVQPGGSLRLSCAAPGNIFRLNGTGWYRQAPGKQRELVAHITSGGNTDYADSVKGRFTISRDNAKNTVYLQMNSLKPEDTAVYYCNTIPFRLSWGQGTQVTVSSDPTPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 290 - protein, 528 aa, Artificial Sequence
  CAR
MGTSLLCWMALCLLGADHADACPYSNPSLCSGGGGSELPTQGTFSNVSTNVSPAKPTTTACPYSNPSLCSGGGGSPAPRPPTPAPTIASQPLSLRPEACRPAAGGAVHTRGLDFACDIYIWAPLAGTCGVLLLSLVITLYCNHRNRRRVCKCPRPVVRAEGRGSLLTCGDVEENPGPMGWSCIILFLVATATGVHSQVQLQESGGGLVQPGGSLRLSCAAPGNIFRLNAMGWYRQAPGKQRELVAHITSGGNTDYADSVKGRFTISRDNAKNTVYLQMNSLKLEDTAVYYCNTIPFRLSWGQGTQVTVSSDPTPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 291 - protein, 528 aa, Artificial Sequence
  CAR
MGTSLLCWMALCLLGADHADACPYSNPSLCSGGGGSELPTQGTFSNVSTNVSPAKPTTTACPYSNPSLCSGGGGSPAPRPPTPAPTIASQPLSLRPEACRPAAGGAVHTRGLDFACDIYIWAPLAGTCGVLLLSLVITLYCNHRNRRRVCKCPRPVVRAEGRGSLLTCGDVEENPGPMGWSCIILFLVATATGVHSQVQLQESGGGLAQAGGSLRLSCAASRSSFSNNAMGWYRQVQGKQRELVAFITKGGVTDYSVSGKGRFTISKDHAKNTVYLQMNSLKPRDTAVYYCNTIPVRSAWGQGTQVTVSADPTPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 292 - protein, 528 aa, Artificial Sequence
  CAR
MGTSLLCWMALCLLGADHADACPYSNPSLCSGGGGSELPTQGTFSNVSTNVSPAKPTTTACPYSNPSLCSGGGGSPAPRPPTPAPTIASQPLSLRPEACRPAAGGAVHTRGLDFACDIYIWAPLAGTCGVLLLSLVITLYCNHRNRRRVCKCPRPVVRAEGRGSLLTCGDVEENPGPMGWSCIILFLVATATGVHSQVQLQESGGGLVQAGGSLRLSCAASGSIFSINAMGWYRQAPGKQRELVAFITKGGVTDYSDSVKGRFTISKDNAKNTVYLQMNSLKPEDTAVYYCNTIPCRSAWGQGTQVTVSSDPTPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 293 - protein, 528 aa, Artificial Sequence
  CAR
MGTSLLCWMALCLLGADHADACPYSNPSLCSGGGGSELPTQGTFSNVSTNVSPAKPTTTACPYSNPSLCSGGGGSPAPRPPTPAPTIASQPLSLRPEACRPAAGGAVHTRGLDFACDIYIWAPLAGTCGVLLLSLVITLYCNHRNRRRVCKCPRPVVRAEGRGSLLTCGDVEENPGPMGWSCIILFLVATATGVHSQVQLQESGGGLVQPGGSLRLSCAAPGSSFRLNGTGWYRQAPGKQRELVAHITSGGSTNYSDSVKGRLTISKDNAKNTVYLQMNSLKPEDTAVYYCNTIPFSRAWGQGTLVTVSSDPTPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 294 - protein, 528 aa, Artificial Sequence
  CAR
MGTSLLCWMALCLLGADHADACPYSNPSLCSGGGGSELPTQGTFSNVSTNVSPAKPTTTACPYSNPSLCSGGGGSPAPRPPTPAPTIASQPLSLRPEACRPAAGGAVHTRGLDFACDIYIWAPLAGTCGVLLLSLVITLYCNHRNRRRVCKCPRPVVRAEGRGSLLTCGDVEENPGPMGWSCIILFLVATATGVHSQVQLQQSGGGLVQAGGSLRLSCAASGRSVSINAMGWYRQAPGKQRELVAIIDRSGNTDYADSVKGRFTISRDNAKKAVYLQMNSLKPEDTAVYYCNTIPYSDSWGQGTQVTISSDPTPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 295 - protein, 529 aa, Artificial Sequence
  CAR
MGTSLLCWMALCLLGADHADACPYSNPSLCSGGGGSELPTQGTFSNVSTNVSPAKPTTTACPYSNPSLCSGGGGSPAPRPPTPAPTIASQPLSLRPEACRPAAGGAVHTRGLDFACDIYIWAPLAGTCGVLLLSLVITLYCNHRNRRRVCKCPRPVVRAEGRGSLLTCGDVEENPGPMGWSCIILFLVATATGVHSQVQLQESGGGLVQPGGSLRLSCAASGRTFSSYAMGWFRRAPGKGREFVATIDGIGGITSYAGSVKGRFTVSKDNAKNTVYLQMNSLKPEDTAVYYCNTIPFRSAWGQGTLVTVSSDPTPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 296 - protein, 528 aa, Artificial Sequence
  CAR
MGTSLLCWMALCLLGADHADACPYSNPSLCSGGGGSELPTQGTFSNVSTNVSPAKPTTTACPYSNPSLCSGGGGSPAPRPPTPAPTIASQPLSLRPEACRPAAGGAVHTRGLDFACDIYIWAPLAGTCGVLLLSLVITLYCNHRNRRRVCKCPRPVVRAEGRGSLLTCGDVEENPGPMGWSCIILFLVATATGVHSQVQLQESGGGLVQAGGSLRLSCAASRSSFSNNAMGWYRQGPGKQRELVAFITKGGVTDYSDSVKGRFTISKDNAKNTVYLQMNSLKPEDTAVYYCNTIPFRSAWGQGTQVTVSSDPTPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 297 - protein, 528 aa, Artificial Sequence
  CAR
MGTSLLCWMALCLLGADHADACPYSNPSLCSGGGGSELPTQGTFSNVSTNVSPAKPTTTACPYSNPSLCSGGGGSPAPRPPTPAPTIASQPLSLRPEACRPAAGGAVHTRGLDFACDIYIWAPLAGTCGVLLLSLVITLYCNHRNRRRVCKCPRPVVRAEGRGSLLTCGDVEENPGPMGWSCIILFLVATATGVHSQVQLQESGGGLVQAGGSLRLSCAASRSSFSNNAMGWYRQVPGKQRELVAFITKGGVTDYSDSVKGRFTISKDNAKNTVYLQMNSLKPEDTAVYYCNAIPFRSAWGQGTQVTVSSDPTPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 298 - protein, 528 aa, Artificial Sequence
  CAR
MGTSLLCWMALCLLGADHADACPYSNPSLCSGGGGSELPTQGTFSNVSTNVSPAKPTTTACPYSNPSLCSGGGGSPAPRPPTPAPTIASQPLSLRPEACRPAAGGAVHTRGLDFACDIYIWAPLAGTCGVLLLSLVITLYCNHRNRRRVCKCPRPVVRAEGRGSLLTCGDVEENPGPMGWSCIILFLVATATGVHSQVQLQESGGGLVQPGGSLRLSCAAPGNNFRLNAMGWYRQAPGKQRELVAHITSGGNTDYADSVKGRFTISRDNAKNTVYLQMNSLKPEDTAVYYCNAIPFRPSWGQGTQVTVSSDPTPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 299 - protein, 528 aa, Artificial Sequence
  CAR
MGTSLLCWMALCLLGADHADACPYSNPSLCSGGGGSELPTQGTFSNVSTNVSPAKPTTTACPYSNPSLCSGGGGSPAPRPPTPAPTIASQPLSLRPEACRPAAGGAVHTRGLDFACDIYIWAPLAGTCGVLLLSLVITLYCNHRNRRRVCKCPRPVVRAEGRGSLLTCGDVEENPGPMGWSCIILFLVATATGVHSQVQLQESGGGLVQPGGSLRLSCAAPGNIFRLNGTGWYRQAPGKQRELVAHITSGGNTDYADSGKGRFTISRDNAKNTVYLQMNSLKPEDTAVYYCNAIPFRRSWGQGTQVTVSSDPTPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 300 - protein, 528 aa, Artificial Sequence
  CAR
MGTSLLCWMALCLLGADHADACPYSNPSLCSGGGGSELPTQGTFSNVSTNVSPAKPTTTACPYSNPSLCSGGGGSPAPRPPTPAPTIASQPLSLRPEACRPAAGGAVHTRGLDFACDIYIWAPLAGTCGVLLLSLVITLYCNHRNRRRVCKCPRPVVRAEGRGSLLTCGDVEENPGPMGWSCIILFLVATATGVHSQVQLQESGGGLVQAGGSLKLSCAASERIFRINAMGWYRQAPGKQRELVAHITSGGNTDYADSVKGRFTISRDNAKNTMYLQMNSLKPEDTAVYYCNAIPFRRSWGQGTQVTVSSDPTPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 301 - protein, 528 aa, Artificial Sequence
  CAR
MGTSLLCWMALCLLGADHADACPYSNPSLCSGGGGSELPTQGTFSNVSTNVSPAKPTTTACPYSNPSLCSGGGGSPAPRPPTPAPTIASQPLSLRPEACRPAAGGAVHTRGLDFACDIYIWAPLAGTCGVLLLSLVITLYCNHRNRRRVCKCPRPVVRAEGRGSLLTCGDVEENPGPMGWSCIILFLVATATGVHSQVQLQQSGGGLVQPGGSLRLSCAAPGNIFRLNGTGWYRQAPGKQRELVAHITSGGNTDYADSVKGRFTISRDNAENTVYLQMNSLKPEDTAVYYCNAVPFRLSWGQGTQVTVSSDPTPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 302 - protein, 529 aa, Artificial Sequence
  VHH
MGTSLLCWMALCLLGADHADACPYSNPSLCSGGGGSELPTQGTFSNVSTNVSPAKPTTTACPYSNPSLCSGGGGSPAPRPPTPAPTIASQPLSLRPEACRPAAGGAVHTRGLDFACDIYIWAPLAGTCGVLLLSLVITLYCNHRNRRRVCKCPRPVVRAEGRGSLLTCGDVEENPGPMGWSCIILFLVATATGVHSQVQLQESGGGLVQPGESLRLSCAASGFTFSTYWMSWVRQAPGKGPEGVSGIDNGGGTTTYADSVKGRFTISRDNAGNTVYLQMNSLKPEDTAVYYCNALPFRLSWGQGTQVTVSSDPTPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 303 - protein, 528 aa, Artificial Sequence
  VHH
MGTSLLCWMALCLLGADHADACPYSNPSLCSGGGGSELPTQGTFSNVSTNVSPAKPTTTACPYSNPSLCSGGGGSPAPRPPTPAPTIASQPLSLRPEACRPAAGGAVHTRGLDFACDIYIWAPLAGTCGVLLLSLVITLYCNHRNRRRVCKCPRPVVRAEGRGSLLTCGDVEENPGPMGWSCIILFLVATATGVHSQVQLQESGGGLVQPGGSLRLSCAAPGNIFRLNGTGWYRQAPGKQGELVAIITSGGNTDYADSVKGRFTISRDNAKNTTYLQMNSLKPEDTAVYYCNATPFRLSWGQGTQVTVSSDPTPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 304 - protein, 528 aa, Artificial Sequence
  CAR
MGTSLLCWMALCLLGADHADACPYSNPSLCSGGGGSELPTQGTFSNVSTNVSPAKPTTTACPYSNPSLCSGGGGSPAPRPPTPAPTIASQPLSLRPEACRPAAGGAVHTRGLDFACDIYIWAPLAGTCGVLLLSLVITLYCNHRNRRRVCKCPRPVVRAEGRGSLLTCGDVEENPGPMGWSCIILFLVATATGVHSQVQLQESGGGLVQPGGSLRLSCAAPGNIFRLNGTGWYRQAPGKQRELVAHITRGGNTDYADAVKGRFTISRDNAKNTVYLQMNSLKPEDTAVYYCNSIPFRLSWGQGTQVTVSSDPTPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 305 - protein, 528 aa, Artificial Sequence
  VHH
MGTSLLCWMALCLLGADHADACPYSNPSLCSGGGGSELPTQGTFSNVSTNVSPAKPTTTACPYSNPSLCSGGGGSPAPRPPTPAPTIASQPLSLRPEACRPAAGGAVHTRGLDFACDIYIWAPLAGTCGVLLLSLVITLYCNHRNRRRVCKCPRPVVRAEGRGSLLTCGDVEENPGPMGWSCIILFLVATATGVHSQVQLQESGGGLVQPGGSLRLSCAAPGNIFRINGTGWYGQAPGKQRELVAHITSGGNTDYEDSVKGRFTISRDNAKNTTYLQMNSLKPEDTAVYYCNAIPFRISWGQGTQVTVSSDPTPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 306 - protein, 528 aa, Artificial Sequence
  VHH
MGTSLLCWMALCLLGADHADACPYSNPSLCSGGGGSELPTQGTFSNVSTNVSPAKPTTTACPYSNPSLCSGGGGSPAPRPPTPAPTIASQPLSLRPEACRPAAGGAVHTRGLDFACDIYIWAPLAGTCGVLLLSLVITLYCNHRNRRRVCKCPRPVVRAEGRGSLLTCGDVEENPGPMGWSCIILFLVATATGVHSQVQLQQSGGGLVQPGGSLRLSCAAPGNNFRLNAMGWYRQAPGKQRELVAHITSGGNTDYADSVKGRFTISRDNAKNTTYLQMNSLKPEDTAVYYCNAIPFRLYWGQGTQVTVSSDPTPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 307 - protein, 529 aa, Artificial Sequence
  VHH
MGTSLLCWMALCLLGADHADACPYSNPSLCSGGGGSELPTQGTFSNVSTNVSPAKPTTTACPYSNPSLCSGGGGSPAPRPPTPAPTIASQPLSLRPEACRPAAGGAVHTRGLDFACDIYIWAPLAGTCGVLLLSLVITLYCNHRNRRRVCKCPRPVVRAEGRGSLLTCGDVEENPGPMGWSCIILFLVATATGVHSQVQLQESGGGLVQPGGSLRLSCLASGFTFSTYWMSWVRQAPGKGPEWVSGIDNGGGTTTYADSVKGRFTISRDNAKNTLNLEMNNLKPEDTAGYYSKAIPFRLSWGQGTQVTVSSDPTPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 308 - protein, 528 aa, Artificial Sequence
  VHH
MGTSLLCWMALCLLGADHADACPYSNPSLCSGGGGSELPTQGTFSNVSTNVSPAKPTTTACPYSNPSLCSGGGGSPAPRPPTPAPTIASQPLSLRPEACRPAAGGAVHTRGLDFACDIYIWAPLAGTCGVLLLSLVITLYCNHRNRRRVCKCPRPVVRAEGRGSLLTCGDVEENPGPMGWSCIILFLVATATGVHSQVQLQESGGGLVQPGGSLRLSCAASGSSFSNNAMGWYRQAPGKQRELVAFITKGGVTDYSDSVRGRFTISRDNAKNTVYLQMNSLKPEDTAVYYCNTIPFRSAWGQETQVTVSSDPTPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 309 - protein, 529 aa, Artificial Sequence
  VHH
MGTSLLCWMALCLLGADHADACPYSNPSLCSGGGGSELPTQGTFSNVSTNVSPAKPTTTACPYSNPSLCSGGGGSPAPRPPTPAPTIASQPLSLRPEACRPAAGGAVHTRGLDFACDIYIWAPLAGTCGVLLLSLVITLYCNHRNRRRVCKCPRPVVRAEGRGSLLTCGDVEENPGPMGWSCIILFLVATATGVHSQVQLQQSGGGLVQPGGSLRLSCLASGFTFSTYWMSWVRQAPGKGPEWVSGIDNGGGTTTYADSVKGRFTISRDNAKNTVYLQMNSLKPEDTAVYYCNTIPFRSAWGQGTQVTVASDPTPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 310 - protein, 528 aa, Artificial Sequence
  VHH
MGTSLLCWMALCLLGADHADACPYSNPSLCSGGGGSELPTQGTFSNVSTNVSPAKPTTTACPYSNPSLCSGGGGSPAPRPPTPAPTIASQPLSLRPEACRPAAGGAVHTRGLDFACDIYIWAPLAGTCGVLLLSLVITLYCNHRNRRRVCKCPRPVVRAEGRGSLLTCGDVEENPGPMGWSCIILFLVATATGVHSQVQLQESGGGLVQPGGSLRLSCAAPGNIFRLNATGWYRQVPGKQRELVAFITKGGVTEHSDSVEGRFTISKDNAKNTVYLQMNSLKPEDTAVYYCNTIPFRSAWGQGTQVTVSSDPTPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 311 - protein, 528 aa, Artificial Sequence
  VHH
MGTSLLCWMALCLLGADHADACPYSNPSLCSGGGGSELPTQGTFSNVSTNVSPAKPTTTACPYSNPSLCSGGGGSPAPRPPTPAPTIASQPLSLRPEACRPAAGGAVHTRGLDFACDIYIWAPLAGTCGVLLLSLVITLYCNHRNRRRVCKCPRPVVRAEGRGSLLTCGDVEENPGPMGWSCIILFLVATATGVHSQVQLQESGGGLVQTGGSLRLSCAASRSSFSNNAMGWYRQVPGKQRELVAFITKGGVTDYSDSVKGRFTISKDNAKNTVYLQMNSLKPEDTAVYYCNTIPFRSAWGQGTQVTVSSDPTPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 312 - protein, 528 aa, Artificial Sequence
  VHH
MGTSLLCWMALCLLGADHADACPYSNPSLCSGGGGSELPTQGTFSNVSTNVSPAKPTTTACPYSNPSLCSGGGGSPAPRPPTPAPTIASQPLSLRPEACRPAAGGAVHTRGLDFACDIYIWAPLAGTCGVLLLSLVITLYCNHRNRRRVCKCPRPVVRAEGRGSLLTCGDVEENPGPMGWSCIILFLVATATGVHSQVQLQESGGGLVQAGGSLRLSCAASGSSFSNNAMGWYRQVPGKQRELVAFITKGGVTDYSDSVKGRFTISKDNAKNTVYLQMNSLKPEDTAVYYCNTIPFRSAWGQGTQVTVSSDPTPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 313 - protein, 528 aa, Artificial Sequence
  VHH
MGTSLLCWMALCLLGADHADACPYSNPSLCSGGGGSELPTQGTFSNVSTNVSPAKPTTTACPYSNPSLCSGGGGSPAPRPPTPAPTIASQPLSLRPEACRPAAGGAVHTRGLDFACDIYIWAPLAGTCGVLLLSLVITLYCNHRNRRRVCKCPRPVVRAEGRGSLLTCGDVEENPGPMGWSCIILFLVATATGVHSQVQLQESGGGLVQAGGSLRLSCAASRSSFGNNAMGWYRQVPGKQRELVAFITKGGVTDYSDSVKGRFTISKDNAKNTVYLQMNSLKPEDTAVYYCNTIPFRSAWGQGTQVTVSSDPTPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 314 - protein, 535 aa, Artificial Sequence
  VHH
MGTSLLCWMALCLLGADHADACPYSNPSLCSGGGGSELPTQGTFSNVSTNVSPAKPTTTACPYSNPSLCSGGGGSPAPRPPTPAPTIASQPLSLRPEACRPAAGGAVHTRGLDFACDIYIWAPLAGTCGVLLLSLVITLYCNHRNRRRVCKCPRPVVRAEGRGSLLTCGDVEENPGPMGWSCIILFLVATATGVHSQVQLQESRGGLVQAGGSLRLSCAASGTIERNNAMAWYRQAPGKQRELVAIITSGGSTNYSDSVKGRFTISRDNAKNTLYLQMNSLKPEDTAVYLWAAGRRFSTRSRDYDYWGQGTQVTVSSDPTPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 315 - protein, 536 aa, Artificial Sequence
  VHH
MGTSLLCWMALCLLGADHADACPYSNPSLCSGGGGSELPTQGTFSNVSTNVSPAKPTTTACPYSNPSLCSGGGGSPAPRPPTPAPTIASQPLSLRPEACRPAAGGAVHTRGLDFACDIYIWAPLAGTCGVLLLSLVITLYCNHRNRRRVCKCPRPVVRAEGRGSLLTCGDVEENPGPMGWSCIILFLVATATGVHSQVQLQESGGGLVQAGGSLSLSCTASGRSFSNYGMGWFRQAPGKEREFVAAIGMVGGLTAYSNSAKGRFTISRDNAKNTVYLQMNSLKPEDTAVYLCAAGRRFSTSSRDYDYWGQGTQVTVSSDPTPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 316 - protein, 538 aa, Artificial Sequence
  VHH
MGTSLLCWMALCLLGADHADACPYSNPSLCSGGGGSELPTQGTFSNVSTNVSPAKPTTTACPYSNPSLCSGGGGSPAPRPPTPAPTIASQPLSLRPEACRPAAGGAVHTRGLDFACDIYIWAPLAGTCGVLLLSLVITLYCNHRNRRRVCKCPRPVVRAEGRGSLLTCGDVEENPGPMGWSCIILFLVATATGVHSQVQLQESGGGLVQAGDSLRLSCAASGRTFSSYAMGWFRQAPGKEREFVAAISRIGGVTTYAGSVQGRFTISRDNAKNTLYLRMNALKPEDTAVYYCAAAGLVSISTTPNDYDYWGQGTQVTVSSDPTPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 317 - protein, 536 aa, Artificial Sequence
  VHH
MGTSLLCWMALCLLGADHADACPYSNPSLCSGGGGSELPTQGTFSNVSTNVSPAKPTTTACPYSNPSLCSGGGGSPAPRPPTPAPTIASQPLSLRPEACRPAAGGAVHTRGLDFACDIYIWAPLAGTCGVLLLSLVITLYCNHRNRRRVCKCPRPVVRAEGRGSLLTCGDVEENPGPMGWSCIILFLVATATGVHSQVQLQESGGGLVQAGGSLSLSCTASGRSFSNYGMGWFRQAPGKEREFVDAIGMVGGLTAYSNSAKGRFTISRDNAKNTLYLQMNSLKPEDTAVYLCAAGRRFSTGSRDYDIWGQGTQVTVSSDPTPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 318 - protein, 536 aa, Artificial Sequence
  VHH
MGTSLLCWMALCLLGADHADACPYSNPSLCSGGGGSELPTQGTFSNVSTNVSPAKPTTTACPYSNPSLCSGGGGSPAPRPPTPAPTIASQPLSLRPEACRPAAGGAVHTRGLDFACDIYIWAPLAGTCGVLLLSLVITLYCNHRNRRRVCKCPRPVVRAEGRGSLLTCGDVEENPGPMGWSCIILFLVATATGVHSQVQLQQSGGGLVQTGGSLRLSCAASGRTFSSYAMGWFRQAPGKEREFVAAISQFGGVTTYADSVQGRFTISRDNAKNTLYLRMNSLKPEDTAVYLCAAGRRFSTGSRDYDIWGQGTQVTVSSDPTPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 319 - protein, 536 aa, Artificial Sequence
  VHH
MGTSLLCWMALCLLGADHADACPYSNPSLCSGGGGSELPTQGTFSNVSTNVSPAKPTTTACPYSNPSLCSGGGGSPAPRPPTPAPTIASQPLSLRPEACRPAAGGAVHTRGLDFACDIYIWAPLAGTCGVLLLSLVITLYCNHRNRRRVCKCPRPVVRAEGRGSLLTCGDVEENPGPMGWSCIILFLVATATGVHSQVQLQESGGGLVQAGGSLSLSCTASGRSFSNYGMGWFRQAPGKEREFVAAIGMVGGLTAYSNSAKGRFTISRDKAKNTLYLQMNSLKPEDTAGYCGAGGRRFSTSSRDYDIWGQGTQVTVSSDPTPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 320 - protein, 536 aa, Artificial Sequence
  VHH
MGTSLLCWMALCLLGADHADACPYSNPSLCSGGGGSELPTQGTFSNVSTNVSPAKPTTTACPYSNPSLCSGGGGSPAPRPPTPAPTIASQPLSLRPEACRPAAGGAVHTRGLDFACDIYIWAPLAGTCGVLLLSLVITLYCNHRNRRRVCKCPRPVVRAEGRGSLLTCGDVEENPGPMGWSCIILFLVATATGVHSQVQLQESGGGLVQAGGSLSLSCTASGRSFSNYGMGWFRQAPGKEREFVAAIGMVGGLTAYSNSAKGRFTISRDNAKNTLYLQMNSLKPEDTAVYLCAAGRRFSTSSREYDIWGQGTQVTVSSDPTPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 321 - protein, 536 aa, Artificial Sequence
  VHH
MGTSLLCWMALCLLGADHADACPYSNPSLCSGGGGSELPTQGTFSNVSTNVSPAKPTTTACPYSNPSLCSGGGGSPAPRPPTPAPTIASQPLSLRPEACRPAAGGAVHTRGLDFACDIYIWAPLAGTCGVLLLSLVITLYCNHRNRRRVCKCPRPVVRAEGRGSLLTCGDVEENPGPMGWSCIILFLVATATGVHSQVQLQESGGGLVQAGGSLSLSCTASGRSFSNYGMGWFRQAPGKEREFVAAIGMVGGLPAYSNSAKGRFTISQDNAKNPLYLQINSLKPEETDVYLCAAGRRLSTSSRDYDIWGQGTQVTVSSDPTPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 322 - protein, 536 aa, Artificial Sequence
  VHH
MGTSLLCWMALCLLGADHADACPYSNPSLCSGGGGSELPTQGTFSNVSTNVSPAKPTTTACPYSNPSLCSGGGGSPAPRPPTPAPTIASQPLSLRPEACRPAAGGAVHTRGLDFACDIYIWAPLAGTCGVLLLSLVITLYCNHRNRRRVCKCPRPVVRAEGRGSLLTCGDVEENPGPMGWSCIILFLVATATGVHSQVQLQESGGGLVQAGGSLSLSCTASGRSFSNYGMGWFRQAPGKEREFVAAISRGGGVSAYSNSAKGRFTISRDNAKNTVYLQMNSLKPEDAAVYFCAAGLRFSTGSRDYDIWGQGTQVTVSSDPTPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 323 - protein, 537 aa, Artificial Sequence
  VHH
MGTSLLCWMALCLLGADHADACPYSNPSLCSGGGGSELPTQGTFSNVSTNVSPAKPTTTACPYSNPSLCSGGGGSPAPRPPTPAPTIASQPLSLRPEACRPAAGGAVHTRGLDFACDIYIWAPLAGTCGVLLLSLVITLYCNHRNRRRVCKCPRPVVRAEGRGSLLTCGDVEENPGPMGWSCIILFLVATATGVHSQVQLQQSGGGLVQAGDSLRLSCAASGRTFRRYAMGWFHQAPGKDREFVAGISRSGGMTGYADSVKGRFTISRDNAKNMVFLQMNSLKPEDTAVYYCAAYVGGFSTTRRDYAYWGQGTQVTVSSDPTPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 324 - protein, 537 aa, Artificial Sequence
  VHH
MGTSLLCWMALCLLGADHADACPYSNPSLCSGGGGSELPTQGTFSNVSTNVSPAKPTTTACPYSNPSLCSGGGGSPAPRPPTPAPTIASQPLSLRPEACRPAAGGAVHTRGLDFACDIYIWAPLAGTCGVLLLSLVITLYCNHRNRRRVCKCPRPVVRAEGRGSLLTCGDVEENPGPMGWSCIILFLVATATGVHSQVQLQESGGGLVQAGDSLRLSCAASGRTFSNSTMGWFHQAPGKERKFVAVISWSGGTTAYAESVKGRFTISRDNAKNTVYLQMNSLKPEDTAVYYCAAARKGWSTRGDDYDYWGQGTQVTVSSDPTPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 325 - protein, 532 aa, Artificial Sequence
  VHH
MGTSLLCWMALCLLGADHADACPYSNPSLCSGGGGSELPTQGTFSNVSTNVSPAKPTTTACPYSNPSLCSGGGGSPAPRPPTPAPTIASQPLSLRPEACRPAAGGAVHTRGLDFACDIYIWAPLAGTCGVLLLSLVITLYCNHRNRRRVCKCPRPVVRAEGRGSLLTCGDVEENPGPMGWSCIILFLVATATGVHSQVQLQESGGGLVQTGGSLRLSCAASGRTVIGWFRQAPGKEREFVAVSSGSGGVTAYASSVEGRFTISRDNVKNIMYLQMNSLKPEDTAIYYCAAALTWSTRPSDFTSWGQGTQVTVSSDPTPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 326 - protein, 537 aa, Artificial Sequence
  VHH
MGTSLLCWMALCLLGADHADACPYSNPSLCSGGGGSELPTQGTFSNVSTNVSPAKPTTTACPYSNPSLCSGGGGSPAPRPPTPAPTIASQPLSLRPEACRPAAGGAVHTRGLDFACDIYIWAPLAGTCGVLLLSLVITLYCNHRNRRRVCKCPRPVVRAEGRGSLLTCGDVEENPGPMGWSCIILFLVATATGVHSQVQLQQSGGGLVQAGGSLRLSCAASGRTYNNYAMGWFRQAPGKEREFVAGISRSGGMTGYAESVKGRFTISRDNAKNMVCLQMNSLKPEDKAVYYCAAYVGGFSTARRDYSYWGQGTQVTVSSDPTPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 327 - protein, 533 aa, Artificial Sequence
  VHH
MGTSLLCWMALCLLGADHADACPYSNPSLCSGGGGSELPTQGTFSNVSTNVSPAKPTTTACPYSNPSLCSGGGGSPAPRPPTPAPTIASQPLSLRPEACRPAAGGAVHTRGLDFACDIYIWAPLAGTCGVLLLSLVITLYCNHRNRRRVCKCPRPVVRAEGRGSLLTCGDVEENPGPMGWSCIILFLVATATGVHSQVQLQQSGGGLVQTGGSLRLSCAASGRTVIGWFRQAPGKEREFVAVSNWSGGVTAYASSVEGRFTISRDNVKNIMYFQMNSLKPEDTAVYYCAAYVGGFSTARRDYSYWGQGTQVTVSSDPTPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 328 - protein, 537 aa, Artificial Sequence
  VHH
MGTSLLCWMALCLLGADHADACPYSNPSLCSGGGGSELPTQGTFSNVSTNVSPAKPTTTACPYSNPSLCSGGGGSPAPRPPTPAPTIASQPLSLRPEACRPAAGGAVHTRGLDFACDIYIWAPLAGTCGVLLLSLVITLYCNHRNRRRVCKCPRPVVRAEGRGSLLTCGDVEENPGPMGWSCIILFLVATATGVHSQVQLQQSGGGLVQPGGSLRLSCAASGRTYNNYAMGWFRQAPGKEREFVAGISRSGGMTGYAESVKGRFTISRDNAKNMVFLQMNSLKPEDTAVYYCAAYVGGFSTARRDYSYWGQGTQVTVSSDPTPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 329 - protein, 537 aa, Artificial Sequence
  VHH
MGTSLLCWMALCLLGADHADACPYSNPSLCSGGGGSELPTQGTFSNVSTNVSPAKPTTTACPYSNPSLCSGGGGSPAPRPPTPAPTIASQPLSLRPEACRPAAGGAVHTRGLDFACDIYIWAPLAGTCGVLLLSLVITLYCNHRNRRRVCKCPRPVVRAEGRGSLLTCGDVEENPGPMGWSCIILFLVATATGVHSQVQLQESGGGLVQAGGSLIVSCATSGRTFSINAMGWFRQAPGKEREFVAGISRSGGMTGYAESVKGRFTISRDNAKNMVFLQMNSLKPEDTAVYYCAAYVGGFSTARRDYSYWGQGTQVSVSSDPTPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 330 - protein, 536 aa, Artificial Sequence
  VHH
MGTSLLCWMALCLLGADHADACPYSNPSLCSGGGGSELPTQGTFSNVSTNVSPAKPTTTACPYSNPSLCSGGGGSPAPRPPTPAPTIASQPLSLRPEACRPAAGGAVHTRGLDFACDIYIWAPLAGTCGVLLLSLVITLYCNHRNRRRVCKCPRPVVRAEGRGSLLTCGDVEENPGPMGWSCIILFLVATATGVHSQVQLQQSGGGLVQAGGSLRLSCAASGRTFSRYAMGWFRQAPGKEREFVAVINGSGGTTAYANSVKGRFTITRDNAKNTLYLQMNSLKPEDTAVYYCAAARIFTTTRNEYDHWGQGTQVTVSSDPTPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 331 - protein, 536 aa, Artificial Sequence
  VHH
MGTSLLCWMALCLLGADHADACPYSNPSLCSGGGGSELPTQGTFSNVSTNVSPAKPTTTACPYSNPSLCSGGGGSPAPRPPTPAPTIASQPLSLRPEACRPAAGGAVHTRGLDFACDIYIWAPLAGTCGVLLLSLVITLYCNHRNRRRVCKCPRPVVRAEGRGSLLTCGDVEENPGPMGWSCIILFLVATATGVHSQVQLQEPGGGLVQPGGSLRLSCAASGFTFSTYWMSWVHQAPGKGPEWVSGIDNGGGTTTYADSVKGRFTISRDNAKNTLYLQMNSLKPEETAIYYWAAARIFSTARNDYDHWGQGTQVTVSSDPTPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 332 - protein, 536 aa, Artificial Sequence
  VHH
MGTSLLCWMALCLLGADHADACPYSNPSLCSGGGGSELPTQGTFSNVSTNVSPAKPTTTACPYSNPSLCSGGGGSPAPRPPTPAPTIASQPLSLRPEACRPAAGGAVHTRGLDFACDIYIWAPLAGTCGVLLLSLVITLYCNHRNRRRVCKCPRPVVRAEGRGSLLTCGDVEENPGPMGWSCIILFLVATATGVHSQVQLQESGGGLVQAGGSLSLSCTASGRSFSNYGMGWFRQAPGKEREFVAAIGMVGGLTAYSNSAKGRFTISRDNAKNTLYLQMNSLKPEDTAVYLVAGGRIFRTSSRDYDIWGQGTQVTVSSDPTPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 333 - protein, 535 aa, Artificial Sequence
  VHH
MGTSLLCWMALCLLGADHADACPYSNPSLCSGGGGSELPTQGTFSNVSTNVSPAKPTTTACPYSNPSLCSGGGGSPAPRPPTPAPTIASQPLSLRPEACRPAAGGAVHTRGLDFACDIYIWAPLAGTCGVLLLSLVITLYCNHRNRRRVCKCPRPVVRAEGRGSLLTCGDVEENPGPMGWSCIILFLVATATGVHSQVQLQESGGGLVQPGGSLRLYCAAPGNIFRLNGTGWYRQAPGKQRELVTHITSGGNTDYADSVKGRFTISRDNAKNTVYLQMNSLKPEDTAIYYCAAARFFTTARNDYDHWGQGTQVTVSSDPTPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 334 - protein, 531 aa, Artificial Sequence
  VHH
MGTSLLCWMALCLLGADHADACPYSNPSLCSGGGGSELPTQGTFSNVSTNVSPAKPTTTACPYSNPSLCSGGGGSPAPRPPTPAPTIASQPLSLRPEACRPAAGGAVHTRGLDFACDIYIWAPLAGTCGVLLLSLVITLYCNHRNRRRVCKCPRPVVRAEGRGSLLTCGDVEENPGPMGWSCIILFLVATATGVHSQVQLQESGGGLVQAGGSLRLSCAASGSIFSINAMGWYRQAPGKQRELVAAITSGGSTNYADSVKGRFTISRDNAKNTVYLQMNSLKPEDTAVYYCNALGGFVPNYGWGQGTQVTVSSDPTPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 335 - protein, 531 aa, Artificial Sequence
  VHH
MGTSLLCWMALCLLGADHADACPYSNPSLCSGGGGSELPTQGTFSNVSTNVSPAKPTTTACPYSNPSLCSGGGGSPAPRPPTPAPTIASQPLSLRPEACRPAAGGAVHTRGLDFACDIYIWAPLAGTCGVLLLSLVITLYCNHRNRRRVCKCPRPVVRAEGRGSLLTCGDVEENPGPMGWSCIILFLVATATGVHSQVQLQESGGGLVQPGGSLRLSCAAPGNIFRLNGTGWYRQAPGKQRELVAHITSGGNTDYADSVKGRFTISRDNAKNTVYLQMNSLKPEDTAVYYCNALGGFVPNYGWGQGTQVTVSSDPTPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 336 - protein, 531 aa, Artificial Sequence
  VHH
MGTSLLCWMALCLLGADHADACPYSNPSLCSGGGGSELPTQGTFSNVSTNVSPAKPTTTACPYSNPSLCSGGGGSPAPRPPTPAPTIASQPLSLRPEACRPAAGGAVHTRGLDFACDIYIWAPLAGTCGVLLLSLVITLYCNHRNRRRVCKCPRPVVRAEGRGSLLTCGDVEENPGPMGWSCIILFLVATATGVHSQVQLQESGGGLVQAGGSLRLSCAASGSIFSINAMGWYRQAPGKQRELVAAITSGGSTNYADSVKGRFTTSRDNAKNTVYLQMNSLKPEDPAVYYCNALGGFVPNDGWGQGTQVTVSSDPTPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 337 - protein, 531 aa, Artificial Sequence
  VHH
MGTSLLCWMALCLLGADHADACPYSNPSLCSGGGGSELPTQGTFSNVSTNVSPAKPTTTACPYSNPSLCSGGGGSPAPRPPTPAPTIASQPLSLRPEACRPAAGGAVHTRGLDFACDIYIWAPLAGTCGVLLLSLVITLYCNHRNRRRVCKCPRPVVRAEGRGSLLTCGDVEENPGPMGWSCIILFLVATATGVHSQVQLQESGGGLVQAGGSLRLSCAASGNNFRLNAMGWYRQAPGKQRELVAHITSGGNTDYADSVKGRFTISRDNAKNTTYLQMNSLKPEDTAVYYCNALGGFAPNYGWGQGTQVTVSSDPTPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 338 - protein, 532 aa, Artificial Sequence
  VHH
MGTSLLCWMALCLLGADHADACPYSNPSLCSGGGGSELPTQGTFSNVSTNVSPAKPTTTACPYSNPSLCSGGGGSPAPRPPTPAPTIASQPLSLRPEACRPAAGGAVHTRGLDFACDIYIWAPLAGTCGVLLLSLVITLYCNHRNRRRVCKCPRPVVRAEGRGSLLTCGDVEENPGPMGWSCIILFLVATATGVHSQVQLQQSGGGLVQAGGSLSLSCTASGRSFSNYGMGWFRQAPGKEREFVAAIGMVGGLTAYSNSAKGRFTISRDNAKNTVYLQMNSLKPEDTAVYYCNALGGFVPNYVWGQGTQVTVSSDPTPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 339 - protein, 531 aa, Artificial Sequence
  VHH
MGTSLLCWMALCLLGADHADACPYSNPSLCSGGGGSELPTQGTFSNVSTNVSPAKPTTTACPYSNPSLCSGGGGSPAPRPPTPAPTIASQPLSLRPEACRPAAGGAVHTRGLDFACDIYIWAPLAGTCGVLLLSLVITLYCNHRNRRRVCKCPRPVVRAEGRGSLLTCGDVEENPGPMGWSCIILFLVATATGVHSQVQLQESGGGLVQAGGSLSLSCTASGRSFSINAMGWYRQAPGKQRELVAAITSGGSTNYADSVKGRFTISRDNAKNTVYLQMNSLKPEDTAVYYCNALGGFVLNYGWGQGTQVTVSSDPTPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 340 - protein, 531 aa, Artificial Sequence
  VHH
MGTSLLCWMALCLLGADHADACPYSNPSLCSGGGGSELPTQGTFSNVSTNVSPAKPTTTACPYSNPSLCSGGGGSPAPRPPTPAPTIASQPLSLRPEACRPAAGGAVHTRGLDFACDIYIWAPLAGTCGVLLLSLVITLYCNHRNRRRVCKCPRPVVRAEGRGSLLTCGDVEENPGPMGWSCIILFLVATATGVHSQVQLQESGGGLVQAGGSLRLSCAASGSIFSINAMGWYRQAPGKQRELVAAITSGGSTNYADPVKGRFTISRDNAKNTVYLQMNSLKPEDTAVYYCNALGGLVPNYGWGQGTQVTVSSDPTPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 341 - protein, 531 aa, Artificial Sequence
  VHH
MGTSLLCWMALCLLGADHADACPYSNPSLCSGGGGSELPTQGTFSNVSTNVSPAKPTTTACPYSNPSLCSGGGGSPAPRPPTPAPTIASQPLSLRPEACRPAAGGAVHTRGLDFACDIYIWAPLAGTCGVLLLSLVITLYCNHRNRRRVCKCPRPVVRAEGRGSLLTCGDVEENPGPMGWSCIILFLVATATGVHSQVQLQESGGGLVQAGGSLRLSCAASGSIFSINAMGWYRQAPGKQRELVAAITSGGSTNYADSVKGRFTISRDNAKNTVYLQMNSLKPEDTAVYYCNALGGFLPNYGWGQGTQVTVSSDPTPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 342 - protein, 531 aa, Artificial Sequence
  VHH
MGTSLLCWMALCLLGADHADACPYSNPSLCSGGGGSELPTQGTFSNVSTNVSPAKPTTTACPYSNPSLCSGGGGSPAPRPPTPAPTIASQPLSLRPEACRPAAGGAVHTRGLDFACDIYIWAPLAGTCGVLLLSLVITLYCNHRNRRRVCKCPRPVVRAEGRGSLLTCGDVEENPGPMGWSCIILFLVATATGVHSQVQLQESGGGLVQAGGSLRLSCAASGSIFSINAMGWYRQAPGKQRELVAAITSGGSTNYADSVKGRFTISRDNAKNTVYLQMNSLKPEDTAVYYCNALGGFVTNYGWGQGTQVAVSSDPTPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 343 - protein, 531 aa, Artificial Sequence
  VHH
MGTSLLCWMALCLLGADHADACPYSNPSLCSGGGGSELPTQGTFSNVSTNVSPAKPTTTACPYSNPSLCSGGGGSPAPRPPTPAPTIASQPLSLRPEACRPAAGGAVHTRGLDFACDIYIWAPLAGTCGVLLLSLVITLYCNHRNRRRVCKCPRPVVRAEGRGSLLTCGDVEENPGPMGWSCIILFLVATATGVHSQVQLQESGGGLVQPGGSLRLSCAASGSIFSINAMGWYRQAPGKQRELVAFITSGGSTNYADSVKGRFTISRDNAKNTVYLQMNSLKPEDTAVYYCNALGGFVPSYGWGQGTQVTVSSDPTPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 344 - protein, 527 aa, Artificial Sequence
  VHH
MGTSLLCWMALCLLGADHADACPYSNPSLCSGGGGSELPTQGTFSNVSTNVSPAKPTTTACPYSNPSLCSGGGGSPAPRPPTPAPTIASQPLSLRPEACRPAAGGAVHTRGLDFACDIYIWAPLAGTCGVLLLSLVITLYCNHRNRRRVCKCPRPVVRAEGRGSLLTCGDVEENPGPMGWSCIILFLVATATGVHSQVQLQQSGGGLVQTGGSLRLSCAASGRTVMGWYRQAPGKQRELVAVITRGGSTNYADSVKGRFTISRDNAKNTVYLQMNSLKPEDTAFYYCNALGGFVPNYGWGQGTQVTVSSDPTPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 345 - protein, 531 aa, Artificial Sequence
  VHH
MGTSLLCWMALCLLGADHADACPYSNPSLCSGGGGSELPTQGTFSNVSTNVSPAKPTTTACPYSNPSLCSGGGGSPAPRPPTPAPTIASQPLSLRPEACRPAAGGAVHTRGLDFACDIYIWAPLAGTCGVLLLSLVITLYCNHRNRRRVCKCPRPVVRAEGRGSLLTCGDVEENPGPMGWSCIILFLVATATGVHSQVQLQESGGGLVQPGGSLRLSCAAPGNIFRLNGTGWYRQAPGKQRELVAHITSGGNTDYADSVKGRFTISRDNAKNTVYLQMNSLKPEDTAVYYCNALGGFVPSYGWGQGTQVTVSSDPTPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 346 - protein, 531 aa, Artificial Sequence
  VHH
MGTSLLCWMALCLLGADHADACPYSNPSLCSGGGGSELPTQGTFSNVSTNVSPAKPTTTACPYSNPSLCSGGGGSPAPRPPTPAPTIASQPLSLRPEACRPAAGGAVHTRGLDFACDIYIWAPLAGTCGVLLLSLVITLYCNHRNRRRVCKCPRPVVRAEGRGSLLTCGDVEENPGPMGWSCIILFLVATATGVHSQVQLQESGGGLVQPGGSLRLSCAAPGNNFRLNAMGWYRQAPGKQRELVAHITSGGNTDYADSVKGRFTISRDNAKNTVYLQMNSLKPEDTAVYYCNALGGFVPSYGWGQGTQVTVSSDPTPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 347 - protein, 531 aa, Artificial Sequence
  VHH
MGTSLLCWMALCLLGADHADACPYSNPSLCSGGGGSELPTQGTFSNVSTNVSPAKPTTTACPYSNPSLCSGGGGSPAPRPPTPAPTIASQPLSLRPEACRPAAGGAVHTRGLDFACDIYIWAPLAGTCGVLLLSLVITLYCNHRNRRRVCKCPRPVVRAEGRGSLLTCGDVEENPGPMGWSCIILFLVATATGVHSQVQLQQSGGGLVQAGGSLRLSCAASGSIFSINAMGWYRQAPGKQRELVAFITSGGSTNYADSVKGRFTISRDNAKNTVYLQMNSLKPEDTAVYYCNALGGFVPSYGWGQGTQVTVSSDPTPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 348 - protein, 531 aa, Artificial Sequence
  VHH
MGTSLLCWMALCLLGADHADACPYSNPSLCSGGGGSELPTQGTFSNVSTNVSPAKPTTTACPYSNPSLCSGGGGSPAPRPPTPAPTIASQPLSLRPEACRPAAGGAVHTRGLDFACDIYIWAPLAGTCGVLLLSLVITLYCNHRNRRRVCKCPRPVVRAEGRGSLLTCGDVEENPGPMGWSCIILFLVATATGVHSQVQLQQSGGGLVQPGGSLRLSCAAPGNIFRLNGTGWYRQAPGKQRELVAHITSGGSTNYADSVKGRFTISRDNAKNTVYLQMNSLKPEDTAVYYCNALGGFVPSYGWGQGTQVTVSSDPTPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 349 - protein, 531 aa, Artificial Sequence
  VHH
MGTSLLCWMALCLLGADHADACPYSNPSLCSGGGGSELPTQGTFSNVSTNVSPAKPTTTACPYSNPSLCSGGGGSPAPRPPTPAPTIASQPLSLRPEACRPAAGGAVHTRGLDFACDIYIWAPLAGTCGVLLLSLVITLYCNHRNRRRVCKCPRPVVRAEGRGSLLTCGDVEENPGPMGWSCIILFLVATATGVHSQVQLQESGGGLVQAGGSLRLSCAASGSIFSINAMGWYRQAPGKQRELVAAITSGGSTNYADSVKGRFTISRDNAKNTVYLQMNSLKPEDTAVYYCNALGGFVPSYGWGQGTQVTVSSDPTPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 350 - protein, 532 aa, Artificial Sequence
  VHH
MGTSLLCWMALCLLGADHADACPYSNPSLCSGGGGSELPTQGTFSNVSTNVSPAKPTTTACPYSNPSLCSGGGGSPAPRPPTPAPTIASQPLSLRPEACRPAAGGAVHTRGLDFACDIYIWAPLAGTCGVLLLSLVITLYCNHRNRRRVCKCPRPVVRAEGRGSLLTCGDVEENPGPMGWSCIILFLVATATGVHSQVQLQESGGGLVQAGGSLSLSCTASGRSFSNYGMGWFRQAPGKEREFVAAIGMVGGLTAYSNSAKGRFTISRDNAKNTLYLQMNSLKPEDTAVYYCNALGGFVPSYGWGQGTQVTVSSDPTPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 351 - protein, 531 aa, Artificial Sequence
  VHH
MGTSLLCWMALCLLGADHADACPYSNPSLCSGGGGSELPTQGTFSNVSTNVSPAKPTTTACPYSNPSLCSGGGGSPAPRPPTPAPTIASQPLSLRPEACRPAAGGAVHTRGLDFACDIYIWAPLAGTCGVLLLSLVITLYCNHRNRRRVCKCPRPVVRAEGRGSLLTCGDVEENPGPMGWSCIILFLVATATGVHSQVQLQESGGGLVQAGGSLRLSCAASGSIFSINAMGWYRQAPGKQRELVAFITSGGSTNYADSVKGRFTISRDNAKNTVYLQMNSLKPEDTAVYYCNALGGFVPSYGWGQGTLVTVSSDPTPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 352 - protein, 531 aa, Artificial Sequence
  VHH
MGTSLLCWMALCLLGADHADACPYSNPSLCSGGGGSELPTQGTFSNVSTNVSPAKPTTTACPYSNPSLCSGGGGSPAPRPPTPAPTIASQPLSLRPEACRPAAGGAVHTRGLDFACDIYIWAPLAGTCGVLLLSLVITLYCNHRNRRRVCKCPRPVVRAEGRGSLLTCGDVEENPGPMGWSCIILFLVATATGVHSQVQLQESGGGLVQPGGSLRLSCAAPGNIFSINAMGWYRQAPGKQRELVAFITSGGSTNYADSVKGRFTISRDNAKNTVYLQMNSLKPEDTAVYYCNALGGFVPSYGWGQGTQVTVSSDPTPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 353 - protein, 534 aa, Artificial Sequence
  VHH
MGTSLLCWMALCLLGADHADACPYSNPSLCSGGGGSELPTQGTFSNVSTNVSPAKPTTTACPYSNPSLCSGGGGSPAPRPPTPAPTIASQPLSLRPEACRPAAGGAVHTRGLDFACDIYIWAPLAGTCGVLLLSLVITLYCNHRNRRRVCKCPRPVVRAEGRGSLLTCGDVEENPGPMGWSCIILFLVATATGVHSQVQLQESGGGLVQPGGSLRLSCLASGFTFSTYWMSWVRQAPGKGPEWVSGIDNGGGTTTYADSVKGRFTISRDNAKNTLNLEMNNLKPEDTALYYCARNPTRGWYSTDYRGQGTQVTVSSDPTPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 354 - protein, 534 aa, Artificial Sequence
  VHH
MGTSLLCWMALCLLGADHADACPYSNPSLCSGGGGSELPTQGTFSNVSTNVSPAKPTTTACPYSNPSLCSGGGGSPAPRPPTPAPTIASQPLSLRPEACRPAAGGAVHTRGLDFACDIYIWAPLAGTCGVLLLSLVITLYCNHRNRRRVCKCPRPVVRAEGRGSLLTCGDVEENPGPMGWSCIILFLVATATGVHSQVQLQESGGGLVQPGGSLRLSCAASGFTFSTYWMSWVRQAPGKGPEWVSGIDNGGGTTTYADSVKGRFTISRDNAKHTLYLQMNTLKPEDTALYYCARNPTRGWYSTDYRGQGTQVTVSSDPTPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 355 - protein, 10 aa, Artificial Sequence
  spacer
GGGGSGGGGS

SEQ ID NO: 356 - protein, 15 aa, Artificial Sequence
  spacer
GGGGSGGGGSGGGGS

SEQ ID NO: 357 - protein, 20 aa, Artificial Sequence
  spacer
GGGGSGGGGSGGGGSGGGGS

SEQ ID NO: 358 - protein, 19 aa, Artificial Sequence
  signal peptide
MGWSCIILFLVATATGVHS

SEQ ID NO: 359 - protein, 20 aa, Artificial Sequence
  2A-like sequence
RAEGRGSLLTCGDVEENPGP

SEQ ID NO: 360 - protein, 8 aa, Artificial Sequence
  CDR1
ISGFGVVT

SEQ ID NO: 361 - protein, 114 aa, Artificial Sequence; identical to SEQ ID NO: 225
  VHH

SEQ ID NO: 362 - protein, 115 aa, Artificial Sequence; identical to SEQ ID NO: 229
  VHH

SEQ ID NO: 363 - protein, 114 aa, Artificial Sequence; identical to SEQ ID NO: 230
  VHH

SEQ ID NO: 364 - protein, 114 aa, Artificial Sequence; identical to SEQ ID NO: 231
  VHH

SEQ ID NO: 365 - protein, 114 aa, Artificial Sequence; identical to SEQ ID NO: 232
  VHH

SEQ ID NO: 366 - protein, 114 aa, Artificial Sequence; identical to SEQ ID NO: 242
  VHH

SEQ ID NO: 367 - protein, 114 aa, Artificial Sequence; identical to SEQ ID NO: 245
  VHH

SEQ ID NO: 368 - protein, 114 aa, Artificial Sequence; identical to SEQ ID NO: 246
  VHH

SEQ ID NO: 369 - protein, 114 aa, Artificial Sequence; identical to SEQ ID NO: 247
  VHH

SEQ ID NO: 370 - protein, 117 aa, Artificial Sequence
  VHH
QVQLQESGGGLVQAGGSLRLSCAASGSIFSINAMGWYRQAPGKQRELVAFITSGGSTNYADSVKGRFTISRDNAKNTVYLQMNSLKPEDTAVYYCNALGGFVPSYGWGQGTQVTVSS

(19) United States
(12) Patent Application Publication
(10) Pub. No.: US 2025/0289891 A1
(43) Pub. Date: Sep. 18, 2025

(54) ANTI-CD307E SINGLE-DOMAIN ANTIBODIES, USES THEREOF IN CAR T-CELL AND FOR THE TREATMENT OF DISEASES

(71) Applicant: AUTOLUS LIMITED, London (GB)

(72) Inventors: Alexander Kinna, London (GB); Mathieu Ferrari, London (GB); Shimobi Onuoha, London (GB); Martin Pulé, London (GB); Tudor IIca, London (GB); Preeta Datta, London (GB); Philliip Wu, London (GB)

(21) Appl. No.: 18/570,278

(22) PCT Filed: Jun. 17, 2022
(86) PCT No.: PCT/GB2022/051550

§ 371 Date: Dec. 14, 2023

(30) Foreign Application Priority Data
Jun. 18, 2021 (GB) 2108773.9
Nov. 16, 2021 (GB) 2116502.2

(51) Int. Cl. C07K 16/28; A61K 40/31; A61K 40/33; A61K 40/42; C12N 15/86

(52) U.S. Cl. CPC C07K 16/283; A61K 40/31; A61K 40/33; A61K 40/421; C12N 15/86; A61K 2239/13; A61K 2239/48; C07K 2317/24; C07K 2317/565; C07K 2317/569; C12N 2740/13043

(57) ABSTRACT

The present invention relates to FcRH5 binding domains. In particular, the invention provides single domain antibodies, antibody conjugates, chimeric antigen receptors (CARs) and immune cell engagers which comprise such binding domains.

### FIELD OF THE INVENTION

The present invention relates to FcRH5 binding domains. In particular, the invention relates to single domain antibodies, antibody conjugates, chimeric antigen receptors (CARs), immune cell engagers which comprise such FcRH5 binding domains. Modalities targeting FcRH5 may be useful in the treatment of cancerous diseases such as multiple myeloma, for example.

### BACKGROUND TO THE INVENTION

Multiple Myeloma (myeloma) is a bone-marrow malignancy of plasma cells. Collections of abnormal plasma cells accumulate in the bone marrow, where they interfere with the production of normal blood cells. Myeloma is the second most common hematological malignancy in the U.S. (after non-Hodgkin lymphoma), and constitutes 13% of haematologic malignancies and 1% of all cancers. The disease is burdensome in terms of suffering as well as medical expenditure since it causes pathological fractures, susceptibility to infection, renal and then bone-marrow failure before death.

Unlike many lymphomas, myeloma is currently incurable. Standard chemotherapy agents used in lymphoma are largely ineffective for myeloma. In addition, since CD20 expression is lost in plasma cells, Rituximab cannot be used against this disease. New agents such as Bortezamib and Lenolidomide are partially effective, but fail to lead to long-lasting remissions.

There is thus a need for alternative agents for the treatment of myeloma which have increased efficacy and improved long-term effects.

The Fc receptor-like 5 (FcRH5), also known as CD307, IRTA2, FcRL5 and BXMAS1, is a member of the immunoglobulin receptor superfamily and the Fc-receptor family. FcRH5 is a type 1 transmembrane protein comprising 9 Ig-like domain in the extracellular region. FcRH5 is exclusively expressed in the B-cell lineage and retained in plasma cells and is currently investigated as a therapeutic target in multiple myeloma.

Several isoforms are present: isoform c (Uniprot Q96RD9-1-v3), canonical structure, contains 9 Ig-like domains in the extracellular region, a transmembrane domain and a cytoplasmic tail; the isoform a contains 8 Ig-like extracellular domains in a soluble form (Uniprot Q96RD9-3); the isoform b is a GPI-anchored protein comprising the first 6 Ig-like domains and a 32aa peptide (Uniprot Q96RD9-4); the isoform d contains the first 2 Ig-like domains only (Uniprot Q96RD9-5).

A number of immunotherapeutic agents have been described for use in cancer treatment, including therapeutic monoclonal antibodies (mAbs), immunoconjugated mAbs, radioconjugated mAbs and bi-specific T-cell engagers.

However, there is still a need for immunotherapeutic agents targeted against FcRH5.

### SUMMARY OF THE INVENTION

The present inventors have surprisingly demonstrated that single domain antibodies comprising the complementarity determining regions (CDRs) described herein can target FcRH5. These single domain FcRH5 binders may be useful in antigen binding entities such as VHH antibodies, antibody conjugates, chimeric antigen receptors (CARs), and immune cell engagers which comprise such FcRH5 binding domains.

Thus, in a first aspect, the present invention provides a single domain antibody (sdAb) comprising a FcRH5 binding domain.

Suitably, the FcRH5 binding domain may comprise:

(i) complementarity determining regions (CDRs)
with the following sequences:
CDR1-
(SEQ ID NO: 1)
RSSFSNNA
CDR2-
(SEQ ID NO: 2)
ITKGGVT
CDR3-
(SEQ ID NO: 3)
NTIPFRSA;
or
(ii) CDRs with the following sequences:
CDR1-
(SEQ ID NO: 4)
GFTFSNYA
CDR2-
(SEQ ID NO: 5)
INSDGGTA
CDR3-
(SEQ ID NO: 6)
AANRGFCAGVRCLEYQY;
or
(iii) CDRs with the following sequences:
CDR1-
(SEQ ID NO: 7)
GRTFSINA
CDR2-
(SEQ ID NO: 8)
IGGSGRVSST
CDR3-
(SEQ ID NO: 9)
AARRDYLPFPPESYDY;
or
(iv) CDRs with the following sequences:
CDR1-
(SEQ ID NO: 7)
GRTFSINA
CDR2-
(SEQ ID NO: 8)
IGGSGRVSST
CDR3-
(SEQ ID NO: 10)
AAGRRTSTNGGDYDY;
or
(v) CDRs with the following sequences:
CDR1-
(SEQ ID NO: 7)
GRTFSINA
CDR2-
(SEQ ID NO: 11)
ISRSGGAT
CDR3-
(SEQ ID NO: 12)
AGTRRAFSTGLRDYDY;
or
(vi) CDRs with the following sequences:
CDR1-
(SEQ ID NO: 13)
GRTFSNST
CDR2-
(SEQ ID NO: 14)
ISWSGGTY
CDR3-
(SEQ ID NO: 15)
AAARKGWSTRGDDYDY;
or
(vii) CDRs with the following sequences:
CDR1-
(SEQ ID NO: 16)
GRTYNNYA
CDR2-
(SEQ ID NO: 17)
ISRSGGMT
CDR3-
(SEQ ID NO: 18)
AAYVGGFSTARRDYSY;
or
(viii) CDRs with the following sequences:
CDR1-
(SEQ ID NO: 19)
GRTFSSYA
CDR2-
(SEQ ID NO: 20)
ISRIGGVT
CDR3-
(SEQ ID NO: 21)
AAAGLVSISTTPNDYDY;
or
(ix) CDRs with the following sequences:
CDR1-
(SEQ ID NO: 22)
GNIFRLNG
CDR2-
(SEQ ID NO: 23)
ITSGGNT
CDR3-
(SEQ ID NO: 24)
NAIPFRL;
or
(x) CDRs with the following sequences:
CDR1-
(SEQ ID NO: 25)
RNIFSLNP
CDR2-
(SEQ ID NO: 26)
ITDGGST
CDR3-
(SEQ ID NO: 27)
NRVGGLQTWA;
or
(xi) CDRs with the following sequences:
CDR1-
(SEQ ID NO: 28)
GRSFSNYG
CDR2-
(SEQ ID NO: 29)
IGMVGGLT
CDR3-
(SEQ ID NO: 30)
AAGRRFSTSSRDYDI;
or
(xii) CDRs with the following sequences:
CDR1-
(SEQ ID NO: 31)
GRTFDSRP
CDR2-
(SEQ ID NO: 32)
VSWRGEST
CDR3-
(SEQ ID NO: 33)
AAGEPYSGTYYYRGRDYDY;
or
(xiii) CDRs with the following sequences:
CDR1-
(SEQ ID NO: 34)
GRTFSMYA
CDR2-
(SEQ ID NO: 35)
ISGSARIT
CDR3-
(SEQ ID NO: 36)
AASSTYTSTSGSSYNY;
or
(xiv) CDRs with the following sequences:
CDR1-
(SEQ ID NO: 37)
GSIFSINA
CDR2-
(SEQ ID NO: 38)
ITSGGST
CDR3-
(SEQ ID NO: 39)
NALGGFVPSYG;
or
(xv) CDRs with the following sequences:
CDR1-
(SEQ ID NO: 155)
GRTSSRAA
CDR2-
(SEQ ID NO: 156)
ISWSGGTT
CDR3-
(SEQ ID NO: 157)
AAARIFTTARNDYDH;
or
(xvi) CDRs with the following sequences:
CDR1-
(SEQ ID NO: 22)
GNIFRLNG
CDR2-
(SEQ ID NO: 23)
ITSGGNT
CDR3-
(SEQ ID NO: 186)
NTIPFRLS;
or
(xvii) CDRs with the following sequences:
CDR1-
(SEQ ID NO: 160)
GNIFRLNA
CDR2-
(SEQ ID NO: 23)
ITSGGNT
CDR3-
(SEQ ID NO: 186)
NTIPFRLS;
or
(xviii) CDRs with the following sequences:
CDR1-
(SEQ ID NO: 1)
RSSFSNNA
CDR2-
(SEQ ID NO: 2)
ITKGGVT
CDR3-
(SEQ ID NO: 187)
NTIPVRSA;
or
(xix) CDRs with the following sequences:
CDR1-
(SEQ ID NO: 37)
GSIFSINA
CDR2-
(SEQ ID NO: 2)
ITKGGVT
CDR3-
(SEQ ID NO: 188)
NTIPCRSA;
or
(xx) CDRs with the following sequences:
CDR1-
(SEQ ID NO: 161)
GSSFRLNG
CDR2-
(SEQ ID NO: 38)
ITSGGST
CDR3-
(SEQ ID NO: 189)
NTIPFSRA;
or
(xxi) CDRs with the following sequences:
CDR1-
(SEQ ID NO: 162)
GRSVSINA
CDR2-
(SEQ ID NO: 176)
IDRSGNT
CDR3-
(SEQ ID NO: 190)
NTIPYSDS;
or
(xxii) CDRs with the following sequences:
CDR1-
(SEQ ID NO: 19)
GRTFSSYA
CDR2-
(SEQ ID NO: 177)
IDGIGGIT
CDR3-
(SEQ ID NO: 3)
NTIPFRSA;
or
(xxiii) CDRs with the following sequences:
CDR1-
(SEQ ID NO: 1)
RSSFSNNA
CDR2-
(SEQ ID NO: 2)
ITKGGVT
CDR3-
(SEQ ID NO: 191)
NAIPFRSA;
or
(xxiv) CDRs with the following sequences:
CDR1-
(SEQ ID NO: 163)
GNNFRLNA
CDR2-
(SEQ ID NO: 23)
ITSGGNT
CDR3-
(SEQ ID NO: 192)
NAIPFRPS;
or
(xxv) CDRs with the following sequences:
CDR1-
(SEQ ID NO: 22)
GNIFRLNG
CDR2-
(SEQ ID NO: 23)
ITSGGNT
CDR3-
(SEQ ID NO: 193)
NAIPFRRS;
or
(xxvi) CDRs with the following sequences:
CDR1-
(SEQ ID NO: 164)
ERIFRINA
CDR2-
(SEQ ID NO: 23)
ITSGGNT
CDR3-
(SEQ ID NO: 193)
NAIPFRRS;
or
(xxvii) CDRs with the following sequences:
CDR1-
(SEQ ID NO: 165)
GFTFSTYW
CDR2-
(SEQ ID NO: 178)
IDNGGGTT
CDR3-
(SEQ ID NO: 194)
NALPFRLS;
or
(xxviii) CDRs with the following sequences:
CDR1-
(SEQ ID NO: 22)
GNIFRLNG
CDR2-
(SEQ ID NO: 23)
ITSGGNT
CDR3-
(SEQ ID NO: 195)
NATPFRLS;
or
(xxix) CDRs with the following sequences:
CDR1-
(SEQ ID NO: 22)
GNIFRLNG
CDR2-
(SEQ ID NO: 179)
ITRGGNT
CDR3-
(SEQ ID NO: 196)
NSIPFRLS;
or
(xxx) CDRs with the following sequences:
CDR1-
(SEQ ID NO: 166)
GNIFRING
CDR2-
(SEQ ID NO: 23)
ITSGGNT
CDR3-
(SEQ ID NO: 197)
NAIPFRIS;
or
(xxxi) CDRs with the following sequences:
CDR1-
(SEQ ID NO: 163)
GNNFRLNA
CDR2-
(SEQ ID NO: 23)
ITSGGNT
CDR3-
(SEQ ID NO: 198)
NAIPFRLY;
or
(xxxii) CDRs with the following sequences:
CDR1-
(SEQ ID NO: 165)
GFTFSTYW
CDR2-
(SEQ ID NO: 178)
IDNGGGTT
CDR3
(SEQ ID NO: 199)
KAIPFRLS;
or
(xxiii) CDRs with the following sequences:
CDR1-
(SEQ ID NO: 167)
GSSFSNNA
CDR2-
(SEQ ID NO: 2)
ITKGGVT
CDR3-
(SEQ ID NO: 3)
NTIPFRSA;
or
(xxxiv) CDRs with the following sequences:
CDR1-
(SEQ ID NO: 165)
GFTFSTYW
CDR2-
(SEQ ID NO: 178)
IDNGGGTT
CDR3-
(SEQ ID NO: 3)
NTIPFRSA;
or
(xxxv) CDRs with the following sequences:
CDR1-
(SEQ ID NO: 160)
GNIFRLNA
CDR2-
(SEQ ID NO: 2)
ITKGGVT
CDR3-
(SEQ ID NO: 3)
NTIPFRSA;
or
(xxxvi) CDRs with the following sequences:
CDR1-
(SEQ ID NO: 168)
RSSFGNNA
CDR2-
(SEQ ID NO: 2)
ITKGGVT
CDR3-
(SEQ ID NO: 3)
NTIPFRSA;
or
(xxxvii) CDRs with the following sequences:
CDR1-
(SEQ ID NO: 169)
GRTFSTYG
CDR2-
(SEQ ID NO: 11)
ISRSGGAT
CDR3-
(SEQ ID NO: 12)
AGTRRAFSTGLRDYDY;
or
(xxxviii) CDRs with the following sequences:
CDR1-
(SEQ ID NO: 170)
GTIERNNA
CDR2-
(SEQ ID NO: 38)
ITSGGST
CDR3-
(SEQ ID NO: 200)
AAGRRFSTRSRDYDY;
or
(xxxix) CDRs with the following sequences:
CDR1-
(SEQ ID NO: 28)
GRSFSNYG
CDR2-
(SEQ ID NO: 29)
IGMVGGLT
CDR3-
(SEQ ID NO: 201)
AAGRRFSTSSRDYDY;
or
(xl) CDRs with the following sequences:
CDR1-
(SEQ ID NO: 28)
GRSFSNYG
CDR2-
(SEQ ID NO: 29)
IGMVGGLT
CDR3-
(SEQ ID NO: 202)
AAGRRFSTGSRDYDI;
or
(xli) CDRs with the following sequences:
CDR1-
(SEQ ID NO: 19)
GRTFSSYA
CDR2-
(SEQ ID NO: 179)
ISQFGGVTT
CDR3-
(SEQ ID NO: 202)
AAGRRFSTGSRDYDI;
or
(xlii) CDRs with the following sequences:
CDR1-
(SEQ ID NO: 28)
GRSFSNYG
CDR2-
(SEQ ID NO: 29)
IGMVGGLT
CDR3-
(SEQ ID NO: 203)
AGGRRFSTSSRDYDI;
or
(xliii) CDRs with the following sequences:
CDR1-
(SEQ ID NO: 28)
GRSFSNYG
CDR2-
(SEQ ID NO: 29)
IGMVGGLT
CDR3-
(SEQ ID NO: 204)
AAGRRFSTSSREYDI;
or
(xliv) CDRs with the following sequences:
CDR1-
(SEQ ID NO: 28)
GRSFSNYG
CDR2-
(SEQ ID NO: 180)
IGMVGGLP
CDR3-
(SEQ ID NO: 205)
AAGRRLSTSSRDYDI;
or
(xlv) CDRs with the following sequences:
CDR1-
(SEQ ID NO: 28)
GRSFSNYG
CDR2-
(SEQ ID NO: 181)
ISRGGGVS
CDR3-
(SEQ ID NO: 206)
AAGLRFSTGSRDYDI;
or
(xlvi) CDRs with the following sequences:
CDR1-
(SEQ ID NO: 171)
GRTFRRYA
CDR2-
(SEQ ID NO: 17)
ISRSGGMT
CDR3-
(SEQ ID NO: 207)
AAYVGGFSTTRRDYAY;
or
(xlvii) CDRs with the following sequences:
CDR1-
(SEQ ID NO: 13)
GRTFSNST
CDR2-
(SEQ ID NO: 156)
ISWSGGTT
CDR3-
(SEQ ID NO: 15)
AAARKGWSTRGDDYDY;
or
(xlviii) CDRs with the following sequences:
CDR1-
(SEQ ID NO: 172)
GRTVI
CDR2-
(SEQ ID NO: 182)
SSGSGGVT
CDR3-
(SEQ ID NO: 208)
AAALTWSTRPSDFTS;
or
(xlix) CDRs with the following sequences:
CDR1-
(SEQ ID NO: 172)
GRTVI
CDR2-
(SEQ ID NO: 183)
SNWSGGVT
CDR3-
(SEQ ID NO: 18)
AAYVGGFSTARRDYSY;
or
(l) CDRs with the following sequences:
CDR1-
(SEQ ID NO: 7)
GRTFSINA
CDR2-
(SEQ ID NO: 17)
ISRSGGMT
CDR3-
(SEQ ID NO: 18)
AAYVGGFSTARRDYSY;
or
(li) CDRs with the following sequences:
CDR1-
(SEQ ID NO: 173)
GRTFSRYA
CDR2-
(SEQ ID NO: 184)
INGSGGT
CDR3-
(SEQ ID NO: 209)
AAARIFTTTRNEYDH;
or
(lii) CDRs with the following sequences:
CDR1-
(SEQ ID NO: 173)
GRTFSRYA
CDR2-
(SEQ ID NO: 184)
INGSGGT
CDR3-
(SEQ ID NO: 209)
AAARIFTTTRNEYDH;
or
(liii) CDRs with the following sequences:
CDR1-
(SEQ ID NO: 165)
GFTFSTYW
CDR2-
(SEQ ID NO: 178)
IDNGGGTT
CDR3-
(SEQ ID NO: 210)
AAARIFSTARNDYDH;
or
(liv) CDRs with the following sequences:
CDR1-
(SEQ ID NO: 28)
GRSFSNYG
CDR2-
(SEQ ID NO: 29)
IGMVGGLT
CDR3-
(SEQ ID NO: 211)
AGGRIFRTSSRDYDI;
or
(lv) CDRs with the following sequences:
CDR1-
(SEQ ID NO: 22)
GNIFRLNG
CDR2-
(SEQ ID NO: 23)
ITSGGNT
CDR3-
(SEQ ID NO: 212)
AAARFFTTARNDYDH;
or
(lvi) CDRs with the following sequences:
CDR1-
(SEQ ID NO: 37)
GSIFSINA
CDR2-
(SEQ ID NO: 38)
ITSGGST
CDR3-
(SEQ ID NO: 213)
NALGGFVPNYG;
or
(lvii) CDRs with the following sequences:
CDR1-
(SEQ ID NO: 22)
GNIFRLNG
CDR2-
(SEQ ID NO: 23)
ITSGGNT
CDR3-
(SEQ ID NO: 213)
NALGGFVPNYG;
or
(lviii) CDRs with the following sequences:
CDR1-
(SEQ ID NO: 37)
GSIFSINA
CDR2-
(SEQ ID NO: 38)
ITSGGST
CDR3-
(SEQ ID NO: 214)
NALGGFVPNDG;
or
(lix) CDRs with the following sequences:
CDR1-
(SEQ ID NO: 163)
GNNFRLNA
CDR2-
(SEQ ID NO: 23)
ITSGGNT
CDR3-
(SEQ ID NO: 215)
NALGGFAPNYG;
or
(lx) CDRs with the following sequences:
CDR1-
(SEQ ID NO: 28)
GRSFSNYG
CDR2-
(SEQ ID NO: 29)
IGMVGGLT
CDR3-
(SEQ ID NO: 216)
NALGGFVPNYV;
or
(lxi) CDRs with the following sequences:
CDR1-
(SEQ ID NO: 174)
GRSFSINA
CDR2-
(SEQ ID NO: 38)
ITSGGST
CDR3-
(SEQ ID NO: 217)
NALGGFVLNYG;
or
(lxii) CDRs with the following sequences:
CDR1-
(SEQ ID NO: 37)
GSIFSINA
CDR2-
(SEQ ID NO: 38)
ITSGGST
CDR3-
(SEQ ID NO: 218)
NALGGLVPNYG;
or
(lxiii) CDRs with the following sequences:
CDR1-
(SEQ ID NO: 37)
GSIFSINA
CDR2-
(SEQ ID NO: 38)
ITSGGST
CDR3-
(SEQ ID NO: 219)
NALGGFLPNYG;
or
(lxiv) CDRs with the following sequences:
CDR1-
(SEQ ID NO: 37)
GSIFSINA
CDR2-
(SEQ ID NO: 38)
ITSGGST
CDR3-
(SEQ ID NO: 220)
NALGGFVTNYG;
or
(lxv) CDRs with the following sequences:
CDR1-
(SEQ ID NO: 37)
GSIFSINA
CDR2-
(SEQ ID NO: 38)
ITSGGST
CDR3-
(SEQ ID NO: 220)
NALGGFVTNYG;
or
(lxvi) CDRs with the following sequences:
CDR1-
(SEQ ID NO: 222)
GRTV
CDR2-
(SEQ ID NO: 185)
ITRGGST
CDR3-
(SEQ ID NO: 213)
NALGGFVPNYG;
or
(lxvii) CDRs with the following sequences:
CDR1-
(SEQ ID NO: 22)
GNIFRLNG
CDR2-
(SEQ ID NO: 23)
ITSGGNT
CDR3-
(SEQ ID NO: 39)
NALGGFVPSYG;
or
(lxviii) CDRs with the following sequences:
CDR1-
(SEQ ID NO: 163)
GNNFRLNA
CDR2-
(SEQ ID NO: 23)
ITSGGNT
CDR3-
(SEQ ID NO: 39)
NALGGFVPSYG;
or
(lxix) CDRs with the following sequences:
CDR1-
(SEQ ID NO: 22)
GNIFRLNG
CDR2-
(SEQ ID NO: 38)
ITSGGST
CDR3-
(SEQ ID NO: 39)
NALGGFVPSYG;
or
(lxx) CDRs with the following sequences:
CDR1-
(SEQ ID NO: 28)
GRSFSNYG
CDR2-
(SEQ ID NO: 29)
IGMVGGLT
CDR3-
(SEQ ID NO: 39)
NALGGFVPSYG;
or
(lxxi) CDRs with the following sequences:
CDR1-
(SEQ ID NO: 175)
GNIFSINA
CDR2-
(SEQ ID NO: 38)
ITSGGST
CDR3-
(SEQ ID NO: 39)
NALGGFVPSYG;
or
(lxxii) CDRs with the following sequences:
CDR1-
(SEQ ID NO: 165)
GFTFSTYW
CDR2-
(SEQ ID NO: 178)
IDNGGGTT
CDR3-
(SEQ ID NO: 221)
ARNPTRGWYSTDY.

One or more of the CDRs may comprise one, two or three amino acid mutations.

The binding domain may be a single domain antibody (sdAb). The sdAb may be a VHH or a heavy chain variable region (VH) domain. The sdAb may be a humanised sdAb.

Suitably, the FcRH5 binding domain may comprise:
(i) a variable heavy chain domain antibody (VHH) having the sequence shown as SEQ ID NO: 40 or a variant having at least 80% sequence identity thereto; or
(ii) a VHH having the sequence shown as SEQ ID NO: 41 or a variant having at least 80% sequence identity thereto; or
(iii) a VHH having the sequence shown as SEQ ID NO: 42 or a variant having at least 80% sequence identity thereto; or
(iv) a VHH having the sequence shown as SEQ ID NO: 43 or a variant having at least 80% sequence identity thereto; or
(v) a VHH having the sequence shown as SEQ ID NO: 44 or a variant having at least 80% sequence identity thereto; or
(vi) a VHH having the sequence shown as SEQ ID NO: 45 or a variant having at least 80% sequence identity thereto; or
(vii) a VHH having the sequence shown as SEQ ID NO: 46 or a variant having at least
(viii) a VHH having the sequence shown as SEQ ID NO: 47 or a variant having at least 80% sequence identity thereto; or
(ix) a VHH having the sequence shown as SEQ ID NO: 48 or a variant having at least 80% sequence identity thereto; or
(x) a VHH having the sequence shown as SEQ ID NO: 49 or a variant having at least 80% sequence identity thereto; or
(xi) a VHH having the sequence shown as SEQ ID NO: 50 or a variant having at least 80% sequence identity thereto; or
(xii) a VHH having the sequence shown as SEQ ID NO: 51 or a variant having at least 80% sequence identity thereto; or
(xiii) a VHH having the sequence shown as SEQ ID NO: 52 or a variant having at least 80% sequence identity thereto; or
(xiv) a VHH having the sequence shown as SEQ ID NO: 53 or a variant having at least 80% sequence identity thereto; or
(xv) a VHH having the sequence shown as SEQ ID NO: 158 or a variant having at least 80% sequence identity thereto; or
(xvi) a VHH having the sequence shown as SEQ ID NO: 223 or a variant having at least 80% sequence identity thereto; or
(xvii) a VHH having the sequence shown as SEQ ID NO: 224 or a variant having at least 80% sequence identity thereto; or
(xviii) a VHH having the sequence shown as SEQ ID NO: 225 or a variant having at least 80% sequence identity thereto; or
(xvix) a VHH having the sequence shown as SEQ ID NO: 226 or a variant having at least 80% sequence identity thereto; or
(xx) a VHH having the sequence shown as SEQ ID NO: 227 or a variant having at least 80% sequence identity thereto; or
(xxi) a VHH having the sequence shown as SEQ ID NO: 228 or a variant having at least 80% sequence identity thereto; or
(xxii) a VHH having the sequence shown as SEQ ID NO: 229 or a variant having at least 80% sequence identity thereto; or
(xxiii) a VHH having the sequence shown as SEQ ID NO: 230 or a variant having at least 80% sequence identity thereto; or
(xxiv) a VHH having the sequence shown as SEQ ID NO: 231 or a variant having at least 80% sequence identity thereto; or
(xxv) a VHH having the sequence shown as SEQ ID NO: 232 or a variant having at least
(xxvi) a VHH having the sequence shown as SEQ ID NO: 233 or a variant having at least 80% sequence identity thereto; or
(xxvii) a VHH having the sequence shown as SEQ ID NO: 234 or a variant having at least 80% sequence identity thereto; or
(xxviii) a VHH having the sequence shown as SEQ ID NO: 235 or a variant having at least 80% sequence identity thereto; or
(xxix) a VHH having the sequence shown as SEQ ID NO: 236 or a variant having at least 80% sequence identity thereto; or
(xxx) a VHH having the sequence shown as SEQ ID NO: 237 or a variant having at least 80% sequence identity thereto; or
(xxxi) a VHH having the sequence shown as SEQ ID NO: 238 or a variant having at least 80% sequence identity thereto; or
(xxxii) a VHH having the sequence shown as SEQ ID NO: 239 or a variant having at least 80% sequence identity thereto; or
(xxxiii) a VHH having the sequence shown as SEQ ID NO: 240 or a variant having at least 80% sequence identity thereto; or
(xxxiv) a VHH having the sequence shown as SEQ ID NO: 241 or a variant having at least 80% sequence identity thereto; or
(xxxv) a VHH having the sequence shown as SEQ ID NO: 242 or a variant having at least 80% sequence identity thereto; or
(xxxvi) a VHH having the sequence shown as SEQ ID NO: 243 or a variant having at least 80% sequence identity thereto; or
(xxxvii) a VHH having the sequence shown as SEQ ID NO: 244 or a variant having at least 80% sequence identity thereto; or
(xxxviii) a VHH having the sequence shown as SEQ ID NO: 245 or a variant having at least 80% sequence identity thereto; or
(xxxix) a VHH having the sequence shown as SEQ ID NO: 246 or a variant having at least 80% sequence identity thereto; or
(xl) a VHH having the sequence shown as SEQ ID NO: 247 or a variant having at least 80% sequence identity thereto; or
(xli) a VHH having the sequence shown as SEQ ID NO: 248 or a variant having at least 80% sequence identity thereto; or
(xlii) a VHH having the sequence shown as SEQ ID NO: 249 or a variant having at least 80% sequence identity thereto; or
(xliii) a VHH having the sequence shown as SEQ ID NO: 250 or a variant having at least
(xliv) a VHH having the sequence shown as SEQ ID NO: 251 or a variant having at least 80% sequence identity thereto; or
(xlv) a VHH having the sequence shown as SEQ ID NO: 252 or a variant having at least 80% sequence identity thereto; or
(xlvi) a VHH having the sequence shown as SEQ ID NO: 253 or a variant having at least 80% sequence identity thereto; or
(xlvii) a VHH having the sequence shown as SEQ ID NO: 254 or a variant having at least 80% sequence identity thereto; or
(xlviii) a VHH having the sequence shown as SEQ ID NO: 255 or a variant having at least 80% sequence identity thereto; or
(xlix) a VHH having the sequence shown as SEQ ID NO: 256 or a variant having at least 80% sequence identity thereto; or
(1) a VHH having the sequence shown as SEQ ID NO: 257 or a variant having at least 80% sequence identity thereto; or
(li) a VHH having the sequence shown as SEQ ID NO: 258 or a variant having at least 80% sequence identity thereto; or
(lii) a VHH having the sequence shown as SEQ ID NO: 259 or a variant having at least 80% sequence identity thereto; or
(liii) a VHH having the sequence shown as SEQ ID NO: 260 or a variant having at least 80% sequence identity thereto; or
(liv) a VHH having the sequence shown as SEQ ID NO: 261 or a variant having at least 80% sequence identity thereto; or
(lv) a VHH having the sequence shown as SEQ ID NO: 262 or a variant having at least 80% sequence identity thereto; or
(lvi) a VHH having the sequence shown as SEQ ID NO: 263 or a variant having at least 80% sequence identity thereto; or
(lvii) a VHH having the sequence shown as SEQ ID NO: 264 or a variant having at least 80% sequence identity thereto; or
(lviii) a VHH having the sequence shown as SEQ ID NO: 265 or a variant having at least 80% sequence identity thereto; or
(lix) a VHH having the sequence shown as SEQ ID NO: 266 or a variant having at least 80% sequence identity thereto; or
(lx) a VHH having the sequence shown as SEQ ID NO: 267 or a variant having at least 80% sequence identity thereto; or
(lxi) a VHH having the sequence shown as SEQ ID NO: 268 or a variant having at least
(lxii) a VHH having the sequence shown as SEQ ID NO: 269 or a variant having at least 80% sequence identity thereto; or
(lxiii) a VHH having the sequence shown as SEQ ID NO: 270 or a variant having at least 80% sequence identity thereto; or
(lxiv) a VHH having the sequence shown as SEQ ID NO: 271 or a variant having at least 80% sequence identity thereto; or
(lxv) a VHH having the sequence shown as SEQ ID NO: 272 or a variant having at least 80% sequence identity thereto; or
(lxvi) a VHH having the sequence shown as SEQ ID NO: 273 or a variant having at least 80% sequence identity thereto; or
(lxvii) a VHH having the sequence shown as SEQ ID NO: 274 or a variant having at least 80% sequence identity thereto; or
(lxviii) a VHH having the sequence shown as SEQ ID NO: 275 or a variant having at least 80% sequence identity thereto; or
(lxix) a VHH having the sequence shown as SEQ ID NO: 276 or a variant having at least 80% sequence identity thereto; or
(lxx) a VHH having the sequence shown as SEQ ID NO: 277 or a variant having at least 80% sequence identity thereto; or
(lxxi) a VHH having the sequence shown as SEQ ID NO: 278 or a variant having at least 80% sequence identity thereto; or
(lxxii) a VHH having the sequence shown as SEQ ID NO: 279 or a variant having at least 80% sequence identity thereto; or
(lxxiii) a VHH having the sequence shown as SEQ ID NO: 280 or a variant having at least 80% sequence identity thereto; or
(lxxiv) a VHH having the sequence shown as SEQ ID NO: 281 or a variant having at least 80% sequence identity thereto; or
(lxxv) a VHH having the sequence shown as SEQ ID NO: 282 or a variant having at least 80% sequence identity thereto; or
(lxxvi) a VHH having the sequence shown as SEQ ID NO: 283 or a variant having at least 80% sequence identity thereto; or
(lxxvii) a VHH having the sequence shown as SEQ ID NO: 284 or a variant having at least 80% sequence identity thereto; or
(lxxviii) a VHH having the sequence shown as SEQ ID NO: 285 or a variant having at least 80% sequence identity thereto; or
(lxxix) a VHH having the sequence shown as SEQ ID NO: 286 or a variant having at least
(lxxx) a VHH having the sequence shown as SEQ ID NO: 287 or a variant having at least 80% sequence identity thereto; or
(lxxxi) a VHH having the sequence shown as SEQ ID NO: 288 or a variant having at least 80% sequence identity thereto.

In a second aspect, the present invention provides a FcRH5 binding molecule comprising the sdAb according to the first aspect of the invention, wherein the FcRH5 binding molecule is selected from an antibody conjugate, a chimeric antigen receptor (CAR), or an immune cell engager, such as a T cell engager molecule, a gamma delta T cell engager molecule, a natural killer (NK) T cell engager molecule, or a NK cell engager molecule.

In a third aspect, the present invention provides an antibody conjugate comprising the sdAb according to the first aspect of the present invention.

Suitably the antibody conjugate may be conjugated to a chemotherapeutic entity, a radionuclide or a detection entity.

In a fourth aspect, the present invention provides a CAR comprising the sdAb according to the first aspect of the present invention.

The CAR may comprise a transmembrane domain comprising a CD8a transmembrane domain; a CD28 transmembrane domain; or a Tyrp1 transmembrane domain.

The CAR may comprise a transmembrane domain comprising one of the sequences selected from the group comprising: SEQ ID NO: 54, SEQ ID NO: 55 or SEQ ID NO: 56, or a variant thereof having at least 80% sequence identity.

The sdAb and the transmembrane domain may be connected by a spacer.

The spacer may comprise one of the following: a human an IgG1 Fc domain; an IgG1 hinge; an IgG1 hinge-CD8 stalk; or a CD8 stalk.

The spacer may comprise one of the sequences selected from the group comprising: SEQ ID NO: 57, SEQ ID NO: 58, SEQ ID NO: 59, SEQ ID NO: 60, or a SEQ ID NO: 61; or a variant thereof having at least 80% sequence identity.

The CAR may comprise an intracellular T cell signalling domain.

The intracellular T cell signalling domain may comprise one or more of the following endodomains: CD28 endodomain; OX40 endodomain; 41BB endodomain; and CD3-Zeta endodomain.

The intracellular T cell signalling domain may comprise one or more of the sequences selected from the group comprising: SEQ ID NO: 67, SEQ ID NO: 68, SEQ ID NO: 69, or SEQ ID NO: 70; or a variant thereof having at least 80% sequence identity.

The CAR may comprise the sequence selected from the group comprising SEQ ID NO: 76-SEQ ID NO: 89, SEQ ID NO: 159 and SEQ ID NO: 289-SEQ ID NO: 354, or a variant thereof which has at least 80% sequence identity thereto but retains the capacity to i) bind FcRH5 and ii) induce T cell signalling.

In a fifth aspect, the present invention provides an immune cell engager molecule which comprises:
(i) a first domain which comprises a sdAb according to the first aspect of the invention; and
(ii) a second domain capable of activating a T cell.

The immune cell engager molecule may further comprise a third domain capable of co-activating a T cell.

The second domain may activate a T cell by binding CD3 on the T cell surface.

The second domain may comprise a CD3-specific antibody or part thereof.

The second domain may comprise the sequence selected from the group comprising SEQ ID NO: 96, SEQ ID NO: 103 or SEQ ID NO: 110 or a variant thereof which has at least 80% sequence identity and binds CD3.

The first and second binding domains may be connected by a spacer or a linker. The second and third binding domains may be connected by a spacer or a linker.

The spacer may comprise an IgG1 hinge or a CD8 stalk.

The spacer may comprise one of the sequences selected from the group comprising: SEQ ID NO: 113 or SEQ ID NO: 114; or a variant thereof having at least 80% sequence identity. The linker may comprise a serine-glycine linker, such as SGGGGS (SEQ ID NO: 116), GGGGS (SEQ ID NO: 117), ((Gly₄)Ser)₂ (GGGGSGGGGS, SEQ ID NO: 355), ((Gly₄)Ser)₃ (GGGGSGGGGSGGGGS, SEQ ID NO: 356) and ((Gly₄)Ser)₄ (GGGGSGGGGSGGGGSGGGGS, SEQ ID NO: 357).

The third domain may be capable of binding CD28.

The third domain may comprise a CD28-specific antibody or part thereof.

The third domain may comprise
(i) a VH region having the sequence shown as SEQ ID NO: 130 and a VL region having the sequence shown as SEQ ID NO: 131; or
(ii) a VH region having the sequence shown as SEQ ID NO: 132 and a VL region having the sequence shown as SEQ ID NO: 133; or
(iii) a VH region having the sequence shown as SEQ ID NO: 134 and a VL region having the sequence shown as SEQ ID NO: 135;
or a variant thereof which has at least 80% sequence identity and binds CD28.

In a sixth aspect, the present invention provides an immune cell engager molecule which comprises:
(i) a first domain which comprises a sdAb according to the first aspect of the invention
(ii) a second domain capable of activating a NK cell.

The immune cell engager molecule may further comprise a third domain capable of activating a NK cell.

The second domain may activate a NK cell by binding CD16 on the NK cell surface.

The second domain may comprise a CD16-specific antibody or part thereof; optionally wherein the second domain comprises:
(i) a VH region having the sequence shown as SEQ ID NO: 142 and a VL region having the sequence shown as SEQ ID NO: 143; or
(ii) a VH region having the sequence shown as SEQ ID NO: 146 and a VL region having the sequence shown as SEQ ID NO: 147; or
(iii) a VH region having the sequence shown as SEQ ID NO: 153 and a VL region having the sequence shown as SEQ ID NO: 154 or a variant thereof which has at least 80% identity and binds CD16.

The third domain may be capable of cross-linking IL-15.

In a seventh aspect, the present invention provides a polynucleotide comprising a nucleic acid sequence encoding an sdAb according to the first aspect of the invention, a CAR according to the fourth aspect of the invention, an immune cell engager molecule according to the fifth aspect of the invention, or an immune cell engager molecule according to the sixth aspect of the invention.

In an eighth aspect, the present invention provides a vector which comprises a polynucleotide according to the seventh aspect of the invention.

In a ninth aspect, the present invention provides a cell which comprises a CAR according to the fourth aspect of the invention.

In a tenth aspect, the present invention provides a cell comprising the polynucleotide according to the seventh aspect of the invention or a vector according to the eighth aspect of the invention.

The cell may be an immune cell, such as a T cell or a NK cell.

In an eleventh aspect, the present invention provides a method for making a cell according to the ninth or tenth aspect of the invention, which comprises the step of introducing a polynucleotide according to the seventh aspect of the invention or a vector according to the eighth aspect of the invention.

In a twelfth aspect, the present invention provides a pharmaceutical composition which comprises a sdAb according to the first aspect of the invention, or an antibody conjugate according to the third aspect of the invention, or an immune cell engager molecule according to the fifth aspect of the invention, or an immune cell engager molecule according to the sixth aspect of the invention, or a polynucleotide according to the seventh aspect of the invention, or a vector according to the eighth aspect of the invention, or a cell according to the ninth or tenth aspect of the invention, together with a pharmaceutically acceptable carrier, diluent or excipient.

In a thirteenth aspect, the present invention provides a sdAb according to the first aspect of the invention, or an antibody conjugate according to the third aspect of the invention, or an immune cell engager molecule according to the fifth aspect of the invention, or an immune cell engager molecule according to the sixth aspect of the invention, or a polynucleotide according to the seventh aspect of the invention, or a vector according to the eighth aspect of the invention, or a cell according to the ninth or tenth aspect of the invention, or a pharmaceutical composition according to the twelfth aspect of the invention, for use as a medicament in the treatment of a disease.

In a fourteenth aspect, the present invention provides a method for treating a disease which comprises the step of administering a sdAb according to the first aspect of the invention, or an antibody conjugate according to the third aspect of the invention, or an immune cell engager molecule according to the fifth aspect of the invention, or an immune cell engager molecule according to the sixth aspect of the invention, or a polynucleotide according to the seventh aspect of the invention, or a vector according to the eighth aspect of the invention, or a cell according to the ninth or tenth aspect of the invention, or a pharmaceutical composition according to the twelfth aspect of the invention to a subject.

In a fifteenth aspect, the present invention provides a use of a sdAb according to the first aspect of the invention, or an antibody conjugate according to the third aspect of the invention, or an immune cell engager molecule according to the fifth aspect of the invention, or an immune cell engager molecule according to the sixth aspect of the invention, or a polynucleotide according to the seventh aspect of the invention, or a vector according to the eighth aspect of the invention, or a cell according to the ninth or tenth aspect of the invention, or a pharmaceutical composition according to the twelfth aspect of the invention in the manufacture of a medicament for treating a disease.

The disease may be a B cell malignancy or a plasma cell disorder.

The B cell malignancy or plasma cell disorder may be selected from the list comprising: leukaemia, hairy cell leukaemia, chronic lymphocytic leukaemia, non-Hodgkins lymphoma, mantle cell lymphoma, EBV-associated lymphoma (Burkitt), lymphoplasmacytic lymphoma, plasmacytoma, solitary plasmocytoma, extramedullary plasmocytoma, plasma cell leukemia, multiple myeloma, macroglobulinemia, amyloidosis, Waldenstrom's macroglobulinemia, solitary bone plasmacytoma, extramedullary plasmacytoma, osteosclerotic myeloma, heavy chain diseases, monoclonal gammopathy of undetermined significance (MGUS), non-IgM MGUS, IgM MGUS, light chain MGUS and smoldering multiple myeloma.

The cancer may be multiple myeloma.

### BRIEF DESCRIPTION OF THE FIGURES

FIG. 1: Schematic of aFCRH5-CAR construction for plate bound antigen assay

FIG. 2: mClover-NUR77 upregulation in Jurkat T cells after stimulation with plate FcRH5 (24 h). Most CAR candidates upregulate NUR77 after 24 h antigenic stimulation. Non-transduced (NT) cells show consistently low levels of mClover expression whilst positive control (aCD3/CD28) coated wells are highly activated.

FIG. 3: mClover-NUR77 upregulation in Jurkat T cells after stimulation with plate FcRH5 (72 h). As expected Nu77-mClover expression is reduced after 72 h compared with initial contact with the antigen. Decreases shown here are in line with positive control stimulation drops. Data presented are representative of duplicate experiments.

FIG. 4: mClover-NUR77 upregulation by anti-FcRH5 VHH CAR transduced Jurkat NR4A1 T cells upon stimulation with plate bound antigen (MFI vs FcRH5 concentration). A) 24 h incubation; B) 72 h incubation.

FIG. 5: Repeated mClover-NUR77 upregulation in Jurkat T cells after stimulation with plate FcRH5_2-fold dilution series (24 h).

FIG. 6: Percentage mClover positive cells after plate bound antigen activation. A) Activation of Jurkat T cells with PBA or aCD3/CD28 stimulation. B) Activation of Jurkat T cells excluding aCD3/CD28 data. PBA=plate bound antigen.

FIG. 7: ELISA assay on FcRH5 Ig8. ELISA assay for soluble anti-FcRH5 VHH antibodies on human FcRH5 Ig8 domain. Binding detected with anti-mouse secondary antibody for FcRH5 Ig8-muFc. A) Titration of anti-FcRH5 VHH-His coating. B) Ranking of clones based on maximum signal at 1 μg/ml VHH coating. Clone 85139 binding epitope falls outside the Ig8 domain.

FIG. 8: A) Representative dot plot of HEK293T NT and HEK293T transfected with FcRH5 Ig8 domain. 33% of cells result transfected with construct expressing human FcRH5 Ig8 domain, containing the eGFP transfection marker. B) Histogram plot of HEK293T NT staining with anti-FcRH5 VHH-His antibodies detected with anti-His AF647 secondary antibody. Blue curve=secondary only. Red curve=VHH-His staining. C) Histogram plot of HEK293T FcRH5 Ig8 staining with anti-FcRH5 VHH-His antibodies detected with anti-His AF647 secondary antibody. Blue curve=secondary only. Red curve=VHH-His staining.

FIG. 9: MADLS analysis of particle dispersion and aggregation profile for anti-FcRH5 binders. A) Particle size distribution. Antibodies show a predominantly mono-dispersed profile. B) Average particle diameter. Size in line with the expected MW of 82 kDa. C) % aggregates based on size.

### DETAILED DESCRIPTION OF THE INVENTION

### Single Domain Antibody

The present invention provides a single domain antibody (sdAb) comprising an FcRH5 binding domain. Thus, the sdAb may be capable of selectively binding to FcRH5.

As used herein, “antibody” means a protein or polypeptide having an antigen binding site or antigen-binding domain which comprises at least one complementarity determining region (CDR). The antibody may comprise 3 CDRs and have an antigen binding site which is equivalent to that of a domain antibody (sdAb).

The term “polypeptide” is used in the conventional sense to mean a series of amino acids, typically L-amino acids, connected one to the other, typically by peptide bonds between the α-amino and carboxyl groups of adjacent amino acids. The term “polypeptide” is used interchangeably with the terms “amino acid sequence”, “peptide” and/or “protein”. The term “residues” is used to refer to amino acids in an amino acid sequence.

A sdAb (i.e. a nanobody) is an antibody fragment comprising of a single monomeric variable antibody domain. The sdAb may be a single chain variable domain which may be a heavy chain variable (VH) domain or light chain variable (VL) domain, having 3 CDRs.

SdAbs have been engineered from heavy-chain antibodies found in camelids to produce variable heavy chain domain antibodies (VHHs). SdAbs have also been engineered from heavy-chain antibodies called immunoglobulin new antigen receptor (IgNAR) found in cartilaginous fishes to produce variable new antigen receptor antibodies (VNARs).

Suitably, the sdAb may be a VHH, a VH domain or a VNAR. Suitably, the sdAb may be a VHH.

The sdAb may be a Humabody® (Crescendo Biologics). A Humabody is an antibody produced by a transgenic mouse that produces heavy-chain-only antibodies with fully human VH domains, without VL domains.

The sdAb may be non-human, humanised or fully human. Suitably, the sdAb may be a humanised sdAb. Suitably, the sdAb may be a fully human sdAb.

The remainder of the polypeptide may be any sequence which provides a suitable scaffold for the antigen binding site and displays it in an appropriate manner for it to bind the antigen.

“Heavy chain variable region” or “VH” refers to the fragment of the heavy chain of an antigen-binding domain or antibody that contains three CDRs interposed between flanking stretches known as framework regions, which are more highly conserved than the CDRs and form a scaffold to support the CDRs. “Light chain variable region” or “VL” refers to the fragment of the light chain of an antigen-binding domain or antibody that contains three CDRs interposed between framework regions.

“Complementarity determining region” or “CDR” with regard to an antigen-binding domain or antibody or antigen-binding fragment thereof refers to a highly variable loop in the variable region of the heavy chain of the light chain of an antibody. CDRs can interact with the antigen conformation and largely determine binding to the antigen (although some framework regions are known to be involved in binding). The heavy chain variable region and the light chain variable region each contain 3 CDRs (heavy chain CDRs 1, 2 and 3 and light chain CDRs 1, 2 and 3, numbered from the amino to the carboxy terminus).

A number of definitions of the CDRs are commonly in use. The Kabat definition is based on sequence variability and is the most commonly used (see http://www.bioinf.org.uk/abs/). The ImMunoGeneTics information system (IMGT) (see http://www.imgt.org) can also be used. According to this system, a complementarity determining region (CDR-IMGT) is a loop region of a variable domain, delimited according to the IMGT unique numbering for V domain. There are three CDR-IMGT in a variable domain: CDR1-IMGT (loop BC), CDR2-IMGT (loop C′C″), and CDR3-IMGT (loop FG). Other definitions of the CDRs have also been developed, such as the Chothia, the AbM and the contact definitions (see http://www.imgt.org). Unless stated otherwise, the CDRs described herein are derived using the IMGT system.

“Humanised antibody” refers to a genetically engineered non-human antibody, which contains human antibody constant domains and non-human variable domains modified to contain a high level of sequence homology to human variable domains. This can be achieved by grafting of the three (or six) non-human antibody complementarity-determining regions (CDRs), which together form the antigen binding site, onto a homologous human acceptor framework region (FR). Non-limiting examples of antibody humanisation methods include CDR grafting, CDR homology (which is based on CDR homology between murine/human sequences rather than framework similarity); resurfacing (i.e. replacing surface residues to obtain a “more human” surface), and germline-based humanisation. For example, in order to fully reconstitute the binding affinity and specificity of the parental antibody, the substitution of framework residues from the parental antibody (i.e. the non-human antibody) into the human framework regions (back-mutations) may be required. Structural homology modelling may help to identify the amino acid residues in the framework regions that are important for the binding properties of the antibody. Thus, a humanised antibody may comprise non-human CDR sequences, primarily human framework regions optionally comprising one or more amino acid back-mutations to the non-human amino acid sequence, and, optionally, fully human constant regions. Optionally, additional amino acid modifications, which are not necessarily back-mutations, may be introduced to obtain a humanized antibody with preferred characteristics, such as affinity and biochemical properties. Humanisation of non-human therapeutic antibodies is performed to minimise its immunogenicity in man while such humanised antibodies at the same time maintain the specificity and binding affinity of the antibody of non-human origin. Exemplary methods for humanisation of VHHs are described in Vincke et al. (Journal of Biological Chemistry; 2009; 284 (5); 3273-3284) and Rossotti et al. (FEBS; 2021; doi: 10.1111/febs/15809).

The sdAb may prove useful in any method which relies on a binding interaction between an antigen-binding domain and a cognate target. Thus, the sdAb may be used as a detection antibody and/or a capture antibody. The sdAb may be used a therapeutic antibody, for example, as a therapeutic antibody that targets FcRH5 protein or a cell expressing FcRH5. A non-limiting example therefore for the application of the sdAb is the use in the treatment of cancers characterized by expression and/or overexpression of FcRH5.

The present invention also encompasses fragments of any sdAb or protein or polypeptide as defined herein. It will be appreciated that a fragment comprises an amino acid sequence that is shorter than the full-length sequence of an sdAb or protein or polypeptide, but retains full biological activity and/or antigenic nature of the full-length sequence of the sdAb or protein or polypeptide. It will also be appreciated that said fragment retains the same binding affinity of the full-length sequence of the sdAb or protein or polypeptide.

### FcRH5 Binding Domain

The binding domains described herein are able to specifically bind to FcRH5.

Suitably, the FcRH5 binding domain may comprise complementarity determining region (CDR) 1, CDR2 and CDR3 sequences.

In one embodiment, the FcRH5 binding domain may comprise the following sequences:

CDR1-
(SEQ ID NO: 1)
RSSFSNNA
CDR2-
(SEQ ID NO: 2)
ITKGGVT
CDR3-
(SEQ ID NO: 3)
NTIPFRSA

In one embodiment, the FcRH5 binding domain may comprise the following sequences:

CDR1-
(SEQ ID NO: 4)
GFTFSNYA
CDR2-
(SEQ ID NO: 5)
INSDGGTA
CDR3-
(SEQ ID NO: 6)
AANRGFCAGVRCLEYQY

In one embodiment, the FcRH5 binding domain may comprise the following sequences:

CDR1-
(SEQ ID NO: 7)
GRTFSINA
CDR2-
(SEQ ID NO: 8)
IGGSGRVSST
CDR3-
(SEQ ID NO: 9)
AARRDYLPFPPESYDY

In one embodiment, the FcRH5 binding domain may comprise the following sequences:

CDR1-
(SEQ ID NO: 7)
GRTFSINA
CDR2-
(SEQ ID NO: 8)
IGGSGRVSST
CDR3-
(SEQ ID NO: 10)
AAGRRTSTNGGDYDY

In one embodiment, the FcRH5 binding domain may comprise the following sequences:

CDR1-
(SEQ ID NO: 7)
GRTFSINA
CDR2-
(SEQ ID NO: 11)
ISRSGGAT
CDR3-
(SEQ ID NO: 12)
AGTRRAFSTGLRDYDY

In one embodiment, the FcRH5 binding domain may comprise the following sequences:

CDR1-
(SEQ ID NO: 13)
GRTFSNST
CDR2-
(SEQ ID NO: 14)
ISWSGGTY
CDR3-
(SEQ ID NO: 15)
AAARKGWSTRGDDYDY

In one embodiment, the FcRH5 binding domain may comprise the following sequences:

CDR1-
(SEQ ID NO: 16)
GRTYNNYA
CDR2-
(SEQ ID NO: 17)
ISRSGGMT
CDR3-
(SEQ ID NO: 18)
AAYVGGFSTARRDYSY

In one embodiment, the FcRH5 binding domain may comprise the following sequences:

CDR1-
(SEQ ID NO: 19)
GRTFSSYA
CDR2-
(SEQ ID NO: 20)
ISRIGGVT
CDR3-
(SEQ ID NO: 21)
AAAGLVSISTTPNDYDY

In one embodiment, the FcRH5 binding domain may comprise the following sequences:

CDR1-
(SEQ ID NO: 22)
GNIFRLNG
CDR2-
(SEQ ID NO: 23)
ITSGGNT
CDR3-
(SEQ ID NO: 24)
NAIPFRL

In one embodiment, the FcRH5 binding domain may comprise the following sequences:

CDR1-
(SEQ ID NO: 25)
RNIFSLNP
CDR2-
(SEQ ID NO: 26)
ITDGGST
CDR3-
(SEQ ID NO: 27)
NRVGGLQTWA

In one embodiment, the FcRH5 binding domain may comprise the following sequences:

CDR1-
(SEQ ID NO: 28)
GRSFSNYG
CDR2-
(SEQ ID NO: 29)
IGMVGGLT
CDR3-
(SEQ ID NO: 30)
AAGRRFSTSSRDYDI

In one embodiment, the FcRH5 binding domain may comprise the following sequences:

CDR1-
(SEQ ID NO: 31)
GRTFDSRP
CDR2-
(SEQ ID NO: 32)
VSWRGEST
CDR3-
(SEQ ID NO: 33)
AAGEPYSGTYYYRGRDYDY

In one embodiment, the FcRH5 binding domain may comprise the following sequences:

CDR1-
(SEQ ID NO: 34)
GRTFSMYA
CDR2-
(SEQ ID NO: 35)
ISGSARIT
CDR3-
(SEQ ID NO: 36)
AASSTYTSTSGSSYNY

In one embodiment, the FcRH5 binding domain may comprise the following sequences:

CDR1-
(SEQ ID NO: 37)
GSIFSINA
CDR2-
(SEQ ID NO: 38)
ITSGGST
CDR3-
(SEQ ID NO: 39)
NALGGFVPSYG

In one embodiment, the FcRH5 binding domain may comprise the following sequences:

CDR1-
(SEQ ID NO: 155)
GRTSSRAA
CDR2-
(SEQ ID NO: 156)
ISWSGGTT
CDR3-
(SEQ ID NO: 157)
AAARIFTTARNDYDH

In one embodiment, the FcRH5 binding domain may comprise the following sequences:

CDR1-
(SEQ ID NO: 22)
GNIFRLNG
CDR2-
(SEQ ID NO: 23)
ITSGGNT
CDR3-
(SEQ ID NO: 186)
NTIPFRLS

In one embodiment, the FcRH5 binding domain may comprise the following sequences:

CDR1-
(SEQ ID NO: 160)
GNIFRLNA
CDR2-
(SEQ ID NO: 23)
ITSGGNT
CDR3-
(SEQ ID NO: 186)
NTIPFRLS

In one embodiment, the FcRH5 binding domain may comprise the following sequences:

CDR1-
(SEQ ID NO: 1)
RSSFSNNA
CDR2-
(SEQ ID NO: 2)
ITKGGVT
CDR3-
(SEQ ID NO: 187)
NTIPVRSA

In one embodiment, the FcRH5 binding domain may comprise the following sequences:

CDR1-
(SEQ ID NO: 37)
GSIFSINA
CDR2-
(SEQ ID NO: 2)
ITKGGVT
CDR3-
(SEQ ID NO: 188)
NTIPCRSA

In one embodiment, the FcRH5 binding domain may comprise the following sequences:

CDR1-
(SEQ ID NO: 161)
GSSFRLNG
CDR2-
(SEQ ID NO: 38)
ITSGGST
CDR3-
(SEQ ID NO: 189)
NTIPFSRA

In one embodiment, the FcRH5 binding domain may comprise the following sequences:

CDR1
(SEQ ID NO: 162)
GRSVSINA
CDR2
(SEQ ID NO: 176)
IDRSGNT
CDR3-
(SEQ ID NO: 190)
NTIPYSDS

In one embodiment, the FcRH5 binding domain may comprise the following sequences:

CDR1-
(SEQ ID NO: 19)
GRTFSSYA
CDR2
(SEQ ID NO: 177)
IDGIGGIT
CDR3-
(SEQ ID NO: 3)
NTIPFRSA

In one embodiment, the FcRH5 binding domain may comprise the following sequences:

CDR1-
(SEQ ID NO: 1)
RSSFSNNA
CDR2-
(SEQ ID NO: 2)
ITKGGVT
CDR3-
(SEQ ID NO: 191)
NAIPFRSA

In one embodiment, the FcRH5 binding domain may comprise the following sequences:

CDR1
(SEQ ID NO: 163)
GNNFRLNA
CDR2-
(SEQ ID NO: 23)
ITSGGNT
CDR3-
(SEQ ID NO: 192)
NAIPFRPS

In one embodiment, the FcRH5 binding domain may comprise the following sequences:

CDR1
(SEQ ID NO: 22)
GNIFRLNG
CDR2-
(SEQ ID NO: 23)
ITSGGNT
CDR3-
(SEQ ID NO: 193)
NAIPFRRS

In one embodiment, the FcRH5 binding domain may comprise the following sequences:

CDR1-
(SEQ ID NO: 164)
ERIFRINA
CDR2-
(SEQ ID NO: 23)
ITSGGNT
CDR3-
(SEQ ID NO: 193)
NAIPFRRS

In one embodiment, the FcRH5 binding domain may comprise the following sequences:

CDR1-
(SEQ ID NO: 165)
GFTFSTYW
CDR2-
(SEQ ID NO: 178)
IDNGGGTT
CDR3-
(SEQ ID NO: 194)
NALPFRLS

In one embodiment, the FcRH5 binding domain may comprise the following sequences:

CDR1-
(SEQ ID NO: 22)
GNIFRLNG
CDR2-
(SEQ ID NO: 23)
ITSGGNT
CDR3-
(SEQ ID NO: 195)
NATPFRLS

In one embodiment, the FcRH5 binding domain may comprise the following sequences:

CDR1-
(SEQ ID NO: 22)
GNIFRLNG
CDR2-
(SEQ ID NO: 179)
ITRGGNT
CDR3-
(SEQ ID NO: 196)
NSIPFRLS

In one embodiment, the FcRH5 binding domain may comprise the following sequences:

CDR1-
(SEQ ID NO: 166)
GNIFRING
CDR2-
(SEQ ID NO: 23)
ITSGGNT
CDR3-
(SEQ ID NO: 197)
NAIPFRIS

In one embodiment, the FcRH5 binding domain may comprise the following sequences:

CDR1-
(SEQ ID NO: 163)
GNNFRLNA
CDR2-
(SEQ ID NO: 23)
ITSGGNT
CDR3-
(SEQ ID NO: 198)
NAIPFRLY

In one embodiment, the FcRH5 binding domain may comprise the following sequences:

CDR1-
(SEQ ID NO: 165)
GFTFSTYW
CDR2-
(SEQ ID NO: 178)
IDNGGGTT
CDR3-
(SEQ ID NO: 199)
KAIPFRLS

In one embodiment, the FcRH5 binding domain may comprise the following sequences:

CDR1-
(SEQ ID NO: 167)
GSSFSNNA
CDR2-
(SEQ ID NO: 2)
ITKGGVT
CDR3-
(SEQ ID NO: 3)
NTIPFRSA

In one embodiment, the FcRH5 binding domain may comprise the following sequences:

CDR1-
(SEQ ID NO: 165)
GFTFSTYW
CDR2-
(SEQ ID NO: 178)
IDNGGGTT
CDR3-
(SEQ ID NO: 3)
NTIPFRSA

In one embodiment, the FcRH5 binding domain may comprise the following sequences:

CDR1-
(SEQ ID NO: 160)
GNIFRLNA
CDR2-
(SEQ ID NO: 2)
ITKGGVT
CDR3-
(SEQ ID NO: 3)
NTIPFRSA

In one embodiment, the FcRH5 binding domain may comprise the following sequences:

(SEQ ID NO: 168)
CDR1-RSSFGNNA
(SEQ ID NO: 2)
CDR2-ITKGGVT
(SEQ ID NO: 3)
CDR3-NTIPFRSA

In one embodiment, the FcRH5 binding domain may comprise the following sequences:

(SEQ ID NO: 169)
CDR1-GRTFSTYG
(SEQ ID NO: 11)
CDR2-ISRSGGAT
(SEQ ID NO: 12)
CDR3-AGTRRAFSTGLRDYDY

In one embodiment, the FcRH5 binding domain may comprise the following sequences:

(SEQ ID NO: 170)
CDR1-GTIERNNA
(SEQ ID NO: 38)
CDR2-ITSGGST
(SEQ ID NO: 200)
CDR3-AAGRRFSTRSRDYDY

In one embodiment, the FcRH5 binding domain may comprise the following sequences:

(SEQ ID NO: 28)
CDR1-GRSFSNYG
(SEQ ID NO: 29)
CDR2-IGMVGGLT
(SEQ ID NO: 201)
CDR3-AAGRRFSTSSRDYDY

In one embodiment, the FcRH5 binding domain may comprise the following sequences:

(SEQ ID NO: 28)
CDR1-GRSFSNYG
(SEQ ID NO: 29)
CDR2-IGMVGGLT
(SEQ ID NO: 202)
CDR3-AAGRRFSTGSRDYDI

In one embodiment, the FcRH5 binding domain may comprise the following sequences:

(SEQ ID NO: 19)
CDR1-GRTFSSYA
(SEQ ID NO: 179)
CDR2-ISQFGGVTT
(SEQ ID NO: 202)
CDR3-AAGRRFSTGSRDYDI

In one embodiment, the FcRH5 binding domain may comprise the following sequences:

(SEQ ID NO: 28)
CDR1-GRSFSNYG
(SEQ ID NO: 29)
CDR2-IGMVGGLT
(SEQ ID NO: 203)
CDR3-AGGRRFSTSSRDYDI

In one embodiment, the FcRH5 binding domain may comprise the following sequences:

```
(SEQ ID NO: 28)
CDR1-GRSFSNYG
  (SEQ ID NO: 29)
CDR2-IGMVGGLT
  (SEQ ID NO: 204)
CDR3-AAGRRFSTSSREYDI
```

In one embodiment, the FcRH5 binding domain may comprise the following sequences:

(SEQ ID NO: 28)
CDR1-GRSFSNYG
(SEQ ID NO: 180)
CDR2-IGMVGGLP
(SEQ ID NO: 205)
CDR3-AAGRRLSTSSRDYDI

In one embodiment, the FcRH5 binding domain may comprise the following sequences:

(SEQ ID NO: 28)
CDR1-GRSFSNYG
(SEQ ID NO: 181)
CDR2-ISRGGGVS
(SEQ ID NO: 206)
CDR3-AAGLRFSTGSRDYDI

In one embodiment, the FcRH5 binding domain may comprise the following sequences:

(SEQ ID NO: 171)
CDR1-GRTFRRYA
(SEQ ID NO: 17)
CDR2-ISRSGGMT
(SEQ ID NO: 207)
CDR3-AAYVGGFSTTRRDYAY

In one embodiment, the FcRH5 binding domain may comprise the following sequences:

(SEQ ID NO: 13)
CDR1-GRTFSNST
(SEQ ID NO: 156)
CDR2-ISWSGGTT
(SEQ ID NO: 15)
CDR3-AAARKGWSTRGDDYDY

In one embodiment, the FcRH5 binding domain may comprise the following sequences:

```
  (SEQ ID NO: 172)
CDR1-GRTVI
  (SEQ ID NO: 182)
CDR2-SSGSGGVT
  (SEQ ID NO: 208)
CDR3-AAALTWSTRPSDFTS
```

In one embodiment, the FcRH5 binding domain may comprise the following sequences:

(SEQ ID NO: 172)
CDR1-GRTVI
(SEQ ID NO: 183)
CDR2-SNWSGGVT
(SEQ ID NO: 18)
CDR3-AAYVGGFSTARRDYSY

```
  (SEQ ID NO: 7)
CDR1-GRTFSINA
(SEQ ID NO: 17)
CDR2-ISRSGGMT
  (SEQ ID NO: 18)
CDR3-AAYVGGFSTARRDYSY
```

In one embodiment, the FcRH5 binding domain may comprise the following sequences:

(SEQ ID NO: 173)
CDR1-GRTFSRYA
(SEQ ID NO: 184)
CDR2-INGSGGT
(SEQ ID NO: 209)
CDR3-AAARIFTTTRNEYDH

In one embodiment, the FcRH5 binding domain may comprise the following sequences:

(SEQ ID NO: 173)
CDR1-GRTFSRYA
(SEQ ID NO: 184)
CDR2-INGSGGT
(SEQ ID NO: 209)
CDR3-AAARIFTTTRNEYDH

In one embodiment, the FcRH5 binding domain may comprise the following sequences:

(SEQ ID NO: 165)
CDR1-GFTFSTYW
(SEQ ID NO: 178)
CDR2-IDNGGGTT
(SEQ ID NO: 210)
CDR3-AAARIFSTARNDYDH

In one embodiment, the FcRH5 binding domain may comprise the following sequences:

(SEQ ID NO: 28)
CDR1-GRSFSNYG
(SEQ ID NO: 29)
CDR2-IGMVGGLT
(SEQ ID NO: 211)
CDR3-AGGRIFRTSSRDYDI

In one embodiment, the FcRH5 binding domain may comprise the following sequences:

(SEQ ID NO: 22)
CDR1-GNIFRLNG
(SEQ ID NO: 23)
CDR2-ITSGGNT
(SEQ ID NO: 212)
CDR3-AAARFFTTARNDYDH

In one embodiment, the FcRH5 binding domain may comprise the following sequences:

(SEQ ID NO: 37)
CDR1-GSIFSINA
(SEQ ID NO: 38)
CDR2-ITSGGST
(SEQ ID NO: 213)
CDR3-NALGGFVPNYG

In one embodiment, the FcRH5 binding domain may comprise the following sequences:

(SEQ ID NO: 22)
CDR1-GNIFRLNG
(SEQ ID NO: 23)
CDR2-ITSGGNT
(SEQ ID NO: 213)
CDR3-NALGGFVPNYG

In one embodiment, the FcRH5 binding domain may comprise the following sequences:

(SEQ ID NO: 37)
CDR1-GSIFSINA
(SEQ ID NO: 38)
CDR2-ITSGGST
(SEQ ID NO: 214)
CDR3-NALGGFVPNDG

In one embodiment, the FcRH5 binding domain may comprise the following sequences:

(SEQ ID NO: 163)
CDR1-GNNFRLNA
(SEQ ID NO: 23)
CDR2-ITSGGNT
(SEQ ID NO: 215)
CDR3-NALGGFAPNYG

In one embodiment, the FcRH5 binding domain may comprise the following sequences:

(SEQ ID NO: 28)
CDR1-GRSFSNYG
(SEQ ID NO: 29)
CDR2-IGMVGGLT
(SEQ ID NO: 216)
CDR3-NALGGFVPNYV

In one embodiment, the FcRH5 binding domain may comprise the following sequences:

(SEQ ID NO: 174)
CDR1-GRSFSINA
(SEQ ID NO: 38)
CDR2-ITSGGST
(SEQ ID NO: 217)
CDR3-NALGGFVLNYG

In one embodiment, the FcRH5 binding domain may comprise the following sequences:

(SEQ ID NO: 37)
CDR1-GSIFSINA
(SEQ ID NO: 38)
CDR2-ITSGGST
(SEQ ID NO: 218)
CDR3-NALGGLVPNYG

In one embodiment, the FcRH5 binding domain may comprise the following sequences:

(SEQ ID NO: 37)
CDR1-GSIFSINA
(SEQ ID NO: 38)
CDR2-ITSGGST
(SEQ ID NO: 219)
CDR3-NALGGFLPNYG

In one embodiment, the FcRH5 binding domain may comprise the following sequences:

(SEQ ID NO: 37)
CDR1-GSIFSINA
(SEQ ID NO: 38)
CDR2-ITSGGST
(SEQ ID NO: 220)
CDR3-NALGGFVTNYG

In one embodiment, the FcRH5 binding domain may comprise the following sequences:

(SEQ ID NO: 37)
CDR1-GSIFSINA
(SEQ ID NO: 38)
CDR2-ITSGGST
(SEQ ID NO: 220)
CDR3-NALGGFVTNYG

In one embodiment, the FcRH5 binding domain may comprise the following sequences:

CDR1-
(SEQ ID NO: 222)
GRTV
CDR2-
(SEQ ID NO: 185)
ITRGGST
CDR3-
(SEQ ID NO: 213)
NALGGFVPNYG

In one embodiment, the FcRH5 binding domain may comprise the following sequences:

CDR1-
(SEQ ID NO: 22)
GNIFRLNG
CDR2-
(SEQ ID NO: 23)
ITSGGNT
CDR3-
(SEQ ID NO: 39)
NALGGFVPSYG

In one embodiment, the FcRH5 binding domain may comprise the following sequences:

CDR1-
(SEQ ID NO: 163)
GNNFRLNA
CDR2-
(SEQ ID NO: 23)
ITSGGNT
CDR3-
(SEQ ID NO: 39)
NALGGFVPSYG

In one embodiment, the FcRH5 binding domain may comprise the following sequences:

CDR1-
(SEQ ID NO: 22)
GNIFRLNG
CDR2-
(SEQ ID NO: 38)
ITSGGST
CDR3-
(SEQ ID NO: 39)
NALGGFVPSYG

In one embodiment, the FcRH5 binding domain may comprise the following sequences:

CDR1-
(SEQ ID NO: 28)
GRSFSNYG
CDR2-
(SEQ ID NO: 29)
IGMVGGLT
CDR3-
(SEQ ID NO: 39)
NALGGFVPSYG

In one embodiment, the FcRH5 binding domain may comprise the following sequences:

CDR1-
(SEQ ID NO: 175)
GNIFSINA
CDR2-
(SEQ ID NO: 38)
ITSGGST
CDR3-
(SEQ ID NO: 39)
NALGGFVPSYG

In one embodiment, the FcRH5 binding domain may comprise the following sequences:

CDR1-
(SEQ ID NO: 165)
GFTFSTYW
CDR2-
(SEQ ID NO: 178)
IDNGGGTT
CDR3-
(SEQ ID NO: 221)
ARNPTRGWYSTDY

One or more of the CDRs may comprise one, two or three amino acid mutations. The FcRH5 binding domain comprising these CDRs may suitably maintain the capacity to bind FcRH5.

Suitably, the FcRH5 binding domain may comprise a VHH.

In one embodiment, the FcRH5 binding domain may comprise a VHH having the sequence shown as SEQ ID NO: 40 or a variant having at least 80%, 85%, 90%, 95%, 98% or 99% sequence identity thereto. Suitably the FcRH5 binding domain may comprise a VHH having the sequence shown as SEQ ID NO: 40.

In one embodiment, the FcRH5 binding domain may comprise a VHH having the sequence shown as SEQ ID NO: 41 or a variant having at least 80%, 85%, 90%, 95%, 98% or 99% sequence identity thereto. Suitably the FcRH5 binding domain may comprise a VHH having the sequence shown as SEQ ID NO: 41.

In one embodiment, the FcRH5 binding domain may comprise a VHH having the sequence shown as SEQ ID NO: 42 or a variant having at least 80%, 85%, 90%, 95%, 98% or 99% sequence identity thereto. Suitably the FcRH5 binding domain may comprise a VHH having the sequence shown as SEQ ID NO: 42.

In one embodiment, the FcRH5 binding domain may comprise a VHH having the sequence shown as SEQ ID NO: 43 or a variant having at least 80%, 85%, 90%, 95%, 98% or 99% sequence identity thereto. Suitably the FcRH5 binding domain may comprise a VHH having the sequence shown as SEQ ID NO: 43.

In one embodiment, the FcRH5 binding domain may comprise a VHH having the sequence shown as SEQ ID NO: 44 or a variant having at least 80%, 85%, 90%, 95%, 98% or 99% sequence identity thereto. Suitably the FcRH5 binding domain may comprise a VHH having the sequence shown as SEQ ID NO: 44.

In one embodiment, the FcRH5 binding domain may comprise a VHH having the sequence shown as SEQ ID NO: 45 or a variant having at least 80%, 85%, 90%, 95%, 98% or 99% the sequence shown as SEQ ID NO: 45.

In one embodiment, the FcRH5 binding domain may comprise a VHH having the sequence shown as SEQ ID NO: 46 or a variant having at least 80%, 85%, 90%, 95%, 98% or 99% sequence identity thereto. Suitably FcRH5 binding domain may comprise a VHH having the sequence shown as SEQ ID NO: 46.

In one embodiment, the FcRH5 binding domain may comprise a VHH having the sequence shown as SEQ ID NO: 47 or a variant having at least 80%, 85%, 90%, 95%, 98% or 99% sequence identity thereto. Suitably the FcRH5 binding domain may comprise a VHH having the sequence shown as SEQ ID NO: 47.

In one embodiment, the FcRH5 binding domain may comprise a VHH having the sequence shown as SEQ ID NO: 48 or a variant having at least 80%, 85%, 90%, 95%, 98% or 99% sequence identity thereto. Suitably the FcRH5 binding domain may comprise a VHH having the sequence shown as SEQ ID NO: 48.

In one embodiment, the FcRH5 binding domain may comprise a VHH having the sequence shown as SEQ ID NO: 49 or a variant having at least 80%, 85%, 90%, 95%, 98% or 99% sequence identity thereto. Suitably the FcRH5 binding domain may comprise a VHH having the sequence shown as SEQ ID NO: 49.

In one embodiment, the FcRH5 binding domain may comprise a VHH having the sequence shown as SEQ ID NO: 50 or a variant having at least 80%, 85%, 90%, 95%, 98% or 99% sequence identity thereto. Suitably the FcRH5 binding domain may comprise a VHH having the sequence shown as SEQ ID NO: 50.

In one embodiment, the FcRH5 binding domain may comprise a VHH having the sequence shown as SEQ ID NO: 51 or a variant having at least 80%, 85%, 90%, 95%, 98% or 99% sequence identity thereto. Suitably the FcRH5 binding domain may comprise a VHH having the sequence shown as SEQ ID NO: 51.

In one embodiment, the FcRH5 binding domain may comprise a VHH having the sequence shown as SEQ ID NO: 52 or a variant having at least 80%, 85%, 90%, 95%, 98% or 99% the sequence shown as SEQ ID NO: 52.

In one embodiment, the FcRH5 binding domain may comprise a VHH having the sequence shown as SEQ ID NO: 53 or a variant having at least 80%, 85%, 90%, 95%, 98% or 99% sequence identity thereto. Suitably the FcRH5 binding domain may comprise a VHH having the sequence shown as SEQ ID NO: 53.

In one embodiment, the FcRH5 binding domain may comprise a VHH having the sequence shown as SEQ ID NO: 158 or a variant having at least 80%, 85%, 90%, 95%, 98% or 99% sequence identity thereto. Suitably the FcRH5 binding domain may comprise a VHH having the sequence shown as SEQ ID NO: 158.

In one embodiment, the FcRH5 binding domain may comprise a VHH having the sequence shown as SEQ ID NO: 223 or a variant having at least 80%, 85%, 90%, 95%, 98% or 99% sequence identity thereto. Suitably the FcRH5 binding domain may comprise a VHH having the sequence shown as SEQ ID NO: 223.

In one embodiment, the FcRH5 binding domain may comprise a VHH having the sequence shown as SEQ ID NO: 224 or a variant having at least 80%, 85%, 90%, 95%, 98% or 99% sequence identity thereto. Suitably the FcRH5 binding domain may comprise a VHH having the sequence shown as SEQ ID NO: 224.

In one embodiment, the FcRH5 binding domain may comprise a VHH having the sequence shown as SEQ ID NO: 225 or a variant having at least 80%, 85%, 90%, 95%, 98% or 99% sequence identity thereto. Suitably the FcRH5 binding domain may comprise a VHH having the sequence shown as SEQ ID NO: 225.

In one embodiment, the FcRH5 binding domain may comprise a VHH having the sequence shown as SEQ ID NO: 226 or a variant having at least 80%, 85%, 90%, 95%, 98% or 99% sequence identity thereto. Suitably the FcRH5 binding domain may comprise a VHH having the sequence shown as SEQ ID NO: 226.

In one embodiment, the FcRH5 binding domain may comprise a VHH having the sequence shown as SEQ ID NO: 227 or a variant having at least 80%, 85%, 90%, 95%, 98% or 99% the sequence shown as SEQ ID NO: 227.

In one embodiment, the FcRH5 binding domain may comprise a VHH having the sequence shown as SEQ ID NO: 228 or a variant having at least 80%, 85%, 90%, 95%, 98% or 99% sequence identity thereto. Suitably the FcRH5 binding domain may comprise a VHH having the sequence shown as SEQ ID NO: 228.

In one embodiment, the FcRH5 binding domain may comprise a VHH having the sequence shown as SEQ ID NO: 229 or a variant having at least 80%, 85%, 90%, 95%, 98% or 99% sequence identity thereto. Suitably the FcRH5 binding domain may comprise a VHH having the sequence shown as SEQ ID NO: 229.

In one embodiment, the FcRH5 binding domain may comprise a VHH having the sequence shown as SEQ ID NO: 230 or a variant having at least 80%, 85%, 90%, 95%, 98% or 99% sequence identity thereto. Suitably the FcRH5 binding domain may comprise a VHH having the sequence shown as SEQ ID NO: 230.

In one embodiment, the FcRH5 binding domain may comprise a VHH having the sequence shown as SEQ ID NO: 231 or a variant having at least 80%, 85%, 90%, 95%, 98% or 99% sequence identity thereto. Suitably the FcRH5 binding domain may comprise a VHH having the sequence shown as SEQ ID NO: 231.

In one embodiment, the FcRH5 binding domain may comprise a VHH having the sequence shown as SEQ ID NO: 232 or a variant having at least 80%, 85%, 90%, 95%, 98% or 99% sequence identity thereto. Suitably the FcRH5 binding domain may comprise a VHH having the sequence shown as SEQ ID NO: 232.

In one embodiment, the FcRH5 binding domain may comprise a VHH having the sequence shown as SEQ ID NO: 233 or a variant having at least 80%, 85%, 90%, 95%, 98% or 99% sequence identity thereto. Suitably the FcRH5 binding domain may comprise a VHH having the sequence shown as SEQ ID NO: 233.

In one embodiment, the FcRH5 binding domain may comprise a VHH having the sequence shown as SEQ ID NO: 234 or a variant having at least 80%, 85%, 90%, 95%, 98% or 99% the sequence shown as SEQ ID NO: 234.

In one embodiment, the FcRH5 binding domain may comprise a VHH having the sequence shown as SEQ ID NO: 235 or a variant having at least 80%, 85%, 90%, 95%, 98% or 99% sequence identity thereto. Suitably the FcRH5 binding domain may comprise a VHH having the sequence shown as SEQ ID NO: 235.

In one embodiment, the FcRH5 binding domain may comprise a VHH having the sequence shown as SEQ ID NO: 236 or a variant having at least 80%, 85%, 90%, 95%, 98% or 99% sequence identity thereto. Suitably the FcRH5 binding domain may comprise a VHH having the sequence shown as SEQ ID NO: 236.

In one embodiment, the FcRH5 binding domain may comprise a VHH having the sequence shown as SEQ ID NO: 237 or a variant having at least 80%, 85%, 90%, 95%, 98% or 99% sequence identity thereto. Suitably the FcRH5 binding domain may comprise a VHH having the sequence shown as SEQ ID NO: 237.

In one embodiment, the FcRH5 binding domain may comprise a VHH having the sequence shown as SEQ ID NO: 238 or a variant having at least 80%, 85%, 90%, 95%, 98% or 99% sequence identity thereto. Suitably the FcRH5 binding domain may comprise a VHH having the sequence shown as SEQ ID NO: 238.

In one embodiment, the FcRH5 binding domain may comprise a VHH having the sequence shown as SEQ ID NO: 239 or a variant having at least 80%, 85%, 90%, 95%, 98% or 99% sequence identity thereto. Suitably the FcRH5 binding domain may comprise a VHH having the sequence shown as SEQ ID NO: 239.

In one embodiment, the FcRH5 binding domain may comprise a VHH having the sequence shown as SEQ ID NO: 240 or a variant having at least 80%, 85%, 90%, 95%, 98% or 99% sequence identity thereto. Suitably the FcRH5 binding domain may comprise a VHH having the sequence shown as SEQ ID NO: 240.

In one embodiment, the FcRH5 binding domain may comprise a VHH having the sequence shown as SEQ ID NO: 241 or a variant having at least 80%, 85%, 90%, 95%, 98% or 99% the sequence shown as SEQ ID NO: 241.

In one embodiment, the FcRH5 binding domain may comprise a VHH having the sequence shown as SEQ ID NO: 242 or a variant having at least 80%, 85%, 90%, 95%, 98% or 99% sequence identity thereto. Suitably the FcRH5 binding domain may comprise a VHH having the sequence shown as SEQ ID NO: 242.

In one embodiment, the FcRH5 binding domain may comprise a VHH having the sequence shown as SEQ ID NO: 243 or a variant having at least 80%, 85%, 90%, 95%, 98% or 99% sequence identity thereto. Suitably the FcRH5 binding domain may comprise a VHH having the sequence shown as SEQ ID NO: 243.

In one embodiment, the FcRH5 binding domain may comprise a VHH having the sequence shown as SEQ ID NO: 244 or a variant having at least 80%, 85%, 90%, 95%, 98% or 99% sequence identity thereto. Suitably the FcRH5 binding domain may comprise a VHH having the sequence shown as SEQ ID NO: 244.

In one embodiment, the FcRH5 binding domain may comprise a VHH having the sequence shown as SEQ ID NO: 245 or a variant having at least 80%, 85%, 90%, 95%, 98% or 99% sequence identity thereto. Suitably the FcRH5 binding domain may comprise a VHH having the sequence shown as SEQ ID NO: 245.

In one embodiment, the FcRH5 binding domain may comprise a VHH having the sequence shown as SEQ ID NO: 246 or a variant having at least 80%, 85%, 90%, 95%, 98% or 99% sequence identity thereto. Suitably the FcRH5 binding domain may comprise a VHH having the sequence shown as SEQ ID NO: 246.

In one embodiment, the FcRH5 binding domain may comprise a VHH having the sequence shown as SEQ ID NO: 247 or a variant having at least 80%, 85%, 90%, 95%, 98% or 99% sequence identity thereto. Suitably the FcRH5 binding domain may comprise a VHH having the sequence shown as SEQ ID NO: 247.

In one embodiment, the FcRH5 binding domain may comprise a VHH having the sequence shown as SEQ ID NO: 248 or a variant having at least 80%, 85%, 90%, 95%, 98% or 99% the sequence shown as SEQ ID NO: 248.

In one embodiment, the FcRH5 binding domain may comprise a VHH having the sequence shown as SEQ ID NO: 249 or a variant having at least 80%, 85%, 90%, 95%, 98% or 99% sequence identity thereto. Suitably the FcRH5 binding domain may comprise a VHH having the sequence shown as SEQ ID NO: 249.

In one embodiment, the FcRH5 binding domain may comprise a VHH having the sequence shown as SEQ ID NO: 250 or a variant having at least 80%, 85%, 90%, 95%, 98% or 99% sequence identity thereto. Suitably the FcRH5 binding domain may comprise a VHH having the sequence shown as SEQ ID NO: 250.

In one embodiment, the FcRH5 binding domain may comprise a VHH having the sequence shown as SEQ ID NO: 251 or a variant having at least 80%, 85%, 90%, 95%, 98% or 99% sequence identity thereto. Suitably the FcRH5 binding domain may comprise a VHH having the sequence shown as SEQ ID NO: 251.

In one embodiment, the FcRH5 binding domain may comprise a VHH having the sequence shown as SEQ ID NO: 252 or a variant having at least 80%, 85%, 90%, 95%, 98% or 99% sequence identity thereto. Suitably the FcRH5 binding domain may comprise a VHH having the sequence shown as SEQ ID NO: 252.

In one embodiment, the FcRH5 binding domain may comprise a VHH having the sequence shown as SEQ ID NO: 253 or a variant having at least 80%, 85%, 90%, 95%, 98% or 99% sequence identity thereto. Suitably the FcRH5 binding domain may comprise a VHH having the sequence shown as SEQ ID NO: 253.

In one embodiment, the FcRH5 binding domain may comprise a VHH having the sequence shown as SEQ ID NO: 254 or a variant having at least 80%, 85%, 90%, 95%, 98% or 99% sequence identity thereto. Suitably the FcRH5 binding domain may comprise a VHH having the sequence shown as SEQ ID NO: 254.

In one embodiment, the FcRH5 binding domain may comprise a VHH having the sequence shown as SEQ ID NO: 255 or a variant having at least 80%, 85%, 90%, 95%, 98% or 99% the sequence shown as SEQ ID NO: 255.

In one embodiment, the FcRH5 binding domain may comprise a VHH having the sequence shown as SEQ ID NO: 256 or a variant having at least 80%, 85%, 90%, 95%, 98% or 99% sequence identity thereto. Suitably the FcRH5 binding domain may comprise a VHH having the sequence shown as SEQ ID NO: 256.

In one embodiment, the FcRH5 binding domain may comprise a VHH having the sequence shown as SEQ ID NO: 257 or a variant having at least 80%, 85%, 90%, 95%, 98% or 99% sequence identity thereto. Suitably the FcRH5 binding domain may comprise a VHH having the sequence shown as SEQ ID NO: 257.

In one embodiment, the FcRH5 binding domain may comprise a VHH having the sequence shown as SEQ ID NO: 258 or a variant having at least 80%, 85%, 90%, 95%, 98% or 99% sequence identity thereto. Suitably the FcRH5 binding domain may comprise a VHH having the sequence shown as SEQ ID NO: 258.

In one embodiment, the FcRH5 binding domain may comprise a VHH having the sequence shown as SEQ ID NO: 259 or a variant having at least 80%, 85%, 90%, 95%, 98% or 99% sequence identity thereto. Suitably the FcRH5 binding domain may comprise a VHH having the sequence shown as SEQ ID NO: 259.

In one embodiment, the FcRH5 binding domain may comprise a VHH having the sequence shown as SEQ ID NO: 260 or a variant having at least 80%, 85%, 90%, 95%, 98% or 99% sequence identity thereto. Suitably the FcRH5 binding domain may comprise a VHH having the sequence shown as SEQ ID NO: 260.

In one embodiment, the FcRH5 binding domain may comprise a VHH having the sequence shown as SEQ ID NO: 261 or a variant having at least 80%, 85%, 90%, 95%, 98% or 99% sequence identity thereto. Suitably the FcRH5 binding domain may comprise a VHH having the sequence shown as SEQ ID NO: 261.

In one embodiment, the FcRH5 binding domain may comprise a VHH having the sequence shown as SEQ ID NO: 262 or a variant having at least 80%, 85%, 90%, 95%, 98% or 99% the sequence shown as SEQ ID NO: 262.

In one embodiment, the FcRH5 binding domain may comprise a VHH having the sequence shown as SEQ ID NO: 263 or a variant having at least 80%, 85%, 90%, 95%, 98% or 99% sequence identity thereto. Suitably the FcRH5 binding domain may comprise a VHH having the sequence shown as SEQ ID NO: 263.

In one embodiment, the FcRH5 binding domain may comprise a VHH having the sequence shown as SEQ ID NO: 264 or a variant having at least 80%, 85%, 90%, 95%, 98% or 99% sequence identity thereto. Suitably the FcRH5 binding domain may comprise a VHH having the sequence shown as SEQ ID NO: 264.

In one embodiment, the FcRH5 binding domain may comprise a VHH having the sequence shown as SEQ ID NO: 265 or a variant having at least 80%, 85%, 90%, 95%, 98% or 99% sequence identity thereto. Suitably the FcRH5 binding domain may comprise a VHH having the sequence shown as SEQ ID NO: 265.

In one embodiment, the FcRH5 binding domain may comprise a VHH having the sequence shown as SEQ ID NO: 266 or a variant having at least 80%, 85%, 90%, 95%, 98% or 99% sequence identity thereto. Suitably the FcRH5 binding domain may comprise a VHH having the sequence shown as SEQ ID NO: 266.

In one embodiment, the FcRH5 binding domain may comprise a VHH having the sequence shown as SEQ ID NO: 267 or a variant having at least 80%, 85%, 90%, 95%, 98% or 99% sequence identity thereto. Suitably the FcRH5 binding domain may comprise a VHH having the sequence shown as SEQ ID NO: 267.

In one embodiment, the FcRH5 binding domain may comprise a VHH having the sequence shown as SEQ ID NO: 268 or a variant having at least 80%, 85%, 90%, 95%, 98% or 99% sequence identity thereto. Suitably the FcRH5 binding domain may comprise a VHH having the sequence shown as SEQ ID NO: 268.

In one embodiment, the FcRH5 binding domain may comprise a VHH having the sequence shown as SEQ ID NO: 269 or a variant having at least 80%, 85%, 90%, 95%, 98% or 99% the sequence shown as SEQ ID NO: 269.

In one embodiment, the FcRH5 binding domain may comprise a VHH having the sequence shown as SEQ ID NO: 270 or a variant having at least 80%, 85%, 90%, 95%, 98% or 99% sequence identity thereto. Suitably the FcRH5 binding domain may comprise a VHH having the sequence shown as SEQ ID NO: 270.

In one embodiment, the FcRH5 binding domain may comprise a VHH having the sequence shown as SEQ ID NO: 271 or a variant having at least 80%, 85%, 90%, 95%, 98% or 99% sequence identity thereto. Suitably the FcRH5 binding domain may comprise a VHH having the sequence shown as SEQ ID NO: 271.

In one embodiment, the FcRH5 binding domain may comprise a VHH having the sequence shown as SEQ ID NO: 272 or a variant having at least 80%, 85%, 90%, 95%, 98% or 99% sequence identity thereto. Suitably the FcRH5 binding domain may comprise a VHH having the sequence shown as SEQ ID NO: 272.

In one embodiment, the FcRH5 binding domain may comprise a VHH having the sequence shown as SEQ ID NO: 273 or a variant having at least 80%, 85%, 90%, 95%, 98% or 99% sequence identity thereto. Suitably the FcRH5 binding domain may comprise a VHH having the sequence shown as SEQ ID NO: 273.

In one embodiment, the FcRH5 binding domain may comprise a VHH having the sequence shown as SEQ ID NO: 274 or a variant having at least 80%, 85%, 90%, 95%, 98% or 99% sequence identity thereto. Suitably the FcRH5 binding domain may comprise a VHH having the sequence shown as SEQ ID NO: 274.

In one embodiment, the FcRH5 binding domain may comprise a VHH having the sequence shown as SEQ ID NO: 275 or a variant having at least 80%, 85%, 90%, 95%, 98% or 99% sequence identity thereto. Suitably the FcRH5 binding domain may comprise a VHH having the sequence shown as SEQ ID NO: 275.

In one embodiment, the FcRH5 binding domain may comprise a VHH having the sequence shown as SEQ ID NO: 276 or a variant having at least 80%, 85%, 90%, 95%, 98% or 99% the sequence shown as SEQ ID NO: 276.

In one embodiment, the FcRH5 binding domain may comprise a VHH having the sequence shown as SEQ ID NO: 277 or a variant having at least 80%, 85%, 90%, 95%, 98% or 99% sequence identity thereto. Suitably the FcRH5 binding domain may comprise a VHH having the sequence shown as SEQ ID NO: 277.

In one embodiment, the FcRH5 binding domain may comprise a VHH having the sequence shown as SEQ ID NO: 278 or a variant having at least 80%, 85%, 90%, 95%, 98% or 99% sequence identity thereto. Suitably the FcRH5 binding domain may comprise a VHH having the sequence shown as SEQ ID NO: 278.

In one embodiment, the FcRH5 binding domain may comprise a VHH having the sequence shown as SEQ ID NO: 279 or a variant having at least 80%, 85%, 90%, 95%, 98% or 99% sequence identity thereto. Suitably the FcRH5 binding domain may comprise a VHH having the sequence shown as SEQ ID NO: 279.

In one embodiment, the FcRH5 binding domain may comprise a VHH having the sequence shown as SEQ ID NO: 280 or a variant having at least 80%, 85%, 90%, 95%, 98% or 99% sequence identity thereto. Suitably the FcRH5 binding domain may comprise a VHH having the sequence shown as SEQ ID NO: 280.

In one embodiment, the FcRH5 binding domain may comprise a VHH having the sequence shown as SEQ ID NO: 281 or a variant having at least 80%, 85%, 90%, 95%, 98% or 99% sequence identity thereto. Suitably the FcRH5 binding domain may comprise a VHH having the sequence shown as SEQ ID NO: 281.

In one embodiment, the FcRH5 binding domain may comprise a VHH having the sequence shown as SEQ ID NO: 282 or a variant having at least 80%, 85%, 90%, 95%, 98% or 99% sequence identity thereto. Suitably the FcRH5 binding domain may comprise a VHH having the sequence shown as SEQ ID NO: 282.

In one embodiment, the FcRH5 binding domain may comprise a VHH having the sequence shown as SEQ ID NO: 283 or a variant having at least 80%, 85%, 90%, 95%, 98% or 99% the sequence shown as SEQ ID NO: 283.

In one embodiment, the FcRH5 binding domain may comprise a VHH having the sequence shown as SEQ ID NO: 284 or a variant having at least 80%, 85%, 90%, 95%, 98% or 99% sequence identity thereto. Suitably the FcRH5 binding domain may comprise a VHH having the sequence shown as SEQ ID NO: 284.

In one embodiment, the FcRH5 binding domain may comprise a VHH having the sequence shown as SEQ ID NO: 285 or a variant having at least 80%, 85%, 90%, 95%, 98% or 99% sequence identity thereto. Suitably the FcRH5 binding domain may comprise a VHH having the sequence shown as SEQ ID NO: 285.

In one embodiment, the FcRH5 binding domain may comprise a VHH having the sequence shown as SEQ ID NO: 286 or a variant having at least 80%, 85%, 90%, 95%, 98% or 99% sequence identity thereto. Suitably the FcRH5 binding domain may comprise a VHH having the sequence shown as SEQ ID NO: 286.

In one embodiment, the FcRH5 binding domain may comprise a VHH having the sequence shown as SEQ ID NO: 287 or a variant having at least 80%, 85%, 90%, 95%, 98% or 99% sequence identity thereto. Suitably the FcRH5 binding domain may comprise a VHH having the sequence shown as SEQ ID NO: 287.

In one embodiment, the FcRH5 binding domain may comprise a VHH having the sequence shown as SEQ ID NO: 288 or a variant having at least 80%, 85%, 90%, 95%, 98% or 99% sequence identity thereto. Suitably the FcRH5 binding domain may comprise a VHH having the sequence shown as SEQ ID NO: 288.

The amino acid sequences for SEQ ID NOs: 40-53, 158 and 223-288 are provided in Table 1 below.

TABLE 1
Examples of FcRH5-specific sdAb (VHH)

| | |
|---|---|
| SEQ ID NO: 40 | QVQLQESGGGLVQAGGSLRLSCAASRSSFSNNAMGWYRQVPGKQRELVAFITKGGVTDY |
| | SDSVKGRFTISKDNAKNTVYLQMNSLKPEDTAVYYCNTIPFRSAWGQGTQVTVS |
| SEQ ID NO: 41 | QVQLQESGGGSVQPGGSLSLSCAASGFTFSNYAMSWVRQAPGKGPEWVAVINSDGGTAS |
| | SAGSVRGRFTISRDNAKNTLYLQMNRLKPEDTAVYYCAANRGFCAGVRCLEYQYWGQGT |
| | QVTVS |
| SEQ ID NO: 42 | QVQLQESGGGLVQAGGSLIVSCAASGRTFSINAMAWFRQAPGKEREFVAAIGGSGRVSS |
| | TSYADFVKGRFTISRDNAKNTVYLRMNNLEPEDTAVYYCAARRDYLPFPPESYDYWGQG |
| | TQVTVA |
| SEQ ID NO: 43 | QVQLQESGGGLVQAGGSLRLSCAASGRTFSTYAMAWFRQAPGKEREFVAAISGFGVVTY |
| | YADSVKGRFTISRDNAKNTLYLQMNGLKPEDTAVYYCAAGRRTSTNGGDYDYWGQGTQV |
| | TVS |
| SEQ ID NO: 44 | QVQLQESGGGLVQAGGSLRLSCAASGRTFSTYGMGWFRQAPGKEREFVAAISRSGGATA |
| | YAASVKGRFTISRDDVKNTLYLQMNSLKPEDTAVYHCAGTRRAFSTGLRDYDYWGQGIQ |
| | VTVS |
| SEQ ID NO: 45 | QVQLQQSGGGLVQAGDSLRLSCAASGRTFSNSTMGWFRQAPGKERKFVAVISWSGGTYA |
| | YAESVKGRFTISRDNAKNTVYLQMNSLKPEDTAVYYCAAARKGWSTRGDDYDYWGQGTQ |
| | VTVS |
| SEQ ID NO: 46 | QVQLQESGGGLVQAGGSLRLSCAASGRTYNNYAMGWFRQAPGKEREFVAGISRSGGMTG |
| | YAESVKGRFTISRDNAKNMVFLQMNSLKPEDTAVYYCAAYVGGFSTARRDYSYWGQGTQ |
| | VTVS |
| SEQ ID NO: 47 | QVQLQESGGGLVQAGDSLRLSCAYSGRTFSSYAMGWFRQAPGKERVFVAAISRIGGVTT |
| | YAESVQGRFTISRDNAKNTLYLQMNALKPEDTAVYYCAAAGLVSISTTPNDYDYWGQGT |
| | QVTVS |
| SEQ ID NO: 48 | QVQLQQSGGGLVQPGGSLRLSCAAPGNIFRLNGTGWYRQAPGKQRELVAHITSGGNTDY |
| | ADSVKGRFTISRDNAKNTVYLQMNSLKPEDTAVYYCNAIPFRLSWGQGTQVTVS |
| SEQ ID NO: 49 | QVQLQESGGGLVQAGGSLRLSCVVSRNIFSLNPMGWYRQAPGKQREMVAIITDGGSTNY |
| | ADSVKGRFTISRDNVKNTVYLQMNALEPEDTAVYYCNRVGGLQTWAWGQGTQVTVSS |
| SEQ ID NO: 50 | QVQLQQSGGGLVQAGGSLSLSCTASGRSFSNYGMGWFRQAPGKEREFVAAIGMVGGLTA |
| | YSNSAKGRFTISRDNAKNTLYLQMNSLKPEDTAVYLCAAGRRFSTSSRDYDIWGQGTQV |
| | TVS |
| SEQ ID NO: 51 | QVQLQESGGGLVQAGDSLRLSCAASGRTFDSRPMGWFRQAPGKEREFVGAVSWRGESTY |
| | YPDSVKGRFTISRDNAKRTVYLQMNSLKPEDTAVYYCAAGEPYSGTYYYRGRDYDYWGQ |
| | GTQVTVS |
| SEQ ID NO: 52 | QVQLQESGGGLVQAEGSLRLSCAASGRTFSMYAMGWFRQAPGREREFVAAISGSARITY |
| | YGQSVKGRFTISRDNAKNTVYLQMNSLKPEDTAVYYCAASSTYTSTSGSSYNYWGQGTQ |
| | VTVS |
| SEQ ID NO: 53 | QVQLQESGGGLVQAGGSLRLSCAASGSIFSINAMGWYRQAPGKQRELVAFITSGGSTNY |
| | ADSVKGRFTISRDNAKNTVYLQMNSLKPEDTAVYYCNALGGFVPSYGWGQGTQVTVS |
| SEQ ID NO: 158 | QVQLQESGGGLVQAGGSLRLSCTASGRTSSRAAMGWFRQAPGKEREFVAVISWSGGTTA |
| | YANSVKGRFTISRDNAKNTLYLQMNSLKPEDTAVYYCAAARIFTTARNDYDHWGQGTQV |
| | TVS |
| SEQ ID NO: 223 | QVQLQESGGGLVQPGGSLRLSCAAPGNIFRLNGTGWYRQAPGKQRELVAHITSGGNTDY |
| | ADSVKGRFTISRDNAKNTVYLQMNSLKPEDTAVYYCNTIPFRLSWGQGTQVTVSS |
| SEQ ID NO: 224 | QVQLQESGGGLVQPGGSLRLSCAAPGNIFRLNAMGWYRQAPGKQRELVAHITSGGNTDY |
| | ADSVKGRFTISRDNAKNTVYLQMNSLKLEDTAVYYCNTIPFRLSWGQGTQVTVSS |
| SEQ ID NO: 225 | QVQLQESGGGLAQAGGSLRLSCAASRSSFSNNAMGWYRQVQGKQRELVAFITKGGVTDY |
| | SVSGKGRFTISKDHAKNTVYLQMNSLKPRDTAVYYCNTIPVRSAWGQGTQVTVSA |
| SEQ ID NO: 226 | QVQLQESGGGLVQAGGSLRLSCAASGSIFSINAMGWYRQAPGKQRELVAFITKGGVTDY |
| | SDSVKGRFTISKDNAKNTVYLQMNSLKPEDTAVYYCNTIPCRSAWGQGTQVTVSS |
| SEQ ID NO: 227 | QVQLQESGGGLVQPGGSLRLSCAAPGSSFRLNGTGWYRQAPGKQRELVAHITSGGSTNY |
| | SDSVKGRLTISKDNAKNTVYLQMNSLKPEDTAVYYCNTIPFSRAWGQGTLVTVSS |
| SEQ ID NO: 228 | QVQLQQSGGGLVQAGGSLRLSCAASGRSVSINAMGWYRQAPGKQRELVAIIDRSGNTDY |
| | ADSVKGRFTISRDNAKKAVYLQMNSLKPEDTAVYYCNTIPYSDSWGQGTQVTISS |
| SEQ ID NO: 229 | QVQLQESGGGLVQPGGSLRLSCAASGRTFSSYAMGWFRRAPGKGREFVATIDGIGGITS |
| | YAGSVKGRFTVSKDNAKNTVYLQMNSLKPEDTAVYYCNTIPFRSAWGQGTLVTVSS |
| SEQ ID NO: 230 | QVQLQESGGGLVQAGGSLRLSCAASRSSFSNNAMGWYRQGPGKQRELVAFITKGGVTDY |
| | SDSVKGRFTISKDNAKNTVYLQMNSLKPEDTAVYYCNTIPFRSAWGQGTQVTVSS |
| SEQ ID NO: 231 | QVQLQESGGGLVQAGGSLRLSCAASRSSFSNNAMGWYRQVPGKQRELVAFITKGGVTDY |
| | SDSVKGRFTISKDNAKNTVYLQMNSLKPEDTAVYYCNAIPFRSAWGQGTQVTVSS |
| SEQ ID NO: 232 | QVQLQESGGGLVQPGGSLRLSCAAPGNNFRLNAMGWYRQAPGKQRELVAHITSGGNTDY |
| | ADSVKGRFTISRDNAKNTVYLQMNSLKPEDTAVYYCNAIPFRPSWGQGTQVTVSS |
| SEQ ID NO: 233 | QVQLQESGGGLVQPGGSLRLSCAAPGNIFRLNGTGWYRQAPGKQRELVAHITSGGNTDY |
| | ADSGKGRFTISRDNAKNTVYLQMNSLKPEDTAVYYCNAIPFRRSWGQGTQVTVSS |
| SEQ ID NO: 234 | QVQLQESGGGLVQAGGSLKLSCAASERIFRINAMGWYRQAPGKQRELVAHITSGGNTDY |
| | ADSVKGRFTISRDNAKNTMYLQMNSLKPEDTAVYYCNAIPFRRSWGQGTQVTVSS |
| SEQ ID NO: 235 | QVQLQQSGGGLVQPGGSLRLSCAAPGNIFRLNGTGWYRQAPGKQRELVAHITSGGNTDY |
| | ADSVKGRFTISRDNAENTVYLQMNSLKPEDTAVYYCNAVPFRLSWGQGTQVTVSS |
| SEQ ID NO: 236 | QVQLQESGGGLVQPGESLRLSCAASGFTFSTYWMSWVRQAPGKGPEGVSGIDNGGGTTT |
| | YADSVKGRFTISRDNAGNTVYLQMNSLKPEDTAVYYCNALPFRLSWGQGTQVTVSS |
| SEQ ID NO: 237 | QVQLQESGGGLVQPGGSLRLSCAAPGNIFRLNGTGWYRQAPGKQGELVAIITSGGNTDY |
| | ADSVKGRFTISRDNAKNTTYLQMNSLKPEDTAVYYCNATPFRLSWGQGTQVTVSS |
| SEQ ID NO: 238 | QVQLQESGGGLVQPGGSLRLSCAAPGNIFRLNGTGWYRQAPGKQRELVAHITRGGNTDY |
| | ADAVKGRFTISRDNAKNTVYLQMNSLKPEDTAVYYCNSIPFRLSWGQGTQVTVSS |
| SEQ ID NO: 239 | QVQLQESGGGLVQPGGSLRLSCAAPGNIFRINGTGWYGQAPGKQRELVAHITSGGNTDY |
| | EDSVKGRFTISRDNAKNTTYLQMNSLKPEDTAVYYCNAIPFRISWGQGTQVTVSS |
| SEQ ID NO: 240 | QVQLQQSGGGLVQPGGSLRLSCAAPGNNFRLNAMGWYRQAPGKQRELVAHITSGGNTDY |
| | ADSVKGRFTISRDNAKNTTYLQMNSLKPEDTAVYYCNAIPFRLYWGQGTQVTVSS |
| SEQ ID NO: 241 | QVQLQESGGGLVQPGGSLRLSCLASGFTFSTYWMSWVRQAPGKGPEWVSGIDNGGGTTT |
| | YADSVKGRFTISRDNAKNTLNLEMNNLKPEDTAGYYSKAIPFRLSWGQGTQVTVSS |
| SEQ ID NO: 242 | QVQLQESGGGLVQPGGSLRLSCAASGSSFSNNAMGWYRQAPGKQRELVAFITKGGVTDY |
| | SDSVRGRFTISRDNAKNTVYLQMNSLKPEDTAVYYCNTIPFRSAWGQETQVTVSS |
| SEQ ID NO: 243 | QVQLQQSGGGLVQPGGSLRLSCLASGFTFSTYWMSWVRQAPGKGPEWVSGIDNGGGTTT |
| | YADSVKGRFTISRDNAKNTVYLQMNSLKPEDTAVYYCNTIPFRSAWGQGTQVTVAS |
| SEQ ID NO: 244 | QVQLQESGGGLVQPGGSLRLSCAAPGNIFRLNATGWYRQVPGKQRELVAFITKGGVTEH |
| | SDSVEGRFTISKDNAKNTVYLQMNSLKPEDTAVYYCNTIPFRSAWGQGTQVTVSS |
| SEQ ID NO: 245 | QVQLQESGGGLVQTGGSLRLSCAASRSSFSNNAMGWYRQVPGKQRELVAFITKGGVTDY |
| | SDSVKGRFTISKDNAKNTVYLQMNSLKPEDTAVYYCNTIPFRSAWGQGTQVTVSS |
| SEQ ID NO: 246 | QVQLQESGGGLVQAGGSLRLSCAASGSSFSNNAMGWYRQVPGKQRELVAFITKGGVTDY |
| | SDSVKGRFTISKDNAKNTVYLQMNSLKPEDTAVYYCNTIPFRSAWGQGTQVTVSS |
| SEQ ID NO: 247 | QVQLQESGGGLVQAGGSLRLSCAASRSSFGNNAMGWYRQVPGKQRELVAFITKGGVTDY |
| | SDSVKGRFTISKDNAKNTVYLQMNSLKPEDTAVYYCNTIPFRSAWGQGTQVTVSS |
| SEQ ID NO: 248 | QVQLQESRGGLVQAGGSLRLSCAASGTIERNNAMAWYRQAPGKQRELVAIITSGGSTNY |
| | SDSVKGRFTISRDNAKNTLYLQMNSLKPEDTAVYLWAAGRRFSTRSRDYDYWGQGTQVT |
| | VSS |
| SEQ ID NO: 249 | QVQLQESGGGLVQAGGSLSLSCTASGRSFSNYGMGWFRQAPGKEREFVAAIGMVGGLTA |
| | YSNSAKGRFTISRDNAKNTVYLQMNSLKPEDTAVYLCAAGRRFSTSSRDYDYWGQGTQV |
| | TVSS |
| SEQ ID NO: 250 | QVQLQESGGGLVQAGDSLRLSCAASGRTFSSYAMGWFRQAPGKEREFVAAISRIGGVTT |
| | YAGSVQGRFTISRDNAKNTLYLRMNALKPEDTAVYYCAAAGLVSISTTPNDYDYWGQGT |
| | QVTVSS |
| SEQ ID NO: 251 | QVQLQESGGGLVQAGGSLSLSCTASGRSFSNYGMGWFRQAPGKEREFVDAIGMVGGLTA |
| | YSNSAKGRFTISRDNAKNTLYLQMNSLKPEDTAVYLCAAGRRFSTGSRDYDIWGQGTQV |
| | TVSS |
| SEQ ID NO: 252 | QVQLQQSGGGLVQTGGSLRLSCAASGRTFSSYAMGWFRQAPGKEREFVAAISQFGGVTT |
| | YADSVQGRFTISRDNAKNTLYLRMNSLKPEDTAVYLCAAGRRFSTGSRDYDIWGQGTQV |
| | TVSS |
| SEQ ID NO: 253 | QVQLQESGGGLVQAGGSLSLSCTASGRSFSNYGMGWFRQAPGKEREFVAAIGMVGGLTA |
| | YSNSAKGRFTISRDKAKNTLYLQMNSLKPEDTAGYCGAGGRRFSTSSRDYDIWGQGTQV |
| | TVSS |
| SEQ ID NO: 254 | QVQLQESGGGLVQAGGSLSLSCTASGRSFSNYGMGWFRQAPGKEREFVAAIGMVGGLTA |
| | YSNSAKGRFTISRDNAKNTLYLQMNSLKPEDTAVYLCAAGRRFSTSSREYDIWGQGTQV |
| | TVSS |
| SEQ ID NO: 255 | QVQLQESGGGLVQAGGSLSLSCTASGRSFSNYGMGWFRQAPGKEREFVAAIGMVGGLPA |
| | YSNSAKGRFTISQDNAKNPLYLQINSLKPEETDVYLCAAGRRLSTSSRDYDIWGQGTQV |
| | TVSS |
| SEQ ID NO: 256 | QVQLQESGGGLVQAGGSLSLSCTASGRSFSNYGMGWFRQAPGKEREFVAAISRGGGVSA |
| | YSNSAKGRFTISRDNAKNTVYLQMNSLKPEDAAVYFCAAGLRFSTGSRDYDIWGQGTQV |
| | TVSS |
| SEQ ID NO: 257 | QVQLQQSGGGLVQAGDSLRLSCAASGRTFRRYAMGWFHQAPGKDREFVAGISRSGGMTG |
| | YADSVKGRFTISRDNAKNMVFLQMNSLKPEDTAVYYCAAYVGGFSTTRRDYAYWGQGTQ |
| | VTVSS |
| SEQ ID NO: 258 | QVQLQESGGGLVQAGDSLRLSCAASGRTFSNSTMGWFHQAPGKERKFVAVISWSGGTTA |
| | YAESVKGRFTISRDNAKNTVYLQMNSLKPEDTAVYYCAAARKGWSTRGDDYDYWGQGTQ |
| | VTVSS |
| SEQ ID NO: 259 | QVQLQESGGGLVQTGGSLRLSCAASGRTVIGWFRQAPGKEREFVAVSSGSGGVTAYASS |
| | VEGRFTISRDNVKNIMYLQMNSLKPEDTAIYYCAAALTWSTRPSDFTSWGQGTQVTVSS |
| SEQ ID NO: 260 | QVQLQQSGGGLVQAGGSLRLSCAASGRTYNNYAMGWFRQAPGKEREFVAGISRSGGMTG |
| | YAESVKGRFTISRDNAKNMVCLQMNSLKPEDKAVYYCAAYVGGFSTARRDYSYWGQGTQ |
| | VTVSS |
| SEQ ID NO: 261 | QVQLQQSGGGLVQTGGSLRLSCAASGRTVIGWFRQAPGKEREFVAVSNWSGGVTAYASS |
| | VEGRFTISRDNVKNIMYFQMNSLKPEDTAVYYCAAYVGGFSTARRDYSYWGQGTQVTVS |
| | S |
| SEQ ID NO: 262 | QVQLQQSGGGLVQPGGSLRLSCAASGRTYNNYAMGWFRQAPGKEREFVAGISRSGGMTG |
| | YAESVKGRFTISRDNAKNMVFLQMNSLKPEDTAVYYCAAYVGGFSTARRDYSYWGQGTQ |
| | VTVSS |
| SEQ ID NO: 263 | QVQLQESGGGLVQAGGSLIVSCATSGRTFSINAMGWFRQAPGKEREFVAGISRSGGMTG |
| | YAESVKGRFTISRDNAKNMVFLQMNSLKPEDTAVYYCAAYVGGFSTARRDYSYWGQGTQ |
| | VSVSS |
| SEQ ID NO: 264 | QVQLQQSGGGLVQAGGSLRLSCAASGRTFSRYAMGWFRQAPGKEREFVAVINGSGGTTA |
| | YANSVKGRFTITRDNAKNTLYLQMNSLKPEDTAVYYCAAARIFTTTRNEYDHWGQGTQV |
| | TVSS |
| SEQ ID NO: 265 | QVQLQEPGGGLVQPGGSLRLSCAASGFTFSTYWMSWVHQAPGKGPEWVSGIDNGGGTTT |
| | YADSVKGRFTISRDNAKNTLYLQMNSLKPEETAIYYWAAARIFSTARNDYDHWGQGTQV |
| | TVSS |
| SEQ ID NO: 266 | QVQLQESGGGLVQAGGSLSLSCTASGRSFSNYGMGWFRQAPGKEREFVAAIGMVGGLTA |
| | YSNSAKGRFTISRDNAKNTLYLQMNSLKPEDTAVYLVAGGRIFRTSSRDYDIWGQGTQV |
| | TVSS |
| SEQ ID NO: 267 | QVQLQESGGGLVQPGGSLRLYCAAPGNIFRLNGTGWYRQAPGKQRELVTHITSGGNTDY |
| | ADSVKGRFTISRDNAKNTVYLQMNSLKPEDTAIYYCAAARFFTTARNDYDHWGQGTQVT |
| | VSS |
| SEQ ID NO: 268 | QVQLQESGGGLVQAGGSLRLSCAASGSIFSINAMGWYRQAPGKQRELVAAITSGGSTNY |
| | ADSVKGRFTISRDNAKNTVYLQMNSLKPEDTAVYYCNALGGFVPNYGWGQGTQVTVSS |
| SEQ ID NO: 269 | QVQLQESGGGLVQPGGSLRLSCAAPGNIFRLNGTGWYRQAPGKQRELVAHITSGGNTDY |
| | ADSVKGRFTISRDNAKNTVYLQMNSLKPEDTAVYYCNALGGFVPNYGWGQGTQVTVSS |
| SEQ ID NO: 270 | QVQLQESGGGLVQAGGSLRLSCAASGSIFSINAMGWYRQAPGKQRELVAAITSGGSTNY |
| | ADSVKGRFTTSRDNAKNTVYLQMNSLKPEDPAVYYCNALGGFVPNDGWGQGTQVTVSS |
| SEQ ID NO: 271 | QVQLQESGGGLVQAGGSLRLSCAASGNNFRLNAMGWYRQAPGKQRELVAHITSGGNTDY |
| | ADSVKGRFTISRDNAKNTTYLQMNSLKPEDTAVYYCNALGGFAPNYGWGQGTQVTVSS |
| SEQ ID NO: 272 | QVQLQQSGGGLVQAGGSLSLSCTASGRSFSNYGMGWFRQAPGKEREFVAAIGMVGGLTA |
| | YSNSAKGRFTISRDNAKNTVYLQMNSLKPEDTAVYYCNALGGFVPNYVWGQGTQVTVSS |
| SEQ ID NO: 273 | QVQLQESGGGLVQAGGSLSLSCTASGRSFSINAMGWYRQAPGKQRELVAAITSGGSTNY |
| | ADSVKGRFTISRDNAKNTVYLQMNSLKPEDTAVYYCNALGGFVLNYGWGQGTQVTVSS |
| SEQ ID NO: 274 | QVQLQESGGGLVQAGGSLRLSCAASGSIFSINAMGWYRQAPGKQRELVAAITSGGSTNY |
| | ADPVKGRFTISRDNAKNTVYLQMNSLKPEDTAVYYCNALGGLVPNYGWGQGTQVTVSS |
| SEQ ID NO: 275 | QVQLQESGGGLVQAGGSLRLSCAASGSIFSINAMGWYRQAPGKQRELVAAITSGGSTNY |
| | ADSVKGRFTISRDNAKNTVYLQMNSLKPEDTAVYYCNALGGFLPNYGWGQGTQVTVSS |
| SEQ ID NO: 276 | QVQLQESGGGLVQAGGSLRLSCAASGSIFSINAMGWYRQAPGKQRELVAAITSGGSTNY |
| | ADSVKGRFTISRDNAKNTVYLQMNSLKPEDTAVYYCNALGGFVTNYGWGQGTQVAVSS |
| SEQ ID NO: 277 | QVQLQESGGGLVQPGGSLRLSCAASGSIFSINAMGWYRQAPGKQRELVAFITSGGSTNY |
| | ADSVKGRFTISRDNAKNTVYLQMNSLKPEDTAVYYCNALGGFVPSYGWGQGTQVTVSS |
| SEQ ID NO: 278 | QVQLQQSGGGLVQTGGSLRLSCAASGRTVMGWYRQAPGKQRELVAVITRGGSTNYADSV |
| | KGRFTISRDNAKNTVYLQMNSLKPEDTAFYYCNALGGFVPNYGWGQGTQVTVSS |
| SEQ ID NO: 279 | QVQLQESGGGLVQPGGSLRLSCAAPGNIFRLNGTGWYRQAPGKQRELVAHITSGGNTDY |
| | ADSVKGRFTISRDNAKNTVYLQMNSLKPEDTAVYYCNALGGFVPSYGWGQGTQVTVSS |
| SEQ ID NO: 280 | QVQLQESGGGLVQPGGSLRLSCAAPGNNFRLNAMGWYRQAPGKQRELVAHITSGGNTDY |
| | ADSVKGRFTISRDNAKNTVYLQMNSLKPEDTAVYYCNALGGFVPSYGWGQGTQVTVSS |
| SEQ ID NO: 281 | QVQLQQSGGGLVQAGGSLRLSCAASGSIFSINAMGWYRQAPGKQRELVAFITSGGSTNY |
| | ADSVKGRFTISRDNAKNTVYLQMNSLKPEDTAVYYCNALGGFVPSYGWGQGTQVTVSS |
| SEQ ID NO: 282 | QVQLQQSGGGLVQPGGSLRLSCAAPGNIFRLNGTGWYRQAPGKQRELVAHITSGGSTNY |
| | ADSVKGRFTISRDNAKNTVYLQMNSLKPEDTAVYYCNALGGFVPSYGWGQGTQVTVSS |
| SEQ ID NO: 283 | QVQLQESGGGLVQAGGSLRLSCAASGSIFSINAMGWYRQAPGKQRELVAAITSGGSTNY |
| | ADSVKGRFTISRDNAKNTVYLQMNSLKPEDTAVYYCNALGGFVPSYGWGQGTQVTVSS |
| SEQ ID NO: 284 | QVQLQESGGGLVQAGGSLSLSCTASGRSFSNYGMGWFRQAPGKEREFVAAIGMVGGLTA |
| | YSNSAKGRFTISRDNAKNTLYLQMNSLKPEDTAVYYCNALGGFVPSYGWGQGTQVTVSS |
| SEQ ID NO: 285 | QVQLQESGGGLVQAGGSLRLSCAASGSIFSINAMGWYRQAPGKQRELVAFITSGGSTNY |
| | ADSVKGRFTISRDNAKNTVYLQMNSLKPEDTAVYYCNALGGFVPSYGWGQGTLVTVSS |
| SEQ ID NO: 286 | QVQLQESGGGLVQPGGSLRLSCAAPGNIFSINAMGWYRQAPGKQRELVAFITSGGSTNY |
| | ADSVKGRFTISRDNAKNTVYLQMNSLKPEDTAVYYCNALGGFVPSYGWGQGTQVTVSS |
| SEQ ID NO: 287 | QVQLQESGGGLVQPGGSLRLSCLASGFTFSTYWMSWVRQAPGKGPEWVSGIDNGGGTTT |
| | YADSVKGRFTISRDNAKNTLNLEMNNLKPEDTALYYCARNPTRGWYSTDYRGQGTQVTV |
| | SS |
| SEQ ID NO: 288 | QVQLQESGGGLVQPGGSLRLSCAASGFTFSTYWMSWVRQAPGKGPEWVSGIDNGGGTTT |
| | YADSVKGRFTISRDNAKHTLYLQMNTLKPEDTALYYCARNPTRGWYSTDYRGQGTQVTV |
| | SS |

Suitably, a VHH described herein may comprise CDR1, CDR2 and CDR3 sequences described herein.

Thus, a FcRH5 binding domain may comprise:
a VHH having CDRs with the following numbering:
   CDR1
   CDR2
   CDR3

The term “variant” refers to a polypeptide that has an equivalent function to the amino acid sequences described herein, but which includes one or more amino acid substitutions, insertions or deletions. Thus, a VHH may comprise a variant having at least 80%, 85%, 90%, 95%, 98% or 99% sequence identity to a sequence described herein.

As used herein, “variant” is synonymous with “mutant” and refers to an amino acid sequence which differs in comparison to the corresponding wild-type sequence. The term “wild-type” is used to mean a protein having an amino acid sequence respectively, which is identical with the native protein respectively.

It may be possible to introduce one or more mutations (substitutions, additions or deletions) into each CDR without negatively affecting binding activity. Each CDR may, for example, have one, two or three amino acid mutations. The VHH region which comprises the FcRH5 binding domain comprising the one or more of the CDRs which comprise one, two or three amino acid mutations may suitably maintain the capacity to bind FcRH5.

The FcRH5 binding domain's capacity to bind FcRH5 may be assessed by determining the binding affinity.

The term “affinity”, as used herein, refers to the strength of interaction between an antibody's antigen binding site and an epitope. Affinity is usually measured as the equilibrium dissociation constant (KD), which is a ratio of the dissociation rate constant (k_{d} or k_{off}) and association rate constant (kₐ or kₒₙ), i.e. k_{d}/kₐ or k_{off}/kₒₙ, between the antibody and the antigen. KD and affinity are inversely related. The term “association rate constant” or “on-rate” or “kₐ” or “kₒₙ”, as used herein, refers to a constant used to characterize how quickly the antibody binds to its target. The term “dissociation rate constant” or “off-rate” or “k_{d}” or “k_{off}”, as used herein, refers to a constant used to characterize how quickly the antibody unbinds or disassociates from its target. KD is measured in M; kₐ is measured in M⁻¹ s⁻¹; and k_{d} is measured in s⁻¹.

A quantitative assessment or measurement of binding affinity (e.g. establishing a KD value) may be determined or measured using methods known in the art, such as by labelled-dependent methods, e.g. direct and indirect ELISA and radioimmunoassay methods, as well as by label-free methods which enable a direct detection and measurement of interactions in real-time, e.g. bio-layer interference and surface plasmon resonance (SPR), for example by using the Biacore® system. In addition to the equilibrium dissociation constant (KD), the association rate constant (Ka (1/Ms)), and the dissociation rate constant (Kd (1/s)) may also be determined.

The KD and the kinetic rate constants of the FcRH5 binding domain may be measured by SPR. The assay may be carried out using different parameters and using a variety of apparatuses that are commercially available. Surface Plasmon Resonance (SPR) experiments may be performed with a Biacore T200 or a Biacore 8k instrument, for example. The KD and the kinetic rate constants may be measured on a Biacore T200 or Biacore 8k instrument, optionally using HBS-P+ or HBSP1 as the running and dilution buffer (GE Healthcare BioSciences), at a flow rate of 30 ml/min at 25° C. Kinetic rate constants may be obtained by curve fitting according to a 1:1 Langmuir binding model.

Methods for determining binding specificity include, but are not limited to, ELISA, western blot, immunohistochemistry, flow cytometry, Förster resonance energy transfer (FRET), phage display libraries, yeast two-hybrid screens, co-immunoprecipitation, bimolecular fluorescence complementation and tandem affinity purification. Binding affinity can also be determined using methods such as fluorescence quenching, isothermal titration calorimetry.

The variants encompassed by the invention have a binding activity that is essential unaltered or improved compared to the corresponding full-length sequence.

Identity comparisons can be conducted by eye, or more usually, with the aid of readily available sequence comparison programs. These commercially available computer programs can calculate % identity between two or more sequences. A suitable computer program for carrying out such an alignment is the GCG Wisconsin Bestfit package (University of Wisconsin, U.S.A.; Devereux et al., 1984, Nucleotide sequences Research 12:387). Examples of other software than can perform sequence comparisons include, but are not limited to, the BLAST package (see Ausubel et al., 1999 ibid—Chapter 18), FASTA (Atschul et al., 1990, J. Mol. Biol., 403-410) and the GENEWORKS suite of comparison tools. Both BLAST and FASTA are available for offline and online searching. For example, the percentage identity between two polypeptide sequences may be readily determined by BLAST which is freely available at http://blast.ncbi.nlm.nih.gov.

Once the software has produced an optimal alignment, it is possible to calculate % identity. The software typically does this as part of the sequence comparison and generates a numerical result.

The sequence may have one or more deletions, insertions or substitutions of amino acid residues which produce a silent change and result in a functionally equivalent molecule. These sequences are encompassed by the present invention. Deliberate amino acid substitutions may be made on the basis of similarity in polarity, charge, solubility, hydrophobicity, hydrophilicity, and/or the amphipathic nature of the residues as long as the activity is retained. For example, negatively charged amino acids include aspartic acid and glutamic acid; positively charged amino acids include lysine and arginine; and amino acids with uncharged polar head groups having similar hydrophilicity values include leucine, isoleucine, valine, glycine, alanine, asparagine, glutamine, serine, threonine, phenylalanine, and tyrosine.

Suitably, a polypeptide or FcRH5 binding molecule comprising the single domain antibody may be an antibody conjugate, a chimeric antigen receptor (CAR), an immune cell engager.

In the context of a FcRH5 binding molecule described above, in order to stimulate cell activation, the FcRH5 binding domain may bind to its cognate antigen (FcRH5) with a certain binding profile (for example, with a required binding affinity).

The utility of antibodies and binding molecules may be limited by solution properties such as solubility, aggregation and thermal stability, which are often related to each other. Poor thermal stability may affect both antibody solubility and aggregation, although antibody solubility and antibody aggregation may also be independent of thermal stability.

A quantitative assessment or measurement of thermal stability may be determined or measured using methods known in the art, such as techniques measuring unfolding temperatures and thermodynamic parameters during protein unfolding, including differential scanning calorimetry (DSC), differential scanning fluorimetry (DSF). The melting temperature (Tm), at which the transition from the folded to unfolded state occurs, is used as a surrogate parameter for the thermal stability of the antibody and binding molecule. For example, DSF may be performed using a Prometheus NT.48 instrument (Nanotemper). The Tm may be performed on a Prometheus NT.48 instrument, scanning at 330 and 350 nm with a temperature ramp of 1° C./min from 20 to 95° C. Melting temperature (Tm) is calculated as first derivative of 350 nm/330 nm ratio.

Protein aggregation is a process in which protein molecules self-associate with each other. Aggregation requires proteins to experience unfolding or partial unfolding. A quantitative assessment or measurement of aggregation may be determined or measured using methods known in the art, such as biochemical assays for monitoring protein aggregates which often rely on ultracentrifugation, size-exclusion chromatography, gel electrophoresis, dynamic light scattering, or turbidity measurements. For example, aggregation propensity and average particle size of antibodies and binding domains may be determined by multi-angle dynamic light scattering (MADLS). MADLS may be performed using a Zetasizer Ultra device and ZS Xplorer software (Malvern Panalytical).

Preferred FcRH5 VHH domains are set forth in Table 12, below:

TABLE 12

| AU | | | | |
|---|---|---|---|---|
| ID# | CDR1 | CDR2 | CDR3 | VHH |
| 60369 | RSSFSNNA | ITKGGVT | NTIPFRSA | QVQLQESGGGLVQAGGSLRLSCAASRSS |
| | (SEQ ID | (SEQ ID | (SEQ ID | FSNNAMGWYRQVPGKQRELVAFITKGGV |
| | NO: 1) | NO: 2) | NO: 3) | TDYSDSVKGRFTISKDNAKNTVYLQMNS |
| | | | | LKPEDTAVYYCNTIPFRSAWGQGTQVTV |
| | | | | S (SEQ ID NO: 40) |
| 60387 | GRTFSTYG | ISRSGGAT | AGTRRAFS | QVQLQESGGGLVQAGGSLRLSCAASGRT |
| | (SEQ ID | (SEQ ID | TGLRDYDY | FSTYGMGWFRQAPGKEREFVAAISRSGG |
| | NO: 169) | NO: 11) | (SEQ ID | ATAYAASVKGRFTISRDDVKNTLYLQMN |
| | | | NO: 12) | SLKPEDTAVYHCAGTRRAFSTGLRDYDY |
| | | | | WGQGIQVTVS (SEQ ID NO: 44) |
| 60460 | GRTYNNYA | ISRSGGMT | AAYVGGFS | QVQLQESGGGLVQAGGSLRLSCAASGRT |
| | (SEQ ID | (SEQ ID | TARRDYSY | YNNYAMGWFRQAPGKEREFVAGISRSGG |
| | NO: 16) | NO: 17) | (SEQ ID | MTGYAESVKGRFTISRDNAKNMVFLQMN |
| | | | NO: 18) | SLKPEDTAVYYCAAYVGGFSTARRDYSY |
| | | | | WGQGTQVTVS (SEQ ID NO: 46) |
| 60462 | GRTSSRAA | ISWSGGTT | AAARIFTT | QVQLQESGGGLVQAGGSLRLSCTASGRT |
| | (SEQ ID | (SEQ ID | ARNDYDH | SSRAAMGWFRQAPGKEREFVAVISWSGG |
| | NO: 155) | NO: 156) | (SEQ ID | TTAYANSVKGRFTISRDNAKNTLYLQMN |
| | | | NO: 157) | SLKPEDTAVYYCAAARIFTTARNDYDHW |
| | | | | GQGTQVTVS (SEQ ID NO: 158) |
| 60471 | GSIFSINA | ITSGGST | NALGGFVP | QVQLQESGGGLVQAGGSLRLSCAASGSI |
| | (SEQ ID | (SEQ ID | SYG | FSINAMGWYRQAPGKQRELVAFITSGGS |
| | NO: 37) | NO: 38) | (SEQ ID | TNYADSVKGRFTISRDNAKNTVYLQMNS |
| | | | NO: 39) | LKPEDTAVYYCNALGGFVPSYGWGQGTQ |
| | | | | VTVS (SEQ ID NO: 53) |

In one embodiment, the FcRH5 binding domain has an affinity (KD) of at least about 1×10⁻⁸ M, 5×10⁻⁹ M, 1×10⁻⁹ M, 5×10⁻¹⁰ M, 1×10⁻¹⁰ M, 5×10⁻¹¹ M, 1×10⁻¹¹ M, 5×10⁻¹² M, 1×10⁻¹² M, 5×10⁻¹³ M, 1×10⁻¹³ M, 5×10⁻¹⁴ M, 1×10⁻¹⁴ M, or 5×10⁻¹⁵ M, or 1×10⁻¹⁵ M, as measured by SPR on a Biacore 8k instrument. In another embodiment, the FcRH5 binding domain has a dissociation rate constant (kₐ or k_{off}) of at least about 5×10⁻³ s⁻¹, 1×10⁻³ s⁻¹, 5×10⁻⁴ s⁻¹, 1×10⁻⁴ s⁻¹, 5×10⁻⁵ s⁻¹, 1×10⁻⁵ s⁻¹, 5×10⁻⁶ s⁻¹, 1×10⁻⁶ s⁻¹, 5×10⁻⁷ s⁻¹, 1×10⁻⁷ s⁻¹, 5×10⁻⁸ s⁻¹, 1×10⁻⁸ s⁻¹, 5×10⁻⁹ s⁻¹, or 1×10⁻⁹ s⁻¹, as measured by SPR on a Biacore 8k instrument. In another embodiment, an FcRH5 binding moiety of the invention has an association rate constant (kₐ or kₒₙ) of at least about 1×10³ M⁻¹s⁻¹, 5×10³ M⁻¹s⁻¹, 1×10⁴ M⁻¹s⁻¹, 5×10⁴ M⁻¹s⁻¹, 1×10⁵ M⁻¹s⁻¹, 5×10⁵ M⁻¹s⁻¹, 1×10⁶ M⁻¹s⁻¹, or 5×10⁶ M⁻¹s⁻¹, as measured by SPR on a Biacore 8k instrument. In another embodiment, an FcRH5 binding domain has a thermal stability Tm as measured by differential scanning fluorimetry (DSF) at 330 and 350 nm with a temperature ramp of 1° C./min from 20 to 95° C. (Tm calculated as first derivative of 350 nm/330 nm ratio) of at least about 59° C., 60° C., 61° C., 62° C., 63° C., 64° C., 65° C., 66° C., 67° C., 68° C., 69° C., 70° C., 71° C., 72° C., 73° C., 74° C., 75° C., 76° C., 77° C., 78° C., 79° C., or 80° C.

In yet another embodiment, an FcRH5 binding domain has an affinity (KD) of at least about 1×10⁻⁸ M, 5×10⁻⁹ M, 1×10⁻⁹ M, 5×10⁻¹⁰ M, 1×10⁻¹⁰ M, 5×10⁻¹¹ M, 1×10⁻¹¹ M, 5×10⁻¹² M, 1×10⁻¹² M, 5×10⁻¹³ M, 1×10⁻¹³ M, 5×10⁻¹⁴ M, 1×10⁻¹⁴ M, 5×10⁻¹⁵ M, or 1×10⁻¹⁵ M, as measured by SPR on a Biacore 8k instrument, and a thermal stability Tm as measured by differential scanning fluorimetry (DSF) at 330 and 350 nm with a temperature ramp of 1° C./min from 20 to 95° C. (Tm calculated as first derivative of 350 nm/330 nm ratio) of at least about 59° C., 60° C., 61° C., 62° C., 63° C., 64° C., 65° C., 66° C., 67° C., 68° C., 69° C., 70° C., 71° C., 72° C., 73° C., 74° C., 75° C., 76° C., 77° C., 78° C., 79° C., or 80° C.

In yet another embodiment, an FcRH5 binding domain has a dissociation rate constant (k_{d} or k_{off}) of at least about 5×10⁻³ s⁻¹, 1×10⁻³ s⁻¹, 5×10⁻⁴ s⁻¹, 1×10⁻⁴ s⁻¹, 5×10⁻⁵ s⁻¹, 1×10⁻⁵ s⁻¹, 5×10⁻⁶ s⁻¹, 1×10⁻⁶ s⁻¹, 5×10⁻⁷ s⁻¹, 1×10⁻⁷ s⁻¹, 5×10⁻⁸ s⁻¹, 1×10⁻⁸ s⁻¹, 5×10⁻⁹ s⁻¹, or 1×10⁻⁹ s⁻¹, as measured by SPR on a Biacore 8k instrument, and a thermal stability Tm as measured by differential scanning fluorimetry (DSF) at 330 and 350 nm with a temperature ramp of 1° C./min from 20 to 95° C. (Tm calculated as first derivative of 350 nm/330 nm ratio) of at least about 59° C., 60° C., 61° C., 62° C., 63° C., 64° C., 65° C., 66° C., 67° C., 68° C., 69° C., 70° C., 71° C., 72° C., 73° C., 74° C., 75° C., 76° C., 77° C., 78° C., 79° C., or 80° C.

In yet another embodiment, an FcRH5 binding domain has an association rate constant (kₐ or kₒₙ) of at least about 1×10³ M⁻¹s⁻¹, 5×10³ M⁻¹s⁻¹, 1×10⁴ M⁻¹s⁻¹, 5×10⁴ M⁻¹s⁻¹, 1×10⁵ M⁻¹s⁻¹, 5×10⁵ M⁻¹s⁻¹, 1×10⁶ M⁻¹s⁻¹, or 5×10⁶ M⁻¹s⁻¹, as measured by SPR on a Biacore 8k instrument, and a thermal stability Tm as measured by differential scanning fluorimetry (DSF) at 330 and 350 nm with a temperature ramp of 1° C./min from 20 to 95° C. (Tm calculated as first derivative of 350 nm/330 nm ratio) of at least about 59° C., 60° C., 61° C., 62° C., 63° C., 64° C., 65° C., 66° C., 67° C., 68° C., 69° C., 70° C., 71° C., 72° C., 73° C., 74° C., 75° C., 76° C., 77° C., 78° C., 79° C., or 80° C.

In another embodiment, an FcRH5 domain has a preferentially monodispersed profile (e.g. single peak) with average particle diameter in line with a predicted molecular weight and an aggregation of less than about 1%, 0.9%, 0.8%, 0.7%, 0.6%, 0.5%, 0.4%, 0.3%, 0.2%, or 0.1%, as determined by multi-angle dynamic light scattering (MADLS) using a Zetasizer Ultra device and ZS Xplorer software (Malvern Panalytical), at 1 mg/ml in PBS at pH 7.4.

### Antibody Conjugate

The present invention provides an antibody conjugate which comprises the sdAb of the invention and a drug, cargo or payload component. The antibody conjugate may be an antibody-drug conjugate (ADC), which is a class of targeted therapeutics that improves both the selectivity and the cytotoxic activity of cancer drugs.

Typically, ADCs have three components: (i) an antibody conjugated to (ii) a linker, which is also conjugated to (iii) a drug, therapeutic entity or payload, such as a cytotoxic or chemotherapeutic drug.

Upon binding to the target antigen on the surface of a cell, the antibody conjugate is internalised and trafficked to the lysosome where the payload is released by either proteolysis of a cleavable linker (e.g., by cathepsin B found in the lysosome) or by proteolytic degradation of the antibody, if attached to the payload via a non-cleavable linker.

Suitably, the drug or payload may be a chemotherapeutic entity, a radionuclide or a detection entity.

The term “therapeutic entity” or “chemotherapeutic entity”, as used herein, may refer to any molecule that inhibits or prevents the function of cells and/or causes destruction of cells (cell death), and/or exerts anti proliferative effects. The cytotoxic or chemotherapeutic drug refers to a drug that is destructive to a cell and reduces the viability of the cell. Suitable cytotoxic or chemotherapeutic drugs will be known in the art, including, without limitation, a tubulin inhibitor, a DNA damaging agent, a topoisomerase I inhibitor, and an RNA polymerase II inhibitor.

The term “radionuclide” may refer to a radioimmunoconjugate which has a unique theranostic (i.e. therapy and diagnostic) potential. For diagnosis purposes, the antibody may be labelled with a radionuclide compatible with imaging procedures, such as single photon emission computed tomography or positron emission tomography (PET). For therapeutic purposes, the choice of the radionuclide largely depends on the size of the tumour to be treated, with high-energy β-emitters, such as 90Y, being suitable for the therapy of larger tumours, and medium-energy β-emitters, such as 131I and 177Lu, being more effective for the treatment of smaller tumours. Radionuclides suitable for use in ARCs are well known in the art and other radionuclides are also contemplated in the present invention. One of the main attractive features of radioimmunotherapy is the crossfire or bystander effect, i.e., the ability to damage cells in close proximity to the site of antibody localisation. In most cases, antibody radiolabeling is accomplished either by iodination of tyrosines or by conjugation of metal chelators, such as diethylenetriaminepentaacetic acid (DTPA) or 1,4,7,10-tetraazacyclododecane-1,4,7,10-tetraacetic acid (DOTA), to the antibody molecule.

For the purposes of investigating the internalisation properties of the antibody conjugate, the payload may be replaced by a detection or detectable entity.

The “detectable entity” may be a fluorescent entity, for example a fluorescent peptide or dye or label. The term “fluorescent entity”, as used herein, refers to a moiety which, following excitation, emits light at a detectable wavelength. Examples of fluorescent entities include, but are not limited to, fluorescein isothiocyanate (FITC), phycoerythrin (PE), allophycocyanin (APC), green fluorescent protein (GFP), enhanced GFP, red fluorescent protein (RFP), blue fluorescent protein (BFP) and mCherry. Particularly advantageous are pH-sensitive dyes or labels for following the internalization of an antibody conjugate. Non-limiting examples of pH sensitive dyes include fluorescein, which exhibits bright fluorescence that is quenched as the pH drops, and pHrodo dyes, which display very low fluorescence at neutral pH and exhibit increasing fluorescence as the pH becomes more acidic.

The antibody conjugate of the invention may comprise at least one therapeutic or payload molecule (including detection entities) conjugated thereto. The antibody conjugate of the invention may comprise any suitable number i.e. 1, 2, 3, 4, 5, 6, 7, 8, 9, 10, or more payload molecules, to achieve a desired therapeutic effect.

Suitably, the antibody conjugate of the invention is a molecule composed of an sdAb described herein, linked (i.e. conjugated) to a biologically active cytotoxic payload or drug, such as an anticancer drug. The linker may be any appropriate linker known in the art. The person skilled in the art will know that such linkers are routinely used in the production of conjugate molecules and would be able to select an appropriate linker.

Such linkers typically have chemically reactive groups at each end. These linkers can form a covalent attachment between two molecules, e.g. the sdAb and the drug or payload. Thus, the sdAb and the drug or payload may both be covalently linked to a linker. Suitably, one region of the linker may bind to the sdAb and another region of the linker may bind to the drug or payload. The linker may form, for example, hydrazone, disulfide or amide bonds between the sdAb and/or the drug or payload.

### Chimeric Antigen Receptor

The present invention provides a chimeric antigen receptor (CAR) comprising a sdAb as defined herein.

Chimeric antigen receptors (CARs), also known as chimeric T cell receptors, artificial T cell receptors and chimeric immunoreceptors, are engineered receptors, which graft an arbitrary specificity onto an immune effector cell. In a classical CAR, the specificity of a monoclonal antibody is grafted on to a T cell. CAR-encoding nucleic acids may be transferred to T cells using, for example, retroviral vectors. In this way, a large number of cancer-specific T cells can be generated for adoptive cell transfer. Phase I clinical studies of this approach show efficacy.

The target-antigen binding domain of a CAR is commonly fused via a spacer and transmembrane domain to a signaling endodomain, wherein said signaling endodomain is capable of directly transducing an activation signal into the T cell activation signalling cascade. When the CAR binds the target-antigen, this results in the transmission of an activating signal to the T cell it is expressed on. Thus, the CAR of the present invention is able to activate the T cell it is expressed on following binding of the CAR to FcRH5 expressed on the surface of target cells.

Suitably, the sdAb as defined herein may be fused via a spacer and transmembrane domain to a signaling endodomain.

### Transmembrane Domain

The CAR of the invention may comprise a transmembrane domain which spans the membrane of a cell. The transmembrane domain may be any protein structure which is thermodynamically stable in a membrane. It may comprise a hydrophobic alpha helix. The transmembrane domain may be derived from CD8, CD28, human Tyrp-1 or human IgG.

The transmembrane domain may be derived from CD8, which gives good receptor stability.

The transmembrane domain may be derived from any type I transmembrane protein. The transmembrane domain may be a synthetic sequence predicted to form a hydrophobic helix.

As used herein, the term “derived from” refers to the origin or source, and may include naturally occurring, recombinant, unpurified, or purified molecules. The term “derived from” encompasses the terms “originated from,” “obtained from,” “obtainable from,” “isolated from,” and “created from.”

The transmembrane domain may comprise the sequence shown as SEQ ID NO: 54.

The transmembrane domain may comprise the sequence shown as SEQ ID NO: 55.

The transmembrane domain may comprise the sequence shown as SEQ ID NO: 56.

(CD8a transmembrane domain)
SEQ ID NO: 54
IYIWAPLAGTCGVLLLSLVIT
(CD28 transmembrane domain)
SEQ ID NO: 55
FWVLVVVGGVLACYSLLVTVAFIIFWV
(Tyrp-1 transmembrane domain)
SEQ ID NO: 56
IIAIAVVGALLLVALIFGTASYLI

The CAR of the invention may comprise a variant of the sequence shown as SEQ ID NO: 54, 55 or 56 having at least 80% sequence identity, provided that the variant sequence retains the capacity to insert into and span the membrane.

The variant sequence may have at least 80%, 85%, 90%, 95%, 98% or 99% sequence identity to SEQ ID NO: 54, provided that the variant sequence retains the capacity to insert into and span the membrane.

The variant sequence may have at least 80%, 85%, 90%, 95%, 98% or 99% sequence identity to SEQ ID NO: 55, provided that the variant sequence retains the capacity to insert into and span the membrane.

The variant sequence may have at least 80%, 85%, 90%, 95%, 98% or 99% sequence identity to SEQ ID NO: 56, provided that the variant sequence retains the capacity to insert into and span the membrane.

### Spacer

The CAR of the present invention may comprise a spacer sequence to connect the sdAb with the transmembrane domain and spatially separate the sdAb from the endodomain. A flexible spacer allows the sdAb to orient in different directions to enable FcRH5 binding.

The spacer sequence may, for example, comprise an IgG1 Fc region, an IgG1 hinge or a CD8 stalk, or a combination thereof. The spacer may alternatively comprise an alternative sequence which has similar length and/or domain spacing properties as an IgG1 Fc region, an IgG1 hinge or a CD8 stalk. The spacer sequence may, for example, comprise a CD2 ectodomain, CD34 ectodomain or COMP.

A human IgG1 spacer may be altered to remove Fc binding motifs.

The spacer of the CAR of the present invention may comprise one or more of the sequences shown as SEQ ID NO: 57, 58, 59, 60, 61, 62, 63 or 64, or a variant thereof having at least 80% sequence identity.

Examples of amino acid sequences for these spacers are given below:

SEQ ID NO: 57 (hinge-CH2CH3 of human IgG1)
AEPKSPDKTHTCPPCPAPPVAGPSVFLFPPKPKDTLMIARTPEVTCV
VVDVSHEDPEVKFNWYVDGVEVHNAKTKPREEQYNSTYRVVSVLTVL
HQDWLNGKEYKCKVSNKALPAPIEKTISKAKGQPREPQVYTLPPSRD
ELTKNQVSLTCLVKGFYPSDIAVEWESNGQPENNYKTTPPVLDSDGS
FFLYSKLTVDKSRWQQGNVFSCSVMHEALHNHYTQKSLSLSPGKKD
SEQ ID NO: 58 (human CD8 stalk):
TTTPAPRPPTPAPTIASQPLSLRPEACRPAAGGAVHTRGLDFACD
SEQ ID NO: 59 (human IgG1 hinge):
AEPKSPDKTHTCPPCPKDPK
SEQ ID NO: 60 (IgG1 Hinge-Fc)
AEPKSPDKTHTCPPCPAPELLGGPSVFLFPPKPKDTLMISRTPEVTC
VVVDVSHEDPEVKFNWYVDGVEVHNAKTKPREEQYNSTYRVVSVLTV
LHQDWLNGKEYKCKVSNKALPAPIEKTISKAKGQPREPQVYTLPPSR
DELTKNQVSLTCLVKGFYPSDIAVEWESNGQPENNYKTTPPVLDSDG
SFFLYSKLTVDKSRWQQGNVFSCSVMHEALHNHYTQKSLSLSPGKKD
PK
SEQ ID NO: 61 (IgG1 Hinge-Fc modified to remove
Fc receptor recognition motifs)
AEPKSPDKTHTCPPCPAPPVA*GPSVFLFPPKPKDTLMIARTPEVTC
VVVDVSHEDPEVKFNWYVDGVEVHNAKTKPREEQYNSTYRVVSVLTV
LHQDWLNGKEYKCKVSNKALPAPIEKTISKAKGQPREPQVYTLPPSR
DELTKNQVSLTCLVKGFYPSDIAVEWESNGQPENNYKTTPPVLDSDG
SFFLYSKLTVDKSRWQQGNVFSCSVMHEALHNHYTQKSLSLSPGKKD
PK
Modified residues are underlined; * denotes a
deletion.
SEQ ID NO: 62 (CD2 ectodomain)
KEITNALETWGALGQDINLDIPSFQMSDDIDDIKWEKTSDKKKIAQF
RKEKETFKEKDTYKLFKNGTLKIKHLKTDDQDIYKVSIYDTKGKNVL
EKIFDLKIQERVSKPKISWTCINTTLTCEVMNGTDPELNLYQDGKHL
KLSQRVITHKWTTSLSAKFKCTAGNKVSKESSVEPVSCPEKGLD
SEQ ID NO: 63 (CD34 ectodomain)
SLDNNGTATPELPTQGTFSNVSTNVSYQETTTPSTLGSTSLHPVSQH
GNEATTNITETTVKFTSTSVITSVYGNTNSSVQSQTSVISTVFTTPA
NVSTPETTLKPSLSPGNVSDLSTTSTSLATSPTKPYTSSSPILSDIK
AEIKCSGIREVKLTQGICLEQNKTSSCAEFKKDRGEGLARVLCGEEQ
ADADAGAQVCSLLLAQSEVRPQCLLLVLANRTEISSKLQLMKKHQSD
LKKLGILDFTEQDVASHQSYSQKT
SEQ ID NO: 64 (COMP)
DLGPQMLRELQETNAALQDVRELLRQQVREITFLKNTVMECDACG

It is possible to truncate the COMP coiled-coil domain at the N-terminus and retain surface expression. The coiled-coil COMP spacer may therefore comprise or consist of a truncated version of SEQ ID NO: 64, which is truncated at the N-terminus. The truncated COMP may comprise the 5 C-terminal amino acids of SEQ ID NO: 64, i.e. the sequence CDACG (SEQ ID NO: 65). The truncated COMP may comprise 5 to 44 amino acids, for example, at least 5, 10, 15, 20, 25, 30, 35 or 40 amino acids. The truncated COMP may correspond to the C-terminus of SEQ ID NO: 64. For example a truncated COMP comprising 20 amino acids may comprise the sequence QQVREITFLKNTVMECDACG (SEQ ID NO: 66). Truncated COMP may retain the cysteine residue(s) involved in multimerisation. Truncated COMP may retain the capacity to form multimers.

A variant sequence may have at least 80%, 85%, 90%, 95%, 98% or 99% sequence identity to SEQ ID NO: 57.

A variant sequence may have at least 80%, 85%, 90%, 95%, 98% or 99% sequence identity to SEQ ID NO: 58.

A variant sequence may have at least 80%, 85%, 90%, 95%, 98% or 99% sequence identity to SEQ ID NO: 59.

A variant sequence may have at least 80%, 85%, 90%, 95%, 98% or 99% sequence identity to SEQ ID NO: 60.

A variant sequence may have at least 80%, 85%, 90%, 95%, 98% or 99% sequence identity to SEQ ID NO: 61.

A variant sequence may have at least 80%, 85%, 90%, 95%, 98% or 99% sequence identity to SEQ ID NO: 62.

A variant sequence may have at least 80%, 85%, 90%, 95%, 98% or 99% sequence identity to SEQ ID NO: 63.

A variant sequence may have at least 80%, 85%, 90%, 95%, 98% or 99% sequence identity to SEQ ID NO: 64.

### Intracellular Signalling Domain (Endodomain)

The endodomain is the signal-transmission portion of the CAR. After antigen recognition, receptors cluster and a signal is transmitted to the cell. The most commonly used endodomain component is that of CD3-zeta which contains 3 ITAMs. This transmits an activation signal to the T cell after antigen is bound. CD3-zeta may not provide a fully competent activation signal and additional co-stimulatory signaling may be needed. For example, chimeric CD28, 41-BB and OX40 can be used with CD3-Zeta to transmit a proliferative/survival signal, or all three can be used together.

The endodomain of the CAR of the present invention may comprise the CD28 endodomain and/or OX40 endodomain and/or 41-BB endodomain and/or CD3-Zeta endodomain.

The intracellular T cell signalling domain (endodomain) of the CAR of the present invention may comprise one or more of the sequence shown as SEQ ID NO: 67, 68, 69, 70, 71, 72 or 119, or a variant thereof having at least 80% sequence identity.

(CD28 endodomain)
SEQ ID NO: 67
RSKRSRLLHSDYMNMTPRRPGPTRKHYQPYAPPRDFAAY
(OX40 endodomain)
SEQ ID NO: 68
RRDQRLPPDAHKPPGGGSFRTPIQEEQADAHSTLAKI
(41-BB endodomain)
SEQ ID NO: 69
KRGRKKLLYIFKQPFMRPVQTTQEEDGCSCRFPEEEEGGCEL
(CD3 zeta endodomain)
SEQ ID NO: 70
RSRVKFSRSADAPAYQQGQNQLYNELNLGRREEYDVLDKRRGRDPEM
GGKPRRKNPQEGLYNELQKDKMAEAYSEIGMKGERRRGKGHDGLYQG
LSTATKDTYDALHMQALPPR
(CD28Z)
SEQ ID NO: 71
RSKRSRLLHSDYMNMTPRRPGPTRKHYQPYAPPRDFAAYRSRVKFSR
SADAPAYQQGQNQLYNELNLGRREEYDVLDKRRGRDPEMGGKPRRKN
PQEGLYNELQKDKMAEAYSEIGMKGERRRGKGHDGLYQGLSTATKDT
YDALHMQALPPR
(CD28OXZ)
SEQ ID NO: 72
RSKRSRLLHSDYMNMTPRRPGPTRKHYQPYAPPRDFAAYRSRDQRLP
PDAHKPPGGGSFRTPIQEEQADAHSTLAKIRVKFSRSADAPAYQQGQ
NQLYNELNLGRREEYDVLDKRRGRDPEMGGKPRRKNPQEGLYNELQK
DKMAEAYSEIGMKGERRRGKGHDGLYQGLSTATKDTYDALHMQALPP
R
(4-1BB-CD3Z)
SEQ ID NO: 119
KRGRKKLLYIFKQPFMRPVQTTQEEDGCSCRFPEEEEGGCELRVKFS
RSADAPAYQQGQNQLYNELNLGRREEYDVLDKRRGRDPEMGGKPRRK
NPQEGLYNELQKDKMAEAYSEIGMKGERRRGKGHDGLYQGLSTATKD
TYDALHMQALPPRA

A variant sequence may have at least 80%, 85%, 90%, 95%, 98% or 99% sequence identity to SEQ ID NO: 67, provided that the sequence provides an effective intracellular T cell signaling domain.

A variant sequence may have at least 80%, 85%, 90%, 95%, 98% or 99% sequence identity to SEQ ID NO: 68, provided that the sequence provides an effective intracellular T cell signaling domain.

A variant sequence may have at least 80%, 85%, 90%, 95%, 98% or 99% sequence identity to SEQ ID NO: 69, provided that the sequence provides an effective intracellular T cell signaling domain.

A variant sequence may have at least 80%, 85%, 90%, 95%, 98% or 99% sequence identity to SEQ ID NO: 70, provided that the sequence provides an effective intracellular T cell signaling domain.

A variant sequence may have at least 80%, 85%, 90%, 95%, 98% or 99% sequence identity to SEQ ID NO: 71, provided that the sequence provides an effective intracellular T cell signaling domain.

A variant sequence may have at least 80%, 85%, 90%, 95%, 98% or 99% sequence identity to SEQ ID NO: 72, provided that the sequence provides an effective intracellular T cell signaling domain.

A variant sequence may have at least 80%, 85%, 90%, 95%, 98% or 99% sequence identity to SEQ ID NO: 119, provided that the sequence provides an effective intracellular T cell signaling domain.

### Signal Peptide

The CAR of the present invention may comprise a signal peptide so that when the CAR is expressed inside a cell, such as a T cell, the nascent protein is directed to the endoplasmic reticulum and subsequently to the cell surface, where it is expressed.

The core of the signal peptide may contain a long stretch of hydrophobic amino acids that has a tendency to form a single alpha-helix. The signal peptide may begin with a short positively charged stretch of amino acids, which helps to enforce proper topology of the polypeptide during translocation. At the end of the signal peptide there is typically a stretch of amino acids that is recognized and cleaved by signal peptidase. Signal peptidase may cleave either during or after completion of translocation to generate a free signal peptide and a mature protein. The free signal peptides are then digested by specific proteases.

The signal peptide may be at the amino terminus of the molecule.

The CAR of the invention may have the general formula:
Signal peptide-sdAb-spacer domain-transmembrane domain-intracellular T cell signaling domain(s).

The signal peptide may comprise the sequence shown as SEQ ID NO: 73 or SEQ ID NO: 358, or a variant thereof having 5, 4, 3, 2 or 1 amino acid mutations (e.g. insertions, substitutions or additions) provided that the signal peptide still functions to cause cell surface expression of the CAR.

SEQ ID NO: 73:
METDTLLLWVLLLWVPGSTG
SEQ ID NO: 358:
MGWSCIILFLVATATGVHS

The signal peptide of SEQ ID NO: 73 or SEQ ID NO: 358 is compact and highly efficient. It is predicted to give about 95% cleavage after the terminal glycine, giving efficient removal by signal peptidase.

### Suicide Genes

Since T cells engraft and are autonomous, a means of selectively deleting CAR T cells in recipients of CAR T cells is desirable. Suicide genes are genetically encodable mechanisms which result in selective destruction of infused T cells in the face of unacceptable toxicity. The earliest clinical experience with suicide genes is with the Herpes Virus Thymidine Kinase (HSV-TK) which renders T cells susceptible to Ganciclovir. HSV-TK is a highly effective suicide gene. However, pre-formed immune responses may restrict its use to clinical settings of considerable immunosuppression such as haploidentical stem cell transplantation. Inducible Caspase 9 (iCasp9) is a suicide gene constructed by replacing the activating domain of Caspase 9 with a modified FKBP12. iCasp9 is activated by an otherwise inert small molecular chemical inducer of dimerization (CID). iCasp9 has been recently tested in the setting of haploidentical HSCT and can abort GvHD. The biggest limitation of iCasp9 is dependence on availability of clinical grade proprietary CID. Both iCasp9 and HSV-TK are intracellular proteins, so when used as the sole transgene, they have been co-expressed with a marker gene to allow selection of transduced cells.

An iCasp9 may comprise the sequence shown as SEQ ID NO: 74 or a variant thereof having at least 80%, 85%, 90%, 95%, 98% or 99% sequence identity.

SEQ ID NO: 74
MLEGVQVETISPGDGRTFPKRGQTCVVHYTGMLEDGKKVDSSRDRNK
PFKFMLGKQEVIRGWEEGVAQMSVGQRAKLTISPDYAYGATGHPGII
PPHATLVFDVELLKLESGGGSGVDGFGDVGALESLRGNADLAYILSM
EPCGHCLIINNVNFCRESGLRTRTGSNIDCEKLRRRFSSLHFMVEVK
GDLTAKKMVLALLELAQQDHGALDCCVVVILSHGCQASHLQFPGAVY
GTDGCPVSVEKIVNIFNGTSCPSLGGKPKLFFIQACGGEQKDHGFEV
ASTSPEDESPGSNPEPDATPFQEGLRTFDQLDAISSLPTPSDIFVSY
STFPGFVSWRDPKSGSWYVETLDDIFEQWAHSEDLQSLLLRVANAVS
VKGIYKQMPGCFNFLRKKLFFKTSAS

Another marker/suicide gene is RQR8 which can be detected with the antibody QBEnd10 and expressing cells lysed with the therapeutic antibody Rituximab.

An RQR8 may comprise the sequence shown as SEQ ID NO: 75 or a variant thereof having at least 80%, 85%, 90%, 95%, 98% or 99% sequence identity.

SEQ ID NO: 75
MGTSLLCWMALCLLGADHADACPYSNPSLCSGGGGSELPTQGTFSN
VSTNVSPAKPTTTACPYSNPSLCSGGGGSPAPRPPTPAPTIASQPL
SLRPEACRPAAGGAVHTRGLDFACDIYIWAPLAGTCGVLLLSLVIT
LYCNHRNRRRVCKCPRPVV

The suicide gene may be expressed in a single polypeptide with the CAR, separated by a co-expression site enabling co-expression of two polypeptides as separate entities. It may be a sequence encoding a cleavage site, such that both polypeptides are joined by a cleavage site. The cleavage site may be self-cleaving, such that when the polypeptide is produced, it is immediately cleaved into individual peptides without the need for any external cleavage activity.

The cleavage site may be any sequence which enables the two polypeptides to become separated.

The term “cleavage” is used herein for convenience, but the cleavage site may cause the peptides to separate into individual entities by a mechanism other than classical cleavage. For example, for the Foot-and-Mouth disease virus (FMDV) 2A self-cleaving peptide (see below), various models have been proposed for to account for the “cleavage” activity: proteolysis by a host-cell proteinase, autoproteolysis or a translational effect (Donnelly et al (2001) J. Gen. Virol. 82:1027-1041). The exact mechanism of such “cleavage” is not important for the purposes of the present invention, as long as the cleavage site, when positioned between nucleic acid sequences which encode proteins, causes the proteins to be expressed as separate entities.

The cleavage site may, for example be a furin cleavage site, a Tobacco Etch Virus (TEV) cleavage site or encode a self-cleaving peptide.

A ‘self-cleaving peptide’ refers to a peptide which functions such that when the polypeptide comprising the proteins and the self-cleaving peptide is produced, it is immediately “cleaved” or separated into distinct and discrete first and second polypeptides without the need for any external cleavage activity.

The self-cleaving peptide may be a 2A self-cleaving peptide from an aphtho- or a cardiovirus. The primary 2A/2B cleavage of the aptho- and cardioviruses is mediated by 2A “cleaving” at its own C-terminus. In apthoviruses, such as FMDV and equine rhinitis A virus, the 2A region is a short section of about 18 amino acids, which, together with the N-terminal residue of protein 2B (a conserved proline residue) represents an autonomous element capable of mediating “cleavage” at its own C-terminus (Donelly et al (2001) as above).

“2A-like” sequences have been found in picornaviruses other than aptho- or cardioviruses, ‘picornavirus-like’ insect viruses, type C rotaviruses and repeated sequences within Trypanosoma spp and a bacterial sequence (Donnelly et al (2001) as above).

The cleavage site may comprise the 2A-like sequence shown as SEQ ID NO: 359 (RAEGRGSLLTCGDVEENPGP).

### FcRH5 CARS

The CAR of the present invention may comprise a sequence selected from the group consisting of the sequences shown as SEQ ID NOs: 76-89, 159 and 289-354 or a variant thereof which has at least 80%, 85%, 90%, 95%, 98% or 99% sequence identity thereto but retains the capacity to i) bind FcRH5 and ii) induce T cell signalling.

The CAR may comprise a CD8 spacer region, a CD8 transmembrane region, a 41BB intracellular signalling domain and a CD3z chain intracellular signalling domain. The CAR may also comprise a RQR8 transduction marker separated by a 2A site.

SEQ ID NO: 76
MGTSLLCWMALCLLGADHADACPYSNPSLCSGGGGSELPTQGTFSNVSTNVSPAKPTTTA
CPYSNPSLCSGGGGSPAPRPPTPAPTIASQPLSLRPEACRPAAGGAVHTRGLDFACDIYI
WAPLAGTCGVLLLSLVITLYCNHRNRRRVCKCPRPVVRAEGRGSLLTCGDVEENPGPMGW
SCIILFLVATATGVHSQVQLQESGGGLVQAGGSLRLSCAASRSSFSNNAMGWYRQVPGKQ
RELVAFITKGGVTDYSDSVKGRFTISKDNAKNTVYLQMNSLKPEDTAVYYCNTIPFRSAW
GQGTQVTVSSDPTPTPAPTIASQPLSLRPEACRPAAGGAVHTRGLDFACDIYIWAPLAGT
CGVLLLSLVITLYCKRGRKKLLYIFKQPFMRPVQTTQEEDGCSCRFPEEEEGGCELRVKF
SRSADAPAYQQGQNQLYNELNLGRREEYDVLDKRRGRDPEMGGKPRRKNPQEGLYNELQK
DKMAEAYSEIGMKGERRRGKGHDGLYQGLSTATKDTYDALHMQALPPR
SEQ ID NO: 77
MGTSLLCWMALCLLGADHADACPYSNPSLCSGGGGSELPTQGTFSNVSTNVSPAKPTTTA
CPYSNPSLCSGGGGSPAPRPPTPAPTIASQPLSLRPEACRPAAGGAVHTRGLDFACDIYI
WAPLAGTCGVLLLSLVITLYCNHRNRRRVCKCPRPVVRAEGRGSLLTCGDVEENPGPMGW
SCIILFLVATATGVHSQVQLQESGGGSVQPGGSLSLSCAASGFTFSNYAMSWVRQAPGKG
PEWVAVINSDGGTASSAGSVRGRFTISRDNAKNTLYLQMNRLKPEDTAVYYCAANRGFCA
GVRCLEYQYWGQGTQVTVSSDPTPTPAPTIASQPLSLRPEACRPAAGGAVHTRGLDFACD
IYIWAPLAGTCGVLLLSLVITLYCKRGRKKLLYIFKQPFMRPVQTTQEEDGCSCRFPEEE
EGGCELRVKFSRSADAPAYQQGQNQLYNELNLGRREEYDVLDKRRGRDPEMGGKPRRKNP
QEGLYNELQKDKMAEAYSEIGMKGERRRGKGHDGLYQGLSTATKDTYDALHMQALPPR
SEQ ID NO: 78
MGTSLLCWMALCLLGADHADACPYSNPSLCSGGGGSELPTQGTFSNVSTNVSPAKPTTTA
CPYSNPSLCSGGGGSPAPRPPTPAPTIASQPLSLRPEACRPAAGGAVHTRGLDFACDIYI
WAPLAGTCGVLLLSLVITLYCNHRNRRRVCKCPRPVVRAEGRGSLLTCGDVEENPGPMGW
SCIILFLVATATGVHSQVQLQESGGGLVQAGGSLIVSCAASGRTFSINAMAWFRQAPGKE
REFVAAIGGSGRVSSTSYADFVKGRFTISRDNAKNTVYLRMNNLEPEDTAVYYCAARRDY
LPFPPESYDYWGQGTQVTVSSDPTPTPAPTIASQPLSLRPEACRPAAGGAVHTRGLDFAC
DIYIWAPLAGTCGVLLLSLVITLYCKRGRKKLLYIFKQPFMRPVQTTQEEDGCSCRFPEE
EEGGCELRVKFSRSADAPAYQQGQNQLYNELNLGRREEYDVLDKRRGRDPEMGGKPRRKN
PQEGLYNELQKDKMAEAYSEIGMKGERRRGKGHDGLYQGLSTATKDTYDALHMQALPPR
SEQ ID NO: 79
MGTSLLCWMALCLLGADHADACPYSNPSLCSGGGGSELPTQGTFSNVSTNVSPAKPTTTA
CPYSNPSLCSGGGGSPAPRPPTPAPTIASQPLSLRPEACRPAAGGAVHTRGLDFACDIYI
WAPLAGTCGVLLLSLVITLYCNHRNRRRVCKCPRPVVRAEGRGSLLTCGDVEENPGPMGW
SCIILFLVATATGVHSQVQLQESGGGLVQAGGSLRLSCAASGRTFSTYAMAWFRQAPGKE
REFVAAISGFGVVTYYADSVKGRFTISRDNAKNTLYLQMNGLKPEDTAVYYCAAGRRTST
NGGDYDYWGQGTQVTVSSDPTTTPAPRPPTPAPTIASQPLSLRPEACRPAAGGAVHTRGL
DFACDIYIWAPLAGTCGVLLLSLVITLYCKRGRKKLLYIFKQPFMRPVQTTQEEDGCSCR
FPEEEEGGCELRVKFSRSADAPAYQQGQNQLYNELNLGRREEYDVLDKRRGRDPEMGGKP
RRKNPQEGLYNELQKDKMAEAYSEIGMKGERRRGKGHDGLYQGLSTATKDTYDALHMQAL
PPRA
SEQ ID NO: 80
MGTSLLCWMALCLLGADHADACPYSNPSLCSGGGGSELPTQGTFSNVSTNVSPAKPTTTA
CPYSNPSLCSGGGGSPAPRPPTPAPTIASQPLSLRPEACRPAAGGAVHTRGLDFACDIYI
WAPLAGTCGVLLLSLVITLYCNHRNRRRVCKCPRPVVRAEGRGSLLTCGDVEENPGPMGW
SCIILFLVATATGVHSQVQLQESGGGLVQAGGSLIVSCATSGRTFSINAMAWFRQAPGKE
REFVAAIGGSGRVSSTSYADFVKGRFTISRDNAKNTVYLRMNNLEPEDTAVYYCAARRNY
LPFPPESYDYWGQGIQVTVSSDPTPTPAPTIASQPLSLRPEACRPAAGGAVHTRGLDFAC
DIYIWAPLAGTCGVLLLSLVITLYCKRGRKKLLYIFKQPFMRPVQTTQEEDGCSCRFPEE
EEGGCELRVKFSRSADAPAYQQGQNQLYNELNLGRREEYDVLDKRRGRDPEMGGKPRRKN
PQEGLYNELQKDKMAEAYSEIGMKGERRRGKGHDGLYQGLSTATKDTYDALHMQALPPR
SEQ ID NO: 81
MGTSLLCWMALCLLGADHADACPYSNPSLCSGGGGSELPTQGTFSNVSTNVSPAKPTTTA
CPYSNPSLCSGGGGSPAPRPPTPAPTIASQPLSLRPEACRPAAGGAVHTRGLDFACDIYI
WAPLAGTCGVLLLSLVITLYCNHRNRRRVCKCPRPVVRAEGRGSLLTCGDVEENPGPMGW
SCIILFLVATATGVHSQVQLQQSGGGLVQAGDSLRLSCAASGRTFSNSTMGWFRQAPGKE
RKFVAVISWSGGTYAYAESVKGRFTISRDNAKNTVYLQMNSLKPEDTAVYYCAAARKGWS
TRGDDYDYWGQGTQVTVSSDPTPTPAPTIASQPLSLRPEACRPAAGGAVHTRGLDFACDI
YIWAPLAGTCGVLLLSLVITLYCKRGRKKLLYIFKQPFMRPVQTTQEEDGCSCRFPEEEE
GGCELRVKFSRSADAPAYQQGQNQLYNELNLGRREEYDVLDKRRGRDPEMGGKPRRKNPQ
EGLYNELQKDKMAEAYSEIGMKGERRRGKGHDGLYQGLSTATKDTYDALHMQALPPR
SEQ ID NO: 82
MGTSLLCWMALCLLGADHADACPYSNPSLCSGGGGSELPTQGTFSNVSTNVSPAKPTTTA
CPYSNPSLCSGGGGSPAPRPPTPAPTIASQPLSLRPEACRPAAGGAVHTRGLDFACDIYI
WAPLAGTCGVLLLSLVITLYCNHRNRRRVCKCPRPVVRAEGRGSLLTCGDVEENPGPMGW
SCIILFLVATATGVHSQVQLQESGGGLVQAGGSLRLSCAASGRTYNNYAMGWFRQAPGKE
REFVAGISRSGGMTGYAESVKGRFTISRDNAKNMVFLQMNSLKPEDTAVYYCAAYVGGFS
TARRDYSYWGQGTQVTVSSDPTPTPAPTIASQPLSLRPEACRPAAGGAVHTRGLDFACDI
YIWAPLAGTCGVLLLSLVITLYCKRGRKKLLYIFKQPFMRPVQTTQEEDGCSCRFPEEEE
GGCELRVKFSRSADAPAYQQGQNQLYNELNLGRREEYDVLDKRRGRDPEMGGKPRRKNPQ
EGLYNELQKDKMAEAYSEIGMKGERRRGKGHDGLYQGLSTATKDTYDALHMQALPPR
SEQ ID NO: 83
MGTSLLCWMALCLLGADHADACPYSNPSLCSGGGGSELPTQGTFSNVSTNVSPAKPTTTA
CPYSNPSLCSGGGGSPAPRPPTPAPTIASQPLSLRPEACRPAAGGAVHTRGLDFACDIYI
WAPLAGTCGVLLLSLVITLYCNHRNRRRVCKCPRPVVRAEGRGSLLTCGDVEENPGPMGW
SCIILFLVATATGVHSQVQLQESGGGLVQAGDSLRLSCAYSGRTFSSYAMGWFRQAPGKE
RVFVAAISRIGGVTTYAESVQGRFTISRDNAKNTLYLQMNALKPEDTAVYYCAAAGLVSI
STTPNDYDYWGQGTQVTVSSDPTPTPAPTIASQPLSLRPEACRPAAGGAVHTRGLDFACD
IYIWAPLAGTCGVLLLSLVITLYCKRGRKKLLYIFKQPFMRPVQTTQEEDGCSCRFPEEE
EGGCELRVKFSRSADAPAYQQGQNQLYNELNLGRREEYDVLDKRRGRDPEMGGKPRRKNP
QEGLYNELQKDKMAEAYSEIGMKGERRRGKGHDGLYQGLSTATKDTYDALHMQALPPR
SEQ ID NO: 84
MGTSLLCWMALCLLGADHADACPYSNPSLCSGGGGSELPTQGTFSNVSTNVSPAKPTTTA
CPYSNPSLCSGGGGSPAPRPPTPAPTIASQPLSLRPEACRPAAGGAVHTRGLDFACDIYI
WAPLAGTCGVLLLSLVITLYCNHRNRRRVCKCPRPVVRAEGRGSLLTCGDVEENPGPMGW
SCIILFLVATATGVHSQVQLQQSGGGLVQPGGSLRLSCAAPGNIFRLNGTGWYRQAPGKQ
RELVAHITSGGNTDYADSVKGRFTISRDNAKNTVYLQMNSLKPEDTAVYYCNAIPFRLSW
GQGTQVTVSSDPTPTPAPTIASQPLSLRPEACRPAAGGAVHTRGLDFACDIYIWAPLAGT
CGVLLLSLVITLYCKRGRKKLLYIFKQPFMRPVQTTQEEDGCSCRFPEEEEGGCELRVKF
SRSADAPAYQQGQNQLYNELNLGRREEYDVLDKRRGRDPEMGGKPRRKNPQEGLYNELQK
DKMAEAYSEIGMKGERRRGKGHDGLYQGLSTATKDTYDALHMQALPPR
SEQ ID NO: 85
MGTSLLCWMALCLLGADHADACPYSNPSLCSGGGGSELPTQGTFSNVSTNVSPAKPTTTA
CPYSNPSLCSGGGGSPAPRPPTPAPTIASQPLSLRPEACRPAAGGAVHTRGLDFACDIYI
WAPLAGTCGVLLLSLVITLYCNHRNRRRVCKCPRPVVRAEGRGSLLTCGDVEENPGPMGW
SCIILFLVATATGVHSQVQLQESGGGLVQAGGSLRLSCVVSRNIFSLNPMGWYRQAPGKQ
REMVAIITNGGSTNYADSVKGRFTISRDNVKNTVYLQMNALKPEDTAVYYCNRVGGLQTW
AWGQGTQVTVSSDPTPTPAPTIASQPLSLRPEACRPAAGGAVHTRGLDFACDIYIWAPLA
GTCGVLLLSLVITLYCKRGRKKLLYIFKQPFMRPVQTTQEEDGCSCRFPEEEEGGCELRV
KFSRSADAPAYQQGQNQLYNELNLGRREEYDVLDKRRGRDPEMGGKPRRKNPQEGLYNEL
QKDKMAEAYSEIGMKGERRRGKGHDGLYQGLSTATKDTYDALHMQALPPR
SEQ ID NO: 86
MGTSLLCWMALCLLGADHADACPYSNPSLCSGGGGSELPTQGTFSNVSTNVSPAKPTTTA
CPYSNPSLCSGGGGSPAPRPPTPAPTIASQPLSLRPEACRPAAGGAVHTRGLDFACDIYI
WAPLAGTCGVLLLSLVITLYCNHRNRRRVCKCPRPVVRAEGRGSLLTCGDVEENPGPMGW
SCIILFLVATATGVHSQVQLQQSGGGLVQAGGSLSLSCTASGRSFSNYGMGWFRQAPGKE
REFVAAIGMVGGLTAYSNSAKGRFTISRDNAKNTLYLQMNSLKPEDTAVYLCAAGRRFST
SSRDYDIWGQGTQVTVSSDPTPTPAPTIASQPLSLRPEACRPAAGGAVHTRGLDFACDIY
IWAPLAGTCGVLLLSLVITLYCKRGRKKLLYIFKQPFMRPVQTTQEEDGCSCRFPEEEEG
GCELRVKFSRSADAPAYQQGQNQLYNELNLGRREEYDVLDKRRGRDPEMGGKPRRKNPQE
GLYNELQKDKMAEAYSEIGMKGERRRGKGHDGLYQGLSTATKDTYDALHMQALPPR
SEQ ID NO: 87
MGTSLLCWMALCLLGADHADACPYSNPSLCSGGGGSELPTQGTFSNVSTNVSPAKPTTTA
CPYSNPSLCSGGGGSPAPRPPTPAPTIASQPLSLRPEACRPAAGGAVHTRGLDFACDIYI
WAPLAGTCGVLLLSLVITLYCNHRNRRRVCKCPRPVVRAEGRGSLLTCGDVEENPGPMGW
SCIILFLVATATGVHSQVQLQESGGGLVQAGDSLRLSCAASGRTFDSRPMGWFRQAPGKE
REFVGAVSWRGESTYYPDSVKGRFTISRDNAKRTVYLQMNSLKPEDTAVYYCAAGEPYSG
TYYYRGRDYDYWGQGTQVTVSSDPTPTPAPTIASQPLSLRPEACRPAAGGAVHTRGLDFA
CDIYIWAPLAGTCGVLLLSLVITLYCKRGRKKLLYIFKQPFMRPVQTTQEEDGCSCRFPE
EEEGGCELRVKFSRSADAPAYQQGQNQLYNELNLGRREEYDVLDKRRGRDPEMGGKPRRK
NPQEGLYNELQKDKMAEAYSEIGMKGERRRGKGHDGLYQGLSTATKDTYDALHMQALPPR
SEQ ID NO: 88
MGTSLLCWMALCLLGADHADACPYSNPSLCSGGGGSELPTQGTFSNVSTNVSPAKPTTTA
CPYSNPSLCSGGGGSPAPRPPTPAPTIASQPLSLRPEACRPAAGGAVHTRGLDFACDIYI
WAPLAGTCGVLLLSLVITLYCNHRNRRRVCKCPRPVVRAEGRGSLLTCGDVEENPGPMGW
SCIILFLVATATGVHSQVQLQESGGGLVQAEGSLRLSCAASGRTFSMYAMGWFRQAPGRE
REFVAAISGSARITYYGQSVKGRFTISRDNAKNTVYLQMNSLKPEDTAVYYCAASSTYTS
TSGSSYNYWGQGTQVTVSSDPTPTPAPTIASQPLSLRPEACRPAAGGAVHTRGLDFACDI
YIWAPLAGTCGVLLLSLVITLYCKRGRKKLLYIFKQPFMRPVQTTQEEDGCSCRFPEEEE
GGCELRVKFSRSADAPAYQQGQNQLYNELNLGRREEYDVLDKRRGRDPEMGGKPRRKNPQ
EGLYNELQKDKMAEAYSEIGMKGERRRGKGHDGLYQGLSTATKDTYDALHMQALPPR
SEQ ID NO: 89
MGTSLLCWMALCLLGADHADACPYSNPSLCSGGGGSELPTQGTFSNVSTNVSPAKPTTTA
CPYSNPSLCSGGGGSPAPRPPTPAPTIASQPLSLRPEACRPAAGGAVHTRGLDFACDIYI
WAPLAGTCGVLLLSLVITLYCNHRNRRRVCKCPRPVVRAEGRGSLLTCGDVEENPGPMGW
SCIILFLVATATGVHSQVQLQESGGGLVQAGGSLRLSCAASGSIFSINAMGWYRQAPGKQ
RELVAFITSGGSTNYADSVKGRFTISRDNAKNTVYLQMNSLKPEDTAVYYCNALGGFVPS
YGWGQGTQVTVSSDPTPTPAPTIASQPLSLRPEACRPAAGGAVHTRGLDFACDIYIWAPL
AGTCGVLLLSLVITLYCKRGRKKLLYIFKQPFMRPVQTTQEEDGCSCRFPEEEEGGCELR
VKFSRSADAPAYQQGQNQLYNELNLGRREEYDVLDKRRGRDPEMGGKPRRKNPQEGLYNE
LQKDKMAEAYSEIGMKGERRRGKGHDGLYQGLSTATKDTYDALHMQALPPR
SEQ ID NO: 159
MGTSLLCWMALCLLGADHADACPYSNPSLCSGGGGSELPTQGTFSNVSTNVSPAKPTTTA
CPYSNPSLCSGGGGSPAPRPPTPAPTIASQPLSLRPEACRPAAGGAVHTRGLDFACDIYI
WAPLAGTCGVLLLSLVITLYCNHRNRRRVCKCPRPVVRAEGRGSLLTCGDVEENPGPMGW
SCIILFLVATATGVHSQVQLQESGGGLVQAGGSLRLSCTASGRTSSRAAMGWFRQAPGKE
REFVAVISWSGGTTAYANSVKGRFTISRDNAKNTLYLQMNSLKPEDTAVYYCAAARIFTT
ARNDYDHWGQGTQVTVSSDPTPTPAPTIASQPLSLRPEACRPAAGGAVHTRGLDFACDIY
IWAPLAGTCGVLLLSLVITLYCKRGRKKLLYIFKQPFMRPVQTTQEEDGCSCRFPEEEEG
GCELRVKFSRSADAPAYQQGQNQLYNELNLGRREEYDVLDKRRGRDPEMGGKPRRKNPQE
GLYNELQKDKMAEAYSEIGMKGERRRGKGHDGLYQGLSTATKDTYDALHMQALPPR
SEQ ID NO: 289
MGTSLLCWMALCLLGADHADACPYSNPSLCSGGGGSELPTQGTFSNVSTNVSPAKPTTTA
CPYSNPSLCSGGGGSPAPRPPTPAPTIASQPLSLRPEACRPAAGGAVHTRGLDFACDIYI
WAPLAGTCGVLLLSLVITLYCNHRNRRRVCKCPRPVVRAEGRGSLLTCGDVEENPGPMGW
SCIILFLVATATGVHSQVQLQESGGGLVQPGGSLRLSCAAPGNIFRLNGTGWYRQAPGKQ
RELVAHITSGGNTDYADSVKGRFTISRDNAKNTVYLQMNSLKPEDTAVYYCNTIPFRLSW
GQGTQVTVSSDPTPTPAPTIASQPLSLRPEACRPAAGGAVHTRGLDFACDIYIWAPLAGT
CGVLLLSLVITLYCKRGRKKLLYIFKQPFMRPVQTTQEEDGCSCRFPEEEEGGCELRVKF
SRSADAPAYQQGQNQLYNELNLGRREEYDVLDKRRGRDPEMGGKPRRKNPQEGLYNELQK
DKMAEAYSEIGMKGERRRGKGHDGLYQGLSTATKDTYDALHMQALPPR
SEQ ID NO: 290
MGTSLLCWMALCLLGADHADACPYSNPSLCSGGGGSELPTQGTFSNVSTNVSPAKPTTTA
CPYSNPSLCSGGGGSPAPRPPTPAPTIASQPLSLRPEACRPAAGGAVHTRGLDFACDIYI
WAPLAGTCGVLLLSLVITLYCNHRNRRRVCKCPRPVVRAEGRGSLLTCGDVEENPGPMGW
SCIILFLVATATGVHSQVQLQESGGGLVQPGGSLRLSCAAPGNIFRLNAMGWYRQAPGKQ
RELVAHITSGGNTDYADSVKGRFTISRDNAKNTVYLQMNSLKLEDTAVYYCNTIPFRLSW
GQGTQVTVSSDPTPTPAPTIASQPLSLRPEACRPAAGGAVHTRGLDFACDIYIWAPLAGT
CGVLLLSLVITLYCKRGRKKLLYIFKQPFMRPVQTTQEEDGCSCRFPEEEEGGCELRVKF
SRSADAPAYQQGQNQLYNELNLGRREEYDVLDKRRGRDPEMGGKPRRKNPQEGLYNELQK
DKMAEAYSEIGMKGERRRGKGHDGLYQGLSTATKDTYDALHMQALPPR
SEQ ID NO: 291
MGTSLLCWMALCLLGADHADACPYSNPSLCSGGGGSELPTQGTFSNVSTNVSPAKPTTTA
CPYSNPSLCSGGGGSPAPRPPTPAPTIASQPLSLRPEACRPAAGGAVHTRGLDFACDIYI
WAPLAGTCGVLLLSLVITLYCNHRNRRRVCKCPRPVVRAEGRGSLLTCGDVEENPGPMGW
SCIILFLVATATGVHSQVQLQESGGGLAQAGGSLRLSCAASRSSFSNNAMGWYRQVQGKQ
RELVAFITKGGVTDYSVSGKGRFTISKDHAKNTVYLQMNSLKPRDTAVYYCNTIPVRSAW
GQGTQVTVSADPTPTPAPTIASQPLSLRPEACRPAAGGAVHTRGLDFACDIYIWAPLAGT
CGVLLLSLVITLYCKRGRKKLLYIFKQPFMRPVQTTQEEDGCSCRFPEEEEGGCELRVKF
SRSADAPAYQQGQNQLYNELNLGRREEYDVLDKRRGRDPEMGGKPRRKNPQEGLYNELQK
DKMAEAYSEIGMKGERRRGKGHDGLYQGLSTATKDTYDALHMQALPPR
SEQ ID NO: 292
MGTSLLCWMALCLLGADHADACPYSNPSLCSGGGGSELPTQGTFSNVSTNVSPAKPTTTA
CPYSNPSLCSGGGGSPAPRPPTPAPTIASQPLSLRPEACRPAAGGAVHTRGLDFACDIYI
WAPLAGTCGVLLLSLVITLYCNHRNRRRVCKCPRPVVRAEGRGSLLTCGDVEENPGPMGW
SCIILFLVATATGVHSQVQLQESGGGLVQAGGSLRLSCAASGSIFSINAMGWYRQAPGKQ
RELVAFITKGGVTDYSDSVKGRFTISKDNAKNTVYLQMNSLKPEDTAVYYCNTIPCRSAW
GQGTQVTVSSDPTPTPAPTIASQPLSLRPEACRPAAGGAVHTRGLDFACDIYIWAPLAGT
CGVLLLSLVITLYCKRGRKKLLYIFKQPFMRPVQTTQEEDGCSCRFPEEEEGGCELRVKF
SRSADAPAYQQGQNQLYNELNLGRREEYDVLDKRRGRDPEMGGKPRRKNPQEGLYNELQK
DKMAEAYSEIGMKGERRRGKGHDGLYQGLSTATKDTYDALHMQALPPR
SEQ ID NO: 293
MGTSLLCWMALCLLGADHADACPYSNPSLCSGGGGSELPTQGTFSNVSTNVSPAKPTTTA
CPYSNPSLCSGGGGSPAPRPPTPAPTIASQPLSLRPEACRPAAGGAVHTRGLDFACDIYI
WAPLAGTCGVLLLSLVITLYCNHRNRRRVCKCPRPVVRAEGRGSLLTCGDVEENPGPMGW
SCIILFLVATATGVHSQVQLQESGGGLVQPGGSLRLSCAAPGSSFRLNGTGWYRQAPGKQ
RELVAHITSGGSTNYSDSVKGRLTISKDNAKNTVYLQMNSLKPEDTAVYYCNTIPFSRAW
GQGTLVTVSSDPTPTPAPTIASQPLSLRPEACRPAAGGAVHTRGLDFACDIYIWAPLAGT
CGVLLLSLVITLYCKRGRKKLLYIFKQPFMRPVQTTQEEDGCSCRFPEEEEGGCELRVKF
SRSADAPAYQQGQNQLYNELNLGRREEYDVLDKRRGRDPEMGGKPRRKNPQEGLYNELQK
DKMAEAYSEIGMKGERRRGKGHDGLYQGLSTATKDTYDALHMQALPPR
SEQ ID NO: 294
MGTSLLCWMALCLLGADHADACPYSNPSLCSGGGGSELPTQGTFSNVSTNVSPAKPTTTA
CPYSNPSLCSGGGGSPAPRPPTPAPTIASQPLSLRPEACRPAAGGAVHTRGLDFACDIYI
WAPLAGTCGVLLLSLVITLYCNHRNRRRVCKCPRPVVRAEGRGSLLTCGDVEENPGPMGW
SCIILFLVATATGVHSQVQLQQSGGGLVQAGGSLRLSCAASGRSVSINAMGWYRQAPGKQ
RELVAIIDRSGNTDYADSVKGRFTISRDNAKKAVYLQMNSLKPEDTAVYYCNTIPYSDSW
GQGTQVTISSDPTPTPAPTIASQPLSLRPEACRPAAGGAVHTRGLDFACDIYIWAPLAGT
CGVLLLSLVITLYCKRGRKKLLYIFKQPFMRPVQTTQEEDGCSCRFPEEEEGGCELRVKF
SRSADAPAYQQGQNQLYNELNLGRREEYDVLDKRRGRDPEMGGKPRRKNPQEGLYNELQK
DKMAEAYSEIGMKGERRRGKGHDGLYQGLSTATKDTYDALHMQALPPR
SEQ ID NO: 295
MGTSLLCWMALCLLGADHADACPYSNPSLCSGGGGSELPTQGTFSNVSTNVSPAKPTTTA
CPYSNPSLCSGGGGSPAPRPPTPAPTIASQPLSLRPEACRPAAGGAVHTRGLDFACDIYI
WAPLAGTCGVLLLSLVITLYCNHRNRRRVCKCPRPVVRAEGRGSLLTCGDVEENPGPMGW
SCIILFLVATATGVHSQVQLQESGGGLVQPGGSLRLSCAASGRTFSSYAMGWFRRAPGKG
REFVATIDGIGGITSYAGSVKGRFTVSKDNAKNTVYLQMNSLKPEDTAVYYCNTIPFRSA
WGQGTLVTVSSDPTPTPAPTIASQPLSLRPEACRPAAGGAVHTRGLDFACDIYIWAPLAG
TCGVLLLSLVITLYCKRGRKKLLYIFKQPFMRPVQTTQEEDGCSCRFPEEEEGGCELRVK
FSRSADAPAYQQGQNQLYNELNLGRREEYDVLDKRRGRDPEMGGKPRRKNPQEGLYNELQ
KDKMAEAYSEIGMKGERRRGKGHDGLYQGLSTATKDTYDALHMQALPPR
SEQ ID NO: 296
MGTSLLCWMALCLLGADHADACPYSNPSLCSGGGGSELPTQGTFSNVSTNVSPAKPTTTA
CPYSNPSLCSGGGGSPAPRPPTPAPTIASQPLSLRPEACRPAAGGAVHTRGLDFACDIYI
WAPLAGTCGVLLLSLVITLYCNHRNRRRVCKCPRPVVRAEGRGSLLTCGDVEENPGPMGW
SCIILFLVATATGVHSQVQLQESGGGLVQAGGSLRLSCAASRSSFSNNAMGWYRQGPGKQ
RELVAFITKGGVTDYSDSVKGRFTISKDNAKNTVYLQMNSLKPEDTAVYYCNTIPFRSAW
GQGTQVTVSSDPTPTPAPTIASQPLSLRPEACRPAAGGAVHTRGLDFACDIYIWAPLAGT
CGVLLLSLVITLYCKRGRKKLLYIFKQPFMRPVQTTQEEDGCSCRFPEEEEGGCELRVKF
SRSADAPAYQQGQNQLYNELNLGRREEYDVLDKRRGRDPEMGGKPRRKNPQEGLYNELQK
DKMAEAYSEIGMKGERRRGKGHDGLYQGLSTATKDTYDALHMQALPPR
SEQ ID NO: 297
MGTSLLCWMALCLLGADHADACPYSNPSLCSGGGGSELPTQGTFSNVSTNVSPAKPTTTA
CPYSNPSLCSGGGGSPAPRPPTPAPTIASQPLSLRPEACRPAAGGAVHTRGLDFACDIYI
WAPLAGTCGVLLLSLVITLYCNHRNRRRVCKCPRPVVRAEGRGSLLTCGDVEENPGPMGW
SCIILFLVATATGVHSQVQLQESGGGLVQAGGSLRLSCAASRSSFSNNAMGWYRQVPGKQ
RELVAFITKGGVTDYSDSVKGRFTISKDNAKNTVYLQMNSLKPEDTAVYYCNAIPFRSAW
GQGTQVTVSSDPTPTPAPTIASQPLSLRPEACRPAAGGAVHTRGLDFACDIYIWAPLAGT
CGVLLLSLVITLYCKRGRKKLLYIFKQPFMRPVQTTQEEDGCSCRFPEEEEGGCELRVKF
SRSADAPAYQQGQNQLYNELNLGRREEYDVLDKRRGRDPEMGGKPRRKNPQEGLYNELQK
DKMAEAYSEIGMKGERRRGKGHDGLYQGLSTATKDTYDALHMQALPPR
SEQ ID NO: 298
MGTSLLCWMALCLLGADHADACPYSNPSLCSGGGGSELPTQGTFSNVSTNVSPAKPTTTA
CPYSNPSLCSGGGGSPAPRPPTPAPTIASQPLSLRPEACRPAAGGAVHTRGLDFACDIYI
WAPLAGTCGVLLLSLVITLYCNHRNRRRVCKCPRPVVRAEGRGSLLTCGDVEENPGPMGW
SCIILFLVATATGVHSQVQLQESGGGLVQPGGSLRLSCAAPGNNFRLNAMGWYRQAPGKQ
RELVAHITSGGNTDYADSVKGRFTISRDNAKNTVYLQMNSLKPEDTAVYYCNAIPFRPSW
GQGTQVTVSSDPTPTPAPTIASQPLSLRPEACRPAAGGAVHTRGLDFACDIYIWAPLAGT
CGVLLLSLVITLYCKRGRKKLLYIFKQPFMRPVQTTQEEDGCSCRFPEEEEGGCELRVKF
SRSADAPAYQQGQNQLYNELNLGRREEYDVLDKRRGRDPEMGGKPRRKNPQEGLYNELQK
DKMAEAYSEIGMKGERRRGKGHDGLYQGLSTATKDTYDALHMQALPPR
SEQ ID NO: 299
MGTSLLCWMALCLLGADHADACPYSNPSLCSGGGGSELPTQGTFSNVSTNVSPAKPTTTA
CPYSNPSLCSGGGGSPAPRPPTPAPTIASQPLSLRPEACRPAAGGAVHTRGLDFACDIYI
WAPLAGTCGVLLLSLVITLYCNHRNRRRVCKCPRPVVRAEGRGSLLTCGDVEENPGPMGW
SCIILFLVATATGVHSQVQLQESGGGLVQPGGSLRLSCAAPGNIFRLNGTGWYRQAPGKQ
RELVAHITSGGNTDYADSGKGRFTISRDNAKNTVYLQMNSLKPEDTAVYYCNAIPFRRSW
GQGTQVTVSSDPTPTPAPTIASQPLSLRPEACRPAAGGAVHTRGLDFACDIYIWAPLAGT
CGVLLLSLVITLYCKRGRKKLLYIFKQPFMRPVQTTQEEDGCSCRFPEEEEGGCELRVKF
SRSADAPAYQQGQNQLYNELNLGRREEYDVLDKRRGRDPEMGGKPRRKNPQEGLYNELQK
DKMAEAYSEIGMKGERRRGKGHDGLYQGLSTATKDTYDALHMQALPPR
SEQ ID NO: 300
MGTSLLCWMALCLLGADHADACPYSNPSLCSGGGGSELPTQGTFSNVSTNVSPAKPTTTA
CPYSNPSLCSGGGGSPAPRPPTPAPTIASQPLSLRPEACRPAAGGAVHTRGLDFACDIYI
WAPLAGTCGVLLLSLVITLYCNHRNRRRVCKCPRPVVRAEGRGSLLTCGDVEENPGPMGW
SCIILFLVATATGVHSQVQLQESGGGLVQAGGSLKLSCAASERIFRINAMGWYRQAPGKQ
RELVAHITSGGNTDYADSVKGRFTISRDNAKNTMYLQMNSLKPEDTAVYYCNAIPFRRSW
GQGTQVTVSSDPTPTPAPTIASQPLSLRPEACRPAAGGAVHTRGLDFACDIYIWAPLAGT
CGVLLLSLVITLYCKRGRKKLLYIFKQPFMRPVQTTQEEDGCSCRFPEEEEGGCELRVKF
SRSADAPAYQQGQNQLYNELNLGRREEYDVLDKRRGRDPEMGGKPRRKNPQEGLYNELQK
DKMAEAYSEIGMKGERRRGKGHDGLYQGLSTATKDTYDALHMQALPPR
SEQ ID NO: 301
MGTSLLCWMALCLLGADHADACPYSNPSLCSGGGGSELPTQGTFSNVSTNVSPAKPTTTA
CPYSNPSLCSGGGGSPAPRPPTPAPTIASQPLSLRPEACRPAAGGAVHTRGLDFACDIYI
WAPLAGTCGVLLLSLVITLYCNHRNRRRVCKCPRPVVRAEGRGSLLTCGDVEENPGPMGW
SCIILFLVATATGVHSQVQLQQSGGGLVQPGGSLRLSCAAPGNIFRLNGTGWYRQAPGKQ
RELVAHITSGGNTDYADSVKGRFTISRDNAENTVYLQMNSLKPEDTAVYYCNAVPFRLSW
GQGTQVTVSSDPTPTPAPTIASQPLSLRPEACRPAAGGAVHTRGLDFACDIYIWAPLAGT
CGVLLLSLVITLYCKRGRKKLLYIFKQPFMRPVQTTQEEDGCSCRFPEEEEGGCELRVKF
SRSADAPAYQQGQNQLYNELNLGRREEYDVLDKRRGRDPEMGGKPRRKNPQEGLYNELQK
DKMAEAYSEIGMKGERRRGKGHDGLYQGLSTATKDTYDALHMQALPPR
SEQ ID NO: 302
MGTSLLCWMALCLLGADHADACPYSNPSLCSGGGGSELPTQGTFSNVSTNVSPAKPTTTA
CPYSNPSLCSGGGGSPAPRPPTPAPTIASQPLSLRPEACRPAAGGAVHTRGLDFACDIYI
WAPLAGTCGVLLLSLVITLYCNHRNRRRVCKCPRPVVRAEGRGSLLTCGDVEENPGPMGW
SCIILFLVATATGVHSQVQLQESGGGLVQPGESLRLSCAASGFTFSTYWMSWVRQAPGKG
PEGVSGIDNGGGTTTYADSVKGRFTISRDNAGNTVYLQMNSLKPEDTAVYYCNALPFRLS
WGQGTQVTVSSDPTPTPAPTIASQPLSLRPEACRPAAGGAVHTRGLDFACDIYIWAPLAG
TCGVLLLSLVITLYCKRGRKKLLYIFKQPFMRPVQTTQEEDGCSCRFPEEEEGGCELRVK
FSRSADAPAYQQGQNQLYNELNLGRREEYDVLDKRRGRDPEMGGKPRRKNPQEGLYNELQ
KDKMAEAYSEIGMKGERRRGKGHDGLYQGLSTATKDTYDALHMQALPPR
SEQ ID NO: 303
MGTSLLCWMALCLLGADHADACPYSNPSLCSGGGGSELPTQGTFSNVSTNVSPAKPTTTA
CPYSNPSLCSGGGGSPAPRPPTPAPTIASQPLSLRPEACRPAAGGAVHTRGLDFACDIYI
WAPLAGTCGVLLLSLVITLYCNHRNRRRVCKCPRPVVRAEGRGSLLTCGDVEENPGPMGW
SCIILFLVATATGVHSQVQLQESGGGLVQPGGSLRLSCAAPGNIFRLNGTGWYRQAPGKQ
GELVAIITSGGNTDYADSVKGRFTISRDNAKNTTYLQMNSLKPEDTAVYYCNATPFRLSW
GQGTQVTVSSDPTPTPAPTIASQPLSLRPEACRPAAGGAVHTRGLDFACDIYIWAPLAGT
CGVLLLSLVITLYCKRGRKKLLYIFKQPFMRPVQTTQEEDGCSCRFPEEEEGGCELRVKF
SRSADAPAYQQGQNQLYNELNLGRREEYDVLDKRRGRDPEMGGKPRRKNPQEGLYNELQK
DKMAEAYSEIGMKGERRRGKGHDGLYQGLSTATKDTYDALHMQALPPR
SEQ ID NO: 304
MGTSLLCWMALCLLGADHADACPYSNPSLCSGGGGSELPTQGTFSNVSTNVSPAKPTTTA
CPYSNPSLCSGGGGSPAPRPPTPAPTIASQPLSLRPEACRPAAGGAVHTRGLDFACDIYI
WAPLAGTCGVLLLSLVITLYCNHRNRRRVCKCPRPVVRAEGRGSLLTCGDVEENPGPMGW
SCIILFLVATATGVHSQVQLQESGGGLVQPGGSLRLSCAAPGNIFRLNGTGWYRQAPGKQ
RELVAHITRGGNTDYADAVKGRFTISRDNAKNTVYLQMNSLKPEDTAVYYCNSIPFRLSW
GQGTQVTVSSDPTPTPAPTIASQPLSLRPEACRPAAGGAVHTRGLDFACDIYIWAPLAGT
CGVLLLSLVITLYCKRGRKKLLYIFKQPFMRPVQTTQEEDGCSCRFPEEEEGGCELRVKF
SRSADAPAYQQGQNQLYNELNLGRREEYDVLDKRRGRDPEMGGKPRRKNPQEGLYNELQK
DKMAEAYSEIGMKGERRRGKGHDGLYQGLSTATKDTYDALHMQALPPR
SEQ ID NO: 305
MGTSLLCWMALCLLGADHADACPYSNPSLCSGGGGSELPTQGTFSNVSTNVSPAKPTTTA
CPYSNPSLCSGGGGSPAPRPPTPAPTIASQPLSLRPEACRPAAGGAVHTRGLDFACDIYI
WAPLAGTCGVLLLSLVITLYCNHRNRRRVCKCPRPVVRAEGRGSLLTCGDVEENPGPMGW
SCIILFLVATATGVHSQVQLQESGGGLVQPGGSLRLSCAAPGNIFRINGTGWYGQAPGKQ
RELVAHITSGGNTDYEDSVKGRFTISRDNAKNTTYLQMNSLKPEDTAVYYCNAIPFRISW
GQGTQVTVSSDPTPTPAPTIASQPLSLRPEACRPAAGGAVHTRGLDFACDIYIWAPLAGT
CGVLLLSLVITLYCKRGRKKLLYIFKQPFMRPVQTTQEEDGCSCRFPEEEEGGCELRVKF
SRSADAPAYQQGQNQLYNELNLGRREEYDVLDKRRGRDPEMGGKPRRKNPQEGLYNELQK
DKMAEAYSEIGMKGERRRGKGHDGLYQGLSTATKDTYDALHMQALPPR
SEQ ID NO: 306
MGTSLLCWMALCLLGADHADACPYSNPSLCSGGGGSELPTQGTFSNVSTNVSPAKPTTTA
CPYSNPSLCSGGGGSPAPRPPTPAPTIASQPLSLRPEACRPAAGGAVHTRGLDFACDIYI
WAPLAGTCGVLLLSLVITLYCNHRNRRRVCKCPRPVVRAEGRGSLLTCGDVEENPGPMGW
SCIILFLVATATGVHSQVQLQQSGGGLVQPGGSLRLSCAAPGNNFRLNAMGWYRQAPGKQ
RELVAHITSGGNTDYADSVKGRFTISRDNAKNTTYLQMNSLKPEDTAVYYCNAIPFRLYW
GQGTQVTVSSDPTPTPAPTIASQPLSLRPEACRPAAGGAVHTRGLDFACDIYIWAPLAGT
CGVLLLSLVITLYCKRGRKKLLYIFKQPFMRPVQTTQEEDGCSCRFPEEEEGGCELRVKF
SRSADAPAYQQGQNQLYNELNLGRREEYDVLDKRRGRDPEMGGKPRRKNPQEGLYNELQK
DKMAEAYSEIGMKGERRRGKGHDGLYQGLSTATKDTYDALHMQALPPR
SEQ ID NO: 307
MGTSLLCWMALCLLGADHADACPYSNPSLCSGGGGSELPTQGTFSNVSTNVSPAKPTTTA
CPYSNPSLCSGGGGSPAPRPPTPAPTIASQPLSLRPEACRPAAGGAVHTRGLDFACDIYI
WAPLAGTCGVLLLSLVITLYCNHRNRRRVCKCPRPVVRAEGRGSLLTCGDVEENPGPMGW
SCIILFLVATATGVHSQVQLQESGGGLVQPGGSLRLSCLASGFTFSTYWMSWVRQAPGKG
PEWVSGIDNGGGTTTYADSVKGRFTISRDNAKNTLNLEMNNLKPEDTAGYYSKAIPFRLS
WGQGTQVTVSSDPTPTPAPTIASQPLSLRPEACRPAAGGAVHTRGLDFACDIYIWAPLAG
TCGVLLLSLVITLYCKRGRKKLLYIFKQPFMRPVQTTQEEDGCSCRFPEEEEGGCELRVK
FSRSADAPAYQQGQNQLYNELNLGRREEYDVLDKRRGRDPEMGGKPRRKNPQEGLYNELQ
KDKMAEAYSEIGMKGERRRGKGHDGLYQGLSTATKDTYDALHMQALPPR
SEQ ID NO: 308
MGTSLLCWMALCLLGADHADACPYSNPSLCSGGGGSELPTQGTFSNVSTNVSPAKPTTTA
CPYSNPSLCSGGGGSPAPRPPTPAPTIASQPLSLRPEACRPAAGGAVHTRGLDFACDIYI
WAPLAGTCGVLLLSLVITLYCNHRNRRRVCKCPRPVVRAEGRGSLLTCGDVEENPGPMGW
SCIILFLVATATGVHSQVQLQESGGGLVQPGGSLRLSCAASGSSFSNNAMGWYRQAPGKQ
RELVAFITKGGVTDYSDSVRGRFTISRDNAKNTVYLQMNSLKPEDTAVYYCNTIPFRSAW
GQETQVTVSSDPTPTPAPTIASQPLSLRPEACRPAAGGAVHTRGLDFACDIYIWAPLAGT
CGVLLLSLVITLYCKRGRKKLLYIFKQPFMRPVQTTQEEDGCSCRFPEEEEGGCELRVKF
SRSADAPAYQQGQNQLYNELNLGRREEYDVLDKRRGRDPEMGGKPRRKNPQEGLYNELQK
DKMAEAYSEIGMKGERRRGKGHDGLYQGLSTATKDTYDALHMQALPPR
SEQ ID NO: 309
MGTSLLCWMALCLLGADHADACPYSNPSLCSGGGGSELPTQGTFSNVSTNVSPAKPTTTA
CPYSNPSLCSGGGGSPAPRPPTPAPTIASQPLSLRPEACRPAAGGAVHTRGLDFACDIYI
WAPLAGTCGVLLLSLVITLYCNHRNRRRVCKCPRPVVRAEGRGSLLTCGDVEENPGPMGW
SCIILFLVATATGVHSQVQLQQSGGGLVQPGGSLRLSCLASGFTFSTYWMSWVRQAPGKG
PEWVSGIDNGGGTTTYADSVKGRFTISRDNAKNTVYLQMNSLKPEDTAVYYCNTIPFRSA
WGQGTQVTVASDPTPTPAPTIASQPLSLRPEACRPAAGGAVHTRGLDFACDIYIWAPLAG
TCGVLLLSLVITLYCKRGRKKLLYIFKQPFMRPVQTTQEEDGCSCRFPEEEEGGCELRVK
FSRSADAPAYQQGQNQLYNELNLGRREEYDVLDKRRGRDPEMGGKPRRKNPQEGLYNELQ
KDKMAEAYSEIGMKGERRRGKGHDGLYQGLSTATKDTYDALHMQALPPR
SEQ ID NO: 310
MGTSLLCWMALCLLGADHADACPYSNPSLCSGGGGSELPTQGTFSNVSTNVSPAKPTTTA
CPYSNPSLCSGGGGSPAPRPPTPAPTIASQPLSLRPEACRPAAGGAVHTRGLDFACDIYI
WAPLAGTCGVLLLSLVITLYCNHRNRRRVCKCPRPVVRAEGRGSLLTCGDVEENPGPMGW
SCIILFLVATATGVHSQVQLQESGGGLVQPGGSLRLSCAAPGNIFRLNATGWYRQVPGKQ
RELVAFITKGGVTEHSDSVEGRFTISKDNAKNTVYLQMNSLKPEDTAVYYCNTIPFRSAW
GQGTQVTVSSDPTPTPAPTIASQPLSLRPEACRPAAGGAVHTRGLDFACDIYIWAPLAGT
CGVLLLSLVITLYCKRGRKKLLYIFKQPFMRPVQTTQEEDGCSCRFPEEEEGGCELRVKF
SRSADAPAYQQGQNQLYNELNLGRREEYDVLDKRRGRDPEMGGKPRRKNPQEGLYNELQK
DKMAEAYSEIGMKGERRRGKGHDGLYQGLSTATKDTYDALHMQALPPR
SEQ ID NO: 311
MGTSLLCWMALCLLGADHADACPYSNPSLCSGGGGSELPTQGTFSNVSTNVSPAKPTTTA
CPYSNPSLCSGGGGSPAPRPPTPAPTIASQPLSLRPEACRPAAGGAVHTRGLDFACDIYI
WAPLAGTCGVLLLSLVITLYCNHRNRRRVCKCPRPVVRAEGRGSLLTCGDVEENPGPMGW
SCIILFLVATATGVHSQVQLQESGGGLVQTGGSLRLSCAASRSSFSNNAMGWYRQVPGKQ
RELVAFITKGGVTDYSDSVKGRFTISKDNAKNTVYLQMNSLKPEDTAVYYCNTIPFRSAW
GQGTQVTVSSDPTPTPAPTIASQPLSLRPEACRPAAGGAVHTRGLDFACDIYIWAPLAGT
CGVLLLSLVITLYCKRGRKKLLYIFKQPFMRPVQTTQEEDGCSCRFPEEEEGGCELRVKF
SRSADAPAYQQGQNQLYNELNLGRREEYDVLDKRRGRDPEMGGKPRRKNPQEGLYNELQK
DKMAEAYSEIGMKGERRRGKGHDGLYQGLSTATKDTYDALHMQALPPR
SEQ ID NO: 312
MGTSLLCWMALCLLGADHADACPYSNPSLCSGGGGSELPTQGTFSNVSTNVSPAKPTTTA
CPYSNPSLCSGGGGSPAPRPPTPAPTIASQPLSLRPEACRPAAGGAVHTRGLDFACDIYI
WAPLAGTCGVLLLSLVITLYCNHRNRRRVCKCPRPVVRAEGRGSLLTCGDVEENPGPMGW
SCIILFLVATATGVHSQVQLQESGGGLVQAGGSLRLSCAASGSSFSNNAMGWYRQVPGKQ
RELVAFITKGGVTDYSDSVKGRFTISKDNAKNTVYLQMNSLKPEDTAVYYCNTIPFRSAW
GQGTQVTVSSDPTPTPAPTIASQPLSLRPEACRPAAGGAVHTRGLDFACDIYIWAPLAGT
CGVLLLSLVITLYCKRGRKKLLYIFKQPFMRPVQTTQEEDGCSCRFPEEEEGGCELRVKF
SRSADAPAYQQGQNQLYNELNLGRREEYDVLDKRRGRDPEMGGKPRRKNPQEGLYNELQK
DKMAEAYSEIGMKGERRRGKGHDGLYQGLSTATKDTYDALHMQALPPR
SEQ ID NO: 313
MGTSLLCWMALCLLGADHADACPYSNPSLCSGGGGSELPTQGTFSNVSTNVSPAKPTTTA
CPYSNPSLCSGGGGSPAPRPPTPAPTIASQPLSLRPEACRPAAGGAVHTRGLDFACDIYI
WAPLAGTCGVLLLSLVITLYCNHRNRRRVCKCPRPVVRAEGRGSLLTCGDVEENPGPMGW
SCIILFLVATATGVHSQVQLQESGGGLVQAGGSLRLSCAASRSSFGNNAMGWYRQVPGKQ
RELVAFITKGGVTDYSDSVKGRFTISKDNAKNTVYLQMNSLKPEDTAVYYCNTIPFRSAW
GQGTQVTVSSDPTPTPAPTIASQPLSLRPEACRPAAGGAVHTRGLDFACDIYIWAPLAGT
CGVLLLSLVITLYCKRGRKKLLYIFKQPFMRPVQTTQEEDGCSCRFPEEEEGGCELRVKF
SRSADAPAYQQGQNQLYNELNLGRREEYDVLDKRRGRDPEMGGKPRRKNPQEGLYNELQK
DKMAEAYSEIGMKGERRRGKGHDGLYQGLSTATKDTYDALHMQALPPR
SEQ ID NO: 314
MGTSLLCWMALCLLGADHADACPYSNPSLCSGGGGSELPTQGTFSNVSTNVSPAKPTTTA
CPYSNPSLCSGGGGSPAPRPPTPAPTIASQPLSLRPEACRPAAGGAVHTRGLDFACDIYI
WAPLAGTCGVLLLSLVITLYCNHRNRRRVCKCPRPVVRAEGRGSLLTCGDVEENPGPMGW
SCIILFLVATATGVHSQVQLQESRGGLVQAGGSLRLSCAASGTIERNNAMAWYRQAPGKQ
RELVAIITSGGSTNYSDSVKGRFTISRDNAKNTLYLQMNSLKPEDTAVYLWAAGRRFSTR
SRDYDYWGQGTQVTVSSDPTPTPAPTIASQPLSLRPEACRPAAGGAVHTRGLDFACDIYI
WAPLAGTCGVLLLSLVITLYCKRGRKKLLYIFKQPFMRPVQTTQEEDGCSCRFPEEEEGG
CELRVKFSRSADAPAYQQGQNQLYNELNLGRREEYDVLDKRRGRDPEMGGKPRRKNPQEG
LYNELQKDKMAEAYSEIGMKGERRRGKGHDGLYQGLSTATKDTYDALHMQALPPR
SEQ ID NO: 315
MGTSLLCWMALCLLGADHADACPYSNPSLCSGGGGSELPTQGTFSNVSTNVSPAKPTTTA
CPYSNPSLCSGGGGSPAPRPPTPAPTIASQPLSLRPEACRPAAGGAVHTRGLDFACDIYI
WAPLAGTCGVLLLSLVITLYCNHRNRRRVCKCPRPVVRAEGRGSLLTCGDVEENPGPMGW
SCIILFLVATATGVHSQVQLQESGGGLVQAGGSLSLSCTASGRSFSNYGMGWFRQAPGKE
REFVAAIGMVGGLTAYSNSAKGRFTISRDNAKNTVYLQMNSLKPEDTAVYLCAAGRRFST
SSRDYDYWGQGTQVTVSSDPTPTPAPTIASQPLSLRPEACRPAAGGAVHTRGLDFACDIY
IWAPLAGTCGVLLLSLVITLYCKRGRKKLLYIFKQPFMRPVQTTQEEDGCSCRFPEEEEG
GCELRVKFSRSADAPAYQQGQNQLYNELNLGRREEYDVLDKRRGRDPEMGGKPRRKNPQE
GLYNELQKDKMAEAYSEIGMKGERRRGKGHDGLYQGLSTATKDTYDALHMQALPPR
SEQ ID NO: 316
MGTSLLCWMALCLLGADHADACPYSNPSLCSGGGGSELPTQGTFSNVSTNVSPAKPTTTA
CPYSNPSLCSGGGGSPAPRPPTPAPTIASQPLSLRPEACRPAAGGAVHTRGLDFACDIYI
WAPLAGTCGVLLLSLVITLYCNHRNRRRVCKCPRPVVRAEGRGSLLTCGDVEENPGPMGW
SCIILFLVATATGVHSQVQLQESGGGLVQAGDSLRLSCAASGRTFSSYAMGWFRQAPGKE
REFVAAISRIGGVTTYAGSVQGRFTISRDNAKNTLYLRMNALKPEDTAVYYCAAAGLVSI
STTPNDYDYWGQGTQVTVSSDPTPTPAPTIASQPLSLRPEACRPAAGGAVHTRGLDFACD
IYIWAPLAGTCGVLLLSLVITLYCKRGRKKLLYIFKQPFMRPVQTTQEEDGCSCRFPEEE
EGGCELRVKFSRSADAPAYQQGQNQLYNELNLGRREEYDVLDKRRGRDPEMGGKPRRKNP
QEGLYNELQKDKMAEAYSEIGMKGERRRGKGHDGLYQGLSTATKDTYDALHMQALPPR
SEQ ID NO: 317
MGTSLLCWMALCLLGADHADACPYSNPSLCSGGGGSELPTQGTFSNVSTNVSPAKPTTTA
CPYSNPSLCSGGGGSPAPRPPTPAPTIASQPLSLRPEACRPAAGGAVHTRGLDFACDIYI
WAPLAGTCGVLLLSLVITLYCNHRNRRRVCKCPRPVVRAEGRGSLLTCGDVEENPGPMGW
SCIILFLVATATGVHSQVQLQESGGGLVQAGGSLSLSCTASGRSFSNYGMGWFRQAPGKE
REFVDAIGMVGGLTAYSNSAKGRFTISRDNAKNTLYLQMNSLKPEDTAVYLCAAGRRFST
GSRDYDIWGQGTQVTVSSDPTPTPAPTIASQPLSLRPEACRPAAGGAVHTRGLDFACDIY
IWAPLAGTCGVLLLSLVITLYCKRGRKKLLYIFKQPFMRPVQTTQEEDGCSCRFPEEEEG
GCELRVKFSRSADAPAYQQGQNQLYNELNLGRREEYDVLDKRRGRDPEMGGKPRRKNPQE
GLYNELQKDKMAEAYSEIGMKGERRRGKGHDGLYQGLSTATKDTYDALHMQALPPR
SEQ ID NO: 318
MGTSLLCWMALCLLGADHADACPYSNPSLCSGGGGSELPTQGTFSNVSTNVSPAKPTTTA
CPYSNPSLCSGGGGSPAPRPPTPAPTIASQPLSLRPEACRPAAGGAVHTRGLDFACDIYI
WAPLAGTCGVLLLSLVITLYCNHRNRRRVCKCPRPVVRAEGRGSLLTCGDVEENPGPMGW
SCIILFLVATATGVHSQVQLQQSGGGLVQTGGSLRLSCAASGRTFSSYAMGWFRQAPGKE
REFVAAISQFGGVTTYADSVQGRFTISRDNAKNTLYLRMNSLKPEDTAVYLCAAGRRFST
GSRDYDIWGQGTQVTVSSDPTPTPAPTIASQPLSLRPEACRPAAGGAVHTRGLDFACDIY
IWAPLAGTCGVLLLSLVITLYCKRGRKKLLYIFKQPFMRPVQTTQEEDGCSCRFPEEEEG
GCELRVKFSRSADAPAYQQGQNQLYNELNLGRREEYDVLDKRRGRDPEMGGKPRRKNPQE
GLYNELQKDKMAEAYSEIGMKGERRRGKGHDGLYQGLSTATKDTYDALHMQALPPR
SEQ ID NO: 319
MGTSLLCWMALCLLGADHADACPYSNPSLCSGGGGSELPTQGTFSNVSTNVSPAKPTTTA
CPYSNPSLCSGGGGSPAPRPPTPAPTIASQPLSLRPEACRPAAGGAVHTRGLDFACDIYI
WAPLAGTCGVLLLSLVITLYCNHRNRRRVCKCPRPVVRAEGRGSLLTCGDVEENPGPMGW
SCIILFLVATATGVHSQVQLQESGGGLVQAGGSLSLSCTASGRSFSNYGMGWFRQAPGKE
REFVAAIGMVGGLTAYSNSAKGRFTISRDKAKNTLYLQMNSLKPEDTAGYCGAGGRRFST
SSRDYDIWGQGTQVTVSSDPTPTPAPTIASQPLSLRPEACRPAAGGAVHTRGLDFACDIY
IWAPLAGTCGVLLLSLVITLYCKRGRKKLLYIFKQPFMRPVQTTQEEDGCSCRFPEEEEG
GCELRVKFSRSADAPAYQQGQNQLYNELNLGRREEYDVLDKRRGRDPEMGGKPRRKNPQE
GLYNELQKDKMAEAYSEIGMKGERRRGKGHDGLYQGLSTATKDTYDALHMQALPPR
SEQ ID NO: 320
MGTSLLCWMALCLLGADHADACPYSNPSLCSGGGGSELPTQGTFSNVSTNVSPAKPTTTA
CPYSNPSLCSGGGGSPAPRPPTPAPTIASQPLSLRPEACRPAAGGAVHTRGLDFACDIYI
WAPLAGTCGVLLLSLVITLYCNHRNRRRVCKCPRPVVRAEGRGSLLTCGDVEENPGPMGW
SCIILFLVATATGVHSQVQLQESGGGLVQAGGSLSLSCTASGRSFSNYGMGWFRQAPGKE
REFVAAIGMVGGLTAYSNSAKGRFTISRDNAKNTLYLQMNSLKPEDTAVYLCAAGRRFST
SSREYDIWGQGTQVTVSSDPTPTPAPTIASQPLSLRPEACRPAAGGAVHTRGLDFACDIY
IWAPLAGTCGVLLLSLVITLYCKRGRKKLLYIFKQPFMRPVQTTQEEDGCSCRFPEEEEG
GCELRVKFSRSADAPAYQQGQNQLYNELNLGRREEYDVLDKRRGRDPEMGGKPRRKNPQE
GLYNELQKDKMAEAYSEIGMKGERRRGKGHDGLYQGLSTATKDTYDALHMQALPPR
SEQ ID NO: 321
MGTSLLCWMALCLLGADHADACPYSNPSLCSGGGGSELPTQGTFSNVSTNVSPAKPTTTA
CPYSNPSLCSGGGGSPAPRPPTPAPTIASQPLSLRPEACRPAAGGAVHTRGLDFACDIYI
WAPLAGTCGVLLLSLVITLYCNHRNRRRVCKCPRPVVRAEGRGSLLTCGDVEENPGPMGW
SCIILFLVATATGVHSQVQLQESGGGLVQAGGSLSLSCTASGRSFSNYGMGWFRQAPGKE
REFVAAIGMVGGLPAYSNSAKGRFTISQDNAKNPLYLQINSLKPEETDVYLCAAGRRLST
SSRDYDIWGQGTQVTVSSDPTPTPAPTIASQPLSLRPEACRPAAGGAVHTRGLDFACDIY
IWAPLAGTCGVLLLSLVITLYCKRGRKKLLYIFKQPFMRPVQTTQEEDGCSCRFPEEEEG
GCELRVKFSRSADAPAYQQGQNQLYNELNLGRREEYDVLDKRRGRDPEMGGKPRRKNPQE
GLYNELQKDKMAEAYSEIGMKGERRRGKGHDGLYQGLSTATKDTYDALHMQALPPR
SEQ ID NO: 322
MGTSLLCWMALCLLGADHADACPYSNPSLCSGGGGSELPTQGTFSNVSTNVSPAKPTTTA
CPYSNPSLCSGGGGSPAPRPPTPAPTIASQPLSLRPEACRPAAGGAVHTRGLDFACDIYI
WAPLAGTCGVLLLSLVITLYCNHRNRRRVCKCPRPVVRAEGRGSLLTCGDVEENPGPMGW
SCIILFLVATATGVHSQVQLQESGGGLVQAGGSLSLSCTASGRSFSNYGMGWFRQAPGKE
REFVAAISRGGGVSAYSNSAKGRFTISRDNAKNTVYLQMNSLKPEDAAVYFCAAGLRFST
GSRDYDIWGQGTQVTVSSDPTPTPAPTIASQPLSLRPEACRPAAGGAVHTRGLDFACDIY
IWAPLAGTCGVLLLSLVITLYCKRGRKKLLYIFKQPFMRPVQTTQEEDGCSCRFPEEEEG
GCELRVKFSRSADAPAYQQGQNQLYNELNLGRREEYDVLDKRRGRDPEMGGKPRRKNPQE
GLYNELQKDKMAEAYSEIGMKGERRRGKGHDGLYQGLSTATKDTYDALHMQALPPR
SEQ ID NO: 323
MGTSLLCWMALCLLGADHADACPYSNPSLCSGGGGSELPTQGTFSNVSTNVSPAKPTTTA
CPYSNPSLCSGGGGSPAPRPPTPAPTIASQPLSLRPEACRPAAGGAVHTRGLDFACDIYI
WAPLAGTCGVLLLSLVITLYCNHRNRRRVCKCPRPVVRAEGRGSLLTCGDVEENPGPMGW
SCIILFLVATATGVHSQVQLQQSGGGLVQAGDSLRLSCAASGRTFRRYAMGWFHQAPGKD
REFVAGISRSGGMTGYADSVKGRFTISRDNAKNMVFLQMNSLKPEDTAVYYCAAYVGGFS
TTRRDYAYWGQGTQVTVSSDPTPTPAPTIASQPLSLRPEACRPAAGGAVHTRGLDFACDI
YIWAPLAGTCGVLLLSLVITLYCKRGRKKLLYIFKQPFMRPVQTTQEEDGCSCRFPEEEE
GGCELRVKFSRSADAPAYQQGQNQLYNELNLGRREEYDVLDKRRGRDPEMGGKPRRKNPQ
EGLYNELQKDKMAEAYSEIGMKGERRRGKGHDGLYQGLSTATKDTYDALHMQALPPR
SEQ ID NO: 324
MGTSLLCWMALCLLGADHADACPYSNPSLCSGGGGSELPTQGTFSNVSTNVSPAKPTTTA
CPYSNPSLCSGGGGSPAPRPPTPAPTIASQPLSLRPEACRPAAGGAVHTRGLDFACDIYI
WAPLAGTCGVLLLSLVITLYCNHRNRRRVCKCPRPVVRAEGRGSLLTCGDVEENPGPMGW
SCIILFLVATATGVHSQVQLQESGGGLVQAGDSLRLSCAASGRTFSNSTMGWFHQAPGKE
RKFVAVISWSGGTTAYAESVKGRFTISRDNAKNTVYLQMNSLKPEDTAVYYCAAARKGWS
TRGDDYDYWGQGTQVTVSSDPTPTPAPTIASQPLSLRPEACRPAAGGAVHTRGLDFACDI
YIWAPLAGTCGVLLLSLVITLYCKRGRKKLLYIFKQPFMRPVQTTQEEDGCSCRFPEEEE
GGCELRVKFSRSADAPAYQQGQNQLYNELNLGRREEYDVLDKRRGRDPEMGGKPRRKNPQ
EGLYNELQKDKMAEAYSEIGMKGERRRGKGHDGLYQGLSTATKDTYDALHMQALPPR
SEQ ID NO: 325
MGTSLLCWMALCLLGADHADACPYSNPSLCSGGGGSELPTQGTFSNVSTNVSPAKPTTTA
CPYSNPSLCSGGGGSPAPRPPTPAPTIASQPLSLRPEACRPAAGGAVHTRGLDFACDIYI
WAPLAGTCGVLLLSLVITLYCNHRNRRRVCKCPRPVVRAEGRGSLLTCGDVEENPGPMGW
SCIILFLVATATGVHSQVQLQESGGGLVQTGGSLRLSCAASGRTVIGWFRQAPGKEREFV
AVSSGSGGVTAYASSVEGRFTISRDNVKNIMYLQMNSLKPEDTAIYYCAAALTWSTRPSD
FTSWGQGTQVTVSSDPTPTPAPTIASQPLSLRPEACRPAAGGAVHTRGLDFACDIYIWAP
LAGTCGVLLLSLVITLYCKRGRKKLLYIFKQPFMRPVQTTQEEDGCSCRFPEEEEGGCEL
RVKFSRSADAPAYQQGQNQLYNELNLGRREEYDVLDKRRGRDPEMGGKPRRKNPQEGLYN
ELQKDKMAEAYSEIGMKGERRRGKGHDGLYQGLSTATKDTYDALHMQALPPR
SEQ ID NO: 326
MGTSLLCWMALCLLGADHADACPYSNPSLCSGGGGSELPTQGTFSNVSTNVSPAKPTTTA
CPYSNPSLCSGGGGSPAPRPPTPAPTIASQPLSLRPEACRPAAGGAVHTRGLDFACDIYI
WAPLAGTCGVLLLSLVITLYCNHRNRRRVCKCPRPVVRAEGRGSLLTCGDVEENPGPMGW
SCIILFLVATATGVHSQVQLQQSGGGLVQAGGSLRLSCAASGRTYNNYAMGWFRQAPGKE
REFVAGISRSGGMTGYAESVKGRFTISRDNAKNMVCLQMNSLKPEDKAVYYCAAYVGGFS
TARRDYSYWGQGTQVTVSSDPTPTPAPTIASQPLSLRPEACRPAAGGAVHTRGLDFACDI
YIWAPLAGTCGVLLLSLVITLYCKRGRKKLLYIFKQPFMRPVQTTQEEDGCSCRFPEEEE
GGCELRVKFSRSADAPAYQQGQNQLYNELNLGRREEYDVLDKRRGRDPEMGGKPRRKNPQ
EGLYNELQKDKMAEAYSEIGMKGERRRGKGHDGLYQGLSTATKDTYDALHMQALPPR
SEQ ID NO: 327
MGTSLLCWMALCLLGADHADACPYSNPSLCSGGGGSELPTQGTFSNVSTNVSPAKPTTTA
CPYSNPSLCSGGGGSPAPRPPTPAPTIASQPLSLRPEACRPAAGGAVHTRGLDFACDIYI
WAPLAGTCGVLLLSLVITLYCNHRNRRRVCKCPRPVVRAEGRGSLLTCGDVEENPGPMGW
SCIILFLVATATGVHSQVQLQQSGGGLVQTGGSLRLSCAASGRTVIGWFRQAPGKEREFV
AVSNWSGGVTAYASSVEGRFTISRDNVKNIMYFQMNSLKPEDTAVYYCAAYVGGFSTARR
DYSYWGQGTQVTVSSDPTPTPAPTIASQPLSLRPEACRPAAGGAVHTRGLDFACDIYIWA
PLAGTCGVLLLSLVITLYCKRGRKKLLYIFKQPFMRPVQTTQEEDGCSCRFPEEEEGGCE
LRVKFSRSADAPAYQQGQNQLYNELNLGRREEYDVLDKRRGRDPEMGGKPRRKNPQEGLY
NELQKDKMAEAYSEIGMKGERRRGKGHDGLYQGLSTATKDTYDALHMQALPPR
SEQ ID NO: 328
MGTSLLCWMALCLLGADHADACPYSNPSLCSGGGGSELPTQGTFSNVSTNVSPAKPTTTA
CPYSNPSLCSGGGGSPAPRPPTPAPTIASQPLSLRPEACRPAAGGAVHTRGLDFACDIYI
WAPLAGTCGVLLLSLVITLYCNHRNRRRVCKCPRPVVRAEGRGSLLTCGDVEENPGPMGW
SCIILFLVATATGVHSQVQLQQSGGGLVQPGGSLRLSCAASGRTYNNYAMGWFRQAPGKE
REFVAGISRSGGMTGYAESVKGRFTISRDNAKNMVFLQMNSLKPEDTAVYYCAAYVGGFS
TARRDYSYWGQGTQVTVSSDPTPTPAPTIASQPLSLRPEACRPAAGGAVHTRGLDFACDI
YIWAPLAGTCGVLLLSLVITLYCKRGRKKLLYIFKQPFMRPVQTTQEEDGCSCRFPEEEE
GGCELRVKFSRSADAPAYQQGQNQLYNELNLGRREEYDVLDKRRGRDPEMGGKPRRKNPQ
EGLYNELQKDKMAEAYSEIGMKGERRRGKGHDGLYQGLSTATKDTYDALHMQALPPR
SEQ ID NO: 329
MGTSLLCWMALCLLGADHADACPYSNPSLCSGGGGSELPTQGTFSNVSTNVSPAKPTTTA
CPYSNPSLCSGGGGSPAPRPPTPAPTIASQPLSLRPEACRPAAGGAVHTRGLDFACDIYI
WAPLAGTCGVLLLSLVITLYCNHRNRRRVCKCPRPVVRAEGRGSLLTCGDVEENPGPMGW
SCIILFLVATATGVHSQVQLQESGGGLVQAGGSLIVSCATSGRTFSINAMGWFRQAPGKE
REFVAGISRSGGMTGYAESVKGRFTISRDNAKNMVFLQMNSLKPEDTAVYYCAAYVGGFS
TARRDYSYWGQGTQVSVSSDPTPTPAPTIASQPLSLRPEACRPAAGGAVHTRGLDFACDI
YIWAPLAGTCGVLLLSLVITLYCKRGRKKLLYIFKQPFMRPVQTTQEEDGCSCRFPEEEE
GGCELRVKFSRSADAPAYQQGQNQLYNELNLGRREEYDVLDKRRGRDPEMGGKPRRKNPQ
EGLYNELQKDKMAEAYSEIGMKGERRRGKGHDGLYQGLSTATKDTYDALHMQALPPR
SEQ ID NO: 330
MGTSLLCWMALCLLGADHADACPYSNPSLCSGGGGSELPTQGTFSNVSTNVSPAKPTTTA
CPYSNPSLCSGGGGSPAPRPPTPAPTIASQPLSLRPEACRPAAGGAVHTRGLDFACDIYI
WAPLAGTCGVLLLSLVITLYCNHRNRRRVCKCPRPVVRAEGRGSLLTCGDVEENPGPMGW
SCIILFLVATATGVHSQVQLQQSGGGLVQAGGSLRLSCAASGRTFSRYAMGWFRQAPGKE
REFVAVINGSGGTTAYANSVKGRFTITRDNAKNTLYLQMNSLKPEDTAVYYCAAARIFTT
TRNEYDHWGQGTQVTVSSDPTPTPAPTIASQPLSLRPEACRPAAGGAVHTRGLDFACDIY
IWAPLAGTCGVLLLSLVITLYCKRGRKKLLYIFKQPFMRPVQTTQEEDGCSCRFPEEEEG
GCELRVKFSRSADAPAYQQGQNQLYNELNLGRREEYDVLDKRRGRDPEMGGKPRRKNPQE
GLYNELQKDKMAEAYSEIGMKGERRRGKGHDGLYQGLSTATKDTYDALHMQALPPR
SEQ ID NO: 331
MGTSLLCWMALCLLGADHADACPYSNPSLCSGGGGSELPTQGTFSNVSTNVSPAKPTTTA
CPYSNPSLCSGGGGSPAPRPPTPAPTIASQPLSLRPEACRPAAGGAVHTRGLDFACDIYI
WAPLAGTCGVLLLSLVITLYCNHRNRRRVCKCPRPVVRAEGRGSLLTCGDVEENPGPMGW
SCIILFLVATATGVHSQVQLQEPGGGLVQPGGSLRLSCAASGFTFSTYWMSWVHQAPGKG
PEWVSGIDNGGGTTTYADSVKGRFTISRDNAKNTLYLQMNSLKPEETAIYYWAAARIFST
ARNDYDHWGQGTQVTVSSDPTPTPAPTIASQPLSLRPEACRPAAGGAVHTRGLDFACDIY
IWAPLAGTCGVLLLSLVITLYCKRGRKKLLYIFKQPFMRPVQTTQEEDGCSCRFPEEEEG
GCELRVKFSRSADAPAYQQGQNQLYNELNLGRREEYDVLDKRRGRDPEMGGKPRRKNPQE
GLYNELQKDKMAEAYSEIGMKGERRRGKGHDGLYQGLSTATKDTYDALHMQALPPR
SEQ ID NO: 332
MGTSLLCWMALCLLGADHADACPYSNPSLCSGGGGSELPTQGTFSNVSTNVSPAKPTTTA
CPYSNPSLCSGGGGSPAPRPPTPAPTIASQPLSLRPEACRPAAGGAVHTRGLDFACDIYI
WAPLAGTCGVLLLSLVITLYCNHRNRRRVCKCPRPVVRAEGRGSLLTCGDVEENPGPMGW
SCIILFLVATATGVHSQVQLQESGGGLVQAGGSLSLSCTASGRSFSNYGMGWFRQAPGKE
REFVAAIGMVGGLTAYSNSAKGRFTISRDNAKNTLYLQMNSLKPEDTAVYLVAGGRIFRT
SSRDYDIWGQGTQVTVSSDPTPTPAPTIASQPLSLRPEACRPAAGGAVHTRGLDFACDIY
IWAPLAGTCGVLLLSLVITLYCKRGRKKLLYIFKQPFMRPVQTTQEEDGCSCRFPEEEEG
GCELRVKFSRSADAPAYQQGQNQLYNELNLGRREEYDVLDKRRGRDPEMGGKPRRKNPQE
GLYNELQKDKMAEAYSEIGMKGERRRGKGHDGLYQGLSTATKDTYDALHMQALPPR
SEQ ID NO: 333
MGTSLLCWMALCLLGADHADACPYSNPSLCSGGGGSELPTQGTFSNVSTNVSPAKPTTTA
CPYSNPSLCSGGGGSPAPRPPTPAPTIASQPLSLRPEACRPAAGGAVHTRGLDFACDIYI
WAPLAGTCGVLLLSLVITLYCNHRNRRRVCKCPRPVVRAEGRGSLLTCGDVEENPGPMGW
SCIILFLVATATGVHSQVQLQESGGGLVQPGGSLRLYCAAPGNIFRLNGTGWYRQAPGKQ
RELVTHITSGGNTDYADSVKGRFTISRDNAKNTVYLQMNSLKPEDTAIYYCAAARFFTTA
RNDYDHWGQGTQVTVSSDPTPTPAPTIASQPLSLRPEACRPAAGGAVHTRGLDFACDIYI
WAPLAGTCGVLLLSLVITLYCKRGRKKLLYIFKQPFMRPVQTTQEEDGCSCRFPEEEEGG
CELRVKFSRSADAPAYQQGQNQLYNELNLGRREEYDVLDKRRGRDPEMGGKPRRKNPQEG
LYNELQKDKMAEAYSEIGMKGERRRGKGHDGLYQGLSTATKDTYDALHMQALPPR
SEQ ID NO: 334
MGTSLLCWMALCLLGADHADACPYSNPSLCSGGGGSELPTQGTFSNVSTNVSPAKPTTTA
CPYSNPSLCSGGGGSPAPRPPTPAPTIASQPLSLRPEACRPAAGGAVHTRGLDFACDIYI
WAPLAGTCGVLLLSLVITLYCNHRNRRRVCKCPRPVVRAEGRGSLLTCGDVEENPGPMGW
SCIILFLVATATGVHSQVQLQESGGGLVQAGGSLRLSCAASGSIFSINAMGWYRQAPGKQ
RELVAAITSGGSTNYADSVKGRFTISRDNAKNTVYLQMNSLKPEDTAVYYCNALGGFVPN
YGWGQGTQVTVSSDPTPTPAPTIASQPLSLRPEACRPAAGGAVHTRGLDFACDIYIWAPL
AGTCGVLLLSLVITLYCKRGRKKLLYIFKQPFMRPVQTTQEEDGCSCRFPEEEEGGCELR
VKFSRSADAPAYQQGQNQLYNELNLGRREEYDVLDKRRGRDPEMGGKPRRKNPQEGLYNE
LQKDKMAEAYSEIGMKGERRRGKGHDGLYQGLSTATKDTYDALHMQALPPR
SEQ ID NO: 335
MGTSLLCWMALCLLGADHADACPYSNPSLCSGGGGSELPTQGTFSNVSTNVSPAKPTTTA
CPYSNPSLCSGGGGSPAPRPPTPAPTIASQPLSLRPEACRPAAGGAVHTRGLDFACDIYI
WAPLAGTCGVLLLSLVITLYCNHRNRRRVCKCPRPVVRAEGRGSLLTCGDVEENPGPMGW
SCIILFLVATATGVHSQVQLQESGGGLVQPGGSLRLSCAAPGNIFRLNGTGWYRQAPGKQ
RELVAHITSGGNTDYADSVKGRFTISRDNAKNTVYLQMNSLKPEDTAVYYCNALGGFVPN
YGWGQGTQVTVSSDPTPTPAPTIASQPLSLRPEACRPAAGGAVHTRGLDFACDIYIWAPL
AGTCGVLLLSLVITLYCKRGRKKLLYIFKQPFMRPVQTTQEEDGCSCRFPEEEEGGCELR
VKFSRSADAPAYQQGQNQLYNELNLGRREEYDVLDKRRGRDPEMGGKPRRKNPQEGLYNE
LQKDKMAEAYSEIGMKGERRRGKGHDGLYQGLSTATKDTYDALHMQALPPR
SEQ ID NO: 336
MGTSLLCWMALCLLGADHADACPYSNPSLCSGGGGSELPTQGTFSNVSTNVSPAKPTTTA
CPYSNPSLCSGGGGSPAPRPPTPAPTIASQPLSLRPEACRPAAGGAVHTRGLDFACDIYI
WAPLAGTCGVLLLSLVITLYCNHRNRRRVCKCPRPVVRAEGRGSLLTCGDVEENPGPMGW
SCIILFLVATATGVHSQVQLQESGGGLVQAGGSLRLSCAASGSIFSINAMGWYRQAPGKQ
RELVAAITSGGSTNYADSVKGRFTTSRDNAKNTVYLQMNSLKPEDPAVYYCNALGGFVPN
DGWGQGTQVTVSSDPTPTPAPTIASQPLSLRPEACRPAAGGAVHTRGLDFACDIYIWAPL
AGTCGVLLLSLVITLYCKRGRKKLLYIFKQPFMRPVQTTQEEDGCSCRFPEEEEGGCELR
VKFSRSADAPAYQQGQNQLYNELNLGRREEYDVLDKRRGRDPEMGGKPRRKNPQEGLYNE
LQKDKMAEAYSEIGMKGERRRGKGHDGLYQGLSTATKDTYDALHMQALPPR
SEQ ID NO: 337
MGTSLLCWMALCLLGADHADACPYSNPSLCSGGGGSELPTQGTFSNVSTNVSPAKPTTTA
CPYSNPSLCSGGGGSPAPRPPTPAPTIASQPLSLRPEACRPAAGGAVHTRGLDFACDIYI
WAPLAGTCGVLLLSLVITLYCNHRNRRRVCKCPRPVVRAEGRGSLLTCGDVEENPGPMGW
SCIILFLVATATGVHSQVQLQESGGGLVQAGGSLRLSCAASGNNFRLNAMGWYRQAPGKQ
RELVAHITSGGNTDYADSVKGRFTISRDNAKNTTYLQMNSLKPEDTAVYYCNALGGFAPN
YGWGQGTQVTVSSDPTPTPAPTIASQPLSLRPEACRPAAGGAVHTRGLDFACDIYIWAPL
AGTCGVLLLSLVITLYCKRGRKKLLYIFKQPFMRPVQTTQEEDGCSCRFPEEEEGGCELR
VKFSRSADAPAYQQGQNQLYNELNLGRREEYDVLDKRRGRDPEMGGKPRRKNPQEGLYNE
LQKDKMAEAYSEIGMKGERRRGKGHDGLYQGLSTATKDTYDALHMQALPPR
SEQ ID NO: 338
MGTSLLCWMALCLLGADHADACPYSNPSLCSGGGGSELPTQGTFSNVSTNVSPAKPTTTA
CPYSNPSLCSGGGGSPAPRPPTPAPTIASQPLSLRPEACRPAAGGAVHTRGLDFACDIYI
WAPLAGTCGVLLLSLVITLYCNHRNRRRVCKCPRPVVRAEGRGSLLTCGDVEENPGPMGW
SCIILFLVATATGVHSQVQLQQSGGGLVQAGGSLSLSCTASGRSFSNYGMGWFRQAPGKE
REFVAAIGMVGGLTAYSNSAKGRFTISRDNAKNTVYLQMNSLKPEDTAVYYCNALGGFVP
NYVWGQGTQVTVSSDPTPTPAPTIASQPLSLRPEACRPAAGGAVHTRGLDFACDIYIWAP
LAGTCGVLLLSLVITLYCKRGRKKLLYIFKQPFMRPVQTTQEEDGCSCRFPEEEEGGCEL
RVKFSRSADAPAYQQGQNQLYNELNLGRREEYDVLDKRRGRDPEMGGKPRRKNPQEGLYN
ELQKDKMAEAYSEIGMKGERRRGKGHDGLYQGLSTATKDTYDALHMQALPPR
SEQ ID NO: 339
MGTSLLCWMALCLLGADHADACPYSNPSLCSGGGGSELPTQGTFSNVSTNVSPAKPTTTA
CPYSNPSLCSGGGGSPAPRPPTPAPTIASQPLSLRPEACRPAAGGAVHTRGLDFACDIYI
WAPLAGTCGVLLLSLVITLYCNHRNRRRVCKCPRPVVRAEGRGSLLTCGDVEENPGPMGW
SCIILFLVATATGVHSQVQLQESGGGLVQAGGSLSLSCTASGRSFSINAMGWYRQAPGKQ
RELVAAITSGGSTNYADSVKGRFTISRDNAKNTVYLQMNSLKPEDTAVYYCNALGGFVLN
YGWGQGTQVTVSSDPTPTPAPTIASQPLSLRPEACRPAAGGAVHTRGLDFACDIYIWAPL
AGTCGVLLLSLVITLYCKRGRKKLLYIFKQPFMRPVQTTQEEDGCSCRFPEEEEGGCELR
VKFSRSADAPAYQQGQNQLYNELNLGRREEYDVLDKRRGRDPEMGGKPRRKNPQEGLYNE
LQKDKMAEAYSEIGMKGERRRGKGHDGLYQGLSTATKDTYDALHMQALPPR
SEQ ID NO: 340
MGTSLLCWMALCLLGADHADACPYSNPSLCSGGGGSELPTQGTFSNVSTNVSPAKPTTTA
CPYSNPSLCSGGGGSPAPRPPTPAPTIASQPLSLRPEACRPAAGGAVHTRGLDFACDIYI
WAPLAGTCGVLLLSLVITLYCNHRNRRRVCKCPRPVVRAEGRGSLLTCGDVEENPGPMGW
SCIILFLVATATGVHSQVQLQESGGGLVQAGGSLRLSCAASGSIFSINAMGWYRQAPGKQ
RELVAAITSGGSTNYADPVKGRFTISRDNAKNTVYLQMNSLKPEDTAVYYCNALGGLVPN
YGWGQGTQVTVSSDPTPTPAPTIASQPLSLRPEACRPAAGGAVHTRGLDFACDIYIWAPL
AGTCGVLLLSLVITLYCKRGRKKLLYIFKQPFMRPVQTTQEEDGCSCRFPEEEEGGCELR
VKFSRSADAPAYQQGQNQLYNELNLGRREEYDVLDKRRGRDPEMGGKPRRKNPQEGLYNE
LQKDKMAEAYSEIGMKGERRRGKGHDGLYQGLSTATKDTYDALHMQALPPR
SEQ ID NO: 341
MGTSLLCWMALCLLGADHADACPYSNPSLCSGGGGSELPTQGTFSNVSTNVSPAKPTTTA
CPYSNPSLCSGGGGSPAPRPPTPAPTIASQPLSLRPEACRPAAGGAVHTRGLDFACDIYI
WAPLAGTCGVLLLSLVITLYCNHRNRRRVCKCPRPVVRAEGRGSLLTCGDVEENPGPMGW
SCIILFLVATATGVHSQVQLQESGGGLVQAGGSLRLSCAASGSIFSINAMGWYRQAPGKQ
RELVAAITSGGSTNYADSVKGRFTISRDNAKNTVYLQMNSLKPEDTAVYYCNALGGFLPN
YGWGQGTQVTVSSDPTPTPAPTIASQPLSLRPEACRPAAGGAVHTRGLDFACDIYIWAPL
AGTCGVLLLSLVITLYCKRGRKKLLYIFKQPFMRPVQTTQEEDGCSCRFPEEEEGGCELR
VKFSRSADAPAYQQGQNQLYNELNLGRREEYDVLDKRRGRDPEMGGKPRRKNPQEGLYNE
LQKDKMAEAYSEIGMKGERRRGKGHDGLYQGLSTATKDTYDALHMQALPPR
SEQ ID NO: 342
MGTSLLCWMALCLLGADHADACPYSNPSLCSGGGGSELPTQGTFSNVSTNVSPAKPTTTA
CPYSNPSLCSGGGGSPAPRPPTPAPTIASQPLSLRPEACRPAAGGAVHTRGLDFACDIYI
WAPLAGTCGVLLLSLVITLYCNHRNRRRVCKCPRPVVRAEGRGSLLTCGDVEENPGPMGW
SCIILFLVATATGVHSQVQLQESGGGLVQAGGSLRLSCAASGSIFSINAMGWYRQAPGKQ
RELVAAITSGGSTNYADSVKGRFTISRDNAKNTVYLQMNSLKPEDTAVYYCNALGGFVTN
YGWGQGTQVAVSSDPTPTPAPTIASQPLSLRPEACRPAAGGAVHTRGLDFACDIYIWAPL
AGTCGVLLLSLVITLYCKRGRKKLLYIFKQPFMRPVQTTQEEDGCSCRFPEEEEGGCELR
VKFSRSADAPAYQQGQNQLYNELNLGRREEYDVLDKRRGRDPEMGGKPRRKNPQEGLYNE
LQKDKMAEAYSEIGMKGERRRGKGHDGLYQGLSTATKDTYDALHMQALPPR
SEQ ID NO: 343
MGTSLLCWMALCLLGADHADACPYSNPSLCSGGGGSELPTQGTFSNVSTNVSPAKPTTTA
CPYSNPSLCSGGGGSPAPRPPTPAPTIASQPLSLRPEACRPAAGGAVHTRGLDFACDIYI
WAPLAGTCGVLLLSLVITLYCNHRNRRRVCKCPRPVVRAEGRGSLLTCGDVEENPGPMGW
SCIILFLVATATGVHSQVQLQESGGGLVQPGGSLRLSCAASGSIFSINAMGWYRQAPGKQ
RELVAFITSGGSTNYADSVKGRFTISRDNAKNTVYLQMNSLKPEDTAVYYCNALGGFVPS
YGWGQGTQVTVSSDPTPTPAPTIASQPLSLRPEACRPAAGGAVHTRGLDFACDIYIWAPL
AGTCGVLLLSLVITLYCKRGRKKLLYIFKQPFMRPVQTTQEEDGCSCRFPEEEEGGCELR
VKFSRSADAPAYQQGQNQLYNELNLGRREEYDVLDKRRGRDPEMGGKPRRKNPQEGLYNE
LQKDKMAEAYSEIGMKGERRRGKGHDGLYQGLSTATKDTYDALHMQALPPR
SEQ ID NO: 344
MGTSLLCWMALCLLGADHADACPYSNPSLCSGGGGSELPTQGTFSNVSTNVSPAKPTTTA
CPYSNPSLCSGGGGSPAPRPPTPAPTIASQPLSLRPEACRPAAGGAVHTRGLDFACDIYI
WAPLAGTCGVLLLSLVITLYCNHRNRRRVCKCPRPVVRAEGRGSLLTCGDVEENPGPMGW
SCIILFLVATATGVHSQVQLQQSGGGLVQTGGSLRLSCAASGRTVMGWYRQAPGKQRELV
AVITRGGSTNYADSVKGRFTISRDNAKNTVYLQMNSLKPEDTAFYYCNALGGFVPNYGWG
QGTQVTVSSDPTPTPAPTIASQPLSLRPEACRPAAGGAVHTRGLDFACDIYIWAPLAGTC
GVLLLSLVITLYCKRGRKKLLYIFKQPFMRPVQTTQEEDGCSCRFPEEEEGGCELRVKFS
RSADAPAYQQGQNQLYNELNLGRREEYDVLDKRRGRDPEMGGKPRRKNPQEGLYNELQKD
KMAEAYSEIGMKGERRRGKGHDGLYQGLSTATKDTYDALHMQALPPR
SEQ ID NO: 345
MGTSLLCWMALCLLGADHADACPYSNPSLCSGGGGSELPTQGTFSNVSTNVSPAKPTTTA
CPYSNPSLCSGGGGSPAPRPPTPAPTIASQPLSLRPEACRPAAGGAVHTRGLDFACDIYI
WAPLAGTCGVLLLSLVITLYCNHRNRRRVCKCPRPVVRAEGRGSLLTCGDVEENPGPMGW
SCIILFLVATATGVHSQVQLQESGGGLVQPGGSLRLSCAAPGNIFRLNGTGWYRQAPGKQ
RELVAHITSGGNTDYADSVKGRFTISRDNAKNTVYLQMNSLKPEDTAVYYCNALGGFVPS
YGWGQGTQVTVSSDPTPTPAPTIASQPLSLRPEACRPAAGGAVHTRGLDFACDIYIWAPL
AGTCGVLLLSLVITLYCKRGRKKLLYIFKQPFMRPVQTTQEEDGCSCRFPEEEEGGCELR
VKFSRSADAPAYQQGQNQLYNELNLGRREEYDVLDKRRGRDPEMGGKPRRKNPQEGLYNE
LQKDKMAEAYSEIGMKGERRRGKGHDGLYQGLSTATKDTYDALHMQALPPR
SEQ ID NO: 346
MGTSLLCWMALCLLGADHADACPYSNPSLCSGGGGSELPTQGTFSNVSTNVSPAKPTTTA
CPYSNPSLCSGGGGSPAPRPPTPAPTIASQPLSLRPEACRPAAGGAVHTRGLDFACDIYI
WAPLAGTCGVLLLSLVITLYCNHRNRRRVCKCPRPVVRAEGRGSLLTCGDVEENPGPMGW
SCIILFLVATATGVHSQVQLQESGGGLVQPGGSLRLSCAAPGNNFRLNAMGWYRQAPGKQ
RELVAHITSGGNTDYADSVKGRFTISRDNAKNTVYLQMNSLKPEDTAVYYCNALGGFVPS
YGWGQGTQVTVSSDPTPTPAPTIASQPLSLRPEACRPAAGGAVHTRGLDFACDIYIWAPL
AGTCGVLLLSLVITLYCKRGRKKLLYIFKQPFMRPVQTTQEEDGCSCRFPEEEEGGCELR
VKFSRSADAPAYQQGQNQLYNELNLGRREEYDVLDKRRGRDPEMGGKPRRKNPQEGLYNE
LQKDKMAEAYSEIGMKGERRRGKGHDGLYQGLSTATKDTYDALHMQALPPR
SEQ ID NO: 347
MGTSLLCWMALCLLGADHADACPYSNPSLCSGGGGSELPTQGTFSNVSTNVSPAKPTTTA
CPYSNPSLCSGGGGSPAPRPPTPAPTIASQPLSLRPEACRPAAGGAVHTRGLDFACDIYI
WAPLAGTCGVLLLSLVITLYCNHRNRRRVCKCPRPVVRAEGRGSLLTCGDVEENPGPMGW
SCIILFLVATATGVHSQVQLQQSGGGLVQAGGSLRLSCAASGSIFSINAMGWYRQAPGKQ
RELVAFITSGGSTNYADSVKGRFTISRDNAKNTVYLQMNSLKPEDTAVYYCNALGGFVPS
YGWGQGTQVTVSSDPTPTPAPTIASQPLSLRPEACRPAAGGAVHTRGLDFACDIYIWAPL
AGTCGVLLLSLVITLYCKRGRKKLLYIFKQPFMRPVQTTQEEDGCSCRFPEEEEGGCELR
VKFSRSADAPAYQQGQNQLYNELNLGRREEYDVLDKRRGRDPEMGGKPRRKNPQEGLYNE
LQKDKMAEAYSEIGMKGERRRGKGHDGLYQGLSTATKDTYDALHMQALPPR
SEQ ID NO: 348
MGTSLLCWMALCLLGADHADACPYSNPSLCSGGGGSELPTQGTFSNVSTNVSPAKPTTTA
CPYSNPSLCSGGGGSPAPRPPTPAPTIASQPLSLRPEACRPAAGGAVHTRGLDFACDIYI
WAPLAGTCGVLLLSLVITLYCNHRNRRRVCKCPRPVVRAEGRGSLLTCGDVEENPGPMGW
SCIILFLVATATGVHSQVQLQQSGGGLVQPGGSLRLSCAAPGNIFRLNGTGWYRQAPGKQ
RELVAHITSGGSTNYADSVKGRFTISRDNAKNTVYLQMNSLKPEDTAVYYCNALGGFVPS
YGWGQGTQVTVSSDPTPTPAPTIASQPLSLRPEACRPAAGGAVHTRGLDFACDIYIWAPL
AGTCGVLLLSLVITLYCKRGRKKLLYIFKQPFMRPVQTTQEEDGCSCRFPEEEEGGCELR
VKFSRSADAPAYQQGQNQLYNELNLGRREEYDVLDKRRGRDPEMGGKPRRKNPQEGLYNE
LQKDKMAEAYSEIGMKGERRRGKGHDGLYQGLSTATKDTYDALHMQALPPR
SEQ ID NO: 349
MGTSLLCWMALCLLGADHADACPYSNPSLCSGGGGSELPTQGTFSNVSTNVSPAKPTTTA
CPYSNPSLCSGGGGSPAPRPPTPAPTIASQPLSLRPEACRPAAGGAVHTRGLDFACDIYI
WAPLAGTCGVLLLSLVITLYCNHRNRRRVCKCPRPVVRAEGRGSLLTCGDVEENPGPMGW
SCIILFLVATATGVHSQVQLQESGGGLVQAGGSLRLSCAASGSIFSINAMGWYRQAPGKQ
RELVAAITSGGSTNYADSVKGRFTISRDNAKNTVYLQMNSLKPEDTAVYYCNALGGFVPS
YGWGQGTQVTVSSDPTPTPAPTIASQPLSLRPEACRPAAGGAVHTRGLDFACDIYIWAPL
AGTCGVLLLSLVITLYCKRGRKKLLYIFKQPFMRPVQTTQEEDGCSCRFPEEEEGGCELR
VKFSRSADAPAYQQGQNQLYNELNLGRREEYDVLDKRRGRDPEMGGKPRRKNPQEGLYNE
LQKDKMAEAYSEIGMKGERRRGKGHDGLYQGLSTATKDTYDALHMQALPPR
SEQ ID NO: 350
MGTSLLCWMALCLLGADHADACPYSNPSLCSGGGGSELPTQGTFSNVSTNVSPAKPTTTA
CPYSNPSLCSGGGGSPAPRPPTPAPTIASQPLSLRPEACRPAAGGAVHTRGLDFACDIYI
WAPLAGTCGVLLLSLVITLYCNHRNRRRVCKCPRPVVRAEGRGSLLTCGDVEENPGPMGW
SCIILFLVATATGVHSQVQLQESGGGLVQAGGSLSLSCTASGRSFSNYGMGWFRQAPGKE
REFVAAIGMVGGLTAYSNSAKGRFTISRDNAKNTLYLQMNSLKPEDTAVYYCNALGGFVP
SYGWGQGTQVTVSSDPTPTPAPTIASQPLSLRPEACRPAAGGAVHTRGLDFACDIYIWAP
LAGTCGVLLLSLVITLYCKRGRKKLLYIFKQPFMRPVQTTQEEDGCSCRFPEEEEGGCEL
RVKFSRSADAPAYQQGQNQLYNELNLGRREEYDVLDKRRGRDPEMGGKPRRKNPQEGLYN
ELQKDKMAEAYSEIGMKGERRRGKGHDGLYQGLSTATKDTYDALHMQALPPR
SEQ ID NO: 351
MGTSLLCWMALCLLGADHADACPYSNPSLCSGGGGSELPTQGTFSNVSTNVSPAKPTTTA
CPYSNPSLCSGGGGSPAPRPPTPAPTIASQPLSLRPEACRPAAGGAVHTRGLDFACDIYI
WAPLAGTCGVLLLSLVITLYCNHRNRRRVCKCPRPVVRAEGRGSLLTCGDVEENPGPMGW
SCIILFLVATATGVHSQVQLQESGGGLVQAGGSLRLSCAASGSIFSINAMGWYRQAPGKQ
RELVAFITSGGSTNYADSVKGRFTISRDNAKNTVYLQMNSLKPEDTAVYYCNALGGFVPS
YGWGQGTLVTVSSDPTPTPAPTIASQPLSLRPEACRPAAGGAVHTRGLDFACDIYIWAPL
AGTCGVLLLSLVITLYCKRGRKKLLYIFKQPFMRPVQTTQEEDGCSCRFPEEEEGGCELR
VKFSRSADAPAYQQGQNQLYNELNLGRREEYDVLDKRRGRDPEMGGKPRRKNPQEGLYNE
LQKDKMAEAYSEIGMKGERRRGKGHDGLYQGLSTATKDTYDALHMQALPPR
SEQ ID NO: 352
MGTSLLCWMALCLLGADHADACPYSNPSLCSGGGGSELPTQGTFSNVSTNVSPAKPTTTA
CPYSNPSLCSGGGGSPAPRPPTPAPTIASQPLSLRPEACRPAAGGAVHTRGLDFACDIYI
WAPLAGTCGVLLLSLVITLYCNHRNRRRVCKCPRPVVRAEGRGSLLTCGDVEENPGPMGW
SCIILFLVATATGVHSQVQLQESGGGLVQPGGSLRLSCAAPGNIFSINAMGWYRQAPGKQ
RELVAFITSGGSTNYADSVKGRFTISRDNAKNTVYLQMNSLKPEDTAVYYCNALGGFVPS
YGWGQGTQVTVSSDPTPTPAPTIASQPLSLRPEACRPAAGGAVHTRGLDFACDIYIWAPL
AGTCGVLLLSLVITLYCKRGRKKLLYIFKQPFMRPVQTTQEEDGCSCRFPEEEEGGCELR
VKFSRSADAPAYQQGQNQLYNELNLGRREEYDVLDKRRGRDPEMGGKPRRKNPQEGLYNE
LQKDKMAEAYSEIGMKGERRRGKGHDGLYQGLSTATKDTYDALHMQALPPR
SEQ ID NO: 353
MGTSLLCWMALCLLGADHADACPYSNPSLCSGGGGSELPTQGTFSNVSTNVSPAKPTTTA
CPYSNPSLCSGGGGSPAPRPPTPAPTIASQPLSLRPEACRPAAGGAVHTRGLDFACDIYI
WAPLAGTCGVLLLSLVITLYCNHRNRRRVCKCPRPVVRAEGRGSLLTCGDVEENPGPMGW
SCIILFLVATATGVHSQVQLQESGGGLVQPGGSLRLSCLASGFTFSTYWMSWVRQAPGKG
PEWVSGIDNGGGTTTYADSVKGRFTISRDNAKNTLNLEMNNLKPEDTALYYCARNPTRGW
YSTDYRGQGTQVTVSSDPTPTPAPTIASQPLSLRPEACRPAAGGAVHTRGLDFACDIYIW
APLAGTCGVLLLSLVITLYCKRGRKKLLYIFKQPFMRPVQTTQEEDGCSCRFPEEEEGGC
ELRVKFSRSADAPAYQQGQNQLYNELNLGRREEYDVLDKRRGRDPEMGGKPRRKNPQEGL
YNELQKDKMAEAYSEIGMKGERRRGKGHDGLYQGLSTATKDTYDALHMQALPPR
SEQ ID NO: 354
MGTSLLCWMALCLLGADHADACPYSNPSLCSGGGGSELPTQGTFSNVSTNVSPAKPTTTA
CPYSNPSLCSGGGGSPAPRPPTPAPTIASQPLSLRPEACRPAAGGAVHTRGLDFACDIYI
WAPLAGTCGVLLLSLVITLYCNHRNRRRVCKCPRPVVRAEGRGSLLTCGDVEENPGPMGW
SCIILFLVATATGVHSQVQLQESGGGLVQPGGSLRLSCAASGFTFSTYWMSWVRQAPGKG
PEWVSGIDNGGGTTTYADSVKGRFTISRDNAKHTLYLQMNTLKPEDTALYYCARNPTRGW
YSTDYRGQGTQVTVSSDPTPTPAPTIASQPLSLRPEACRPAAGGAVHTRGLDFACDIYIW
APLAGTCGVLLLSLVITLYCKRGRKKLLYIFKQPFMRPVQTTQEEDGCSCRFPEEEEGGC
ELRVKFSRSADAPAYQQGQNQLYNELNLGRREEYDVLDKRRGRDPEMGGKPRRKNPQEGL
YNELQKDKMAEAYSEIGMKGERRRGKGHDGLYQGLSTATKDTYDALHMQALPPR

The sequences shown as SEQ ID NO: 76-SEQ ID NO: 89, SEQ ID NO: 159 and SEQ ID NO: 289-SEQ ID NO: 354 include the sequences of RQR8 (SEQ ID NO: 75), 2A-like sequence (SEQ ID NO: 359) and the signal peptide of SEQ ID NO: 358. An aspect of the present invention is also a CAR comprising a sequence selected from the group consisting of the sequences shown as SEQ ID NOs: 76-89, 159 and 289-354 minus the sequences of RQR8 (SEQ ID NO: 75), 2A-like sequence (SEQ ID NO: 359) and, optionally, the signal peptide of SEQ ID NO: 358, or a variant of said CAR which has at least 80%, 85%, 90%, 95%, 98% or 99% sequence identity thereto but retains the capacity to i) bind FcRH5 and ii) induce T cell signalling.

### Immune Cell Engagers

Immune cell engager molecules are a class of antibody-type molecules that have been developed, primarily for the use as anti-cancer drugs. They direct immune effector cells of a host's immune system against a target cell, such as a cancer cell. In these immune cell engager molecules, at least one binding domain binds to the immune cell via, for example, a receptor expressed on the immune cell, and another binding domain binds to a target cell such as a tumour cell (e.g. via a tumour specific molecule). Since the immune cell engager molecule binds both the target cell and the immune cell, it brings the target cell into proximity with the immune cell, so that the immune cell can exert its effect, for example, a cytotoxic effect on a cancer cell. The formation of the immune cell: immune cell engager: cancer cell complex induces signalling in the immune cell leading to, for example, the release of cytotoxic mediators. Ideally, the agent only induces the desired signalling in the presence of the target cell, leading to selective killing.

Thus, an immune cell engager molecule which of the present invention brings a FcRH5-expressing cell (for example, a FcRH5+ cancer cell) into proximity with an immune cell, so that the immune cell can exert its effect on the cancer cell. The requirement of co-localisation via binding of the FcRH5 immune cell engager molecule may lead to selective killing of FcRH5-positive cells. Suitably, an immune cell engager molecule of the present invention is able to activate an immune cell following binding of the immune cell engager molecule to FcRH5 expressed on the surface of target cells.

The immune cell engager may be multivalent and may comprise multiple copies of the same immune cell binding domain.

The immune cell engager may comprise multiple immune cell binding domains wherein each immune cell binding domain has a different target molecule expressed on the immune cell.

The immune cell engager may be a T cell engager, an NK cell engager, a B cell engager, a dendritic cell engager, or a macrophage cell engager. In some embodiments, the immune cell engager binds to and activates an immune cell, e.g., an effector cell. In some embodiments, the immune cell engager binds to, but does not activate, an immune cell, e.g., an effector cell.

The immune cell engager may be capable of binding, for example, a T cell (i.e. an alpha beta T cell), an NKT cell, a gamma delta T cell or an NK cell.

### Bi-Specific T Cell Engagers (Bites) and Tri-Specific T Cell Engagers

In one embodiment, the present invention provides a T cell engager molecule which is a bi-specific T cell engager (BiTE) which comprises a sdAb as described herein as a first domain, and a T cell activating domain as a second domain. A T cell activating domain is a domain capable of activating a T cell.

Bi-specific T cell engaging molecules typically comprise a binding domain which binds to a T cell via, for example, the CD3 receptor, and the other to a target cell such as a tumour cell (e.g. via a tumour specific molecule). Since the bi-specific molecule binds both the target cell and the T cell, it brings the target cell into proximity with the T cell, so that the T cell can exert its effect, for example, a cytotoxic effect on a cancer cell. The formation of the T cell:bi-specific Ab:cancer cell complex induces signalling in the T cell leading to, for example, the release of cytotoxic mediators. Ideally, the agent only induces the desired signalling in the presence of the target cell, leading to selective killing.

Thus, a T cell engager molecule which is a bi-specific molecule of the present invention brings a FcRH5-expressing cell (for example, a FcRH5+ cancer cell) into proximity with a T cell, so that the T cell can exert its effect on the cancer cell. The requirement of co-localisation via binding of the FcRH5 bi-specific molecule suitably leads to selective killing of FcRH5-positive cells. In other words, a bi-specific molecule of the present invention is able to activate T cells following binding of the bi-specific molecule to FcRH5 expressed on the surface of target cells.

BiTEs are commonly made by fusing an anti-CD3 scFv to an anti-target antigen scFv via a short five residue peptide linker (e.g. GGGGS (SEQ ID NO: 117)).

In one embodiment, the present invention provides a T cell engager molecule which is a tri-specific T cell engager which comprises a sdAb as described herein as a first domain, a T cell activating domain as a second domain, and another T cell co-activating domain as a third domain. A T cell activating domain is a domain capable of activating a T cell.

A tri-specific T cell engager molecule may comprise the same first and second domains described herein for a bi-specific T cell engager molecule, with the addition of a third domain which is also capable of co-activating a T cell. Accordingly, a tri-specific T cell engager molecule may have a similar effect and function as a bi-specific T cell engager molecule as described herein.

### Second Domain-T Cell Activating Domain

The second domain of the bi-specific T cell engager molecule of the present invention or tri-specific T cell engager molecule of the present invention is capable of activating T cells. T cells have a T cell-receptor (TCR) at the cell surface which recognises antigenic peptides when presented by an MHC molecule on the surface of an antigen presenting cell. Such antigen recognition results in the phosphorylation of immunoreceptor tyrosine-based activation motifs (ITAMs) by Src family kinases, triggering recruitment of further kinases which results in T cell activation including Ca²⁺ release.

The second domain may cause T cell activation by triggering the same pathway triggered by antigen-specific recognition by the TCR. Thus, the second domain may induce T cell signalling and result in T cell activation.

### Cluster of Differentiation 3 (CD3)

The second domain of the bi-specific T cell engager molecule of the invention or tri-specific T cell engager molecule of the invention may bind CD3.

CD3 is a protein complex composed of four distinct chains: a CD3γ chain, a CD3δ chain, and two CD3ε chains. CD3 associates with the T cell receptor (TCR) and the ζ-chain on the surface of a T cell to generate an activation signal. The TCR, ζ-chain, and CD3 molecule together comprise the TCR complex.

Clustering of CD3 on T cells, e.g. by immobilized anti-CD3-antibodies, leads to T cell activation similar to the engagement of the T cell receptor, but independent from its clone typical specificity.

Due to its central role in modulating T cell activity, there have been attempts to develop molecules that are capable of binding TCR/CD3. Much of this work has focused on the generation of antibodies that are specific for the human CD3 antigen.

The second domain may comprise an antibody or part thereof which specifically binds CD3, such as OKT3, WT32, anti-leu-4, UCHT-1, SPV-3TA, TR66, SPV-T3B or affinity tuned variants thereof.

As used herein in the context of a second or third domain of an immune cell engager, “antibody” means a polypeptide having an antigen binding site which comprises at least one complementarity determining region CDR. The antibody may comprise 3 CDRs and have an antigen binding site which is equivalent to that of a domain antibody (dAb). The antibody may comprise 6 CDRs and have an antigen binding site which is equivalent to that of a classical antibody molecule. The remainder of the polypeptide may be any sequence which provides a suitable scaffold for the antigen binding site and displays it in an appropriate manner for it to bind the antigen. The antibody may be a whole immunoglobulin molecule or a part thereof such as a Fab, F(ab)′₂, Fv, single chain Fv (ScFv) fragment, Nanobody or single chain variable domain (which may be a VH or VL chain, having 3 CDRs). The antibody may be a bifunctional antibody. The antibody may be non-human, chimeric, humanised or fully human.

Alternatively the second domain may comprise a CD3-binding molecule which is not derived from or based on an immunoglobulin. A number of “antibody mimetic” designed repeat proteins (DRPs) have been developed to exploit the binding abilities of non-antibody polypeptides. Such molecules include ankyrin or leucine-rich repeat proteins e.g. DARPins (Designed Ankyrin Repeat Proteins), Anticalins, Avimers and Versabodies.

The second domain of the bi-specific T cell engager molecule of the invention or tri-specific T cell engager molecule of the invention may comprise all or part of the monoclonal antibody OKT3, which was the first monoclonal antibody approved by the FDA. OKT3 is available from ATCC CRL 8001. The antibody sequences are published in U.S. Pat. No. 7,381,803.

The second domain may comprise one or more CDRs from OKT3. The second binding domain may comprise CDR3 from the heavy-chain of OKT3 and/or CDR3 from the light chain of OKT3. The second binding domain may comprise all 6 CDRs from OKT3, as shown below.

Heavy Chain
CDR1:
(SEQ ID NO: 90)
KASGYTFTRYTMH
CDR2:
(SEQ ID NO: 91)
INPSRGYTNYNQKFKD
CDR3:
(SEQ ID NO: 92)
YYDDHYCLDY
Light Chain
CDR1:
(SEQ ID NO: 93)
SASSSVSYMN
CDR2:
(SEQ ID NO: 94)
RWIYDTSKLAS
CDR3:
(SEQ ID NO: 95)
QQWSSNPFT

Suitably, one or more of the CDRs may comprise comprises one, two or three amino acid mutations.

The second binding domain may comprise a scFv which comprises the CDR sequences from OKT3. The second binding domain may comprise the scFv sequence shown below as SEQ ID NO: 96 or a variant thereof having at least 80%, 85%, 90%, 95%, 98% or 99% sequence identity thereto, which retains the capacity to bind CD3.

SEQ ID NO: 96
QVQLQQSGAELARPGASVKMSCKASGYTFTRYTMHWVKQRPGQGLEWIGY
INPSRGYTNYNQKFKDKATLTTDKSSSTAYMQLSSLTSEDSAVYYCARYY
DDHYCLDYWGQGTTLTVSSSGGGGSGGGGSGGGGSQIVLTQSPAIMSASP
GEKVTMTCSASSSVSYMNWYQQKSGTSPKRWIYDTSKLASGVPAHFRGSG
SGTSYSLTISGMEAEDAATYYCQQWSSNPFTFGSGTKLEINR

The second domain may comprise one or more CDRs from UCHT1. The second binding domain may comprise CDR3 from the heavy-chain of UCHT1 and/or CDR3 from the light chain of UCHT1. The second binding domain may comprise all 6 CDRs from UCHT1, as shown below.

Heavy Chain
CDR1:
(SEQ ID NO: 97)
GYSFTGYT
CDR2:
(SEQ ID NO: 98)
INPYKGVS
CDR3:
(SEQ ID NO: 99)
ARSGYYGDSDWYFDV
Light Chain
CDR1:
(SEQ ID NO: 100)
QDIRNY
CDR2:
(SEQ ID NO: 101)
YTS
CDR3:
(SEQ ID NO: 102)
QQGNTLPWT

Suitably, one or more of the CDRs may comprise comprises one, two or three amino acid mutations.

The second binding domain may comprise a scFv which comprises the CDR sequences from UCHT1. The second binding domain may comprise the scFv sequence shown below as SEQ ID NO: 103 or a variant thereof having at least 80%, 85%, 90%, 95%, 98% or 99% sequence identity thereto, which retains the capacity to bind CD3.

SEQ ID NO: 103
DIQMTQSPSSLSASVGNRVTITCRASQDIRNYLNWYQQKPGKAPKLLIYY
TSRLESGVPSRFSGSGSGTDYTLTISSLQPEDFATYYCQQGNTLPWTFGQ
GTKVEIKSGGGGSGGGGSGGGGSEVQLVESGGGLVQPGGSLRLSCAASGY
SFTGYTMNWVRQAPGKGLEWVALINPYKGVSTYNQKFKDRFTISVDKSKN
TAYLQMNSLRAEDTAVYYCARSGYYGDSDWYFDVWGQGTLVTVSS

The second domain may comprise one or more CDRs from YTH. The second binding domain may comprise CDR3 from the heavy-chain of YTH and/or CDR3 from the light chain of YTH. The second binding domain may comprise all 6 CDRs from YTH, as shown below.

Heavy Chain
CDR1:
(SEQ ID NO: 104)
GFTFSSFP
CDR2:
(SEQ ID NO: 105)
ISTSGGRT
CDR3:
(SEQ ID NO: 106)
AKFRQYSGGFDY
Light Chain
CDR1:
(SEQ ID NO: 107)
SGNIENNY
CDR2:
(SEQ ID NO: 108)
DDD
CDR3:
(SEQ ID NO: 109)
HSYVSSFNV

Suitably, one or more of the CDRs may comprise comprises one, two or three amino acid mutations.

The second binding domain may comprise a scFv which comprises the CDR sequences from YTH. The second binding domain may comprise the scFv sequence shown below as SEQ ID NO: 110 or a variant thereof having at least 80%, 85%, 90%, 95%, 98% or 99% sequence identity thereto, which retains the capacity to bind CD3.

SEQ ID NO: 110
DFMLTQPHSVSESPGKTVIISCTLSSGNIENNYVHWYQQRPGRAPTTVIF
DDDKRPDGVPDRFSGSIDRSSNSASLTISGLQTEDEADYYCHSYVSSFNV
FGGGTKLTVLSGGGGSGGGGSGGGGSEVQLLESGGGLVQPGGSLRLSCAA
SGFTFSSFPMAWVRQAPGKGLEWVSTISTSGGRTYYRDSVKGRFTISRDN
SKNTLYLQMNSLRAEDTAVYYCAKFRQYSGGFDYWGQGTLVTVSS

A variant sequence of SEQ ID NOs: 96, 103 or 110 may have at least 80%, 85%, 90%, 95%, 98% or 99% sequence identity and have equivalent or improved CD3 binding and/or TCR activation capabilities compared to the sequence shown as SEQ ID NO: 96, 103 or 110.

The second binding domain of the tri-specific T cell engager molecule of the invention may comprise a VH region having the sequence shown as SEQ ID NO: 120 or a variant having at least 80%, 85%, 90%, 95%, 98% or 99% sequence identity thereto which retains the capacity to bind CD3; and a VL region having the sequence shown as SEQ ID NO: 121 or a variant of having at least 80%, 85%, 90%, 95%, 98% or 99% sequence identity thereto which retains the capacity to bind CD3. Suitably, the second binding domain may comprise a VH region having the sequence shown as SEQ ID NO: 120; and a VL region having the sequence shown as SEQ ID NO: 121.

VH domain
(SEQ ID NO: 120)
QVQLVESGGGVVQPGRSLRLSCAASGFTFTKAWMHWVRQAPGKQL
EWVAQIKDKSNSYATYYADSVKGRFTISRDDSKNTLYLQMNSLRA
EDTAVYYCRGVYYALSPFDYWGQGTLVTVSS
VL domain
(SEQ ID NO: 121)
DIVMTQTPLSLSVTPGQPASISCKSSQSLVHNNANTYLSWYLQKP
GQSPQSLIYKVSNRFSGVPDRFSGSGSGTDFTLKISRVEAEDVGV
YYCGQGTQYPFTFGSGTKVEIK

The second binding domain of the tri-specific T cell engager molecule of the invention may comprise a VH region having the sequence shown as SEQ ID NO: 122 or a variant having at least 80%, 85%, 90%, 95%, 98% or 99% sequence identity thereto which retains the capacity to bind CD3; and a VL region having the sequence shown as SEQ ID NO: 123 or a variant of having at least 80%, 85%, 90%, 95%, 98% or 99% sequence identity thereto which retains the capacity to bind CD3. Suitably, the second binding domain may comprise a VH region having the sequence shown as SEQ ID NO: 122; and a VL region having the sequence shown as SEQ ID NO: 123.

VH domain
(SEQ ID NO: 122)
QVQLVESGGGVVQPGRSLRLSCAASGFTFTKAWMHWVRQAPGKGL
EWVAQIKDKSNSYATYYADSVKGRFTISRDNSKNTLYLQMNSLRA
EDTAVYYCRGVYYALSPFDYWGQGTLVTVSS
VL domain
(SEQ ID NO: 123)
DIVMTQTPLSLSVTPGQPASISCKSSQSLVHNNGNTYLSWYLQKP
GQSPQLLIYKVSNRFSGVPDRFSGSGSGTDFTLKISRVEAEDVGV
YYCGQGTQYPFTFGGGTKVEIK

### Third Domain-T Cell Co-Activating Domain

The third domain of the tri-specific T cell engager molecule of the present invention is capable of co-activating T cells.

The third domain may cause T cell activation by triggering the same pathway triggered by co-stimulation of a T cell. Thus, the third domain may result in T cell activation.

The first and second domains of the tri-specific T cell engager molecule of the present invention may comprise the first and second domains described herein for the bi-specific T cell engager molecule of the invention.

### CD28

The third domain of the tri-specific T cell engager molecule of the invention may bind CD28.

The third domain may comprise an antibody or part thereof which specifically binds CD28, such as TGN1412 or affinity tuned variants thereof.

The third domain of the tri-specific T cell engager molecule of the invention may comprise all or part of the monoclonal antibody TGN1412.

The third domain may comprise one or more CDRs from TGN1412. The second binding domain may comprise CDR3 from the heavy-chain of TGN1412 and/or CDR3 from the light chain of TGN1412. The second binding domain may comprise all 6 CDRs from TGN1412, as shown below.

Heavy Chain
CDR1:
(SEQ ID NO: 124)
GYTFTSYY
CDR2:
(SEQ ID NO: 125)
IYPGNVNT
CDR3:
(SEQ ID NO: 126)
TRSHYGLDWNFDV
Light Chain
CDR1:
(SEQ ID NO: 127)
QNIYVW
CDR2:
(SEQ ID NO: 128)
KA
CDR3:
(SEQ ID NO: 129)
QQGQTYPYT

Suitably, one or more of the CDRs may comprise comprises one, two or three amino acid mutations.

The third binding domain may comprise a scFv which comprises the CDR sequences from TGN1412.

The third binding domain may comprise a VH region having the sequence shown as SEQ ID NO: 130 or a variant having at least 80%, 85%, 90%, 95%, 98% or 99% sequence identity thereto which retains the capacity to bind CD28; and a VL region having the sequence shown as SEQ ID NO: 131 or a variant of having at least 80%, 85%, 90%, 95%, 98% or 99% sequence identity thereto which retains the capacity to bind CD28. Suitably, the third binding domain may comprise a VH region having the sequence shown as SEQ ID NO: 130; and a VL region having the sequence shown as SEQ ID NO: 131.

VH domain
SEQ ID NO: 130
QVQLVQSGAEVKKPGASVKVSCKASGYTFTSYYIHWVRQAPGQGL
EWIGCIYPGNVNTNYNEKFKDRATLTVDTSISTAYMELSRLRSDD
TAVYFCTRSHYGLDWNFDVWGQGTTVTVSS
VL domain
SEQ ID NO: 131
DIQMTQSPSSLSASVGDRVTITCHASQNIYVWLNWYQQKPGKAPK
LLIYKASNLHTGVPSRFSGSGSGTDFTLTISSLQPEDFATYYCQQ
GQTYPYTFGGGTKVEIKRTV

The third binding domain of the tri-specific T cell engager molecule of the invention may comprise a VH region having the sequence shown as SEQ ID NO: 132 or a variant having at least 80%, 85%, 90%, 95%, 98% or 99% sequence identity thereto which retains the capacity to bind CD28; and a VL region having the sequence shown as SEQ ID NO: 133 or a variant of having at least 80%, 85%, 90%, 95%, 98% or 99% sequence identity thereto which retains the capacity to bind CD28. Suitably, the third binding domain may comprise a VH region having the sequence shown as SEQ ID NO: 132; and a VL region having the sequence shown as SEQ ID NO: 133.

VH domain
(SEQ ID NO: 132)
QVQLVQSGAEVVKPGASVKVSCKASGYTFTSYYIHWVRQAPGQGL
EWIGSIYPGNVNTNYAQKFQGRATLTVDTSISTAYMELSRLRSDD
TAVYYCTRSHYGLDWNFDVWGKGTTVTVSS
VL domain
(SEQ ID NO: 133)
DIQMTQSPSSLSASVGDRVTITCQASQNIYVWLNWYQQKPGKAPK
LLIYKASNLHTGVPSRFSGSGSGTDFTLTISSLQPEDIATYYCQQ
GQTYPYTFGQGTKLEIK

The third binding domain of the tri-specific T cell engager molecule of the invention may comprise a VH region having the sequence shown as SEQ ID NO: 134 or a variant having at least 80%, 85%, 90%, 95%, 98% or 99% sequence identity thereto which retains the capacity to bind CD28; and a VL region having the sequence shown as SEQ ID NO: 135 or a variant of having at least 80%, 85%, 90%, 95%, 98% or 99% sequence identity thereto which retains the capacity to bind CD28. Suitably, the third binding domain may comprise a VH region having the sequence shown as SEQ ID NO: 134; and a VL region having the sequence shown as SEQ ID NO: 135.

VH domain
(SEQ ID NO: 134)
QVQLQESGPGLVKPSQTLSLTCTVSGFSLSDYGVHWVRQPPGKGL
EWLGVIWAGGGTNYNPSLKSRKTISKDTSKNQVSLKLSSVTAADT
AVYYCARDKGYSYYYSMDYWGQGTTVTVSS
VL domain
(SEQ ID NO: 135)
DIVLTQSPASLAVSPGQRATITCRASESVEYYVTSLMQWYQQKPG
QPPKLLIFAASNVESGVPARFSGSGSGTDFTLTINPVEANDVANY
YCQQSRKVPYTFGQGTKLEIK

### Secretion Signal Peptide

The bi-specific T cell engager molecule of the invention or tri-specific T cell engager molecule of the invention may comprise a signal peptide to aid in its production. The signal peptide may cause the bi-specific T cell engager molecule or tri-specific T cell engager molecule to be secreted by a host cell, such that the molecule can be harvested from the host cell supernatant.

The core of the signal peptide may contain a long stretch of hydrophobic amino acids that has a tendency to form a single alpha-helix. The signal peptide may begin with a short positively charged stretch of amino acids, which helps to enforce proper topology of the polypeptide during translocation. At the end of the signal peptide there is typically a stretch of amino acids that is recognized and cleaved by signal peptidase. Signal peptidase may cleave either during or after completion of translocation to generate a free signal peptide and a mature protein. The free signal peptides are then digested by specific proteases.

The signal peptide may be at the amino terminus of the molecule.

The bi-specific T cell engager molecule may have the general formula:

Signal peptide-first domain-second domain.

The tri-specific T cell engager molecule may have the general formula:

Signal peptide-first domain-second domain-third domain.

The signal peptide may comprise the SEQ ID NO: 111 or 112 or a variant thereof having 5, 4, 3, 2 or 1 amino acid mutations (insertions, substitutions or additions) provided that the signal peptide still functions to cause secretion of the molecule.

SEQ ID NO: 111:
METDTLLLWVLLLWVPGSTG
SEQ ID NO: 112:
MGTSLLCWMALCLLGADHADG

The signal peptides of SEQ ID NO: 111 and 112 are compact and highly efficient. They are predicted to give about 95% cleavage after the terminal glycine, giving efficient removal by signal peptidase.

### Spacer

The bi-specific T cell engager molecule of the invention or tri-specific T cell engager molecule of the invention may comprise a spacer or linker sequence to connect the first domain with the second domain and/or to connect the second domain with the third domain in order to spatially separate the domains.

The spacer sequence may, for example, comprise an IgG1 hinge or a CD8 stalk. The spacer or linker may alternatively comprise an alternative linker sequence which has similar length and/or domain spacing properties as an IgG1 hinge or a CD8 stalk.

The spacer may be a short spacer, for example a spacer which comprises less than 100, less than 80, less than 60 or less than 45 amino acids. The spacer may be or comprise an IgG1 hinge or a CD8 stalk or a modified version thereof.

Examples of amino acid sequences for
these spacers are given below:
SEQ ID NO: 113 (IgG1 hinge):
AEPKSPDKTHTCPPCPKDPKSGGGGS
SEQ ID NO: 114 (CD8 stalk):
TTTPAPRPPTPAPTIASQPLSLRPEACRPAAGGAVHTRGLDFACD

The CD8 stalk has a sequence such that it may induce the formation of homodimers. If this is not desired, one or more cysteine residues may be substituted or removed from the CD8 stalk sequence.

The bi-specific T cell engager molecule of the invention may include a spacer which comprises or consists of the sequence shown as SEQ ID NO: 113 or 114 or a variant thereof having at least 80%, 85%, 90%, 95%, 98% or 99% sequence identity, provided that the variant sequence is a molecule which causes approximately equivalent spacing of the first and second domains and/or that the variant sequence causes homodimerisation of the bi-specific molecule.

The tri-specific T cell engager molecule of the invention may include one or more spacers which comprises or consists of the sequence shown as SEQ ID NO: 113 or 114 or a variant thereof having at least 80%, 85%, 90%, 95%, 98% or 99% sequence identity, provided that the variant sequence is a molecule which causes approximately equivalent spacing of the first and second domains and/or second and third domains and/or that the variant sequence causes homodimerisation of the tri-specific molecule.

The spacer may also comprise one or more linker motifs to introduce a chain-break. A chain break separate two distinct domains but allows orientation in different angles. Such sequences include the sequence SDP, and the sequence SGGGSDP (SEQ ID NO: 115).

The bi-specific T cell engager molecule of the invention or the tri-specific T cell engager molecule of the invention may include a linker which comprises a serine-glycine linker. The linker may be composed of several modules of serine-glycine, such as such as SGGGGS (SEQ ID NO: 116), GGGGS (SEQ ID NO: 117), ((Gly₄)Ser)₂ (GGGGSGGGGS, SEQ ID NO: 355), ((Gly₄)Ser)₃ (GGGGSGGGGSGGGGS, SEQ ID NO: 356) and ((Gly₄)Ser)₄ (GGGGGGGGSGGGGSGGGGS, SEQ ID NO: 357).

The bi-specific T cell engager molecule of the invention may have the general formula:

Signal peptide-first domain-spacer/linker-second domain.

The tri-specific T cell engager molecule of the invention may have the general formula:

Signal peptide-first domain-spacer/linker-second domain-spacer/linker-third domain.

### Gamma Delta Cell Engagers

The immune cell engager of the present invention may be a molecule that engages a gamma delta T cell. Examples of gamma delta cells include Vγ9Vδ2 T cells.

The second domain of the gamma delta T cell engager may bind CD3 as described herein.

The second domain of the gamma delta T cell engager may bind to the Vγ9 chain of the Vγ9Vδ2+ γδ T cell receptor. Suitably antibodies capable of binding to Vγ9Vδ2 T cells are described in WO2020/159368, for example.

### NKT Cell Engagers

An immune cell engager of the present invention may be a molecule that engages a NKT cell.

The second domain of the NKT T cell engager may bind CD3 as described herein.

### Bi-Specific Killer Cell Engagers (Bikes) and Tri-Specific Killer Cell Engagers (Trikes)

In one embodiment, the present invention provides a NK cell engager molecule which is a bi-specific killer cell engager molecule (BIKE) which comprises a sdAb as described herein as a first domain, and a NK cell activating domain as a second domain. A NK cell activating domain is a domain capable of activating a NK cell.

The BIKE or TRIKE may comprise multiple copies of the second domain, and thus may encompass multiple copies of a NK cell activating domain. The BIKE or TRIKE may comprise 1, 2, 3, 4, 5 or more of the same NK cell activating domain. For example, the NK cell engager may comprise 3 CD16-binding domains which each bind to CD16.

Bi-specific killer cell engaging molecules are a class of bi-specific antibody-type molecules that have been developed, primarily for the use as anti-cancer drugs. They direct a host's immune system, more specifically the NK cells' cytotoxic activity, against a target cell, such as a cancer cell. In these bi-specific killer cell engager molecules, one binding domain binds to a NK cell via, for example, the CD16 receptor, and the other to a target cell such as a tumour cell (e.g. via a tumour specific molecule). Since the bi-specific killer cell engager molecule binds both the target cell and the NK cell, it brings the target cell into proximity with the NK cell, so that the NK cell can exert its effect, for example, a cytotoxic effect on a cancer cell. The formation of the NK cell: bi-specific Ab: cancer cell complex induces signalling in the NK cell leading to, for example, the release of cytotoxic mediators. Ideally, the agent only induces the desired signalling in the presence of the target cell, leading to selective killing.

Thus, a NK cell engager of the present invention brings a FcRH5-expressing cell (for example, a FcRH5+ cancer cell) into proximity with a NK cell, so that the NK cell can exert its effect on the cancer cell. The requirement of co-localisation via binding of the FcRH5 bi-specific killer cell engager molecule leads to selective killing of FcRH5-positive cells. In other words, a NK cell engager molecule of the present invention is able to activate NK cells following binding of the NK cell engager molecule to FcRH5 expressed on the surface of target cells.

Suitably, the second domain activates a NK cell by binding CD16 on the NK cell surface.

Suitably, the second domain comprises a CD16-specific antibody or part thereof.

The second domain may comprise an antibody or part thereof which specifically binds CD16, such as 3G8 or LSIV21 or affinity tuned variants thereof.

The second domain of the NK cell engager molecule of the invention may comprise one or more CDRs from murine 3G8. The second binding domain may comprise CDR3 from the heavy-chain of murine 3G8 and/or CDR3 from the light chain of murine 3G8. The second binding domain may comprise all 6 CDRs from murine 3G8, as shown below.

Heavy Chain
CDR1:
(SEQ ID NO: 136)
GFSLRTSGMG
CDR2:
(SEQ ID NO: 137)
IWWDDDK
CDR3:
(SEQ ID NO: 138)
AQINPAWFAY
Light Chain
CDR1:
(SEQ ID NO: 139)
QSVDFDGDSF
CDR2:
(SEQ ID NO: 140)
TT
CDR3:
(SEQ ID NO: 141)
QQSNEDPYT

Suitably, one or more of the CDRs may comprise comprises one, two or three amino acid mutations.

The second domain of the NK cell engager molecule may comprise a scFv which comprises the CDR sequences from murine 3G8.

The second domain of the NK cell engager molecule may comprise a VH region having the sequence shown as SEQ ID NO: 142 or a variant having at least 80%, 85%, 90%, 95%, 98% or 99% sequence identity thereto which retains the capacity to bind CD16; and a VL region having the sequence shown as SEQ ID NO: 143 or a variant of having at least 80%, 85%, 90%, 95%, 98% or 99% sequence identity thereto which retains the capacity to bind CD16. Suitably, the third binding domain may comprise a VH region having the sequence shown as SEQ ID NO: 142; and a VL region having the sequence shown as SEQ ID NO: 143.

VH domain
SEQ ID NO: 142
QVTLKESGPGILQPSQTLSLTCSFSGFSLRTSGMGVGWIRQPSGK
GLEWLAHIWWDDDKRYNPALKSRLTISKDTSSNQVFLKIASVDTA
DTATYYCAQINPAWFAYWGQGTLVTVSA
VL domain
SEQ ID NO: 143
DIVLTQSPASLAVSLGQRATISCKASQSVDFDGDSFMNWYQQKPG
QPPKLLIYTTSNLESGIPARFSASGSGTDFTLNIHPVEEEDTATY
YCQQSNEDPYTFGGGTKLELK

The second domain of the NK cell engager molecule of the invention may comprise one or more CDRs from humanised 3G8. The second binding domain may comprise CDR3 from the heavy-chain of humanised 3G8 and/or CDR3 from the light chain of humanised 3G8. The second binding domain may comprise all 6 CDRs from humanised 3G8, as shown below.

### Heavy Chain

CDR1:
(SEQ ID NO: 144)
GFSLSTSGMG
CDR2:
(SEQ ID NO: 137)
IWWDDDK
CDR3:
(SEQ ID NO: 145)
ARINPAWFAY
Light Chain
CDR1:
(SEQ ID NO: 139)
QSVDFDGDSF
CDR2:
(SEQ ID NO: 140)
TT
CDR3:
(SEQ ID NO: 141)
QQSNEDPYT

Suitably, one or more of the CDRs may comprise comprises one, two or three amino acid mutations.

The second domain of the NK cell engager molecule may comprise a scFv which comprises the CDR sequences from humanised 3G8.

The second domain of the NK cell engager molecule may comprise a VH region having the sequence shown as SEQ ID NO: 146 or a variant having at least 80%, 85%, 90%, 95%, 98% or 99% sequence identity thereto which retains the capacity to bind CD16; and a VL region having the sequence shown as SEQ ID NO: 147 or a variant of having at least 80%, 85%, 90%, 95%, 98% or 99% sequence identity thereto which retains the capacity to bind CD16. Suitably, the third binding domain may comprise a VH region having the sequence shown as SEQ ID NO: 146; and a VL region having the sequence shown as SEQ ID NO: 147.

VH domain
SEQ ID NO: 146
QVTLKESGPALVKPTQTLTLTCTFSGFSLSTSGMGVGWIRQPPGK
ALEWLAHIWWDDDKRYNPALKSRLTISKDTSKNQVVLTMTNMDPV
DTATYYCARINPAWFAYWGQGTLVTVSS
VL domain
SEQ ID NO: 147
DIVMTQSPDSLAVSLGERATINCKASQSVDFDGDSFMNWYQQKPG
QPPKLLIYTTSNLESGVPDRFSGSGSGTDFTLTIRPLQAEDVAVY
YCQQSNEDPYTFGQGTKLEIK

The second domain of the NK cell engager molecule of the invention may comprise one or more CDRs from LSIV21. The second binding domain may comprise CDR3 from the heavy-chain of LSIV21 and/or CDR3 from the light chain of LSIV21. The second binding domain may comprise all 6 CDRs from murine LSIV21, as shown below.

### Heavy Chain

CDR1:
(SEQ ID NO: 124)
GYTFTSYY
CDR2:
(SEQ ID NO: 148)
INPSGGST
CDR3:
(SEQ ID NO: 149)
ARGSAYYYDFADY
Light Chain
CDR1:
(SEQ ID NO: 150)
NIGSKN
CDR2:
(SEQ ID NO: 151)
QD
CDR3:
(SEQ ID NO: 152)
QVWDNYSVL

Suitably, one or more of the CDRs may comprise comprises one, two or three amino acid mutations.

The second domain of the NK cell engager molecule may comprise a scFv which comprises the CDR sequences from LSIV21.

The second domain of the NK cell engager molecule may comprise a VH region having the sequence shown as SEQ ID NO: 153 or a variant having at least 80%, 85%, 90%, 95%, 98% or 99% sequence identity thereto which retains the capacity to bind CD16; and a VL region having the sequence shown as SEQ ID NO: 154 or a variant of having at least 80%, 85%, 90%, 95%, 98% or 99% sequence identity thereto which retains the capacity to bind CD16. Suitably, the third binding domain may comprise a VH region having the sequence shown as SEQ ID NO: 153; and a VL region having the sequence shown as SEQ ID NO: 154.

VH domain
SEQ ID NO: 153
QVQLVQSGAEVKKPGESLKVSCKASGYTFTSYYMHWVRQAPGQGL
EWMGIINPSGGSTSYAQKFQGRVTMTRDTSTSTVYMELSSLRSED
TAVYYCARGSAYYYDFADYWGQGTLVTVSS
VL domain
SEQ ID NO: 154
SYVLTQPSSVSVAPGQTATISCGGHNIGSKNVHWYQQRPGQSPVL
VIYQDNKRPSGIPERFSGSNSGNTATLTISGTQAMDEADYYCQVW
DNYSVLFGGGTKLTVL

The present invention also provides a NK cell engager which is a tri-specific killer cell engager molecule. In addition to the first and second domains described herein for BiKEs, a TriKE additionally comprises another NK cell activating domain as a third domain. A NK cell activating domain is a domain capable of activating a NK cell. Accordingly, a TriKE may have a similar effect and function as a BIKE as described herein.

A third domain of a TriKE may be a domain that provides an additional activation signal to a NK cell.

Suitably, the third domain is capable of cross-linking IL-15.

BiKEs and TriKEs are commonly made by fusing an anti-CD16 scFv to an anti-target antigen scFv via a short five residue peptide linker (GGGGS (SEQ ID NO: 117)).

The BiKE of the invention or TriKE of the invention may comprise a spacer or linker sequence as described in the context of the BiTE of the invention or the tri-specific T cell engager molecule of the invention to connect the first domain with the second domain and/or to connect the second domain with the third domain in order to spatially separate the domains.

The BIKE of the invention or TriKE of the invention may comprise a signal peptide as described herein to aid in its production.

The bi-specific NK cell engager molecule of the invention may have the general formula:

Signal peptide-first domain-spacer/linker-second domain.

The tri-specific NK cell engager molecule of the invention may have the general formula:

Signal peptide-first domain-spacer/linker-second domain-spacer/linker-third domain.

### Polynucleotide

In an aspect the present invention provides a nucleic acid sequence which encodes a sdAb of the present invention.

In one aspect the present invention provides a nucleic acid sequence which encodes a CAR of the present invention.

In one aspect the present invention provides a nucleic acid sequence which encodes an immune cell engager molecule of the present invention.

Due to the redundancy of the genetic code, variations in nucleic acid sequences are possible that encode for the same polypeptide. These sequences are encompassed by the present invention. Therefore multiple polynucleotides are envisaged, each with a different nucleic acid sequence but which encodes a polypeptide according to the invention or a further polypeptide as described herein. It is possible to design and produce such nucleic acid sequences without difficulty.

The nucleic acid sequence may be an RNA or DNA sequence or a variant thereof. The term “polynucleotide” includes an RNA or DNA sequence. It may be single or double stranded. It may, for example, be genomic, recombinant, mRNA or cDNA.

The nucleotide sequence may be codon optimised for production in the host cell of choice.

As used herein, “variant” is synonymous with “mutant” and refers to a polynucleotide or amino acid sequence which differs in comparison to the corresponding wild-type sequence. The term “wild-type” is used to mean a gene or protein having a polynucleotide or amino acid sequence respectively, which is identical with the native gene or protein respectively.

Suitably, the nucleic acid sequence may be operably linked to a heterologous sequence, such as a promoter or regulatory sequence, forming an expression cassette.

The expression cassette may comprise one or more control sequences. Control sequences are sequences that control and regulate transcription and, where appropriate, the translation of said sdAb, CAR or immune cell engager, and include promoter sequences, transcriptional regulators encoding sequences, ribosome binding sequences (RBS) and/or transcription terminating sequences. The expression cassette of the present invention may additionally include an enhancer, which may be adjacent to or distant from the promoter sequence and can function to increase transcription from the same. The expression control sequence may be functional in prokaryotic cells or in eukaryotic cells and organisms, such as mammalian cells. The expression cassette may comprise a promoter. Any promoter may be used in this methodology. In general, it is advantageous to employ a strong promoter functional in eukaryotic cells. The strong promoter may be, but not limited to, the immediate early cytomegalovirus promoter (CMV-IE) of human or murine origin, or optionally having another origin such as the rat or guinea pig.

In more general terms, the promoter has either a viral, or a cellular origin. A strong viral promoter other than CMV-IE that may be usefully employed in the practice of the invention is the early/late promoter of the SV40 virus or the LTR promoter of the Rous sarcoma virus. A strong cellular promoter that may be usefully employed in the practice of the invention is the promoter of a gene of the cytoskeleton, such as e.g. the desmin promoter (Kwissa et al., 2000), or the actin promoter (Miyazaki et al., 1989).

The promoter may be constitutive promoter. The promoter may be a tissue specific promoter.

### Vector

The present invention also provides a vector which comprises a nucleic acid sequence according to the present invention. For example, the vector of the invention may comprise a polynucleotide comprising a nucleic acid sequence that encodes a FcRH5 binding molecule of the invention, such as an sdAb of the invention, or a CAR of the invention, or an immune cell cell engager molecule of the invention. Such a vector may be used to introduce the nucleic acid sequence into a host cell so that it expresses and produces a FcRH5 binding molecule of the invention.

The vector may be any agent capable of delivering or maintaining nucleic acid in a host cell, and includes viral vectors, plasmids, naked nucleic acids, nucleic acids complexed with polypeptide or other molecules and nucleic acids immobilised onto solid phase particles. The vector may, for example, be a plasmid or a viral vector, such as a retroviral vector or a lentiviral vector.

The vector may comprise the nucleic acid sequence encoding the sdAb, CAR, bi-specific T cell engager molecule, tri-specific T cell engager molecule, bi-specific killer cell engager molecule, or tri-specific killer cell engager molecule according to the invention, operably linked to a heterologous sequence, such as a promoter or regulatory sequence. In general, it is advantageous to employ a strong promoter functional in eukaryotic cells. The strong promoter may be, but not limited to, the immediate early cytomegalovirus promoter (CMV-IE) of human or murine origin, or optionally having another origin such as the rat or guinea pig.

In more general terms, the promoter has either a viral, or a cellular origin. A strong viral promoter other than CMV-IE that may be usefully employed in the practice of the invention is the early/late promoter of the SV40 virus or the LTR promoter of the Rous sarcoma virus. A strong cellular promoter that may be usefully employed in the practice of the invention is the promoter of a gene of the cytoskeleton, such as e.g. the desmin promoter (Kwissa et al., 2000), or the actin promoter (Miyazaki et al., 1989).

The promoter may be constitutive promoter. The promoter may be a tissue specific promoter.

The vector may be capable of transfecting or transducing a cell. In one aspect, the vector may be capable of transfecting or transducing a T cell or an NK cell.

The vector may also comprise a nucleic acid sequence encoding a suicide gene, such as iCasp9 or RQR8.

### Cell

The invention also provides a host cell which comprises a nucleic acid or a vector according to the invention.

The host cell may be capable of producing an sdAb of the invention. The host cell may be capable of producing an immune cell engager of the invention. The host cell may be capable of producing and/or expressing and/or may comprise a CAR of the invention.

The host cell may be a bacterial, fungal, yeast, plant or animal cell. Suitably the sdAb or immune cell engager of the invention may be produced in a bacterial, fungal, yeast, plant or animal cell. Suitably, the host cell may be a mammalian cell, such as the human embryonic kidney cell line 293.

The cell may be any eukaryotic cell capable of expressing a CAR at the cell surface, such as an immunological cell. In particular, the cell may be an immune cell such as a T cell or NK cell.

T cells or T lymphocytes are a type of lymphocyte that play a central role in cell-mediated immunity. They can be distinguished from other lymphocytes, such as B cells and natural killer cells (NK cells), by the presence of a T-cell receptor (TCR) on the cell surface. There are various types of T cell, as summarised below.

Helper T helper cells (TH cells) assist other white blood cells in immunologic processes, including maturation of B cells into plasma cells and memory B cells, and activation of cytotoxic T cells and macrophages. TH cells express CD4 on their surface. TH cells become activated when they are presented with peptide antigens by MHC class II molecules on the surface of antigen presenting cells (APCs). These cells can differentiate into one of several subtypes, including TH1, TH2, TH3, TH17, Th9, or TFH, which secrete different cytokines to facilitate different types of immune responses.

Cytotoxic T cells (TC cells, or CTLs) destroy virally infected cells and tumor cells, and are also implicated in transplant rejection. CTLs express the CD8 at their surface. These cells recognize their targets by binding to antigen associated with MHC class I, which is present on the surface of all nucleated cells. Through IL-10, adenosine and other molecules secreted by regulatory T cells, the CD8+ cells can be inactivated to an anergic state, which prevent autoimmune diseases such as experimental autoimmune encephalomyelitis.

Memory T cells are a subset of antigen-specific T cells that persist long-term after an infection has resolved. They quickly expand to large numbers of effector T cells upon re-exposure to their cognate antigen, thus providing the immune system with “memory” against past infections. Memory T cells comprise three subtypes: central memory T cells (TCM cells) and two types of effector memory T cells (TEM cells and TEMRA cells). Memory cells may be either CD4+ or CD8+. Memory T cells typically express the cell surface protein CD45RO.

Regulatory T cells (Treg cells), formerly known as suppressor T cells, are crucial for the maintenance of immunological tolerance. Their major role is to shut down T cell-mediated immunity toward the end of an immune reaction and to suppress auto-reactive T cells that escaped the process of negative selection in the thymus.

Two major classes of CD4+ Treg cells have been described—naturally occurring Treg cells and adaptive Treg cells.

Naturally occurring Treg cells (also known as CD4+CD25+FoxP3+ Treg cells) arise in the thymus and have been linked to interactions between developing T cells with both myeloid (CD11c+) and plasmacytoid (CD123+) dendritic cells that have been activated with TSLP. Naturally occurring Treg cells can be distinguished from other T cells by the presence of an intracellular molecule called FoxP3. Mutations of the FOXP3 gene can prevent regulatory T cell development, causing the fatal autoimmune disease IPEX.

Adaptive Treg cells (also known as Tr1 cells or Th3 cells) may originate during a normal immune response.

Gamma delta T cells (γδ T cells) are T cells that have a TCR that comprised of one γ (gamma) chain and one δ (delta) chain. Gamma delta T cells are typically less common than αβ T cells. In humans, in 95% of T cells the TCR consists of an alpha (α) chain and a beta (β) chain (encoded by TRA and TRB, respectively). However, in about 5% of T cells the TCR consists of gamma and delta (γ/δ) chains (encoded by TRG and TRD, respectively). Gamma delta T cells are abundant in the gut mucosa. Examples of gamma delta cells include Vγ9Vδ2 T cells.

γδ TCRs are MHC independent and may detect markers of cellular stress expressed by tumours. The γδ TCR may be capable of binding to a phosphoantigen/butyrophilin 3A1 complex; major histocompatibility complex class I chain-related A (MICA); major histocompatibility complex class I chain-related B (MICB); NKG2D ligand 1-6 (ULBP 1-6); CD1c; CD1d; endothelial protein C receptor (EPCR); lipohexapeptides; phycoreythrin or histidyl-tRNA-synthase.

Natural killer T (NKT) cells are a heterogeneous group of T cells that share properties of both T cells and natural killer cells. Many of these cells recognize the non-polymorphic CD1d molecule, an antigen-presenting molecule that binds self and foreign lipids and glycolipids.

The cell of the invention may be any of the T cell types mentioned above, in particular a CTL.

Natural killer (NK) cells are a type of cytolytic cell which forms part of the innate immune system. NK cells provide rapid responses to innate signals from virally infected cells in an MHC independent manner.

NK cells (belonging to the group of innate lymphoid cells) are defined as large granular lymphocytes (LGL) and constitute the third kind of cells differentiated from the common lymphoid progenitor generating B and T lymphocytes. NK cells are known to differentiate and mature in the bone marrow, lymph node, spleen, tonsils and thymus where they then enter into the circulation.

The CAR cells of the invention may be any of the cell types mentioned above.

Suitably the T cell or NK cell may produce and/or express and/or comprise the CAR of the invention.

Suitably, the host cell may be a T cell. Suitably, the host cell may be a NK cell.

A T cell capable of expressing a CAR according to the invention may be made by transducing or transfecting a T cell with CAR-encoding nucleic acid. A NK cell capable of expressing a CAR according to the invention may be made by transducing or transfecting a NK cell with a CAR-encoding nucleic acid.

Cells may either be created ex vivo either from a patient's own peripheral blood (1st party), or in the setting of a haematopoietic stem cell transplant from donor peripheral blood (2nd party), or peripheral blood from an unconnected donor (3rd party). The cell may be from a peripheral blood mononuclear cell (PBMC) sample from the patient or a donor.

Alternatively, cells may be derived from ex vivo differentiation of inducible progenitor cells or embryonic progenitor cells to, for example, T or NK cells. Alternatively, an immortalized T-cell line which retains its lytic function and could act as a therapeutic may be used.

The cells may be activated and/or expanded prior to being transduced with the CAR-encoding nucleic acid, for example by treatment with an anti-CD3 monoclonal antibody.

The present invention also provides a method for making a cell according to the invention, which comprises the step of introducing a polynucleotide according to the invention or a vector according to the invention into said cell. The polynucleotide or vector may, for example, be introduced by transduction or transfection in vitro or ex vivo.

Said cell is then capable of expressing and/or producing an sdAb of the invention, or a CAR of the invention, or an immune cell engager of the invention when the host cell is cultured under conditions suitable for production of the molecule. The molecule can then be harvested from the host cell or supernatant.

Antibodies and immune cell engagers of the invention produced in a cell as set out above can be produced either intracellularly (e.g. in the cytosol, in the periplasma or in inclusion bodies) and then isolated from the host cells and optionally further purified; or they can be produced extracellularly (e.g. in the medium in which the host cells are cultured) and then isolated from the culture medium and optionally further purified.

### Pharmaceutical Composition

The present invention also relates to a pharmaceutical composition comprising a polynucleotide or a vector of the invention together with a pharmaceutically acceptable carrier, diluent or excipient, and optionally one or more further pharmaceutically active polypeptides and/or compounds.

The present invention also relates to a pharmaceutical composition comprising an sdAb of the invention together with a pharmaceutically acceptable carrier, diluent or excipient, and optionally one or more further pharmaceutically active polypeptides and/or compounds.

The present invention also relates to a pharmaceutical composition comprising an antibody conjugate of the invention together with a pharmaceutically acceptable carrier, diluent or excipient, and optionally one or more further pharmaceutically active polypeptides and/or compounds.

The present invention also relates to a pharmaceutical composition comprising a CAR-expressing cell of the invention together with a pharmaceutically acceptable carrier, diluent or excipient, and optionally one or more further pharmaceutically active polypeptides and/or compounds.

The present invention also relates to a pharmaceutical composition comprising an immune cell engager of the invention together with a pharmaceutically acceptable carrier, diluent or excipient, and optionally one or more further pharmaceutically active polypeptides and/or compounds.

The pharmaceutical composition may be administered to a patient.

The pharmaceutical composition may enable delivery and/or maintenance of a polynucleotide of the invention in a host cell or subject. Such delivery suitably enables expression of an sdAb, or a further product of the invention, in a host cell or subject. The polynucleotide may be DNA, RNA, or mRNA. The pharmaceutical composition may comprise a viral vector, plasmid, naked nucleic acid, nucleic acid complexed with polypeptide or other molecules and nucleic acids immobilised onto solid phase particles. The vector may, for example, be a plasmid or a viral vector, such as a retroviral vector or a lentiviral vector. Other delivery agents include delivery nanoparticles and lipid nanoparticles (LNP).

The form of administration may be for example, be in a form suitable for oral administration, or for parenteral administration (such as by intravenous, intramuscular or subcutaneous injection or intravenous infusion), or intratumorally.

Suitably, such formulations may, for example, be in a form suitable for intravenous infusion.

### Medical Use

The present invention provides an sdAb of the invention for use as a medicament in the treatment of a disease.

The present invention provides an antibody conjugate of the invention for use as a medicament in the treatment of a disease.

The present invention provides a cell of the invention expressing a CAR of the invention for use as a medicament in the treatment of a disease.

The present invention provides an immune cell engager of the invention for use as a medicament in the treatment of a disease.

The present invention provides a polynucleotide of the invention for use as a medicament in the treatment of a disease.

The present invention provides a vector of the invention for use as a medicament in the treatment of a disease.

The present invention provides a pharmaceutical composition of the invention for use as a medicament in the treatment of a disease.

Suitably the disease may be a disease associated with FcRH5 expression. FcRH5 is known to be expressed on differentiated B cells and plasma cells.

Suitably the disease may be a cancerous disease, in particular a cancerous disease associated with FcRH5 expression.

The disease may be a B cell malignancy.

The disease may be leukaemia or non-Hodgkins lymphoma.

The leukaemia may be hairy cell leukaemia or chronic lymphocytic leukaemia.

The non-Hodgkins lymphoma may be mantle cell lymphoma, EBV-associated lymphoma (Burkitt) or lymphoplasmacytic lymphoma.

Suitably the disease may be a plasma cell disorder or plasma cell dyscrasias, in particular a plasma cell disorder or plasma cell dyscrasias associated with FcRH5 expression.

The disease may be a plasma cell disorder or plasma cell dyscrasias selected from: plasmacytoma, solitary plasmocytoma, extramedullary plasmocytoma, plasma cell leukemia, multiple myeloma, macroglobulinemia, amyloidosis, Waldenstrom's macroglobulinemia, solitary bone plasmacytoma, extramedullary plasmacytoma, osteosclerotic myeloma, heavy chain diseases, monoclonal gammopathy of undetermined significance (MGUS), non-IgM MGUS, IgM MGUS, light chain MGUS and smoldering multiple myeloma.

The disease may be multiple myeloma.

Suitably, the sdAb of the invention, the antibody conjugate of the invention, the cell expressing a CAR of the invention, the immune cell engager of the invention, the polynucleotide of the invention, the vector of the invention and/or the pharmaceutical composition of the invention may be used for the treatment of a cancerous disease associated with FcRH5 expression.

Suitably, the sdAb of the invention, the antibody conjugate of the invention, the cell expressing a CAR of the invention, the immune cell engager of the invention, the polynucleotide of the invention, the vector of the invention and/or the pharmaceutical composition of the invention may be used for the treatment of multiple myeloma.

Cells expressing a CAR molecule of the present invention are capable of killing cancer cells. CAR-expressing cells may either be created ex vivo either from a patient's own peripheral blood (1st party), or in the setting of a haematopoietic stem cell transplant from donor peripheral blood (2nd party), or peripheral blood from an unconnected donor (3rd party). Alternatively, CAR cells may be derived from ex-vivo differentiation of inducible progenitor cells or embryonic progenitor cells to T cells or NK cells. In these instances, CAR cells are generated by introducing DNA or RNA coding for the CAR by one of many means including transduction with a viral vector, transfection with DNA or RNA.

The present invention also relates to a method for the treatment of a disease which comprises the step of administering the sdAb of the invention, the antibody conjugate of the invention, the cell expressing a CAR of the invention, the immune cell engager of the invention, the polynucleotide of the invention, the vector of the invention and/or the pharmaceutical composition of the invention to a subject.

In this respect, the sdAb, antibody conjugate, CAR-expressing cell, immune cell engager polynucleotide, vector and/or pharmaceutical composition may be administered to a subject having an existing disease or condition in order to lessen, reduce or improve at least one symptom associated with the disease and/or to slow down, reduce or block the progression of the disease. For example, the method of the invention may cause or promote T cell or NK cell mediated killing of FcRH5-expressing cells, such as cancer cells.

The present invention also relates to the use of the sdAb of the invention, the antibody conjugate of the invention, the cell expressing a CAR of the invention, the immune cell engager of the invention, the polynucleotide of the invention, the vector of the invention and/or the pharmaceutical composition of the invention in the manufacture of a medicament for treating a disease.

### Aspects of the Invention

Further aspects of the invention are described in the following numbered paragraphs (paras):

1. A single domain antibody (sdAb) comprising a FcRH5 binding domain.

2. The sdAb according to para 1 wherein the FcRH5 binding domain comprises:

(i)
complementarity determining regions (CDRs)
with the following sequences:
CDR1-
(SEQ ID NO: 1)
RSSFSNNA
CDR2-
(SEQ ID NO: 2)
ITKGGVT
CDR3-
(SEQ ID NO: 3)
NTIPFRSA;
or
(ii)
CDRs with the following sequences:
CDR1-
(SEQ ID NO: 4)
GFTFSNYA
CDR2-
(SEQ ID NO: 5)
INSDGGTA
CDR3-
(SEQ ID NO: 6)
AANRGFCAGVRCLEYQY;
or
(iii)
CDRs with the following sequences:
CDR1-
(SEQ ID NO: 7)
GRTFSINA
CDR2-
(SEQ ID NO: 8)
IGGSGRVSST
CDR3-
(SEQ ID NO: 9)
AARRDYLPFPPESYDY;
or
(iv)
CDRs with the following sequences:
CDR1-
(SEQ ID NO: 7)
GRTFSINA
CDR2-
(SEQ ID NO: 8)
IGGSGRVSST
CDR3-
(SEQ ID NO: 10)
AAGRRTSTNGGDYDY;
or
(v)
CDRs with the following sequences:
CDR1-
(SEQ ID NO: 7)
GRTFSINA
CDR2-
(SEQ ID NO: 11)
ISRSGGAT
CDR3-
(SEQ ID NO: 12)
AGTRRAFSTGLRDYDY;
or
(vi)
CDRs with the following sequences:
CDR1-
(SEQ ID NO: 13)
GRTFSNST
CDR2-
(SEQ ID NO: 14)
ISWSGGTY
CDR3-
(SEQ ID NO: 15)
AAARKGWSTRGDDYDY;
or
(vii)
CDRs with the following sequences:
CDR1-
(SEQ ID NO: 16)
GRTYNNYA
CDR2-
(SEQ ID NO: 17)
ISRSGGMT
CDR3-
(SEQ ID NO: 18)
AAYVGGFSTARRDYSY;
or
(viii)
CDRs with the following sequences:
CDR1-
(SEQ ID NO: 19)
GRTFSSYA
CDR2-
(SEQ ID NO: 20)
ISRIGGVT
CDR3-
(SEQ ID NO: 21)
AAAGLVSISTTPNDYDY;
or
(ix)
CDRs with the following sequences:
CDR1-
(SEQ ID NO: 22)
GNIFRLNG
CDR2-
(SEQ ID NO: 23)
ITSGGNT
CDR3-
(SEQ ID NO: 24)
NAIPFRL;
or
(x)
CDRs with the following sequences:
CDR1-
(SEQ ID NO: 25)
RNIFSLNP
CDR2-
(SEQ ID NO: 26)
ITDGGST
CDR3-
(SEQ ID NO: 27)
NRVGGLQTWA;
or
(xi)
CDRs with the following sequences:
CDR1-
(SEQ ID NO: 28)
GRSFSNYG
CDR2-
(SEQ ID NO: 29)
IGMVGGLT
CDR3-
(SEQ ID NO: 30)
AAGRRFSTSSRDYDI;
or
(xii)
CDRs with the following sequences:
CDR1-
(SEQ ID NO: 31)
GRTFDSRP
CDR2-
(SEQ ID NO: 32)
VSWRGEST
CDR3-
(SEQ ID NO: 33)
AAGEPYSGTYYYRGRDYDY;
or
(xiii)
CDRs with the following sequences:
CDR1-
(SEQ ID NO: 34)
GRTFSMYA
CDR2-
(SEQ ID NO: 35)
ISGSARIT
CDR3-
(SEQ ID NO: 36)
AASSTYTSTSGSSYNY;
or
(xiv)
CDRs with the following sequences:
CDR1-
(SEQ ID NO: 37)
GSIFSINA
CDR2-
(SEQ ID NO: 38)
ITSGGST
CDR3-
(SEQ ID NO: 39)
NALGGFVPSYG;
or
(xv)
CDRs with the following sequences:
CDR1-
(SEQ ID NO: 155)
GRTSSRAA
CDR2-
(SEQ ID NO: 156)
ISWSGGTT
CDR3-
(SEQ ID NO: 157)
AAARIFTTARNDYDH;
or
(xvi)
CDRs with the following sequences:
CDR1-
(SEQ ID NO: 22)
GNIFRLNG
CDR2-
(SEQ ID NO: 23)
ITSGGNT
CDR3-
(SEQ ID NO: 186)
NTIPFRLS;
or
(xvii)
CDRs with the following sequences:
CDR1-
(SEQ ID NO: 160)
GNIFRLNA
CDR2-
(SEQ ID NO: 23)
ITSGGNT
CDR3-
(SEQ ID NO: 186)
NTIPFRLS;
or
(xviii)
CDRs with the following sequences:
CDR1-
(SEQ ID NO: 1)
RSSFSNNA
CDR2-
(SEQ ID NO: 2)
ITKGGVT
CDR3-
(SEQ ID NO: 187)
NTIPVRSA;
or
(xix)
CDRs with the following sequences:
CDR1-
(SEQ ID NO: 37)
GSIFSINA
CDR2-
(SEQ ID NO: 2)
ITKGGVT
CDR3-
(SEQ ID NO: 188)
NTIPCRSA;
or
(xx)
CDRs with the following sequences:
CDR1-
(SEQ ID NO: 161)
GSSFRLNG
CDR2-
(SEQ ID NO: 38)
ITSGGST
CDR3-
(SEQ ID NO: 189)
NTIPFSRA;
or
(xxi)
CDRs with the following sequences:
CDR1-
(SEQ ID NO: 162)
GRSVSINA
CDR2-
(SEQ ID NO: 176)
IDRSGNT
CDR3-
(SEQ ID NO: 190)
NTIPYSDS;
or
(xxii)
CDRs with the following sequences:
CDR1-
(SEQ ID NO: 19)
GRTFSSYA
CDR2-
(SEQ ID NO: 177)
IDGIGGIT
CDR3-
(SEQ ID NO: 3)
NTIPFRSA;
or
(xxiii)
CDRs with the following sequences:
CDR1-
(SEQ ID NO: 1)
RSSFSNNA
CDR2-
(SEQ ID NO: 2)
ITKGGVT
CDR3-
(SEQ ID NO: 191)
NAIPFRSA;
or
(xxiv)
CDRs with the following sequences:
CDR1-
(SEQ ID NO: 163)
GNNFRLNA
CDR2-
(SEQ ID NO: 23)
ITSGGNT
CDR3-
(SEQ ID NO: 192)
NAIPFRPS;
or
(xxv)
CDRs with the following sequences:
CDR1-
(SEQ ID NO: 22)
GNIFRLNG
CDR2-
(SEQ ID NO: 23)
ITSGGNT
CDR3-
(SEQ ID NO: 193)
NAIPFRRS;
or
(xxvi)
CDRs with the following sequences:
CDR1-
(SEQ ID NO: 164)
ERIFRINA
CDR2-
(SEQ ID NO: 23)
ITSGGNT
CDR3-
(SEQ ID NO: 193)
NAIPFRRS;
or
(xxvii)
CDRs with the following sequences:
CDR1-
(SEQ ID NO: 165)
GFTFSTYW
CDR2-
(SEQ ID NO: 178)
IDNGGGTT
CDR3-
(SEQ ID NO: 194)
NALPFRLS;
or
(xxviii)
CDRs with the following sequences:
CDR1-
(SEQ ID NO: 22)
GNIFRLNG
CDR2-
(SEQ ID NO: 23)
ITSGGNT
CDR3-
(SEQ ID NO: 195)
NATPFRLS;
or
(xxix)
CDRs with the following sequences:
CDR1-
(SEQ ID NO: 22)
GNIFRLNG
CDR2-
(SEQ ID NO: 179)
ITRGGNT
CDR3-
(SEQ ID NO: 196)
NSIPFRLS;
or
(xxx)
CDRs with the following sequences:
CDR1-
(SEQ ID NO: 166)
GNIFRING
CDR2-
(SEQ ID NO: 23)
ITSGGNT
CDR3-
(SEQ ID NO: 197)
NAIPFRIS;
or
(xxxi)
CDRs with the following sequences:
CDR1-
(SEQ ID NO: 163)
GNNFRLNA
CDR2-
(SEQ ID NO: 23)
ITSGGNT
CDR3-
NAIPFRLY;
(SEQ ID NO: 198)
or
(xxxii)
CDRs with the following sequences:
CDR1-
(SEQ ID NO: 165)
GFTFSTYW
CDR2-
(SEQ ID NO: 178)
IDNGGGTT
CDR3-
(SEQ ID NO: 199)
KAIPFRLS;
or
(xxiii)
CDRs with the following sequences:
CDR1-
(SEQ ID NO: 167)
GSSFSNNA
CDR2-
(SEQ ID NO: 2)
ITKGGVT
CDR3-
(SEQ ID NO: 3)
NTIPFRSA;
or
(xxxiv)
CDRs with the following sequences:
CDR1-
(SEQ ID NO: 165)
GFTFSTYW
CDR2-
(SEQ ID NO: 178)
IDNGGGTT
CDR3-
(SEQ ID NO: 3)
NTIPFRSA;
or
(xxxv)
CDRs with the following sequences:
CDR1-
(SEQ ID NO: 160)
GNIFRLNA
CDR2-
(SEQ ID NO: 2)
ITKGGVT
CDR3-
(SEQ ID NO: 3)
NTIPFRSA;
or
(xxxvi)
CDRs with the following sequences:
CDR1-
(SEQ ID NO: 168)
RSSFGNNA
CDR2-
(SEQ ID NO: 2)
ITKGGVT
CDR3-
(SEQ ID NO: 3)
NTIPFRSA;
or
(xxxvii)
CDRs with the following sequences:
CDR1-
(SEQ ID NO: 169)
GRTFSTYG
CDR2-
(SEQ ID NO: 11)
ISRSGGAT
CDR3-
(SEQ ID NO: 12)
AGTRRAFSTGLRDYDY;
or
(xxxviii)
CDRs with the following sequences:
CDR1-
(SEQ ID NO: 170)
GTIERNNA
CDR2-
ITSGGST
(SEQ ID NO: 38)
CDR3-
(SEQ ID NO: 200)
AAGRRFSTRSRDYDY;
or
(xxxix)
CDRs with the following sequences:
CDR1-
(SEQ ID NO: 28)
GRSFSNYG
CDR2-
(SEQ ID NO: 29)
IGMVGGLT
CDR3-
(SEQ ID NO: 201)
AAGRRFSTSSRDYDY;
or
(xl)
CDRs with the following sequences:
CDR1-
(SEQ ID NO: 28)
GRSFSNYG
CDR2-
(SEQ ID NO: 29)
IGMVGGLT
CDR3-
(SEQ ID NO: 202)
AAGRRFSTGSRDYDI;
or
(xli)
CDRs with the following sequences:
CDR1-
(SEQ ID NO: 19)
GRTFSSYA
CDR2-
(SEQ ID NO: 179)
ISQFGGVTT
CDR3-
(SEQ ID NO: 202)
AAGRRFSTGSRDYDI;
or
(xlii)
CDRs with the following sequences:
CDR1-
(SEQ ID NO: 28)
GRSFSNYG
CDR2-
(SEQ ID NO: 29)
IGMVGGLT
CDR3-
(SEQ ID NO: 203)
AGGRRFSTSSRDYDI;
or
(xliii)
CDRs with the following sequences:
CDR1-
(SEQ ID NO: 28)
GRSFSNYG
CDR2-
(SEQ ID NO: 29)
IGMVGGLT
CDR3-
(SEQ ID NO: 204)
AAGRRFSTSSREYDI;
or
(xliv)
CDRs with the following sequences:
CDR1-
(SEQ ID NO: 28)
GRSFSNYG
CDR2-
(SEQ ID NO: 180)
IGMVGGLP
CDR3-
(SEQ ID NO: 205)
AAGRRLSTSSRDYDI;
or
(xlv)
CDRs with the following sequences:
CDR1-
(SEQ ID NO: 28)
GRSFSNYG
CDR2-
(SEQ ID NO: 181)
ISRGGGVS
CDR3-
(SEQ ID NO: 206)
AAGLRFSTGSRDYDI;
or
(xlvi)
CDRs with the following sequences:
CDR1-
(SEQ ID NO: 171)
GRTFRRYA
CDR2-
(SEQ ID NO: 17)
ISRSGGMT
CDR3-
(SEQ ID NO: 207)
AAYVGGFSTTRRDYAY;
or
(xlvii)
CDRs with the following sequences:
CDR1-
(SEQ ID NO: 13)
GRTFSNST
CDR2-
(SEQ ID NO: 156)
ISWSGGTT
CDR3-
(SEQ ID NO: 15)
AAARKGWSTRGDDYDY;
or
(xlviii)
CDRs with the following sequences:
CDR1-
(SEQ ID NO: 172)
GRTVI
CDR2-
(SEQ ID NO: 182)
SSGSGGVT
CDR3-
(SEQ ID NO: 208)
AAALTWSTRPSDFTS;
or
(xlix)
CDRs with the following sequences:
CDR1-
(SEQ ID NO: 172)
GRTVI
CDR2-
(SEQ ID NO: 183)
SNWSGGVT
CDR3-
(SEQ ID NO: 18)
AAYVGGFSTARRDYSY;
or
(l)
CDRs with the following sequences:
CDR1-
(SEQ ID NO: 7)
GRTFSINA
CDR2-
(SEQ ID NO: 17)
ISRSGGMT
CDR3-
(SEQ ID NO: 18)
AAYVGGFSTARRDYSY;
or
(li)
CDRs with the following sequences:
CDR1-
(SEQ ID NO: 173)
GRTFSRYA
CDR2-
(SEQ ID NO: 184)
INGSGGT
CDR3-
(SEQ ID NO: 209)
AAARIFTTTRNEYDH;
or
(lii)
CDRs with the following sequences:
CDR1-
(SEQ ID NO: 173)
GRTFSRYA
CDR2-
(SEQ ID NO: 184)
INGSGGT
CDR3-
(SEQ ID NO: 209)
AAARIFTTTRNEYDH;
or
(liii)
CDRs with the following sequences:
CDR1-
(SEQ ID NO: 165)
GFTFSTYW
CDR2-
(SEQ ID NO: 178)
IDNGGGTT
CDR3-
(SEQ ID NO: 210)
AAARIFSTARNDYDH;
or
(liv)
CDRs with the following sequences:
CDR1-
(SEQ ID NO: 28)
GRSFSNYG
CDR2-
(SEQ ID NO: 29)
IGMVGGLT
CDR3-
(SEQ ID NO: 211)
AGGRIFRTSSRDYDI;
or
(lv)
CDRs with the following sequences:
CDR1-
(SEQ ID NO: 22)
GNIFRLNG
CDR2-
(SEQ ID NO: 23)
ITSGGNT
CDR3-
(SEQ ID NO: 212)
AAARFFTTARNDYDH;
or
(lvi)
CDRs with the following sequences:
CDR1-
(SEQ ID NO: 37)
GSIFSINA
CDR2-
(SEQ ID NO: 38)
ITSGGST
CDR3-
(SEQ ID NO: 213)
NALGGFVPNYG;
or
(lvii)
CDRs with the following sequences:
CDR1-
(SEQ ID NO: 22)
GNIFRLNG
CDR2-
(SEQ ID NO: 23)
ITSGGNT
CDR3-
(SEQ ID NO: 213)
NALGGFVPNYG;
or
(lviii)
CDRs with the following sequences:
CDR1-
(SEQ ID NO: 37)
GSIFSINA
CDR2-
(SEQ ID NO: 38)
ITSGGST
CDR3-
(SEQ ID NO: 214)
NALGGFVPNDG;
or
(lix)
CDRs with the following sequences:
CDR1-
(SEQ ID NO: 163)
GNNFRLNA
CDR2-
(SEQ ID NO: 23)
ITSGGNT
CDR3-
(SEQ ID NO: 215)
NALGGFAPNYG;
or
(lx)
CDRs with the following sequences:
CDR1-
(SEQ ID NO: 28)
GRSFSNYG
CDR2-
(SEQ ID NO: 29)
IGMVGGLT
CDR3-
(SEQ ID NO: 216)
NALGGFVPNYV;
or
(lxi)
CDRs with the following sequences:
CDR1-
(SEQ ID NO: 174)
GRSFSINA
CDR2-
(SEQ ID NO: 38)
ITSGGST
CDR3-
(SEQ ID NO: 217)
NALGGFVLNYG;
or
(lxii)
CDRs with the following sequences:
CDR1-
(SEQ ID NO: 37)
GSIFSINA
CDR2-
(SEQ ID NO: 38)
ITSGGST
CDR3-
(SEQ ID NO: 218)
NALGGLVPNYG;
or
(lxiii)
CDRs with the following sequences:
CDR1-
(SEQ ID NO: 37)
GSIFSINA
CDR2-
(SEQ ID NO: 38)
ITSGGST
CDR3-
(SEQ ID NO: 219)
NALGGFLPNYG;
or
(lxiv)
CDRs with the following sequences:
CDR1-
(SEQ ID NO: 37)
GSIFSINA
CDR2-
(SEQ ID NO: 38)
ITSGGST
CDR3-
(SEQ ID NO: 220)
NALGGFVTNYG;
or
(lxv)
CDRs with the following sequences:
CDR1-
(SEQ ID NO: 37)
GSIFSINA
CDR2-
(SEQ ID NO: 38)
ITSGGST
CDR3-
(SEQ ID NO: 220)
NALGGFVTNYG;
or
(lxvi)
CDRs with the following sequences:
CDR1-
(SEQ ID NO: 222)
GRTV
CDR2-
(SEQ ID NO: 185)
ITRGGST
CDR3-
(SEQ ID NO: 213)
NALGGFVPNYG;
or
(lxvii)
CDRs with the following sequences:
CDR1-
(SEQ ID NO: 22)
GNIFRLNG
CDR2-
(SEQ ID NO: 23)
ITSGGNT
CDR3-
(SEQ ID NO: 39)
NALGGFVPSYG;
or
(lxviii)
CDRs with the following sequences:
CDR1-
(SEQ ID NO: 163)
GNNFRLNA
CDR2-
(SEQ ID NO: 23)
ITSGGNT
CDR3-
(SEQ ID NO: 39)
NALGGFVPSYG;
or
(lxix)
CDRs with the following sequences:
CDR1-
(SEQ ID NO: 22)
GNIFRLNG
CDR2-
(SEQ ID NO: 38)
ITSGGST
CDR3-
(SEQ ID NO: 39)
NALGGFVPSYG;
or
(lxx)
CDRs with the following sequences:
CDR1-
(SEQ ID NO: 28)
GRSFSNYG
CDR2-
(SEQ ID NO: 29)
IGMVGGLT
CDR3-
(SEQ ID NO: 39)
NALGGFVPSYG;
or
(lxxi)
CDRs with the following sequences:
CDR1-
(SEQ ID NO: 175)
GNIFSINA
CDR2-
(SEQ ID NO: 38)
ITSGGST
CDR3-
(SEQ ID NO: 39)
NALGGFVPSYG;
or
(lxxii)
CDRs with the following sequences:
CDR1-
(SEQ ID NO: 165)
GFTFSTYW
CDR2-
(SEQ ID NO: 178)
IDNGGGTT
CDR3-
(SEQ ID NO: 221)
ARNPTRGWYSTDY;

optionally wherein one or more of the CDRs comprises one, two or three amino acid mutations.

3. The sdAb according to any preceding para wherein the sdAb is a VHH or a heavy chain variable region (VH) domain.

4. The sdAb according to any preceding para, wherein the FcRH5 binding domain comprises:
(i) a variable heavy chain domain antibody (VHH) having the sequence shown as SEQ ID NO: 40 or a variant having at least 80% sequence identity thereto; or
(ii) a VHH having the sequence shown as SEQ ID NO: 41 or a variant having at least 80% sequence identity thereto; or
(iii) a VHH having the sequence shown as SEQ ID NO: 42 or a variant having at least 80% sequence identity thereto; or
(iv) a VHH having the sequence shown as SEQ ID NO: 43 or a variant having at least 80% sequence identity thereto; or
(v) a VHH having the sequence shown as SEQ ID NO: 44 or a variant having at least 80% sequence identity thereto; or
(vi) a VHH having the sequence shown as SEQ ID NO: 45 or a variant having at least 80% sequence identity thereto; or
(vii) a VHH having the sequence shown as SEQ ID NO: 46 or a variant having at least 80% sequence identity thereto; or
(viii) a VHH having the sequence shown as SEQ ID NO: 47 or a variant having at least 80% sequence identity thereto; or
(ix) a VHH having the sequence shown as SEQ ID NO: 48 or a variant having at least 80% sequence identity thereto; or
(x) a VHH having the sequence shown as SEQ ID NO: 49 or a variant having at least 80% sequence identity thereto; or
(xi) a VHH having the sequence shown as SEQ ID NO: 50 or a variant having at least 80% sequence identity thereto; or
(xii) a VHH having the sequence shown as SEQ ID NO: 51 or a variant having at least 80% sequence identity thereto; or
(xiii) a VHH having the sequence shown as SEQ ID NO: 52 or a variant having at least 80% sequence identity thereto; or
(xiv) a VHH having the sequence shown as SEQ ID NO: 53 or a variant having at least 80% sequence identity thereto; or
(xv) a VHH having the sequence shown as SEQ ID NO: 158 or a variant having at least
(xvi) a VHH having the sequence shown as SEQ ID NO: 223 or a variant having at least 80% sequence identity thereto; or
(xvii) a VHH having the sequence shown as SEQ ID NO: 224 or a variant having at least 80% sequence identity thereto; or
(xviii) a VHH having the sequence shown as SEQ ID NO: 225 or a variant having at least 80% sequence identity thereto; or
(xvix) a VHH having the sequence shown as SEQ ID NO: 226 or a variant having at least 80% sequence identity thereto; or
(xx) a VHH having the sequence shown as SEQ ID NO: 227 or a variant having at least 80% sequence identity thereto; or
(xxi) a VHH having the sequence shown as SEQ ID NO: 228 or a variant having at least 80% sequence identity thereto; or
(xxii) a VHH having the sequence shown as SEQ ID NO: 229 or a variant having at least 80% sequence identity thereto; or
(xxiii) a VHH having the sequence shown as SEQ ID NO: 230 or a variant having at least 80% sequence identity thereto; or
(xxiv) a VHH having the sequence shown as SEQ ID NO: 231 or a variant having at least 80% sequence identity thereto; or
(xxv) a VHH having the sequence shown as SEQ ID NO: 232 or a variant having at least 80% sequence identity thereto; or
(xxvi) a VHH having the sequence shown as SEQ ID NO: 233 or a variant having at least 80% sequence identity thereto; or
(xxvii) a VHH having the sequence shown as SEQ ID NO: 234 or a variant having at least 80% sequence identity thereto; or
(xxviii) a VHH having the sequence shown as SEQ ID NO: 235 or a variant having at least 80% sequence identity thereto; or
(xxix) a VHH having the sequence shown as SEQ ID NO: 236 or a variant having at least 80% sequence identity thereto; or
(xxx) a VHH having the sequence shown as SEQ ID NO: 237 or a variant having at least 80% sequence identity thereto; or
(xxxi) a VHH having the sequence shown as SEQ ID NO: 238 or a variant having at least 80% sequence identity thereto; or
(xxxii) a VHH having the sequence shown as SEQ ID NO: 239 or a variant having at least 80% sequence identity thereto; or
(xxxiii) a VHH having the sequence shown as SEQ ID NO: 240 or a variant having at least
(xxxiv) a VHH having the sequence shown as SEQ ID NO: 241 or a variant having at least 80% sequence identity thereto; or
(xxxv) a VHH having the sequence shown as SEQ ID NO: 242 or a variant having at least 80% sequence identity thereto; or
(xxxvi) a VHH having the sequence shown as SEQ ID NO: 243 or a variant having at least 80% sequence identity thereto; or
(xxxvii) a VHH having the sequence shown as SEQ ID NO: 244 or a variant having at least 80% sequence identity thereto; or
(xxxviii) a VHH having the sequence shown as SEQ ID NO: 245 or a variant having at least 80% sequence identity thereto; or
(xxxix) a VHH having the sequence shown as SEQ ID NO: 246 or a variant having at least 80% sequence identity thereto; or
(xl) a VHH having the sequence shown as SEQ ID NO: 247 or a variant having at least 80% sequence identity thereto; or
(xli) a VHH having the sequence shown as SEQ ID NO: 248 or a variant having at least 80% sequence identity thereto; or
(xlii) a VHH having the sequence shown as SEQ ID NO: 249 or a variant having at least 80% sequence identity thereto; or
(xliii) a VHH having the sequence shown as SEQ ID NO: 250 or a variant having at least 80% sequence identity thereto; or
(xliv) a VHH having the sequence shown as SEQ ID NO: 251 or a variant having at least 80% sequence identity thereto; or
(xlv) a VHH having the sequence shown as SEQ ID NO: 252 or a variant having at least 80% sequence identity thereto; or
(xlvi) a VHH having the sequence shown as SEQ ID NO: 253 or a variant having at least 80% sequence identity thereto; or
(xlvii) a VHH having the sequence shown as SEQ ID NO: 254 or a variant having at least 80% sequence identity thereto; or
(xlviii) a VHH having the sequence shown as SEQ ID NO: 255 or a variant having at least 80% sequence identity thereto; or
(xlix) a VHH having the sequence shown as SEQ ID NO: 256 or a variant having at least 80% sequence identity thereto; or
(l) a VHH having the sequence shown as SEQ ID NO: 257 or a variant having at least 80% sequence identity thereto; or
(li) a VHH having the sequence shown as SEQ ID NO: 258 or a variant having at least
(lii) a VHH having the sequence shown as SEQ ID NO: 259 or a variant having at least 80% sequence identity thereto; or
(liii) a VHH having the sequence shown as SEQ ID NO: 260 or a variant having at least 80% sequence identity thereto; or
(liv) a VHH having the sequence shown as SEQ ID NO: 261 or a variant having at least 80% sequence identity thereto; or
(lv) a VHH having the sequence shown as SEQ ID NO: 262 or a variant having at least 80% sequence identity thereto; or
(lvi) a VHH having the sequence shown as SEQ ID NO: 263 or a variant having at least 80% sequence identity thereto; or
(lvii) a VHH having the sequence shown as SEQ ID NO: 264 or a variant having at least 80% sequence identity thereto; or
(lviii) a VHH having the sequence shown as SEQ ID NO: 265 or a variant having at least 80% sequence identity thereto; or
(lix) a VHH having the sequence shown as SEQ ID NO: 266 or a variant having at least 80% sequence identity thereto; or
(lx) a VHH having the sequence shown as SEQ ID NO: 267 or a variant having at least 80% sequence identity thereto; or
(lxi) a VHH having the sequence shown as SEQ ID NO: 268 or a variant having at least 80% sequence identity thereto; or
(lxii) a VHH having the sequence shown as SEQ ID NO: 269 or a variant having at least 80% sequence identity thereto; or
(lxiii) a VHH having the sequence shown as SEQ ID NO: 270 or a variant having at least 80% sequence identity thereto; or
(lxiv) a VHH having the sequence shown as SEQ ID NO: 271 or a variant having at least 80% sequence identity thereto; or
(lxv) a VHH having the sequence shown as SEQ ID NO: 272 or a variant having at least 80% sequence identity thereto; or
(lxvi) a VHH having the sequence shown as SEQ ID NO: 273 or a variant having at least 80% sequence identity thereto; or
(lxvii) a VHH having the sequence shown as SEQ ID NO: 274 or a variant having at least 80% sequence identity thereto; or
(lxviii) a VHH having the sequence shown as SEQ ID NO: 275 or a variant having at least 80% sequence identity thereto; or
(lxix) a VHH having the sequence shown as SEQ ID NO: 276 or a variant having at least
(lxx) a VHH having the sequence shown as SEQ ID NO: 277 or a variant having at least 80% sequence identity thereto; or
(lxxi) a VHH having the sequence shown as SEQ ID NO: 278 or a variant having at least 80% sequence identity thereto; or
(lxxii) a VHH having the sequence shown as SEQ ID NO: 279 or a variant having at least 80% sequence identity thereto; or
(lxxiii) a VHH having the sequence shown as SEQ ID NO: 280 or a variant having at least 80% sequence identity thereto; or
(lxxiv) a VHH having the sequence shown as SEQ ID NO: 281 or a variant having at least 80% sequence identity thereto; or
(lxxv) a VHH having the sequence shown as SEQ ID NO: 282 or a variant having at least 80% sequence identity thereto; or
(lxxvi) a VHH having the sequence shown as SEQ ID NO: 283 or a variant having at least 80% sequence identity thereto; or
(lxxvii) a VHH having the sequence shown as SEQ ID NO: 284 or a variant having at least 80% sequence identity thereto; or
(lxxviii) a VHH having the sequence shown as SEQ ID NO: 285 or a variant having at least 80% sequence identity thereto; or
(lxxix) a VHH having the sequence shown as SEQ ID NO: 286 or a variant having at least 80% sequence identity thereto; or
(lxxx) a VHH having the sequence shown as SEQ ID NO: 287 or a variant having at least 80% sequence identity thereto; or
(lxxxi) a VHH having the sequence shown as SEQ ID NO: 288 or a variant having at least 80% sequence identity thereto.

5. The sdAb according to any preceding para wherein the sdAb is a humanised sdAb.

6. A FcRH5 binding molecule comprising the sdAb according to any preceding para, wherein the FcRH5 binding molecule is selected from an antibody conjugate, a chimeric antigen receptor (CAR), or an immune cell engager, such as a T cell engager molecule, a gamma delta T cell engager molecule, a natural killer (NK) T cell engager molecule, or a NK cell engager molecule.

7. An antibody conjugate comprising the sdAb according to any one of paras 1 to 5.

8. The antibody conjugate according to para 7, wherein the antibody conjugate may be conjugated to a chemotherapeutic entity, a radionuclide or a detection entity.

9. A CAR comprising the sdAb according to any one of paras 1 to 5.

10. The CAR according to para 9, which comprises a transmembrane domain comprising a CD8a transmembrane domain; a CD28 transmembrane domain; or a Tyrp1 transmembrane domain.

11. The CAR according to para 9 or 10, which comprises a transmembrane domain comprising one of the sequences selected from the group comprising: SEQ ID NO: 54, SEQ ID NO: 55 or SEQ ID NO: 56, or a variant thereof having at least 80% sequence identity.

12. The CAR according to any one of paras 9 to 11, wherein the sdAb and the transmembrane domain are connected by a spacer.

13. The CAR according to para 12, wherein the spacer comprises one of the following: a human an IgG1 Fc domain; an IgG1 hinge; an IgG1 hinge-CD8 stalk; or a CD8 stalk.

14. The CAR according to para 12 or 13, wherein the spacer comprises one of the sequences selected from the group comprising: SEQ ID NO: 57, SEQ ID NO: 58, SEQ ID NO: 59, SEQ ID NO: 60, or a SEQ ID NO: 61; or a variant thereof having at least 80% sequence identity.

15. The CAR according to any one of paras 9 to 14 which also comprises an intracellular T cell signalling domain.

16. The CAR according to para 15 wherein the intracellular T cell signalling domain comprises one or more of the following endodomains: CD28 endodomain; OX40 endodomain; 41BB endodomain; and CD3-Zeta endodomain.

17. The CAR according to para 15 or 16, wherein the intracellular T cell signalling domain comprises one or more of the sequences selected from the group comprising: SEQ ID NO: 67, SEQ ID NO: 68, SEQ ID NO: 69, or SEQ ID NO: 70; or a variant thereof having at least 80% sequence identity.

18. The CAR according to any one of paras 9 to 17, which comprises the sequence selected from the group comprising SEQ ID NO: 76-SEQ ID NO: 89, SEQ ID NO: 159 and SEQ ID NO: 289-SEQ ID NO: 354, or a variant thereof which has at least 80% sequence identity thereto but retains the capacity to i) bind FcRH5 and ii) induce T cell signalling.

19. An immune cell engager molecule which comprises:
(i) a first domain which comprises a sdAb according to any one of paras 1 to 5; and
(ii) a second domain capable of activating a T cell.

20. The immune cell engager molecule according to para 19 further comprising:
(iii) a third domain capable of binding a T cell.

21. The immune cell engager molecule according to para 19 or 20, wherein the second domain activates a T cell by binding CD3 on the T cell surface.

22. The immune cell engager molecule according to para 21, wherein the second domain comprises a CD3-specific antibody or part thereof.

23. The immune cell engager molecule according to any one of paras 19 to 22, wherein the second domain comprises the sequence selected from the group comprising SEQ ID NO: 96, SEQ ID NO: 103 or SEQ ID NO: 110 or a variant thereof which has at least 80% sequence identity and binds CD3.

24. The immune cell engager molecule according to any of paras 19 to 23, wherein the first and second binding domains are connected by a spacer or a linker.

25. The immune cell engager molecule according to any of paras 19 to 24, wherein the second and third binding domains are connected by a spacer or a linker.

26. The immune cell engager molecule according to para 24 or 25 wherein the spacer comprises an IgG1 hinge or a CD8 stalk.

27. The immune cell engager molecule according to any one of para 24 to 26, wherein the spacer comprises one of the sequences selected from the group consisting of: SEQ ID NO: 113 or SEQ ID NO: 114; or a variant thereof having at least 80% sequence identity.

27a. The immune cell engager molecule according to para 24 or 25 wherein the linker comprises one of the sequences selected from the group consisting of SGGGGS (SEQ ID NO: 116), GGGGS (SEQ ID NO: 117), ((Gly4)Ser)2 (GGGGSGGGGS, SEQ ID NO: 355), ((Gly4)Ser)3 (GGGGGGGGSGGGGS, SEQ ID NO: 356) and ((Gly4)Ser)4 (GGGGGGGGSGGGGSGGGGS, SEQ ID NO: 357).

28. The immune cell engager molecule according to para 20, wherein the third domain is capable of binding CD28.

29. The immune cell engager molecule according to para 28, wherein the third domain comprises a CD28-specific antibody or part thereof.

30. The immune cell engager molecule according to any one of paras 28 to 29, wherein the third domain comprises
(i) a VH region having the sequence shown as SEQ ID NO: 130 and a VL region having the sequence shown as SEQ ID NO: 131; or
(ii) a VH region having the sequence shown as SEQ ID NO: 132 and a VL region having the sequence shown as SEQ ID NO: 133; or
(iii) a VH region having the sequence shown as SEQ ID NO: 134 and a VL region having the sequence shown as SEQ ID NO: 135;
or a variant thereof which has at least 80% sequence identity and binds CD28.

31. An immune cell engager molecule which comprises:
(i) a first domain which comprises a sdAb according to any one of paras 1 to 5; and
(ii) a second domain capable of activating a NK cell.

32. The immune cell engager molecule according to para 31 further comprising:
(ii) a third domain capable of activating a NK cell.

33. The immune cell activator molecule according to para 31 or 32, wherein the second domain activates a NK cell by binding CD16 on the NK cell surface.

34. The immune cell activator molecule according to para 33 wherein the second domain comprises a CD16-specific antibody or part thereof; optionally wherein the second domain comprises:
(i) a VH region having the sequence shown as SEQ ID NO: 142 and a VL region having the sequence shown as SEQ ID NO: 143; or
(ii) a VH region having the sequence shown as SEQ ID NO: 146 and a VL region having the sequence shown as SEQ ID NO: 147; or
(iii) a VH region having the sequence shown as SEQ ID NO: 153 and a VL region having the sequence shown as SEQ ID NO: 154;
or a variant thereof which has at least 80% identity and binds CD16.

35. An immune cell activator molecule according to para 32, wherein the third domain is capable of cross-linking IL-15.

36. A polynucleotide comprising a nucleic acid sequence encoding an sdAb according to any one of paras 1 to 5, a CAR according to any one of paras 9 to 18, a immune cell engager molecule according to any one of paras 19 to 30, or a immune cell engager molecule according to any of paras 31 to 35.

37. A vector which comprises a polynucleotide according to para 36.

38. A cell which comprises a CAR according to any of paras 9 to 18.

39. A cell comprising the polynucleotide according to para 36 or a vector according to para 37.

40. The cell according to para 38 or 39 wherein the cell is a T cell or a NK cell.

41. A method for making a cell according to any one of paras 38 to 40, which comprises the step of introducing a polynucleotide according to para 36 or a vector according to para 37 into said cell.

42. A pharmaceutical composition which comprises a sdAb according to any one of paras 1 to 5, or an antibody conjugate according to para 7 or 8, or an immune cell engager molecule according to any of paras 19 to 30, or an immune cell engager molecule according to any of paras 31 to 35, or a polynucleotide according to para 36, or a vector according to para 37, or a cell according to any one of paras 38 to 40, together with a pharmaceutically acceptable carrier, diluent or excipient.

43. A sdAb according to any one of paras 1 to 5, or an antibody conjugate according to para 7 or 8, or an immune cell engager molecule according to any of paras 19 to 30, or an immune cell engager molecule according to any of paras 31 to 35, or a vector according to para 37, or a cell according to any one of paras 38 to 40, or a pharmaceutical composition according to para 42 for use as a medicament in the treatment of a disease.

44. A method for treating a disease which comprises the step of administering a sdAb according to any one of paras 1 to 5, or an antibody conjugate according to para 7 or 8, or an immune cell engager according to any of paras 19 to 30, or an immune cell engager molecule according to any of paras 31 to 35, or a vector according to para 37, or a cell according to any one of paras 38 to 40, or a pharmaceutical composition according to para 42 to a subject.

45. A use of a sdAb according to any one of paras 1 to 5, or an antibody conjugate according to para 7 or 8, or an immune cell engager molecule according to any of paras 19 to 30, or an immune cell engager molecule according to any of paras 31 to 35, or a vector according to para 37, or a cell according to any one of paras 38 to 40, or a pharmaceutical composition according to para 42 in the manufacture of a medicament for treating a disease.

46. The sdAb, or the antibody conjugate, or the immune cell engager, or the immune cell engager molecule, or the vector, or the cell, or the pharmaceutical composition for use according to para 43, or the method according to para 44; or the use according to para 45; wherein the disease is a B cell malignancy or a plasma cell disorder.

47. The sdAb, or the antibody conjugate, or the immune cell engager molecule, or the immune cell engager molecule, or the vector, or the cell, or the pharmaceutical composition for use; the method; or the use according to para 46, wherein the a B cell malignancy or plasma cell disorder is selected from the list comprising: leukaemia, hairy cell leukaemia, chronic lymphocytic leukaemia, non-Hodgkins lymphoma, mantle cell lymphoma, EBV-associated lymphoma (Burkitt), lymphoplasmacytic lymphoma, plasmacytoma, solitary plasmocytoma, extramedullary plasmocytoma, plasma cell leukemia, multiple myeloma, macroglobulinemia, amyloidosis, Waldenstrom's macroglobulinemia, solitary bone plasmacytoma, extramedullary plasmacytoma, osteosclerotic myeloma, heavy chain diseases, monoclonal gammopathy of undetermined significance (MGUS), non-IgM MGUS, IgM MGUS, light chain MGUS and smoldering multiple myeloma.

48. The sdAb, or the antibody conjugate, or the immune cell engager molecule, or the immune cell engager molecule, or the vector, or the cell, or the pharmaceutical composition for use; the method; or the use according to para 47 wherein the cancer is multiple myeloma. This disclosure is not limited by the exemplary methods and materials disclosed herein, and any methods and materials similar or equivalent to those described herein can be used in the practice or testing of embodiments of this disclosure. Numeric ranges are inclusive of the numbers defining the range. Unless otherwise indicated, any nucleic acid sequences are written left to right in 5′ to 3′ orientation; amino acid sequences are written left to right in amino to carboxy orientation, respectively.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within this disclosure. The upper and lower limits of these smaller ranges may independently be included or excluded in the range, and each range where either, neither or both limits are included in the smaller ranges is also encompassed within this disclosure, subject to any specifically excluded limit in the stated range. Where the stated range includes one or both of the limits, ranges excluding either or both of those included limits are also included in this disclosure.

It must be noted that as used herein and in the appended claims, the singular forms “a”, “an”, and “the” include plural referents unless the context clearly dictates otherwise.

The terms “comprising”, “comprises” and “comprised of” as used herein are synonymous with “including”, “includes” or “containing”, “contains”, and are inclusive or open-ended and do not exclude additional, non-recited members, elements or method steps. The terms “comprising”, “comprises” and “comprised of” also include the term “consisting of”.

The publications discussed herein are provided solely for their disclosure prior to the filing date of the present application. Nothing herein is to be construed as an admission that such publications constitute prior art to the claims appended hereto.

The invention will now be further described by way of Examples, which are meant to serve to assist one of ordinary skill in the art in carrying out the invention and are not intended in any way to limit the scope of the invention.

### EXAMPLES

The constructs used in the following experiments in the Examples can be identified by the following CDR1-3 and VHH sequences:

TABLE 2
Constructs and corresponding sequences

| AU ID# | CDR1 | CDR2 | CDR3 | VHH |
|---|---|---|---|---|
| 60369 | RSSFSNNA | ITKGGVT | NTIPFRSA | QVQLQESGGGLVQAGGSLRLSCAASRSS |
| | | | | FSNNAMGWYRQVPGKQRELVAFITKGGV |
| | | | | TDYSDSVKGRFTISKDNAKNTVYLQMNSL |
| | | | | KPEDTAVYYCNTIPFRSAWGQGTQVTVS |
| 60370 | GFTFSNYA | INSDGGTA | AANRGFCAGVRCLEYQY | QVQLQESGGGSVQPGGSLSLSCAASGFT |
| | | | | FSNYAMSWVRQAPGKGPEWVAVINSDG |
| | | | | GTASSAGSVRGRFTISRDNAKNTLYLQMN |
| | | | | RLKPEDTAVYYCAANRGFCAGVRCLEYQ |
| | | | | YWGQGTQVTVS |
| 60382 | GRTFSINA | IGGSGRVSST | AARRDYLPFPPESYDY | QVQLQESGGGLVQAGGSLIVSCAASGRT |
| | | | | FSINAMAWFRQAPGKEREFVAAIGGSGR |
| | | | | VSSTSYADFVKGRFTISRDNAKNTVYLRM |
| | | | | NNLEPEDTAVYYCAARRDYLPFPPESYDY |
| | | | | WGQGTQVTVA |
| 60385 | ISGFGVVT | IGGSGRVSST | AAGRRTSTNGGDYDY | QVQLQESGGGLVQAGGSLRLSCAASGRT |
| | | | | FSTYAMAWFRQAPGKEREFVAAISGFGV |
| | | | | VTYYADSVKGRFTISRDNAKNTLYLQMNG |
| | | | | LKPEDTAVYYCAAGRRTSTNGGDYDYWG |
| | | | | QGTQVTVS |
| 60387 | GRTFSTYG | ISRSGGAT | AGTRRAFSTGLRDYDY | QVQLQESGGGLVQAGGSLRLSCAASGRT |
| | | | | FSTYGMGWFRQAPGKEREFVAAISRSGG |
| | | | | ATAYAASVKGRFTISRDDVKNTLYLQMNS |
| | | | | LKPEDTAVYHCAGTRRAFSTGLRDYDYW |
| | | | | GQGIQVTVS |
| 60390 | GRTFSNST | ISWSGGTY | AAARKGWSTRGDDYDY | QVQLQQSGGGLVQAGDSLRLSCAASGRT |
| | | | | FSNSTMGWFRQAPGKERKFVAVISWSGG |
| | | | | TYAYAESVKGRFTISRDNAKNTVYLQMNS |
| | | | | LKPEDTAVYYCAAARKGWSTRGDDYDY |
| | | | | WGQGTQVTVS |
| 60460 | GRTYNNYA | ISRSGGMT | AAYVGGFSTARRDYSY | QVQLQESGGGLVQAGGSLRLSCAASGRT |
| | | | | YNNYAMGWFRQAPGKEREFVAGISRSG |
| | | | | GMTGYAESVKGRFTISRDNAKNMVFLQM |
| | | | | NSLKPEDTAVYYCAAYVGGFSTARRDYS |
| | | | | YWGQGTQVTVS |
| 60462 | GRTSSRAA | ISWSGGTT | AAARIFTTARNDYDH | QVQLQESGGGLVQAGGSLRLSCTASGRT |
| | | | | SSRAAMGWFRQAPGKEREFVAVISWSGG |
| | | | | TTAYANSVKGRFTISRDNAKNTLYLQMN |
| | | | | SLKPEDTAVYYCAAARIFTTARNDYDHWG |
| | | | | QGTQVTVS |
| 60463 | GRTFSSYA | ISRIGGVT | AAAGLVSISTTPNDYDY | QVQLQESGGGLVQAGDSLRLSCAYSGRT |
| | | | | FSSYAMGWFRQAPGKERVFVAAISRIGG |
| | | | | VTTYAESVQGRFTISRDNAKNTLYLQMNA |
| | | | | LKPEDTAVYYCAAAGLVSISTTPNDYDYW |
| | | | | GQGTQVTVS |
| 60464 | GNIFRLNG | ITSGGNT | NAIPFRL | QVQLQQSGGGLVQPGGSLRLSCAAPGNI |
| | | | | FRLNGTGWYRQAPGKQRELVAHITSGGN |
| | | | | TDYADSVKGRFTISRDNAKNTVYLQMNSL |
| | | | | KPEDTAVYYCNAIPFRLSWGQGTQVTVS |
| 60465 | RNIFSLNP | ITDGGST | NRVGGLQTWA | QVQLQESGGGLVQAGGSLRLSCVVSRNI |
| | | | | FSLNPMGWYRQAPGKQREMVAIITDGGS |
| | | | | TNYADSVKGRFTISRDNVKNTVYLQMNAL |
| | | | | EPEDTAVYYCNRVGGLQTWAWGQGTQV |
| | | | | TVSS |
| 60466 | GRSFSNYG | IGMVGGLT | AAGRRFSTSSRDYDI | QVQLQQSGGGLVQAGGSLSLSCTASGRS |
| | | | | FSNYGMGWFRQAPGKEREFVAAIGMVG |
| | | | | GLTAYSNSAKGRFTISRDNAKNTLYLQMN |
| | | | | SLKPEDTAVYLCAAGRRFSTSSRDYDIWG |
| | | | | QGTQVTVS |
| 60467 | GRTFDSRP | VSWRGEST | AAGEPYSGTYYYRGRDYD | QVQLQESGGGLVQAGDSLRLSCAASGRT |
| | | | Y | FDSRPMGWFRQAPGKEREFVGAVSWRG |
| | | | | ESTYYPDSVKGRFTISRDNAKRTVYLQMN |
| | | | | SLKPEDTAVYYCAAGEPYSGTYYYRGRD |
| | | | | YDYWGQGTQVTVS |
| 60470 | GRTFSMYA | ISGSARIT | AASSTYTSTSGSSYNY | QVQLQESGGGLVQAEGSLRLSCAASGRT |
| | | | | FSMYAMGWFRQAPGREREFVAAISGSAR |
| | | | | ITYYGQSVKGRFTISRDNAKNTVYLQMNS |
| | | | | LKPEDTAVYYCAASSTYTSTSGSSYNYW |
| | | | | GQGTQVTVS |
| 60471 | GSIFSINA | ITSGGST | NALGGFVPSYG | QVQLQESGGGLVQAGGSLRLSCAASGSI |
| | | | | FSINAMGWYRQAPGKQRELVAFITSGGST |
| | | | | NYADSVKGRFTISRDNAKNTVYLQMNSLK |
| | | | | PEDTAVYYCNALGGFVPSYGWGQGTQV |
| | | | | TVS |

### Example 1—Generation and Biophysical Characterisation of Anti-FcRH5 Binding Domains

Table 3 details the DNA constructs and cell lines used in this Example.

TABLE 3
List of constructs in study

| AU Plasmid | | |
|---|---|---|
| Number | Plasmid name | Description |
| AU60369 | AbVec.aFcRH5.dAb-MIgG2a-Fc.Clone.B03 | Anti-FcRH5 |
| AU60370 | AbVec.aFcRH5.dAb-MIgG2a-Fc.Clone.B04 | Anti-FcRH5 |
| AU60382 | AbVec.aFcRH5.dAb-MIgG2a-Fc.Clone.B07 | Anti-FcRH5 |
| AU60385 | AbVec.aFcRH5.dAb-MIgG2a-Fc.Clone.B07 | Anti-FcRH5 |
| AU60387 | AbVec.aFcRH5.dAb-MIgG2a-Fc.Clone.C01 | Anti-FcRH5 |
| AU60390 | AbVec.aFcRH5.dAb-MIgG2a-Fc.Clone.C06 | Anti-FcRH5 |
| AU60460 | AbVec.aFcRH5.dAb-MIgG2a-Fc.Clone.C10 | Anti-FcRH5 |
| AU60462 | AbVec.aFcRH5.dAb-MIgG2a-Fc.Clone.E04 | Anti-FcRH5 |
| AU60463 | AbVec.aFcRH5.dAb-MIgG2a-Fc.Clone.E07 | Anti-FcRH5 |
| AU60464 | AbVec.aFcRH5.dAb-MIgG2a-Fc.Clone.E11 | Anti-FcRH5 |
| AU60465 | AbVec.aFcRH5.dAb-MIgG2a-Fc.Clone.E12 | Anti-FcRH5 |
| AU60466 | AbVec.aFcRH5.dAb-MIgG2a-Fc.Clone.F01 | Anti-FcRH5 |
| AU60467 | AbVec.aFcRH5.dAb-MIgG2a-Fc.Clone.F03 | Anti-FcRH5 |
| AU60470 | AbVec.aFcRH5.dAb-MIgG2a-Fc.Clone.F11 | Anti-FcRH5 |
| AU60471 | AbVec.aFcRH5.dAb-MIgG2a-Fc.Clone.F12 | Anti-FcRH5 |

Anti-FcRH5 VHH-Fc carrying a murine IgG2a Fc domain, were expressed by transient transfection in expi-CHO cells and purified with Protein A. The biophysical properties of these binders were characterised.

### Differential Scanning Fluorimetry to Determine Thermal Stability

Purified antibodies were loaded onto glass capillaries (Nanotemper) in duplicate. Scanning at 330 and 350 nm was performed using Prometheus NT.48 instrument (Nanotemper) with a temperature ramp of 1° C./min from 20 to 95° C. Melting temperature (Tm) calculated as first derivative of 350 nm/330 nm ratio.

### Results

Unfolding was monitored from 20 to 95° C. with 1° C./min ramp. Data represented as First derivative of 350 nm/330 nm ratio (Table 4).

TABLE 4
Thermal stability (Tm)

| Clone | Tm (° C.) |
|---|---|
| 60369 | 69.76 |
| 60370 | 68.67 |
| 60382 | 70.34 |
| 60385 | 66.95 |
| 60387 | 76.65 |
| 60390 | 70.71 |
| 60460 | 72.90 |
| 60462 | 74.38 |
| 60463 | 74.34 |
| 60464 | 59.67 |
| 60465 | 62.57 |
| 60466 | 61.20 |
| 60467 | 62.33 |
| 60470 | 64.11 |
| 60471 | 69.76 |

### Multi-Angle Dynamic Light Scattering to Determine Aggregation

Aggregation propensity and average particle size of the test proteins was determined using a Zetasizer Ultra device and ZS Xplorer software (Malvern Panalytical) by MADLS. Samples were loaded into a low volume quartz cuvette (Malvern Panalytical-ZEN2112) at a concentration of 1 mg/ml. Triplicate measurements were taken for each sample.

### Results

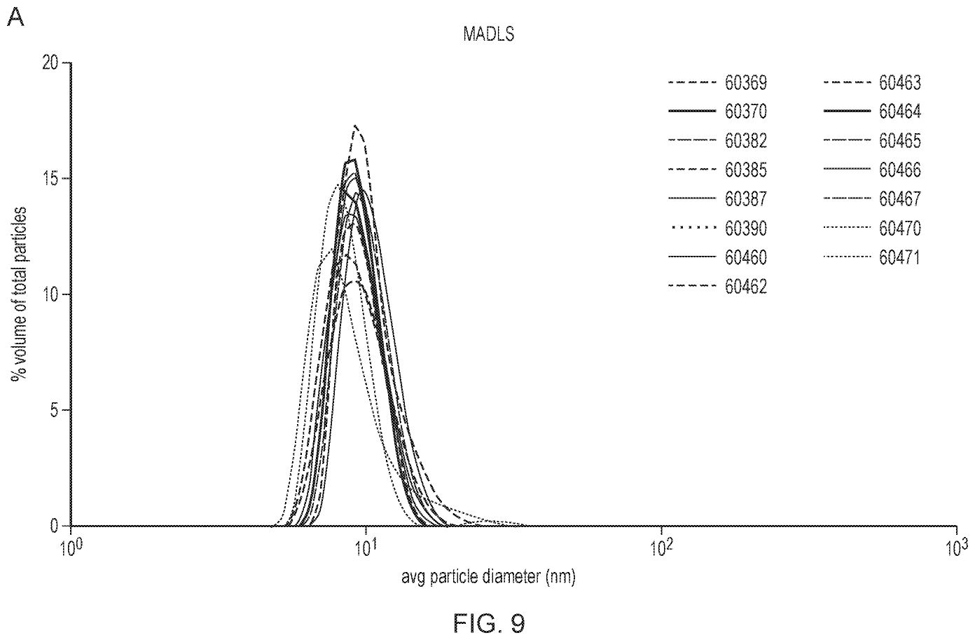

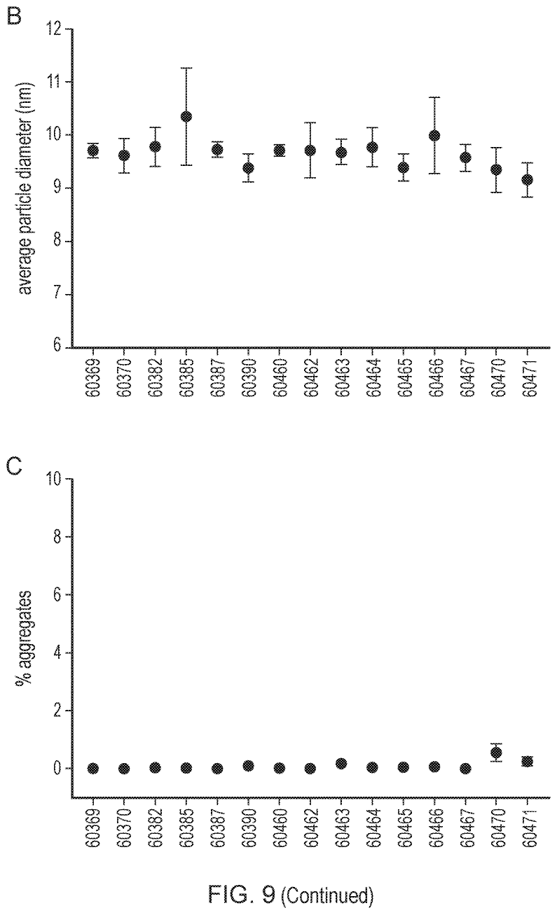

Particle dispersion and aggregation profile of purified anti-FcRH5 antibodies was determined using multi-angle dynamic light scattering (MADLS) on a Zetasizer Ultra instrument. Antibodies were tested at 1 mg/ml in PBS at pH 7.4. All antibodies tested showed a preferentially monodispersed profile with average particle diameter in line with a predicted molecular weight of 82 kDa (FIG. 9).

### Surface Plasmon Resonance (SPR)

Recombinant anti-FcRH5 antibodies in mouse IgG2a format were captured on flow cell 2 on a Series S CM5 chip (GE Healthcare), functionalised with anti-mouse capture kit according to manufacturer's recommendations (GE Healthcare). Antibodies were captured to a density of 160 RU using a Biacore 8k instrument. HBS-P+ buffer was used as running buffer in all experimental conditions. Recombinant purified FcRH5 Ig-8 domain at known concentrations (500 nM with 2-fold serial dilutions) used as the ‘analyte’ and injected over the respective flow cells with 150s contact time and 500s dissociation at 30 μl/minute of flow rate with a constant temperature of 25° C. In each experiment, flow cell 1 was unmodified and used for reference subtraction. A ‘0 concentration’ sensogram of buffer alone was used as a double reference subtraction to factor for drift. Data were fit to a 1:1 Langmuir binding model. Since a capture system was used, a local maximal analytical response (Rmax) parameter was used for the data fitting in each case.

### Results

Binding affinity and kinetic profile of anti-FcRH5 VHH for target antigen FcRH5 Ig8 domain was determined via surface plasmon resonance on a Biacore 8k instrument.

TABLE 5
SPR kinetic measurements

| | Analyte | 1:1 binding ka | kd | KD |
|---|---|---|---|---|
| Clone | Solution | (1/Ms) | (1/s) | (M) |
| 60369 | FcRH5 Ig8 | 5.16e+5 | 2.46e−3 | 4.77e−9 |
| 60370 | FcRH5 Ig8 | 7.68e+4 | 2.80e−4 | 3.64e−9 |
| 60382 | FcRH5 Ig8 | 1.19e+5 | 5.15e−4 | 4.34e−9 |
| 60385 | FcRH5 Ig8 | 5.31e+4 | 3.48e−4 | 6.56e−9 |
| 60387 | FcRH5 Ig8 | 4.86e+4 | 1.15e−8* | 2.36e−13* |
| 60390 | FcRH5 Ig8 | 5.00e+4 | 4.97e−8* | 9.93e−13* |
| 60460 | FcRH5 Ig8 | 5.08e+4 | 3.40e−5 | 6.69e−10 |
| 60462 | FcRH5 Ig8 | 5.05e+4 | 2.93e−4 | 5.80e−9 |
| 60463 | FcRH5 Ig8 | 5.38e+4 | 1.61e−4 | 2.99e−9 |
| 60464 | FcRH5 Ig8 | 5.96e+5 | 2.89e−3 | 4.84e−9 |
| 60465 | FcRH5 Ig8 | 6.73e+5 | 3.06e−3 | 4.54e−9 |
| 60466 | FcRH5 Ig8 | 1.50e+5 | 4.69e−4 | 3.14e−9 |
| 60467 | FcRH5 Ig8 | N/A | N/A | N/A |
| 60470 | FcRH5 Ig8 | 7.08e+4 | 1.81e−5 | 2.55e−10 |
| 60471 | FcRH5 Ig8 | 3.30e+5 | 2.50e−4 | 7.57e−10 |

*kd outside instrument limits

### Example 2—Anti-FcRH5 VHH-CAR Activation Assay Via NUR77 Upregulation in Jurkat T Cell Line

### Method

### Constructs and Cell Lines

Table 6 details the VHH DNA ID's, corresponding CAR constructs and viral supernatants used in this Example.

TABLE 6
List of constructs in study

| VHH-Fc (AU#) | CAR construct (AU#) | Viral Supernatant (AU#) |
|---|---|---|
| AU60369 | 66841 | 85026 |
| AU60370 | 66842 | 85027 |
| AU60382 | 66843 | 85028 |
| AU60385 | 66823 | 85014 |
| AU60387 | 66825 | 85015 |
| AU60390 | 66828 | 85016 |
| AU60460 | 66829 | 85017 |
| AU60462 | 66831 | 85018 |
| AU60463 | 66832 | 85019 |
| AU60464 | 66833 | 85020 |
| AU60465 | 66834 | 85021 |
| AU60466 | 66835 | 85022 |
| AU60467 | 66836 | 85023 |
| AU60470 | 66839 | 85024 |
| AU60471 | 66840 | 85025 |

### Car Construction

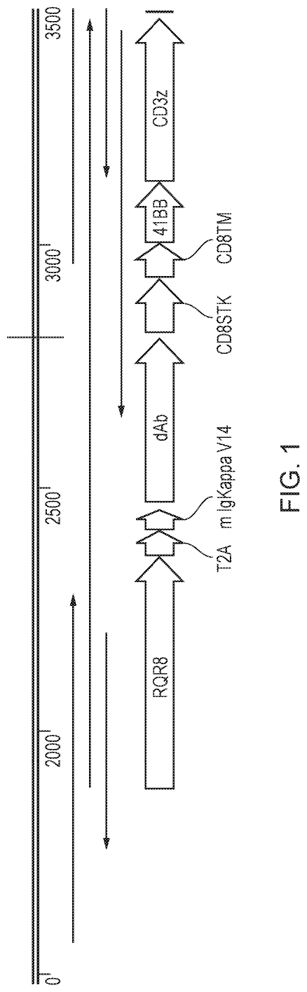

VHH's were cloned into a gamma retroviral vector encompassing a CD8 spacer and transmembrane region with a 41BB and CD3z chain intracellular signalling domain along with an RQR8 transduction marker separated by a 2A cleavage peptide (FIG. 1).

### Plate Bound Antigen CAR Stimulation Assay

Sterile ELISA plates were coated with 100 μl target antigen in PBS overnight at 4° C. with top concentration of 10 μg/ml and 1:2 or 1:5 serial dilutions were performed 6 times; wells were also coated with PBS only for background control. On the day of experiment, coated ELISA plates were washed 3× with PBS prior to use, and transduced cells were counted and prepared at a concentration of 8×10⁵ cells/ml. Cells were plated at 8×10⁴ cells/well (100 μl cell suspension) and returned to incubator for 24 h and 72 h.

### Flow Cytometry

Cells from the ELISA plates were collected and transferred into a U-Bottom plate. Cells were centrifuged at 1000 g for 2 minutes and supernatants were discarded. Cells were stained with anti-CD34 antibody (1:100 dilution) in the dark at room temperature for 20 minutes and washed twice with PBS to remove any remaining unbound antibodies. Cells were resuspended with 100 μl Sytox dead cell stain (1:1000 dilution) and analysed on FACS machine to assess for mClover expression on transduced cells. The gating strategy are as followed: cells were gated on singlets, live cells, and cell activation were assessed based on RQR8 and mClover expression.

### Results

Experiment 1_Preliminary Nur-77/mClover Activation Assay (10, 2 and 0.4 μg/ml Antigen)

### Activation of NUR77 Pathway by Plate Bound Antigen

Plate bound antigen exposure (assay described above) can be used to assess CAR activation in a controlled manner. Here, 3 concentrations of antigen (5fold diluted) were used to assess CAR activation using a fluorescent mClover linked NUR77 as proxy for stimulated CAR T cells. The assay was performed at 24 h and 72 h post activation.

24 h Antigen Exposure (10, 2, 0.4 μg/ml Coating)

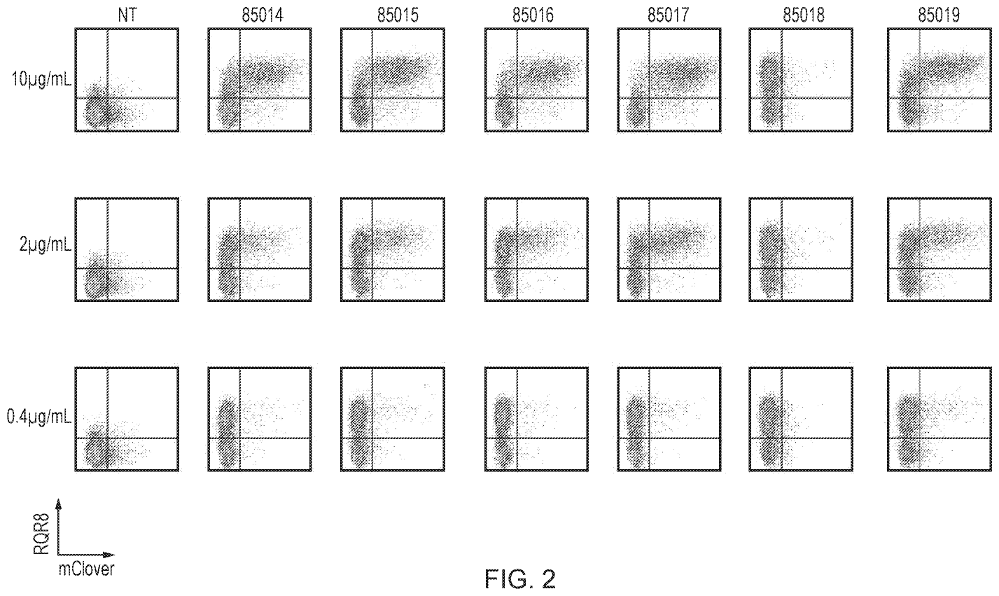

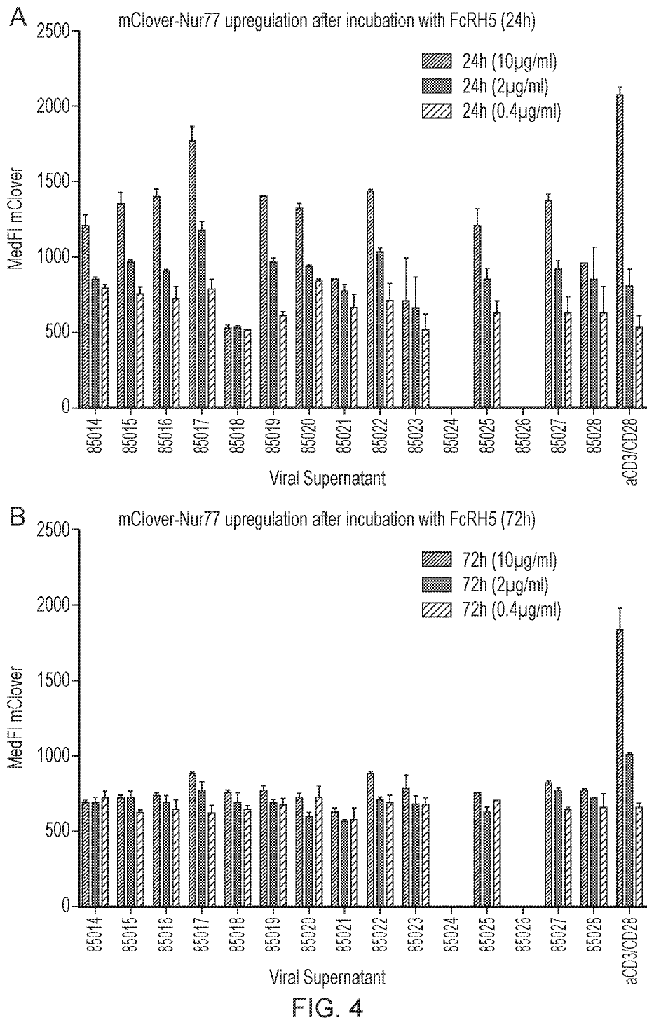

All transduced Jurkat T cells underwent upregulation of mClover-NUR77 in response to plate bound FcRH5 at 10 μg/ml after 24 h exposure other than cells transduced with supes 85018, 85021 and 85023 (FIG. 2). There was a concentration dependent reduction in mClover detection from 10 μg/ml down to 0.4 μg/ml with a maximum antigen stimulation of 45.45% (aCD3/CD28 positive control 78.98) (FIG. 4).

72 h Antigen Exposure (10, 2, 0.4 μg/ml Coating)

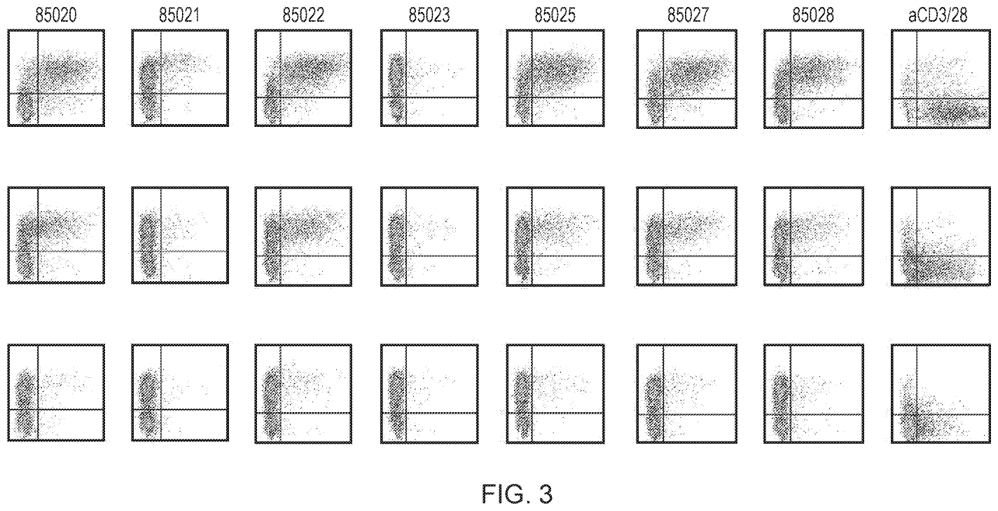

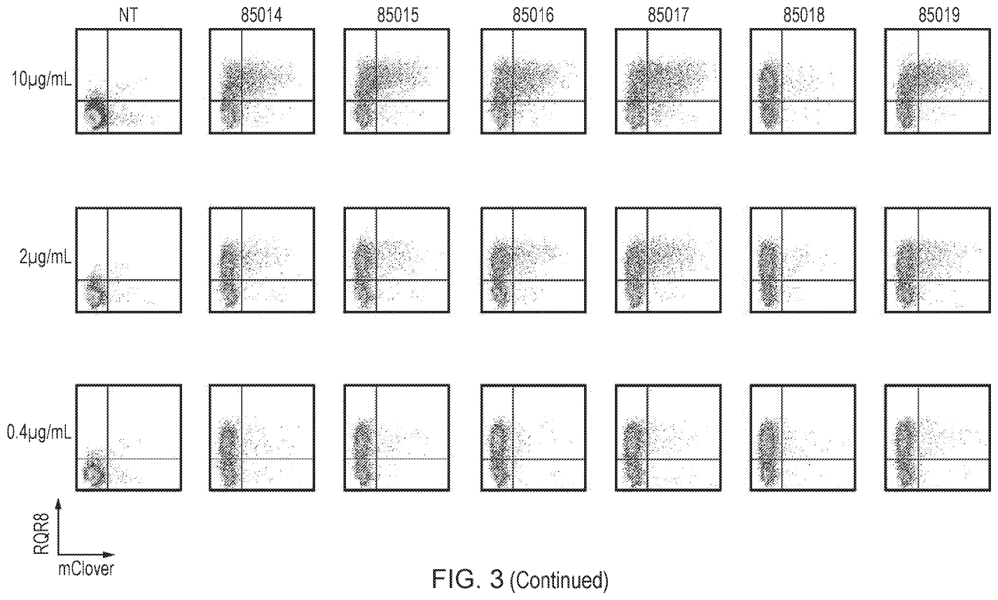

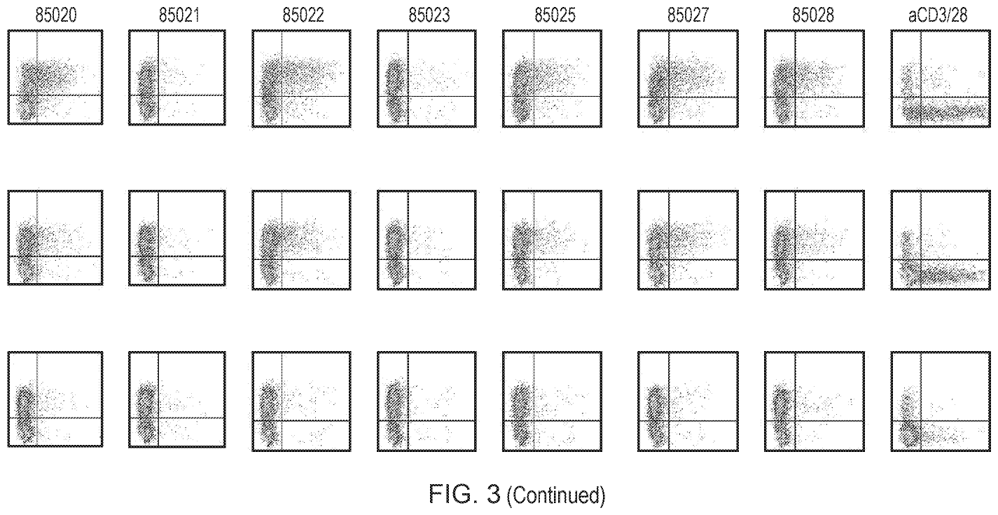

After 72 h antigen (FIG. 3) exposure NUR77-mClover expression is expected to decrease slightly due to its role as an early T cell activation marker. Positive control wells (aCD3/CD28) have reduced from ˜78% activated to less than 57% and NT cells show no mClover upregulation. Similarly, FcRH5 activated CAR T cells have also reduced in mClover expression at all antigen concentrations tested here. However activated T cells are still present across both 10 and 2 μg/ml coated wells respectively (FIG. 4).

TABLE 7
VHH and corresponding CAR constructs in
order of mClover upregulation after 24 h

| | | Reroviral | Nur77-mClover | |
|---|---|---|---|---|
| | | supernatant | positive cells after | SPR Affinity |
| VHH-Fc | CAR ID | Nu77 assay | 24 h 10 μg/ml | (on FcRH5 Ig8 |
| (AU#) | (AU#) | (AU#) | (% upregulation) | domain) |
| AU60466 | 66835 | 85022 | 45.45 | 3.14E−09 |
| AU60460 | 66829 | 85017 | 44.48 | 6.69E−10 |
| AU60471 | 66840 | 85025 | 40.74 | 7.57E−10 |
| AU60387 | 66825 | 85015 | 38.95 | 2.36e−13* |
| AU60463 | 66832 | 85019 | 38.41 | 2.99E−09 |
| AU60370 | 66842 | 85027 | 36.84 | 3.64E−09 |
| AU60390 | 66828 | 85016 | 34.2 | 9.93e−13* |
| AU60382 | 66843 | 85028 | 29.67 | 4.34E−09 |
| AU60464 | 66833 | 85020 | 26.63 | 4.84E−09 |
| AU60385 | 66823 | 85014 | 26.46 | 6.56E−09 |
| AU60465 | 66834 | 85021 | 4.2 | 4.54E−09 |
| AU60462 | 66831 | 85018 | 2.59 | 5.80E−09 |
| AU60467 | 66836 | 85023 | 8.98E−01 | N/A |
| AU60369 | 66841 | 85026 | Possible tonic | 4.77e−9 |
| AU60470 | 66839 | 85024 | Possible tonic | 2.55E−10 |

### Experiment 2_Nur77-mClover Activation Assay, Plate Antigen 2-Fold Dilution Series.

Preliminary plate bound antigen-based CAR engagement assays showed that anti-FCRH5 VHH-CARs could activate Nur77 pathways. Here the activation assay was repeated with a reduced 2-fold (rather than 5-fold) dilution series of FcRH5 concentrations.

Activation of NUR77 Pathway by Plate Bound Antigen Repeated with 2-Fold Dilutions of FcRH5.

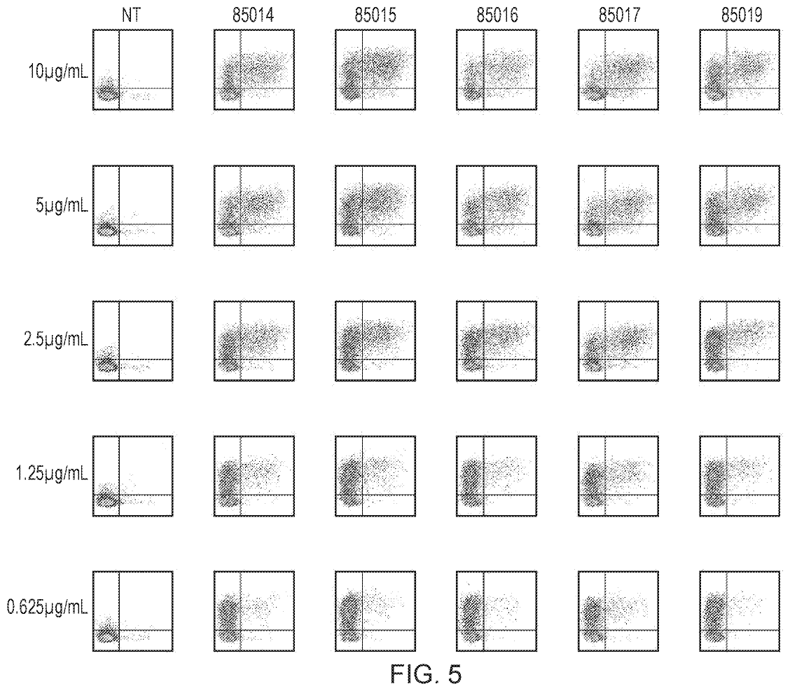

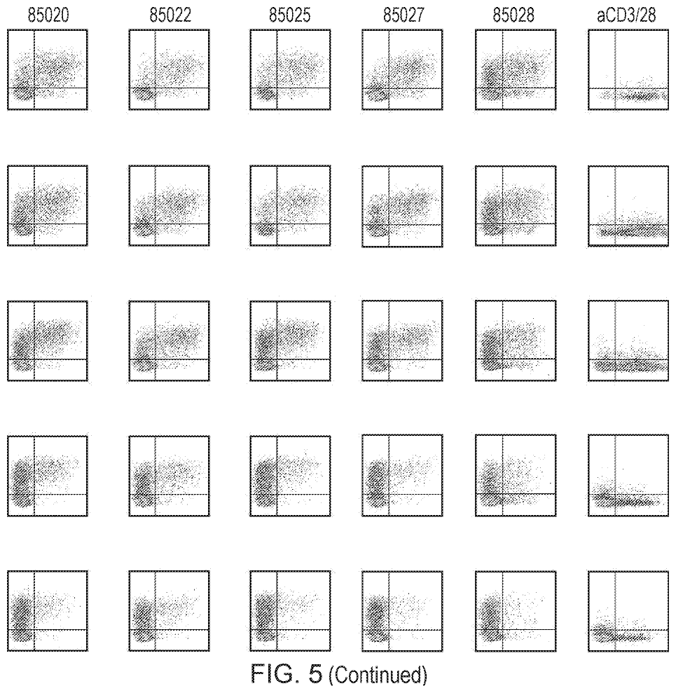

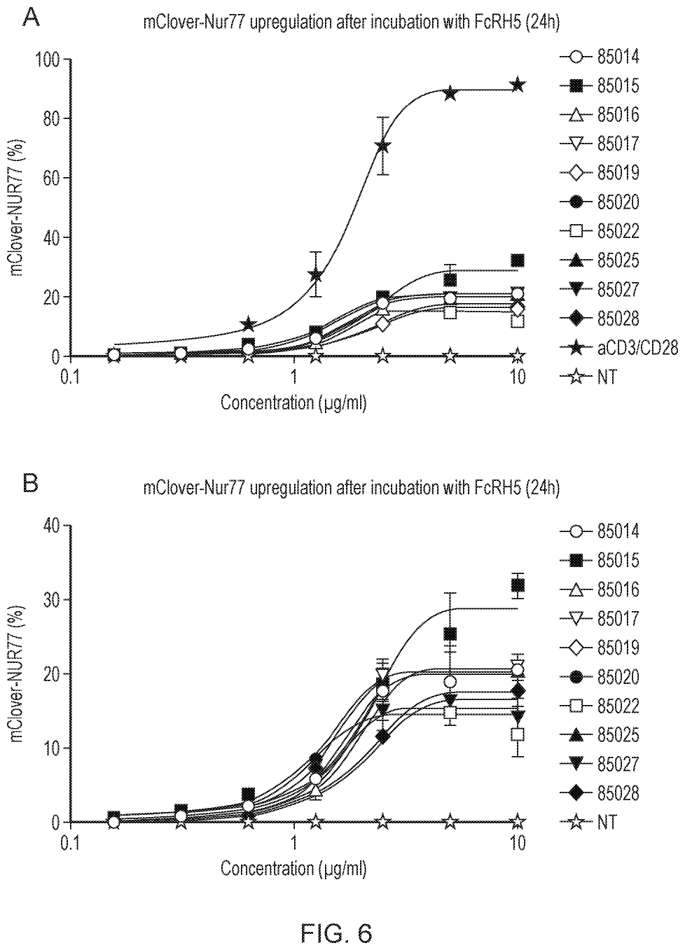

There was a concentration dependant activation of Nur77 across all tested CAR constructs indicating CAR engagement (FIG. 5-6). Below 0.625 μg/ml (0.0625 μg/well) there was no activation correlating with previous work suggesting a reduction in binding at 0.4 μg/ml (Error! Reference source not found.7). At 10 μg/ml activation was similar across CARs however at lower concentrations of antigen there was a reduction in binding. T cells transduced with 85017, 85020 and 85022 showed the most activated population at both 1.25 and 0.625 μg/ml. Activation of cells vs concentration of FcRH5 (Error! Reference source not found.8) shows differentiation of constructs and the calculated EC50's are reported in Table 8.

TABLE 8
EC50 (μg/ml) of percentage Nur77
activation by plate bound FcRH5 antigen.

| pmol ID | pmol ID | SUPE #for | EC50 |
|---|---|---|---|
| (Abvec-Fc) | (CAR) | CD69 assay | (FcRh5 coating μg/ml) |
| AU60385 | 66823 | 85014 | 1.536 |
| AU60387 | 66825 | 85015 | 2.418 |
| AU60390 | 66828 | 85016 | 1.804 |
| AU60460 | 66829 | 85017 | 1.428 |
| AU60463 | 66832 | 85019 | 2.00 |
| AU60464 | 66833 | 85020 | 1.315 |
| AU60466 | 66835 | 85022 | 0.9765 |
| AU60471 | 66840 | 85025 | 1.622 |
| AU60370 | 66842 | 85027 | 1.455 |
| AU60382 | 66843 | 85028 | 1.972 |

### Example 3—Generation and Biophysical Characterisation of Soluble Anti-FcRH5 VHH-his Binding Domains

### Method

### Constructs and Cell Lines

Table 9 details the DNA constructs and cell lines used in this Example.

TABLE 9
List of constructs in study

| | Original Fc- | | |
|---|---|---|---|
| AU Plasmid | tagged construct | | |
| No. | (AU plasmid No.) | Plasmid name | Description |
| 85127 | AU60369 | AbVec.aFcRH5_Clone.B03_dAb-H6H6 | Anti-FcRH5 VHH-His |
| 85128 | AU60370 | AbVec.aFcRH5_Clone.B04_dAb-H6H6 | Anti-FcRH5 VHH-His |
| 85129 | AU60382 | AbVec.aFcRH5_Clone.B07_dAb-H6H6 | Anti-FcRH5 VHH-His |
| 85130 | AU60385 | AbVec.aFcRH5_Clone.B11_dAb-H6H6 | Anti-FcRH5 VHH-His |
| 85131 | AU60387 | AbVec.aFcRH5_Clone.C01_dAb-H6H6 | Anti-FcRH5 VHH-His |
| 85132 | AU60390 | AbVec.aFcRH5_Clone.C06_dAb-H6H6 | Anti-FcRH5 VHH-His |
| 85133 | AU60460 | AbVec.aFcRH5_Clone.C10_dAb-H6H6 | Anti-FcRH5 VHH-His |
| 85134 | AU60462 | AbVec.aFcRH5_Clone.E04_dAb-H6H6 | Anti-FcRH5 VHH-His |
| 85135 | AU60463 | AbVec.aFcRH5_Clone.E07_dAb-H6H6 | Anti-FcRH5 VHH-His |
| 85136 | AU60464 | AbVec.aFcRH5_Clone.E11_dAb-H6H6 | Anti-FcRH5 VHH-His |
| 85137 | AU60465 | AbVec.aFcRH5_Clone.E12_dAb-H6H6 | Anti-FcRH5 VHH-His |
| 85138 | AU60466 | AbVec.aFcRH5_Clone.F01_dAb-H6H6 | Anti-FcRH5 VHH-His |
| 85139 | AU60467 | AbVec.aFcRH5_Clone.F03_dAb-H6H6 | Anti-FcRH5 VHH-His |
| 85140 | AU60470 | AbVec.aFcRH5_Clone.F11_dAb-H6H6 | Anti-FcRH5 VHH-His |
| 85141 | AU60471 | AbVec.aFcRH5_Clone.F12_dAb-H6H6 | Anti-FcRH5 VHH-His |

Antibodies were expressed by transient transfection in expi-CHO cells. Anti-FcRH5 VHH-His carrying a dual 6×His tag, were expressed by transient transfection in expi-CHO cells using plasmids listed in Table 9, and were purified from the supernatants using a Ni-beads suspension.

### Differential Scanning Fluorimetry to Determine Thermal Stability

Purified antibodies were loaded onto glass capillaries (Nanotemper) in duplicate. Scanning at 330 and 350 nm was performed using Prometheus NT.48 instrument (Nanotemper) with a temperature ramp of 1° C./min from 20 to 95° C. Melting temperature (Tm) calculated as first derivative of 350 nm/330 nm ratio.

### Results

Clone 85134 was excluded from the run. Clones 85138 and 85139 did not show unfolding events at the range of temperature. Results are shown in Table 10.

TABLE 10
Thermal stability (Tm)

| Clone | Tm (° C.) |
|---|---|
| 85127 | 68.76 |
| 85128 | 64.14 |
| 85129 | 74.60 |
| 85130 | 74.60 |
| 85131 | 79.5 |
| 85132 | 71.70 |
| 85133 | 75.76 |
| 85134 | N/A |
| 85135 | 79.17 |
| 85136 | 59.17 |
| 85137 | 59.1 |
| 85138 | — |
| 85139 | — |
| 85140 | 72.68 |
| 85141 | 71.28 |

### ELISA

Recombinant purified anti-FcRH5 VHH-His antibodies were coated at 1 μg/ml in 50 μl/well with 3-fold serial dilutions on 96-well ELISA plates (Nunc Maxisorp) overnight at 4° C. Plates were blocked with 200 μl/well of PBS 2% BSA for 1 h at RT on a plate shaker. Plates were washed 3 times with 250 μl/well of PBS 0.05% Tween20 and incubated with 50 μl/well of recombinant FcRH5 Ig8 domain fused to murine IgG2a Fc in PBS 0.5% BSA for 1 h at RT on a plate shaker. Plates were washed 3 times with 250 μl/well of PBS 0.05% Tween20 and incubated with 50 μl/well of secondary anti-mouse antibody HRP conjugated (Jackson ImmunoResearch—115-035-166) at 1:5000 dilution in PBS 0.5% BSA for 1 h at RT on a plate shaker. Plates were washed four times in PBS 0.05% Tween20 and binding revealed with 45 μl/well of 1-Step Ultra TMB reagent (Thermo scientific) for 3 minutes at RT. Reaction was blocked with 45 μl/well of 1M H₂SO₄ and absorbance read at OD 450 nm on a Varioskan Lux plate reader (Thermo scientific).

### Flow Cytometry

Binding for FcRH5 was determined via flow cytometry on HEK293T cells and HEK293T cells transiently transfected to express the extracellular Ig8 domain of FcRH5 fused to human CD19 transmembrane domain and cytoplasmic tail (AU45283). Construct contains the eGFP protein as transfection marker. 5×10⁴ cells/well were washed twice in FACS buffer (PBS 2% FCS) and stained with 50 μl of primary antibody (Table 8) at 2 μg/ml for 30 on ice. Cells were washed twice in FACS buffer to remove unbound antibodies and stained with 50 μl/well of anti-His secondary antibody conjugated to Alexa fluor 647 (Santa Cruz Biotechnology-sc-53073AF647) diluted 1:100, for 30 minutes in ice. Cells were then washed in FACS buffer and stained with 100 μl/well of Sytox blue for viability determination (Thermo Fisher Scientific-S34857), according to manufacturer's recommendations. Cells were acquired on a MACSQuant 10 instrument. Cells were first gated on singlets, then population was defined based of SSC/FSC appearance and live cells identified based on Sytox blue staining.

### Results

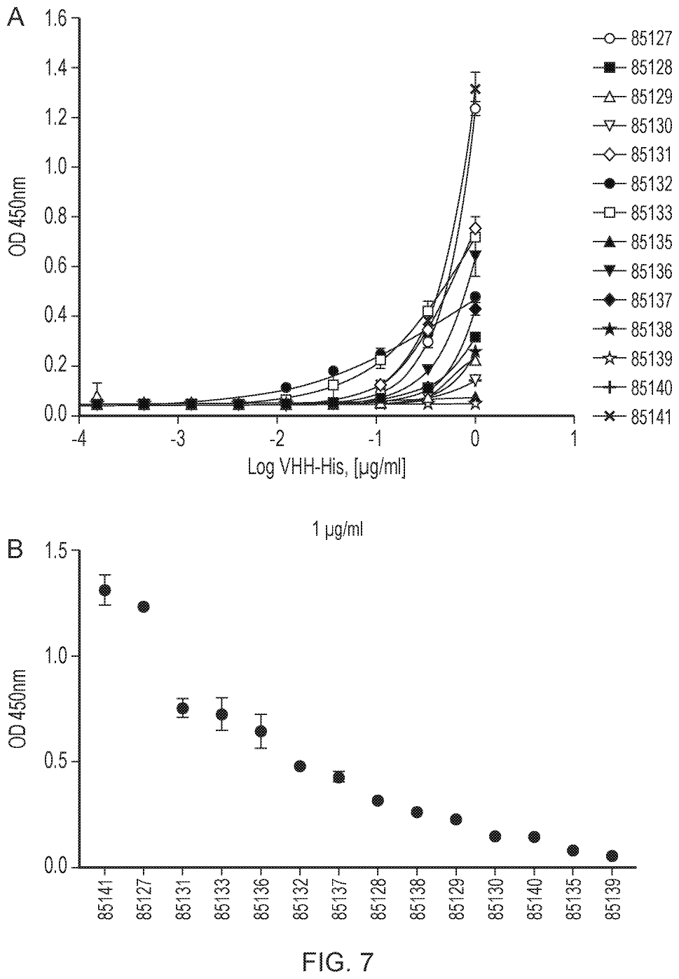

Binding capacity of the soluble anti-FcRH5 VHH antibodies for human FcRH5 Ig8 domain (with murine IgG2a Fc tag) was determined via ELISA assay (FIG. 7). VHH domains were coated on the plate at a concentration of 1 μg/ml with 3-fold serial dilutions. Binding to FcRH5 Ig8-Fc was detected with anti-mouse HRP secondary antibody.

With the exception of clone 85139 (non-Ig8 binder), all clones showed binding to the FcRH5 Ig8 domain. Clones 85141 and 85127 showed the strongest binding interaction.

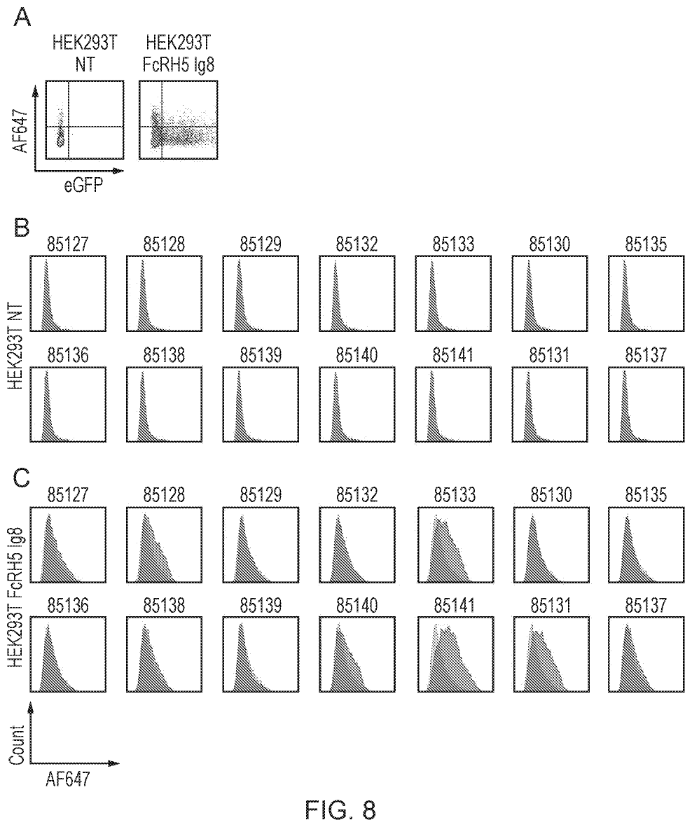

Binding of monomeric soluble VHH-His antibody domains to FcRH5 Ig8 expressing cells was investigated via flow cytometry (FIG. 8). HEK293T cells were transiently transfected to express the FcRH5 Ig8 domain and eGFP marker. Untransfected HEK293T (NT) were included as control. Bound VHH-His were detected with secondary anti-His antibody conjugated to Alexa fluor 647. Positive staining was measured as comparison to a primary antibody omission condition. Low transfection efficiency was detected in the HEK293T FcRH5 Ig8 population. Despite low protein expression, several clones showed specific binding to FcRH5.

### Example 4—Generation and Biophysical Characterisation of Soluble Anti-FcRH5 VHH-Fc and Anti-FcRH5 VHH-his Binding Domains

The VHH sequences of the constructs that are used in Example 4 can be identified by the following CDR1-3 and VHH sequences:

TABLE 11
Constructs and corresponding sequences

| AU ID# | CDR1 | CDR2 | CDR3 | VHH |
|---|---|---|---|---|
| 60369 | RSSFSNNA | ITKGGVT | NTIPFRSA | QVQLQESGGGLVQAGGSLRLSCAASRSSFSNNAMGWYRQVP |
| | | | | GKQRELVAFITKGGVTDYSDSVKGRFTISKDNAKNTVYLQMNSL |
| | | | | KPEDTAVYYCNTIPFRSAWGQGTQVTVSS |
| 88678 | GNIFRLNG | ITSGGNT | NTIPFRLS | QVQLQESGGGLVQPGGSLRLSCAAPGNIFRLNGTGWYRQAPG |
| | | | | KQRELVAHITSGGNTDYADSVKGRFTISRDNAKNTVYLQMNSLK |
| | | | | PEDTAVYYCNTIPFRLSWGQGTQVTVSS |
| 88679 | GNIFRLNA | ITSGGNT | NTIPFRLS | QVQLQESGGGLVQPGGSLRLSCAAPGNIFRLNAMGWYRQAPG |
| | | | | KQRELVAHITSGGNTDYADSVKGRFTISRDNAKNTVYLQMNSLK |
| | | | | LEDTAVYYCNTIPFRLSWGQGTQVTVSS |
| 88680 | RSSFSNNA | ITKGGVT | NTIPVRSA | QVQLQESGGGLAQAGGSLRLSCAASRSSFSNNAMGWYRQVQ |
| | | | | GKQRELVAFITKGGVTDYSVSGKGRFTISKDHAKNTVYLQMNSL |
| | | | | KPRDTAVYYCNTIPVRSAWGQGTQVTVSA |
| 88681 | GSIFSINA | ITKGGVT | NTIPCRSA | QVQLQESGGGLVQAGGSLRLSCAASGSIFSINAMGWYRQAPG |
| | | | | KQRELVAFITKGGVTDYSDSVKGRFTISKDNAKNTVYLQMNSLK |
| | | | | PEDTAVYYCNTIPCRSAWGQGTQVTVSS |
| 88682 | GSSFRLNG | ITSGGST | NTIPFSRA | QVQLQESGGGLVQPGGSLRLSCAAPGSSFRLNGTGWYRQAP |
| | | | | GKQRELVAHITSGGSTNYSDSVKGRLTISKDNAKNTVYLQMNSL |
| | | | | KPEDTAVYYCNTIPFSRAWGQGTLVTVSS |
| 88683 | GRSVSINA | IDRSGNT | NTIPYSDS | QVQLQQSGGGLVQAGGSLRLSCAASGRSVSINAMGWYRQAP |
| | | | | GKQRELVAIIDRSGNTDYADSVKGRFTISRDNAKKAVYLQMNSL |
| | | | | KPEDTAVYYCNTIPYSDSWGQGTQVTISS |
| 88684 | GRTFSSYA | IDGIGGIT | NTIPFRSA | QVQLQESGGGLVQPGGSLRLSCAASGRTFSSYAMGWFRRAP |
| | | | | GKGREFVATIDGIGGITSYAGSVKGRFTVSKDNAKNTVYLQMNS |
| | | | | LKPEDTAVYYCNTIPFRSAWGQGTLVTVSS |
| 88685 | RSSFSNNA | ITKGGVT | NTIPFRSA | QVQLQESGGGLVQAGGSLRLSCAASRSSFSNNAMGWYRQGP |
| | | | | GKQRELVAFITKGGVTDYSDSVKGRFTISKDNAKNTVYLQMNSL |
| | | | | KPEDTAVYYCNTIPFRSAWGQGTQVTVSS |
| 88686 | RSSFSNNA | ITKGGVT | NAIPFRSA | QVQLQESGGGLVQAGGSLRLSCAASRSSFSNNAMGWYRQVP |
| | | | | GKQRELVAFITKGGVTDYSDSVKGRFTISKDNAKNTVYLQMNSL |
| | | | | KPEDTAVYYCNAIPFRSAWGQGTQVTVSS |
| 88687 | GNNFRLNA | ITSGGNT | NAIPFRPS | QVQLQESGGGLVQPGGSLRLSCAAPGNNFRLNAMGWYRQAP |
| | | | | GKQRELVAHITSGGNTDYADSVKGRFTISRDNAKNTVYLQMNS |
| | | | | LKPEDTAVYYCNAIPFRPSWGQGTQVTVSS |
| 88688 | GNIFRLNG | ITSGGNT | NAIPFRRS | QVQLQESGGGLVQPGGSLRLSCAAPGNIFRLNGTGWYRQAPG |
| | | | | KQRELVAHITSGGNTDYADSGKGRFTISRDNAKNTVYLQMNSL |
| | | | | KPEDTAVYYCNAIPFRRSWGQGTQVTVSS |
| 88689 | ERIFRINA | ITSGGNT | NAIPFRRS | QVQLQESGGGLVQAGGSLKLSCAASERIFRINAMGWYRQAPG |
| | | | | KQRELVAHITSGGNTDYADSVKGRFTISRDNAKNTMYLQMNSL |
| | | | | KPEDTAVYYCNAIPFRRSWGQGTQVTVSS |
| 88690 | GNIFRLNG | ITSGGNT | NAIPFRRS | QVQLQQSGGGLVQPGGSLRLSCAAPGNIFRLNGTGWYRQAPG |
| | | | | KQRELVAHITSGGNTDYADSVKGRFTISRDNAENTVYLQMNSLK |
| | | | | PEDTAVYYCNAVPFRLSWGQGTQVTVSS |
| 88691 | GFTFSTYW | IDNGGGTT | NALPFRLS | QVQLQESGGGLVQPGESLRLSCAASGFTFSTYWMSWVRQAP |
| | | | | GKGPEGVSGIDNGGGTTTYADSVKGRFTISRDNAGNTVYLQMN |
| | | | | SLKPEDTAVYYCNALPFRLSWGQGTQVTVSS |
| 88692 | GNIFRLNG | ITSGGNT | NATPFRLS | QVQLQESGGGLVQPGGSLRLSCAAPGNIFRLNGTGWYRQAPG |
| | | | | KQGELVAIITSGGNTDYADSVKGRFTISRDNAKNTTYLQMNSLK |
| | | | | PEDTAVYYCNATPFRLSWGQGTQVTVSS |
| 88693 | GNIFRLNG | ITRGGNT | NSIPFRLS | QVQLQESGGGLVQPGGSLRLSCAAPGNIFRLNGTGWYRQAPG |
| | | | | KQRELVAHITRGGNTDYADAVKGRFTISRDNAKNTVYLQMNSL |
| | | | | KPEDTAVYYCNSIPFRLSWGQGTQVTVSS |
| 88694 | GNIFRING | ITSGGNT | NAIPFRIS | QVQLQESGGGLVQPGGSLRLSCAAPGNIFRINGTGWYGQAPG |
| | | | | KQRELVAHITSGGNTDYEDSVKGRFTISRDNAKNTTYLQMNSLK |
| | | | | PEDTAVYYCNAIPFRISWGQGTQVTVSS |
| 88695 | GNNFRLNA | ITSGGNT | NAIPFRLY | QVQLQQSGGGLVQPGGSLRLSCAAPGNNFRLNAMGWYRQAP |
| | | | | GKQRELVAHITSGGNTDYADSVKGRFTISRDNAKNTTYLQMNS |
| | | | | LKPEDTAVYYCNAIPFRLYWGQGTQVTVSS |
| 88696 | GFTFSTYW | IDNGGGTT | KAIPFRLS | QVQLQESGGGLVQPGGSLRLSCLASGFTFSTYWMSWVRQAP |
| | | | | GKGPEWVSGIDNGGGTTTYADSVKGRFTISRDNAKNTLNLEMN |
| | | | | NLKPEDTAGYYSKAIPFRLSWGQGTQVTVSS |
| 88697 | GSSFSNNA | ITKGGVT | NTIPFRSA | QVQLQESGGGLVQPGGSLRLSCAASGSSFSNNAMGWYRQAP |
| | | | | GKQRELVAFITKGGVTDYSDSVRGRFTISRDNAKNTVYLQMNSL |
| | | | | KPEDTAVYYCNTIPFRSAWGQETQVTVSS |
| 88698 | GFTFSTYW | IDNGGGTT | NTIPFRSA | QVQLQQSGGGLVQPGGSLRLSCLASGFTFSTYWMSWVRQAP |
| | | | | GKGPEWVSGIDNGGGTTTYADSVKGRFTISRDNAKNTVYLQMN |
| | | | | SLKPEDTAVYYCNTIPFRSAWGQGTQVTVAS |
| 88699 | GNIFRLNA | ITKGGVT | NTIPFRSA | QVQLQESGGGLVQPGGSLRLSCAAPGNIFRLNATGWYRQVPG |
| | | | | KQRELVAFITKGGVTEHSDSVEGRFTISKDNAKNTVYLQMNSLK |
| | | | | PEDTAVYYCNTIPFRSAWGQGTQVTVSS |
| 88700 | RSSFSNNA | ITKGGVT | NTIPFRSA | QVQLQESGGGLVQTGGSLRLSCAASRSSFSNNAMGWYRQVP |
| | | | | GKQRELVAFITKGGVTDYSDSVKGRFTISKDNAKNTVYLQMNSL |
| | | | | KPEDTAVYYCNTIPFRSAWGQGTQVTVSS |
| 88701 | GSSFSNNA | ITKGGVT | NTIPFRSA | QVQLQESGGGLVQAGGSLRLSCAASGSSFSNNAMGWYRQVP |
| | | | | GKQRELVAFITKGGVTDYSDSVKGRFTISKDNAKNTVYLQMNSL |
| | | | | KPEDTAVYYCNTIPFRSAWGQGTQVTVSS |
| 88702 | RSSFGNNA | ITKGGVT | NTIPFRSA | QVQLQESGGGLVQAGGSLRLSCAASRSSFGNNAMGWYRQVP |
| | | | | GKQRELVAFITKGGVTDYSDSVKGRFTISKDNAKNTVYLQMNSL |
| | | | | KPEDTAVYYCNTIPFRSAWGQGTQVTVSS |
| 60387 | GRTFSTYG | ISRSGGAT | AGTRRAFS | QVQLQESGGGLVQAGGSLRLSCAASGRTFSTYGMGWFRQAP |
| | | | TGLRDYDY | GKEREFVAAISRSGGATAYAASVKGRFTISRDDVKNTLYLQMNS |
| | | | | LKPEDTAVYHCAGTRRAFSTGLRDYDYWGQGIQVTVS |
| 88704 | GTIERNNA | ITSGGST | AAGRRFST | QVQLQESRGGLVQAGGSLRLSCAASGTIERNNAMAWYRQAPG |
| | | | RSRDYDY | KQRELVAIITSGGSTNYSDSVKGRFTISRDNAKNTLYLQMNSLK |
| | | | | PEDTAVYLWAAGRRFSTRSRDYDYWGQGTQVTVSS |
| 88705 | GRSFSNYG | IGMVGGLT | AAGRRFST | QVQLQESGGGLVQAGGSLSLSCTASGRSFSNYGMGWFRQAP |
| | | | SSRDYDY | GKEREFVAAIGMVGGLTAYSNSAKGRFTISRDNAKNTVYLQMN |
| | | | | SLKPEDTAVYLCAAGRRFSTSSRDYDYWGQGTQVTVSS |
| 88706 | GRTFSSYA | ISRIGGVT | AAAGLVSIS | QVQLQESGGGLVQAGDSLRLSCAASGRTFSSYAMGWFRQAP |
| | | | TTPNDYDY | GKEREFVAAISRIGGVTTYAGSVQGRFTISRDNAKNTLYLRMNA |
| | | | | LKPEDTAVYYCAAAGLVSISTTPNDYDYWGQGTQVTVSS |
| 88707 | GRSFSNYG | IGMVGGLT | AAGRRFST | QVQLQESGGGLVQAGGSLSLSCTASGRSFSNYGMGWFRQAP |
| | | | GSRDYDI | GKEREFVDAIGMVGGLTAYSNSAKGRFTISRDNAKNTLYLQMN |
| | | | | SLKPEDTAVYLCAAGRRFSTGSRDYDIWGQGTQVTVSS |
| 88708 | GRTFSSYA | ISQFGGVT | AAGRRFST | QVQLQQSGGGLVQTGGSLRLSCAASGRTFSSYAMGWFRQAP |
| | | T | GSRDYDI | GKEREFVAAISQFGGVTTYADSVQGRFTISRDNAKNTLYLRMNS |
| | | | | LKPEDTAVYLCAAGRRFSTGSRDYDIWGQGTQVTVSS |
| 88709 | GRSFSNYG | IGMVGGLT | AGGRRFST | QVQLQESGGGLVQAGGSLSLSCTASGRSFSNYGMGWFRQAP |
| | | | SSRDYDI | GKEREFVAAIGMVGGLTAYSNSAKGRFTISRDKAKNTLYLQMN |
| | | | | SLKPEDTAGYCGAGGRRFSTSSRDYDIWGQGTQVTVSS |
| 88710 | GRSFSNYG | IGMVGGLT | AAGRRFST | QVQLQESGGGLVQAGGSLSLSCTASGRSFSNYGMGWFRQAP |
| | | | SSREYDI | GKEREFVAAIGMVGGLTAYSNSAKGRFTISRDNAKNTLYLQMN |
| | | | | SLKPEDTAVYLCAAGRRFSTSSREYDIWGQGTQVTVSS |
| 88711 | GRSFSNYG | IGMVGGLP | AAGRRLST | QVQLQESGGGLVQAGGSLSLSCTASGRSFSNYGMGWFRQAP |
| | | | SSRDYDI | GKEREFVAAIGMVGGLPAYSNSAKGRFTISQDNAKNPLYLQINS |
| | | | | LKPEETDVYLCAAGRRLSTSSRDYDIWGQGTQVTVSS |
| 88712 | GRSFSNYG | ISRGGGVS | AAGLRFST | QVQLQESGGGLVQAGGSLSLSCTASGRSFSNYGMGWFRQAP |
| | | | GSRDYDI | GKEREFVAAISRGGGVSAYSNSAKGRFTISRDNAKNTVYLQMN |
| | | | | SLKPEDAAVYFCAAGLRFSTGSRDYDIWGQGTQVTVSS |
| 60460 | GRTYNNYA | ISRSGGMT | AAYVGGFS | QVQLQESGGGLVQAGGSLRLSCAASGRTYNNYAMGWFRQAP |
| | | | TARRDYSY | GKEREFVAGISRSGGMTGYAESVKGRFTISRDNAKNMVFLQMN |
| | | | | SLKPEDTAVYYCAAYVGGFSTARRDYSYWGQGTQVTVS |
| 88714 | GRTFRRYA | ISRSGGMT | AAYVGGFS | QVQLQQSGGGLVQAGDSLRLSCAASGRTFRRYAMGWFHQAP |
| | | | TTRRDYAY | GKDREFVAGISRSGGMTGYADSVKGRFTISRDNAKNMVFLQMN |
| | | | | SLKPEDTAVYYCAAYVGGFSTTRRDYAYWGQGTQVTVSS |
| 88715 | GRTFSNST | ISWSGGTT | AAARKGW | QVQLQESGGGLVQAGDSLRLSCAASGRTFSNSTMGWFHQAP |
| | | | STRGDDYD | GKERKFVAVISWSGGTTAYAESVKGRFTISRDNAKNTVYLQMN |
| | | | Y | SLKPEDTAVYYCAAARKGWSTRGDDYDYWGQGTQVTVSS |
| 88716 | GRTVI | SSGSGGVT | AAALTWST | QVQLQESGGGLVQTGGSLRLSCAASGRTVIGWFRQAPGKERE |
| | | | RPSDFTS | FVAVSSGSGGVTAYASSVEGRFTISRDNVKNIMYLQMNSLKPE |
| | | | | DTAIYYCAAALTWSTRPSDFTSWGQGTQVTVSS |
| 88717 | GRTYNNYA | ISRSGGMT | AAYVGGFS | QVQLQQSGGGLVQAGGSLRLSCAASGRTYNNYAMGWFRQAP |
| | | | TARRDYSY | GKEREFVAGISRSGGMTGYAESVKGRFTISRDNAKNMVCLQMN |
| | | | | SLKPEDKAVYYCAAYVGGFSTARRDYSYWGQGTQVTVSS |
| 88718 | GRTVI | SNWSGGV | AAYVGGFS | QVQLQQSGGGLVQTGGSLRLSCAASGRTVIGWFRQAPGKERE |
| | | T | TARRDYSY | FVAVSNWSGGVTAYASSVEGRFTISRDNVKNIMYFQMNSLKPE |
| | | | | DTAVYYCAAYVGGFSTARRDYSYWGQGTQVTVSS |
| 88719 | GRTYNNYA | ISRSGGMT | AAYVGGFS | QVQLQQSGGGLVQPGGSLRLSCAASGRTYNNYAMGWFRQAP |
| | | | TARRDYSY | GKEREFVAGISRSGGMTGYAESVKGRFTISRDNAKNMVFLQMN |
| | | | | SLKPEDTAVYYCAAYVGGFSTARRDYSYWGQGTQVTVSS |
| 88720 | GRTFSINA | ISRSGGMT | AAYVGGFS | QVQLQESGGGLVQAGGSLIVSCATSGRTFSINAMGWFRQAPG |
| | | | TARRDYSY | KEREFVAGISRSGGMTGYAESVKGRFTISRDNAKNMVFLQMNS |
| | | | | LKPEDTAVYYCAAYVGGFSTARRDYSYWGQGTQVSVSS |
| 60462 | GRTSSRAA | ISWSGGTT | AAARIFTTA | QVQLQESGGGLVQAGGSLRLSCTASGRTSSRAAMGWFRQAP |
| | | | RNDYDH | GKEREFVAVISWSGGTTAYANSVKGRFTISRDNAKNTLYLQMN |
| | | | | SLKPEDTAVYYCAAARIFTTARNDYDHWGQGTQVTVS |
| 88722 | GRTFSRYA | INGSGGT | AAARIFTTT | QVQLQQSGGGLVQAGGSLRLSCAASGRTFSRYAMGWFRQAP |
| | | | RNEYDH | GKEREFVAVINGSGGTTAYANSVKGRFTITRDNAKNTLYLQMNS |
| | | | | LKPEDTAVYYCAAARIFTTTRNEYDHWGQGTQVTVSS |
| 88723 | GFTFSTYW | IDNGGGTT | AAARIFSTA | QVQLQEPGGGLVQPGGSLRLSCAASGFTFSTYWMSWVHQAP |
| | | | RNDYDH | GKGPEWVSGIDNGGGTTTYADSVKGRFTISRDNAKNTLYLQMN |
| | | | | SLKPEETAIYYWAAARIFSTARNDYDHWGQGTQVTVSS |
| 88724 | GRSFSNYG | IGMVGGLT | AGGRIFRT | QVQLQESGGGLVQAGGSLSLSCTASGRSFSNYGMGWFRQAP |
| | | | SSRDYDI | GKEREFVAAIGMVGGLTAYSNSAKGRFTISRDNAKNTLYLQMN |
| | | | | SLKPEDTAVYLVAGGRIFRTSSRDYDIWGQGTQVTVSS |
| 88725 | GNIFRLNG | ITSGGNT | AAARFFTT | QVQLQESGGGLVQPGGSLRLYCAAPGNIFRLNGTGWYRQAPG |
| | | | ARNDYDH | KQRELVTHITSGGNTDYADSVKGRFTISRDNAKNTVYLQMNSLK |
| | | | | PEDTAIYYCAAARFFTTARNDYDHWGQGTQVTVSS |
| 60471 | GSIFSINA | ITSGGST | NALGGFVP | QVQLQESGGGLVQAGGSLRLSCAASGSIFSINAMGWYRQAPG |
| | | | SYG | KQRELVAFITSGGSTNYADSVKGRFTISRDNAKNTVYLQMNSLK |
| | | | | PEDTAVYYCNALGGFVPSYGWGQGTQVTVS |
| 88727 | GSIFSINA | ITSGGST | NALGGFVP | QVQLQESGGGLVQAGGSLRLSCAASGSIFSINAMGWYRQAPG |
| | | | NYG | KQRELVAAITSGGSTNYADSVKGRFTISRDNAKNTVYLQMNSLK |
| | | | | PEDTAVYYCNALGGFVPNYGWGQGTQVTVSS |
| 88728 | GNIFRLNG | ITSGGNT | NALGGFVP | QVQLQESGGGLVQPGGSLRLSCAAPGNIFRLNGTGWYRQAPG |
| | | | NYG | KQRELVAHITSGGNTDYADSVKGRFTISRDNAKNTVYLQMNSLK |
| | | | | PEDTAVYYCNALGGFVPNYGWGQGTQVTVSS |
| 60471 | GSIFSINA | ITSGGST | NALGGFVP | QVQLQESGGGLVQAGGSLRLSCAASGSIFSINAMGWYRQAPG |
| | | | SYG | KQRELVAFITSGGSTNYADSVKGRFTISRDNAKNTVYLQMNSLK |
| | | | | PEDTAVYYCNALGGFVPSYGWGQGTQVTVSS |
| 88730 | GSIFSINA | ITSGGST | NALGGFVP | QVQLQESGGGLVQAGGSLRLSCAASGSIFSINAMGWYRQAPG |
| | | | NDG | KQRELVAAITSGGSTNYADSVKGRFTTSRDNAKNTVYLQMNSL |
| | | | | KPEDPAVYYCNALGGFVPNDGWGQGTQVTVSS |
| 88731 | GNNFRLNA | ITSGGNT | NALGGFAP | QVQLQESGGGLVQAGGSLRLSCAASGNNFRLNAMGWYRQAP |
| | | | NYG | GKQRELVAHITSGGNTDYADSVKGRFTISRDNAKNTTYLQMNS |
| | | | | LKPEDTAVYYCNALGGFAPNYGWGQGTQVTVSS |
| 88732 | GRSFSNYG | IGMVGGLT | NALGGFVP | QVQLQQSGGGLVQAGGSLSLSCTASGRSFSNYGMGWFRQAP |
| | | | NYV | GKEREFVAAIGMVGGLTAYSNSAKGRFTISRDNAKNTVYLQMN |
| | | | | SLKPEDTAVYYCNALGGFVPNYVWGQGTQVTVSS |
| 88733 | GRSFSINA | ITSGGST | NALGGFVL | QVQLQESGGGLVQAGGSLSLSCTASGRSFSINAMGWYRQAPG |
| | | | NYG | KQRELVAAITSGGSTNYADSVKGRFTISRDNAKNTVYLQMNSLK |
| | | | | PEDTAVYYCNALGGFVLNYGWGQGTQVTVSS |
| 88734 | GSIFSINA | ITSGGST | NALGGLVP | QVQLQESGGGLVQAGGSLRLSCAASGSIFSINAMGWYRQAPG |
| | | | NYG | KQRELVAAITSGGSTNYADPVKGRFTISRDNAKNTVYLQMNSLK |
| | | | | PEDTAVYYCNALGGLVPNYGWGQGTQVTVSS |
| 88735 | GSIFSINA | ITSGGST | NALGGFLP | QVQLQESGGGLVQAGGSLRLSCAASGSIFSINAMGWYRQAPG |
| | | | NYG | KQRELVAAITSGGSTNYADSVKGRFTISRDNAKNTVYLQMNSLK |
| | | | | PEDTAVYYCNALGGFLPNYGWGQGTQVTVSS |
| 88736 | GSIFSINA | ITSGGST | NALGGFVT | QVQLQESGGGLVQAGGSLRLSCAASGSIFSINAMGWYRQAPG |
| | | | NYG | KQRELVAAITSGGSTNYADSVKGRFTISRDNAKNTVYLQMNSLK |
| | | | | PEDTAVYYCNALGGFVTNYGWGQGTQVAVSS |
| 88737 | GSIFSINA | ITSGGST | NALGGFVP | QVQLQESGGGLVQPGGSLRLSCAASGSIFSINAMGWYRQAPG |
| | | | SYG | KQRELVAFITSGGSTNYADSVKGRFTISRDNAKNTVYLQMNSLK |
| | | | | PEDTAVYYCNALGGFVPSYGWGQGTQVTVSS |
| 88738 | GRTV | ITRGGST | NALGGFVP | QVQLQQSGGGLVQTGGSLRLSCAASGRTVMGWYRQAPGKQR |
| | | | NYG | ELVAVITRGGSTNYADSVKGRFTISRDNAKNTVYLQMNSLKPED |
| | | | | TAFYYCNALGGFVPNYGWGQGTQVTVSS |
| 60471 | GSIFSINA | ITSGGST | NALGGFVP | QVQLQESGGGLVQAGGSLRLSCAASGSIFSINAMGWYRQAPG |
| | | | SYG | KQRELVAFITSGGSTNYADSVKGRFTISRDNAKNTVYLQMNSLK |
| | | | | PEDTAVYYCNALGGFVPSYGWGQGTQVTVSS |
| 88740 | GNIFRLNG | ITSGGNT | NALGGFVP | QVQLQESGGGLVQPGGSLRLSCAAPGNIFRLNGTGWYRQAPG |
| | | | SYG | KQRELVAHITSGGNTDYADSVKGRFTISRDNAKNTVYLQMNSLK |
| | | | | PEDTAVYYCNALGGFVPSYGWGQGTQVTVSS |
| 88741 | GNNFRLNA | ITSGGNT | NALGGFVP | QVQLQESGGGLVQPGGSLRLSCAAPGNNFRLNAMGWYRQAP |
| | | | SYG | GKQRELVAHITSGGNTDYADSVKGRFTISRDNAKNTVYLQMNS |
| | | | | LKPEDTAVYYCNALGGFVPSYGWGQGTQVTVSS |
| 88742 | GSIFSINA | ITSGGST | NALGGFVP | QVQLQQSGGGLVQAGGSLRLSCAASGSIFSINAMGWYRQAPG |
| | | | SYG | KQRELVAFITSGGSTNYADSVKGRFTISRDNAKNTVYLQMNSLK |
| | | | | PEDTAVYYCNALGGFVPSYGWGQGTQVTVSS |
| 88743 | GNIFRLNG | ITSGGST | NALGGFVP | QVQLQQSGGGLVQPGGSLRLSCAAPGNIFRLNGTGWYRQAPG |
| | | | SYG | KQRELVAHITSGGSTNYADSVKGRFTISRDNAKNTVYLQMNSLK |
| | | | | PEDTAVYYCNALGGFVPSYGWGQGTQVTVSS |
| 88744 | GSIFSINA | ITSGGST | NALGGFVP | QVQLQESGGGLVQAGGSLRLSCAASGSIFSINAMGWYRQAPG |
| | | | SYG | KQRELVAAITSGGSTNYADSVKGRFTISRDNAKNTVYLQMNSLK |
| | | | | PEDTAVYYCNALGGFVPSYGWGQGTQVTVSS |
| 88745 | GRSFSNYG | IGMVGGLT | NALGGFVP | QVQLQESGGGLVQAGGSLSLSCTASGRSFSNYGMGWFRQAP |
| | | | SYG | GKEREFVAAIGMVGGLTAYSNSAKGRFTISRDNAKNTLYLQMN |
| | | | | SLKPEDTAVYYCNALGGFVPSYGWGQGTQVTVSS |
| 88746 | GSIFSINA | ITSGGST | NALGGFVP | QVQLQESGGGLVQAGGSLRLSCAASGSIFSINAMGWYRQAPG |
| | | | SYG | KQRELVAFITSGGSTNYADSVKGRFTISRDNAKNTVYLQMNSLK |
| | | | | PEDTAVYYCNALGGFVPSYGWGQGTLVTVSS |
| 88747 | GNIFSINA | ITSGGST | NALGGFVP | QVQLQESGGGLVQPGGSLRLSCAAPGNIFSINAMGWYRQAPG |
| | | | SYG | KQRELVAFITSGGSTNYADSVKGRFTISRDNAKNTVYLQMNSLK |
| | | | | PEDTAVYYCNALGGFVPSYGWGQGTQVTVSS |
| 88748 | GFTFSTYW | IDNGGGTT | ARNPTRG | QVQLQESGGGLVQPGGSLRLSCLASGFTFSTYWMSWVRQAP |
| | | | WYSTDY | GKGPEWVSGIDNGGGTTTYADSVKGRFTISRDNAKNTLNLEMN |
| | | | | NLKPEDTALYYCARNPTRGWYSTDYRGQGTQVTVSS |
| 88749 | GFTFSTYW | IDNGGGTT | ARNPTRG | QVQLQESGGGLVQPGGSLRLSCAASGFTFSTYWMSWVRQAP |
| | | | WYSTDY | GKGPEWVSGIDNGGGTTTYADSVKGRFTISRDNAKHTLYLQMN |
| | | | | TLKPEDTALYYCARNPTRGWYSTDYRGQGTQVTVSS |

### Preparation of Soluble Anti-FcRH5 VHH-Fc Binding Domains

Anti-FcRH5 VHH-Fc carrying a murine IgG2a Fc domain, are expressed by transient transfection in expi-CHO cells and purified with Protein A. The biophysical properties of these binders are characterised.

### Differential Scanning Fluorimetry to Determine Thermal Stability

Purified antibodies are loaded onto glass capillaries (Nanotemper) in duplicate. Scanning at 330 and 350 nm is performed using Prometheus NT.48 instrument (Nanotemper) with a temperature ramp of 1° C./min from 20 to 95° C. Melting temperature (Tm) is calculated as first derivative of 350 nm/330 nm ratio.

### Multi-Angle Dynamic Light Scattering to Determine Aggregation

Aggregation propensity and average particle size of the test proteins is determined using a Zetasizer Ultra device and ZS Xplorer software (Malvern Panalytical) by MADLS. Samples are loaded into a low volume quartz cuvette (Malvern Panalytical-ZEN2112) at a concentration of 1 mg/ml. Triplicate measurements are taken for each sample.

### Surface Plasmon Resonance (SPR)

Recombinant anti-FcRH5 antibodies in mouse IgG2a format are captured on flow cell 2 on a Series S CM5 chip (GE Healthcare), functionalised with anti-mouse capture kit according to manufacturer's recommendations (GE Healthcare). Antibodies are captured to a density of 160 RU using a Biacore 8k instrument. HBS-P+ buffer is used as running buffer in all experimental conditions. Recombinant purified FcRH5 Ig-8 domain at known concentrations (500 nM with 2-fold serial dilutions) is used as the ‘analyte’ and injected over the respective flow cells with 150s contact time and 500s dissociation at 30 μl/minute of flow rate with a constant temperature of 25° C. In each experiment, flow cell 1 is unmodified and used for reference subtraction. A ‘0 concentration’ sensogram of buffer alone is used as a double reference subtraction to factor for drift. Data are fit to a 1:1 Langmuir binding model. Since a capture system is used, a local maximal analytical response (Rmax) parameter is used for the data fitting in each case.

### Preparation of Soluble Anti-FcRH5 VHH-his Binding Domains

Antibodies are expressed by transient transfection in expi-CHO cells. Anti-FcRH5 VHH-His carrying a dual 6×His tag, are expressed by transient transfection in expi-CHO, and are purified from the supernatants using a Ni-beads suspension.

### ELISA

Recombinant purified anti-FcRH5 VHH-His antibodies are coated at 1 μg/ml in 50 μl/well with 3-fold serial dilutions on 96-well ELISA plates (Nunc Maxisorp) overnight at 4° C. Plates are blocked with 200 μl/well of PBS 2% BSA for 1 h at RT on a plate shaker. Plates are washed 3 times with 250 μl/well of PBS 0.05% Tween20 and incubated with 50 μl/well of recombinant FcRH5 Ig8 domain fused to murine IgG2a Fc in PBS 0.5% BSA for 1 h at RT on a plate shaker. Plates are washed 3 times with 250 μl/well of PBS 0.05% Tween20 and incubated with 50 μl/well of secondary anti-mouse antibody HRP conjugated (Jackson ImmunoResearch-115-035-166) at 1:5000 dilution in PBS 0.5% BSA for 1 h at RT on a plate shaker. Plates are washed four times in PBS 0.05% Tween20 and binding revealed with 45 μl/well of 1-Step Ultra TMB reagent (Thermo scientific) for 3 minutes at RT. Reaction is blocked with 45 μl/well of 1M H₂SO₄ and absorbance read at OD 450 nm on a Varioskan Lux plate reader (Thermo scientific).

### Flow Cytometry

Binding for FcRH5 is determined via flow cytometry on HEK293T cells and HEK293T cells transiently transfected to express the extracellular Ig8 domain of FcRH5 fused to human CD19 transmembrane domain and cytoplasmic tail (AU45283). Construct contains the eGFP protein as transfection marker. 5×10⁴ cells/well are washed twice in FACS buffer (PBS 2% FCS) and stained with 50 μl of primary antibody (Table 8) at 2 μg/ml for 30 on ice. Cells are washed twice in FACS buffer to remove unbound antibodies and stained with 50 μl/well of anti-His secondary antibody conjugated to Alexa fluor 647 (Santa Cruz Biotechnology-sc-53073AF647) diluted 1:100, for 30 minutes in ice. Cells are then washed in FACS buffer and stained with 100 μl/well of Sytox blue for viability determination (Thermo Fisher Scientific—S34857), according to manufacturer's recommendations. Cells are acquired on a MACSQuant 10 instrument. Cells are first gated on singlets, then population is defined based of SSC/FSC appearance and live cells identified based on Sytox blue staining.

### Example 5—Anti-FcRH5 VHH-CAR Activation Assay Via NUR77 Upregulation in Jurkat T Cell Line

VHH's shown in Table 11 are cloned into a gamma retroviral vector encompassing a CD8 spacer and transmembrane region with a 41BB and CD3z chain intracellular signalling domain along with an RQR8 transduction marker separated by a 2A cleavage peptide (FIG. 1).

Plate bound antigen CAR stimulation assay and flow cytometry are carried out following the methods described in Example 2.

This application claims the benefit of United Kingdom application No. 2108773.9 filed on 18th June 2021, and United Kingdom application No. 2116502.2 filed on 16th November 2021. These applications are incorporated herein by reference in their entirety.

All publications mentioned in the above specification are herein incorporated by reference. Various modifications and variations of the described methods and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention which are obvious to those skilled in molecular biology or related fields are intended to be within the scope of the following claims.

1. A single domain antibody (sdAb) comprising a FcRH5 binding domain.

2. The sdAb according to claim 1 wherein the FcRH5 binding domain comprises:
complementarity determining regions (CDRs)
with the following sequences:
CDR1-
(SEQ ID NO: 1)
RSSFSNNA
CDR2-
(SEQ ID NO: 2)
ITKGGVT
CDR3-
(SEQ ID NO: 3)
NTIPFRSA;
or
(ii)
CDRs with the following sequences:
CDR1-
(SEQ ID NO: 4)
GFTFSNYA
CDR2-
(SEQ ID NO: 5)
INSDGGTA
CDR3-
(SEQ ID NO: 6)
AANRGFCAGVRCLEYQY;
or
(iii)
CDRs with the following sequences:
CDR1-
(SEQ ID NO: 7)
GRTFSINA
CDR2-
(SEQ ID NO: 8)
IGGSGRVSST
CDR3-
(SEQ ID NO: 9)
AARRDYLPFPPESYDY;
or
(iv)
CDRs with the following sequences:
CDR1-
(SEQ ID NO: 7)
GRTFSINA
CDR2-
(SEQ ID NO: 8)
IGGSGRVSST
CDR3-
(SEQ ID NO: 10)
AAGRRTSTNGGDYDY;
or
(v)
CDRs with the following sequences:
CDR1-
(SEQ ID NO: 7)
GRTFSINA
CDR2-
(SEQ ID NO: 11)
ISRSGGAT
CDR3-
(SEQ ID NO: 12)
AGTRRAFSTGLRDYDY;
or
(vi)
CDRs with the following sequences:
CDR1-
(SEQ ID NO: 13)
GRTFSNST
CDR2-
(SEQ ID NO: 14)
ISWSGGTY
CDR3-
(SEQ ID NO: 15)
AAARKGWSTRGDDYDY;
or
(vii)
CDRs with the following sequences:
CDR1-
(SEQ ID NO: 16)
GRTYNNYA
CDR2-
(SEQ ID NO: 17)
ISRSGGMT
(viii)
CDRs with the following sequences:
CDR1-
(SEQ ID NO: 19)
GRTFSSYA
CDR2-
(SEQ ID NO: 20)
ISRIGGVT
CDR3-
(SEQ ID NO: 21)
AAAGLVSISTTPNDYDY;
or
(ix)
CDRs with the following sequences:
CDR1-
(SEQ ID NO: 22)
GNIFRLNG
CDR2-
(SEQ ID NO: 23)
ITSGGNT
CDR3-
(SEQ ID NO: 24)
NAIPFRL;
or
(x)
CDRs with the following sequences:
CDR1-
(SEQ ID NO: 25)
RNIFSLNP
CDR2-
(SEQ ID NO: 26)
ITDGGST
CDR3-
(SEQ ID NO: 27)
NRVGGLQTWA;
or
(xi)
CDRs with the following sequences:
CDR1-
(SEQ ID NO: 28)
GRSFSNYG
CDR2-
(SEQ ID NO: 29)
IGMVGGLT
CDR3-
(SEQ ID NO: 30)
AAGRRFSTSSRDYDI;
or
(xii)
CDRs with the following sequences:
CDR1-
(SEQ ID NO: 31)
GRTFDSRP
CDR2-
(SEQ ID NO: 32)
VSWRGEST
CDR3-
(SEQ ID NO: 33)
AAGEPYSGTYYYRGRDYDY;
(xiii)
CDRs with the following sequences:
CDR1-
(SEQ ID NO: 34)
GRTFSMYA
CDR2-
(SEQ ID NO: 35)
ISGSARIT
CDR3-
(SEQ ID NO: 36)
AASSTYTSTSGSSYNY;
or
(xiv)
CDRs with the following sequences:
CDR1-
(SEQ ID NO: 37)
GSIFSINA
CDR2-
(SEQ ID NO: 38)
ITSGGST
CDR3-
(SEQ ID NO: 39)
NALGGFVPSYG;
or
(xv)
CDRs with the following sequences:
CDR1-
(SEQ ID NO: 155)
GRTSSRAA
CDR2-
(SEQ ID NO: 156)
ISWSGGTT
CDR3-
(SEQ ID NO: 157)
AAARIFTTARNDYDH;
or
(xvi)
CDRs with the following sequences:
CDR1-
(SEQ ID NO: 22)
GNIFRLNG
CDR2-
(SEQ ID NO: 23)
ITSGGNT
CDR3-
(SEQ ID NO: 186)
NTIPFRLS;
or
(xvii)
CDRs with the following sequences:
CDR1-
(SEQ ID NO: 160)
GNIFRLNA
CDR2-
(SEQ ID NO: 23)
ITSGGNT
CDR3-
(SEQ ID NO: 186)
NTIPFRLS;
or
(xviii)
CDRs with the following sequences:
CDR1-
(SEQ ID NO: 1)
RSSFSNNA
CDR2-
(SEQ ID NO: 2)
ITKGGVT
CDR3-
(SEQ ID NO: 187)
NTIPVRSA;
or
(xix)
CDRs with the following sequences:
CDR1-
(SEQ ID NO: 37)
GSIFSINA
CDR2-
(SEQ ID NO: 2)
ITKGGVT
CDR3-
(SEQ ID NO: 188)
NTIPCRSA;
or
(xx)
CDRs with the following sequences:
CDR1-
(SEQ ID NO: 161)
GSSFRLNG
CDR2-
(SEQ ID NO: 38)
ITSGGST
CDR3-
(SEQ ID NO: 189)
NTIPFSRA;
or
(xxi)
CDRs with the following sequences:
CDR1-
(SEQ ID NO: 162)
GRSVSINA
CDR2-
(SEQ ID NO: 176)
IDRSGNT
CDR3-
(SEQ ID NO: 190)
NTIPYSDS;
or
(xxii)
CDRs with the following sequences:
CDR1-
(SEQ ID NO: 19)
GRTFSSYA
CDR2-
(SEQ ID NO: 177)
IDGIGGIT
CDR3-
(SEQ ID NO: 3)
NTIPFRSA;
or
(xxiii)
CDRs with the following sequences:
CDR1-
(SEQ ID NO: 1)
RSSFSNNA
CDR2-
(SEQ ID NO: 2)
ITKGGVT
CDR3-
(SEQ ID NO: 191)
NAIPFRSA;
or
(xxiv)
CDRs with the following sequences:
CDR1-
(SEQ ID NO: 163)
GNNFRLNA
CDR2-
(SEQ ID NO: 23)
ITSGGNT
CDR3-
(SEQ ID NO: 192)
NAIPFRPS;
or
(xxv)
CDRs with the following sequences:
CDR1-
(SEQ ID NO: 22)
GNIFRLNG
CDR2-
(SEQ ID NO: 23)
ITSGGNT
CDR3-
(SEQ ID NO: 193)
NAIPFRRS;
or
(xxvi)
CDRs with the following sequences:
CDR1-
(SEQ ID NO: 164)
ERIFRINA
CDR2-
(SEQ ID NO: 23)
ITSGGNT
CDR3-
(SEQ ID NO: 193)
NAIPFRRS;
or
(xxvii)
CDRs with the following sequences:
CDR1-
(SEQ ID NO: 165)
GFTFSTYW
CDR2-
(SEQ ID NO: 178)
IDNGGGTT
CDR3-
(SEQ ID NO: 194)
NALPFRLS;
or
(xxviii)
CDRs with the following sequences:
CDR1-
(SEQ ID NO: 22)
GNIFRLNG
CDR2-
(SEQ ID NO: 23)
ITSGGNT
CDR3-
(SEQ ID NO: 195)
NATPFRLS;
or
(xxix)
CDRs with the following sequences:
CDR1-
(SEQ ID NO: 22)
GNIFRLNG
CDR2-
(SEQ ID NO: 179)
ITRGGNT
CDR3-
(SEQ ID NO: 196)
NSIPFRLS;
or
(xxx)
CDRs with the following sequences:
CDR1-
(SEQ ID NO: 166)
GNIFRING
CDR2-
(SEQ ID NO: 23)
ITSGGNT
CDR3-
(SEQ ID NO: 197)
NAIPFRIS;
or
(xxxi)
CDRs with the following sequences:
CDR1-
(SEQ ID NO: 163)
GNNFRLNA
CDR2-
(SEQ ID NO: 23)
ITSGGNT
CDR3-
(SEQ ID NO: 198)
NAIPFRLY;
or
(xxxii)
CDRs with the following sequences:
CDR1-
(SEQ ID NO: 165)
GFTFSTYW
CDR2-
(SEQ ID NO: 178)
IDNGGGTT
CDR3-
(SEQ ID NO: 199)
KAIPFRLS;
or
(xxiii)
CDRs with the following sequences:
CDR1-
(SEQ ID NO: 167)
GSSFSNNA
CDR2-
(SEQ ID NO: 2)
ITKGGVT
CDR3-
(SEQ ID NO: 3)
NTIPFRSA;
or
(xxxiv)
CDRs with the following sequences:
CDR1-
(SEQ ID NO: 165)
GFTFSTYW
CDR2-
(SEQ ID NO: 178)
IDNGGGTT
CDR3-
(SEQ ID NO: 3)
NTIPFRSA;
or
(xxxv)
CDRs with the following sequences:
CDR1-
(SEQ ID NO: 160)
GNIFRLNA
CDR2-
(SEQ ID NO: 2)
ITKGGVT
CDR3-
(SEQ ID NO: 3)
NTIPFRSA;
or
(xxxvi)
CDRs with the following sequences:
CDR1-
(SEQ ID NO: 168)
RSSFGNNA
CDR2-
(SEQ ID NO: 2)
ITKGGVT
CDR3-
(SEQ ID NO: 3)
NTIPFRSA;
or
(xxxvii)
CDRs with the following sequences:
CDR1-
(SEQ ID NO: 169)
GRTFSTYG
CDR2-
(SEQ ID NO: 11)
ISRSGGAT
CDR3-
(SEQ ID NO: 12)
AGTRRAFSTGLRDYDY;
or
(xxxviii)
CDRs with the following sequences:
CDR1-
(SEQ ID NO: 170)
GTIERNNA
CDR2-
(SEQ ID NO: 38)
ITSGGST
CDR3-
(SEQ ID NO: 200)
AAGRRFSTRSRDYDY;
or
(xxxix)
CDRs with the following sequences:
CDR1-
(SEQ ID NO: 28)
GRSFSNYG
CDR2-
(SEQ ID NO: 29)
IGMVGGLT
CDR3-
(SEQ ID NO: 201)
AAGRRFSTSSRDYDY;
or
(xl)
CDRs with the following sequences:
CDR1-
(SEQ ID NO: 28)
GRSFSNYG
CDR2-
(SEQ ID NO: 29)
IGMVGGLT
CDR3-
(SEQ ID NO: 202)
AAGRRFSTGSRDYDI;
or
(xli)
CDRs with the following sequences:
CDR1-
(SEQ ID NO: 19)
GRTFSSYA
CDR2-
(SEQ ID NO: 179)
ISQFGGVTT
CDR3-
(SEQ ID NO: 202)
AAGRRFSTGSRDYDI;
or
(xlii)
CDRs with the following sequences:
CDR1-
(SEQ ID NO: 28)
GRSFSNYG
CDR2-
(SEQ ID NO: 29)
IGMVGGLT
CDR3-
(SEQ ID NO: 203)
AGGRRFSTSSRDYDI;
or
(xliii)
CDRs with the following sequences:
CDR1-
(SEQ ID NO: 28)
GRSFSNYG
CDR2-
(SEQ ID NO: 29)
IGMVGGLT
CDR3-
(SEQ ID NO: 204)
AAGRRFSTSSREYDI;
or
(xliv)
CDRs with the following sequences:
CDR1-
(SEQ ID NO: 28)
GRSFSNYG
CDR2-
(SEQ ID NO: 180)
IGMVGGLP
CDR3-
(SEQ ID NO: 205)
AAGRRLSTSSRDYDI;
or
(xlv)
CDRs with the following sequences:
CDR1-
(SEQ ID NO: 28)
GRSFSNYG
CDR2-
(SEQ ID NO: 181)
ISRGGGVS
CDR3-
(SEQ ID NO: 206)
AAGLRFSTGSRDYDI;
or
(xlvi)
CDRs with the following sequences:
CDR1-
(SEQ ID NO: 171)
GRTFRRYA
CDR2-
(SEQ ID NO: 17)
ISRSGGMT
CDR3-
(SEQ ID NO: 207)
AAYVGGFSTTRRDYAY;
or
(xlvii)
CDRs with the following sequences:
CDR1-
(SEQ ID NO: 13)
GRTFSNST
CDR2-
(SEQ ID NO: 156)
ISWSGGTT
CDR3-
(SEQ ID NO: 15)
AAARKGWSTRGDDYDY;
or
(xlviii)
CDRs with the following sequences:
CDR1-
(SEQ ID NO: 172)
GRTVI
CDR2-
(SEQ ID NO: 182)
SSGSGGVT
CDR3-
(SEQ ID NO: 208)
AAALTWSTRPSDFTS;
or
(xlix)
CDRs with the following sequences:
CDR1-
(SEQ ID NO: 172)
GRTVI
CDR2-
(SEQ ID NO: 183)
SNWSGGVT
CDR3-
(SEQ ID NO: 18)
AAYVGGFSTARRDYSY;
or
(l)
CDRs with the following sequences:
CDR1-
(SEQ ID NO: 7)
GRTFSINA
CDR2-
(SEQ ID NO: 17)
ISRSGGMT
CDR3-
(SEQ ID NO: 18)
AAYVGGFSTARRDYSY;
or
(li)
CDRs with the following sequences:
CDR1-
(SEQ ID NO: 173)
GRTFSRYA
CDR2-
(SEQ ID NO: 184)
INGSGGT
CDR3-
(SEQ ID NO: 209)
AAARIFTTTRNEYDH;
or
(lii)
CDRs with the following sequences:
CDR1-
(SEQ ID NO: 173)
GRTFSRYA
CDR2-
(SEQ ID NO: 184)
INGSGGT
CDR3-
(SEQ ID NO: 209)
AAARIFTTTRNEYDH;
or
(liii)
CDRs with the following sequences:
CDR1-
(SEQ ID NO: 165)
GFTFSTYW
CDR2-
(SEQ ID NO: 178)
IDNGGGTT
CDR3-
(SEQ ID NO: 210)
AAARIFSTARNDYDH;
or
(liv)
CDRs with the following sequences:
CDR1-
(SEQ ID NO: 28)
GRSFSNYG
CDR2-
(SEQ ID NO: 29)
IGMVGGLT
CDR3-
(SEQ ID NO: 211)
AGGRIFRTSSRDYDI;
or
(lv)
CDRs with the following sequences:
CDR1-
(SEQ ID NO: 22)
GNIFRLNG
CDR2-
(SEQ ID NO: 23)
ITSGGNT
CDR3-
(SEQ ID NO: 212)
AAARFFTTARNDYDH;
or
(lvi)
CDRs with the following sequences:
CDR1-
(SEQ ID NO: 37)
GSIFSINA
CDR2-
(SEQ ID NO: 38)
ITSGGST
CDR3-
(SEQ ID NO: 213)
NALGGFVPNYG;
or
(lvii)
CDRs with the following sequences:
CDR1-
(SEQ ID NO: 22)
GNIFRLNG
CDR2-
(SEQ ID NO: 23)
ITSGGNT
CDR3-
(SEQ ID NO: 213)
NALGGFVPNYG;
or
(lviii)
CDRs with the following sequences:
CDR1-
(SEQ ID NO: 37)
GSIFSINA
CDR2-
(SEQ ID NO: 38)
ITSGGST
CDR3-
(SEQ ID NO: 214)
NALGGFVPNDG;
or
(lix)
CDRs with the following sequences:
CDR1-
(SEQ ID NO: 163)
GNNFRLNA
CDR2-
(SEQ ID NO: 23)
ITSGGNT
CDR3-
(SEQ ID NO: 215)
NALGGFAPNYG;
or
(lx)
CDRs with the following sequences:
CDR1-
(SEQ ID NO: 28)
GRSFSNYG
CDR2-
(SEQ ID NO: 29)
IGMVGGLT
CDR3-
(SEQ ID NO: 216)
NALGGFVPNYV;
or
(lxi)
CDRs with the following sequences:
CDR1-
(SEQ ID NO: 174)
GRSFSINA
CDR2-
(SEQ ID NO: 38)
ITSGGST
CDR3-
(SEQ ID NO: 217)
NALGGFVLNYG;
or
(lxii)
CDRs with the following sequences:
CDR1-
(SEQ ID NO: 37)
GSIFSINA
CDR2-
(SEQ ID NO: 38)
ITSGGST
CDR3-
(SEQ ID NO: 218)
NALGGLVPNYG;
or
(lxiii)
CDRs with the following sequences:
CDR1-
(SEQ ID NO: 37)
GSIFSINA
CDR2-
(SEQ ID NO: 38)
ITSGGST
CDR3-
(SEQ ID NO: 219)
NALGGFLPNYG;
or
(lxiv)
CDRs with the following sequences:
CDR1-
(SEQ ID NO: 37)
GSIFSINA
CDR2-
(SEQ ID NO: 38)
ITSGGST
CDR3-
(SEQ ID NO: 220)
NALGGFVTNYG;
or
(lxv)
CDRs with the following sequences:
CDR1-
(SEQ ID NO: 37)
GSIFSINA
CDR2-
(SEQ ID NO: 38)
ITSGGST
CDR3-
(SEQ ID NO: 220)
NALGGFVTNYG;
or
(lxvi)
CDRs with the following sequences:
CDR1-
(SEQ ID NO: 222)
GRTV
CDR2-
(SEQ ID NO: 185)
ITRGGST
CDR3-
(SEQ ID NO: 213)
NALGGFVPNYG;
or
(lxvii)
CDRs with the following sequences:
CDR1-
(SEQ ID NO: 22)
GNIFRLNG
CDR2-
(SEQ ID NO: 23)
ITSGGNT
CDR3-
(SEQ ID NO: 39)
NALGGFVPSYG;
or
(lxviii)
CDRs with the following sequences:
CDR1-
(SEQ ID NO: 163)
GNNFRLNA
CDR2-
(SEQ ID NO: 23)
ITSGGNT
CDR3-
(SEQ ID NO: 39)
NALGGFVPSYG;
or
(lxix)
CDR1-
(SEQ ID NO: 22)
GNIFRLNG
CDR2-
ITSGGST
(SEQ ID NO: 38)
CDR3-
(SEQ ID NO: 39)
NALGGFVPSYG;
or
(lxx)
CDRs with the following sequences:
CDR1-
(SEQ ID NO: 28)
GRSFSNYG
CDR2-
(SEQ ID NO: 29)
IGMVGGLT
CDR3-
(SEQ ID NO: 39)
NALGGFVPSYG;
or
(lxxi)
CDRs with the following sequences:
CDR1-
(SEQ ID NO: 175)
GNIFSINA
CDR2-
(SEQ ID NO: 38)
ITSGGST
CDR3-
(SEQ ID NO: 39)
NALGGFVPSYG;
or
(lxxii)
CDRs with the following sequences:
CDR1-
(SEQ ID NO: 165)
GFTFSTYW
CDR2-
(SEQ ID NO: 178)
IDNGGGTT
CDR3-
(SEQ ID NO: 221)
ARNPTRGWYSTDY.
optionally wherein one or more of the CDRs comprises one, two or three amino acid mutations.

3. The sdAb according to claim 2, wherein the FcRH5 binding domain comprises:
(i) a variable heavy chain domain antibody (VHH) having the sequence shown as SEQ ID NO: 40 or a variant having at least 80% sequence identity thereto; or
(ii) a VHH having the sequence shown as SEQ ID NO: 41 or a variant having at least 80% sequence identity thereto; or
(iii) a VHH having the sequence shown as SEQ ID NO: 42 or a variant having at least 80% sequence identity thereto; or
(iv) a VHH having the sequence shown as SEQ ID NO: 43 or a variant having at least 80% sequence identity thereto; or
(v) a VHH having the sequence shown as SEQ ID NO: 44 or a variant having at least 80% sequence identity thereto; or
(vi) a VHH having the sequence shown as SEQ ID NO: 45 or a variant having at least 80% sequence identity thereto; or
(vii) a VHH having the sequence shown as SEQ ID NO: 46 or a variant having at least
(viii) a VHH having the sequence shown as SEQ ID NO: 47 or a variant having at least 80% sequence identity thereto; or
(ix) a VHH having the sequence shown as SEQ ID NO: 48 or a variant having at least 80% sequence identity thereto; or
(x) a VHH having the sequence shown as SEQ ID NO: 49 or a variant having at least 80% sequence identity thereto; or
(xi) a VHH having the sequence shown as SEQ ID NO: 50 or a variant having at least 80% sequence identity thereto; or
(xii) a VHH having the sequence shown as SEQ ID NO: 51 or a variant having at least 80% sequence identity thereto; or
(xiii) a VHH having the sequence shown as SEQ ID NO: 52 or a variant having at least 80% sequence identity thereto; or
(xiv) a VHH having the sequence shown as SEQ ID NO: 53 or a variant having at least 80% sequence identity thereto;
(xv) a VHH having the sequence shown as SEQ ID NO: 158 or a variant having at least 80% sequence identity thereto; or
(xvi) a VHH having the sequence shown as SEQ ID NO: 223 or a variant having at least 80% sequence identity thereto; or
(xvii) a VHH having the sequence shown as SEQ ID NO: 224 or a variant having at least 80% sequence identity thereto; or
(xviii) a VHH having the sequence shown as SEQ ID NO: 225 or a variant having at least 80% sequence identity thereto; or
(xvix) a VHH having the sequence shown as SEQ ID NO: 226 or a variant having at least 80% sequence identity thereto; or
(xx) a VHH having the sequence shown as SEQ ID NO: 227 or a variant having at least 80% sequence identity thereto; or
(xxi) a VHH having the sequence shown as SEQ ID NO: 228 or a variant having at least 80% sequence identity thereto; or
(xxii) a VHH having the sequence shown as SEQ ID NO: 229 or a variant having at least 80% sequence identity thereto; or
(xxiii) a VHH having the sequence shown as SEQ ID NO: 230 or a variant having at least 80% sequence identity thereto; or
(xxiv) a VHH having the sequence shown as SEQ ID NO: 231 or a variant having at least (xxv) a VHH having the sequence shown as SEQ ID NO: 232 or a variant having at least 80% sequence identity thereto; or
(xxvi) a VHH having the sequence shown as SEQ ID NO: 233 or a variant having at least 80% sequence identity thereto; or
(xxvii) a VHH having the sequence shown as SEQ ID NO: 234 or a variant having at least 80% sequence identity thereto; or
(xxviii) a VHH having the sequence shown as SEQ ID NO: 235 or a variant having at least 80% sequence identity thereto; or
(xxix) a VHH having the sequence shown as SEQ ID NO: 236 or a variant having at least 80% sequence identity thereto; or
(xxx) a VHH having the sequence shown as SEQ ID NO: 237 or a variant having at least 80% sequence identity thereto; or
(xxxi) a VHH having the sequence shown as SEQ ID NO: 238 or a variant having at least 80% sequence identity thereto; or
(xxxii) a VHH having the sequence shown as SEQ ID NO: 239 or a variant having at least 80% sequence identity thereto; or
(xxxiii) a VHH having the sequence shown as SEQ ID NO: 240 or a variant having at least 80% sequence identity thereto; or
(xxxiv) a VHH having the sequence shown as SEQ ID NO: 241 or a variant having at least 80% sequence identity thereto; or
(xxxv) a VHH having the sequence shown as SEQ ID NO: 242 or a variant having at least 80% sequence identity thereto; or
(xxxvi) a VHH having the sequence shown as SEQ ID NO: 243 or a variant having at least 80% sequence identity thereto; or
(xxxvii) a VHH having the sequence shown as SEQ ID NO: 244 or a variant having at least 80% sequence identity thereto; or
(xxxviii) a VHH having the sequence shown as SEQ ID NO: 245 or a variant having at least 80% sequence identity thereto; or
(xxxix) a VHH having the sequence shown as SEQ ID NO: 246 or a variant having at least 80% sequence identity thereto; or
(xl) a VHH having the sequence shown as SEQ ID NO: 247 or a variant having at least 80% sequence identity thereto; or
(xli) a VHH having the sequence shown as SEQ ID NO: 248 or a variant having at least (xlii) a VHH having the sequence shown as SEQ ID NO: 249 or a variant having at least 80% sequence identity thereto; or
(xliii) a VHH having the sequence shown as SEQ ID NO: 250 or a variant having at least 80% sequence identity thereto; or
(xliv) a VHH having the sequence shown as SEQ ID NO: 251 or a variant having at least 80% sequence identity thereto; or
(xlv) a VHH having the sequence shown as SEQ ID NO: 252 or a variant having at least 80% sequence identity thereto; or
(xlvi) a VHH having the sequence shown as SEQ ID NO: 253 or a variant having at least 80% sequence identity thereto; or
(xlvii) a VHH having the sequence shown as SEQ ID NO: 254 or a variant having at least 80% sequence identity thereto; or
(xlviii) a VHH having the sequence shown as SEQ ID NO: 255 or a variant having at least 80% sequence identity thereto; or
(xlix) a VHH having the sequence shown as SEQ ID NO: 256 or a variant having at least 80% sequence identity thereto; or
(1) a VHH having the sequence shown as SEQ ID NO: 257 or a variant having at least 80% sequence identity thereto; or
(li) a VHH having the sequence shown as SEQ ID NO: 258 or a variant having at least 80% sequence identity thereto; or
(lii) a VHH having the sequence shown as SEQ ID NO: 259 or a variant having at least 80% sequence identity thereto; or
(liii) a VHH having the sequence shown as SEQ ID NO: 260 or a variant having at least 80% sequence identity thereto; or
(liv) a VHH having the sequence shown as SEQ ID NO: 261 or a variant having at least 80% sequence identity thereto; or
(lv) a VHH having the sequence shown as SEQ ID NO: 262 or a variant having at least 80% sequence identity thereto; or
(lvi) a VHH having the sequence shown as SEQ ID NO: 263 or a variant having at least 80% sequence identity thereto; or
(lvii) a VHH having the sequence shown as SEQ ID NO: 264 or a variant having at least 80% sequence identity thereto; or
(lviii) a VHH having the sequence shown as SEQ ID NO: 265 or a variant having at least
(lix) a VHH having the sequence shown as SEQ ID NO: 266 or a variant having at least 80% sequence identity thereto; or
(lx) a VHH having the sequence shown as SEQ ID NO: 267 or a variant having at least 80% sequence identity thereto; or
(lxi) a VHH having the sequence shown as SEQ ID NO: 268 or a variant having at least 80% sequence identity thereto; or
(lxii) a VHH having the sequence shown as SEQ ID NO: 269 or a variant having at least 80% sequence identity thereto; or
(lxiii) a VHH having the sequence shown as SEQ ID NO: 270 or a variant having at least 80% sequence identity thereto; or
(lxiv) a VHH having the sequence shown as SEQ ID NO: 271 or a variant having at least 80% sequence identity thereto; or
(lxv) a VHH having the sequence shown as SEQ ID NO: 272 or a variant having at least 80% sequence identity thereto; or
(lxvi) a VHH having the sequence shown as SEQ ID NO: 273 or a variant having at least 80% sequence identity thereto; or
(lxvii) a VHH having the sequence shown as SEQ ID NO: 274 or a variant having at least 80% sequence identity thereto; or
(lxviii) a VHH having the sequence shown as SEQ ID NO: 275 or a variant having at least 80% sequence identity thereto; or
(lxix) a VHH having the sequence shown as SEQ ID NO: 276 or a variant having at least 80% sequence identity thereto; or
(lxx) a VHH having the sequence shown as SEQ ID NO: 277 or a variant having at least 80% sequence identity thereto; or
(lxxi) a VHH having the sequence shown as SEQ ID NO: 278 or a variant having at least 80% sequence identity thereto; or
(lxxii) a VHH having the sequence shown as SEQ ID NO: 279 or a variant having at least 80% sequence identity thereto; or
(lxxiii) a VHH having the sequence shown as SEQ ID NO: 280 or a variant having at least 80% sequence identity thereto; or
(lxxiv) a VHH having the sequence shown as SEQ ID NO: 281 or a variant having at least 80% sequence identity thereto; or
(lxxv) a VHH having the sequence shown as SEQ ID NO: 282 or a variant having at least (lxxvi) a VHH having the sequence shown as SEQ ID NO: 283 or a variant having at least 80% sequence identity thereto; or
(lxxvii) a VHH having the sequence shown as SEQ ID NO: 284 or a variant having at least 80% sequence identity thereto; or
(lxxviii) a VHH having the sequence shown as SEQ ID NO: 285 or a variant having at least 80% sequence identity thereto; or
(lxxix) a VHH having the sequence shown as SEQ ID NO: 286 or a variant having at least 80% sequence identity thereto; or
(lxxx) a VHH having the sequence shown as SEQ ID NO: 287 or a variant having at least 80% sequence identity thereto; or
(lxxxi) a VHH having the sequence shown as SEQ ID NO: 288 or a variant having at least 80% sequence identity thereto.

4. The sdAb according to claim 2, wherein the sdAb is a humanised sdAb.

5. A FcRH5 binding molecule comprising the sdAb according to claim 2, wherein the FcRH5 binding molecule is selected from an antibody conjugate, a chimeric antigen receptor (CAR), or an immune cell engager.

6. A CAR comprising the sdAb according to claim 2.

7. The CAR according claim 6, which comprises the sequence selected from the group comprising SEQ ID NO: 76-SEQ ID NO: 89, SEQ ID NO: 159 and SEQ ID NO: 289-SEQ ID NO: 354, or a variant thereof which has at least 80% sequence identity thereto but retains the capacity to i) bind FcRH5 and ii) induce T cell signalling.

8. An immune cell engager molecule which comprises:
(i) a first domain which comprises a sdAb according to claim 2; and
(ii) a second domain capable of activating a T cell.

9. A polynucleotide comprising a nucleic acid sequence encoding an sdAb according to claim 2, or encoding a CAR comprising the sdAb, or encoding an immune cell engagement molecule comprising the sdAb.

10. A vector which comprises a polynucleotide according to claim 9.

11. A cell which comprises a CAR according to claim 6, or comprises a polynucleotide comprising a nucleotide sequence encoding the CAR, or comprises a vector that comprises the polynucleotide.

12. A pharmaceutical composition which comprises a sdAb according to claim 2, or an FcRH5 binding molecule that comprises the sdAb, or an immune cell engager molecule that comprises the sdAb, or, a polynucleotide that comprises a nucleic acid sequence encoding the sdAb, or a vector that comprises the polynucleotide, or a cell that comprises the polynucleotide or the vector, together with a pharmaceutically acceptable carrier, diluent or excipient.

13. (canceled)

14. A method for treating a disease which comprises the step of administering to a subject the sdAb according to claim 2, an FcRH5 binding molecule that comprises the sdAb, an immune cell engager molecule that comprises the sdAb, a polynucleotide that comprises a nucleic acid sequence encoding the sdAb, a vector that comprises the polynucleotide, a cell that comprises the polynucleotide or the vector.

15. (canceled)

16. The method according to claim 14, wherein the disease is a B cell malignancy or a plasma cell disorder.

17. The method according to claim 16, wherein the a B cell malignancy or plasma cell disorder is selected from the list comprising: leukaemia, hairy cell leukaemia, chronic lymphocytic leukaemia, non-Hodgkins lymphoma, mantle cell lymphoma, EBV-associated lymphoma (Burkitt), lymphoplasmacytic lymphoma, plasmacytoma, solitary plasmocytoma, extramedullary plasmocytoma, plasma cell leukemia, multiple myeloma, macroglobulinemia, amyloidosis, Waldenstrom's macroglobulinemia, solitary bone plasmacytoma, extramedullary plasmacytoma, osteosclerotic myeloma, heavy chain diseases, monoclonal gammopathy of undetermined significance (MGUS), non-IgM MGUS, IgM MGUS, light chain MGUS and smoldering multiple myeloma.

18. The method according to claim 16, wherein the B cell malignancy or plasma cell disorder is multiple myeloma.

19. The FcRH5 binding molecule that comprises a T cell engager molecule, a gamma delta T cell engager molecule, a natural killer (NK) T cell engager molecule, or a NK cell engager molecule.

20. The immune cell engager molecule according to claim 8 which further comprises:
(iii) a third domain capable of co-activating a T cell.
